(19) United States
(12) Patent Application Publication
(10) Pub. No.: US 2024/0190941 A1
(43) Pub. Date: Jun. 13, 2024

(54) PEPTIDE-MHC-IMMUNOGLOBULIN MULTIMERS AND METHODS OF USE THEREOF

(71) Applicant: REPERTOIRE IMMUNE MEDICINES, INC., Cambridge, MA (US)

(72) Inventors: Thomas J. MALIA, Cambridge, MA (US); Joanna F. SWAIN, Cambridge, MA (US); Andrew G. HAMEL, Cambridge, MA (US); Ellen M. WHITE, Cambridge, MA (US)

(21) Appl. No.: 18/554,625

(22) PCT Filed: Apr. 7, 2022
(86) PCT No.: PCT/US2022/023898

§ 371 Date: Oct. 9, 2023

Related U.S. Application Data
(60) Provisional application No. 63/171,799, filed on Apr. 7, 2021.

(51) Int. Cl. C07K 14/74; A61P 37/06; C07K 14/52; C07K 14/705; C07K 16/28; C12N 15/63; G01N 33/53

(52) U.S. Cl. CPC C07K 14/70539; A61P 37/06; C07K 14/52; C07K 14/70596; C07K 16/2803; C12N 15/63; G01N 33/53; C07K 2319/30

(57) ABSTRACT

Multimers are provided in which a Major Histocompatibility Complex (MHC) class I or class II molecule, optionally complexed with an MHC-binding peptide, is attached to a multimerization moiety from immunoglobulin M or A (IgM or IgA) to thereby create a multivalent peptide-MHC-Ig multimer composition. The multimers can be used, for example, to determine the binding specificity of T cell receptors, such as for particular MHC-peptide complexes. The multimers can also be used, for example, to modulate an immune response in a subject by administering the multimer to the subject. Methods of making the peptide-MHC-Ig multimers are also provided.

### CROSS REFERENCE TO RELATED APPLICATIONS

This application claims the benefit of and U.S. priority to Provisional Patent Application No. 63/171,799, filed on Apr. 7, 2021, the disclosure of which is incorporated herein by reference in its entirety for all purposes.

### BACKGROUND

The identification of peptides recognized by individual T cells is important for the understanding and treatment of immune-related diseases, as well as vaccine development and development of other prophylactic agents for the prevention of diseases. Techniques for the detection of antigen-responsive T cells exploit the interaction between a given TCR and its cognate peptide-MHC (pMHC) recognition motif. T cell detection using multimerized pMHC molecules has become the preferred method for detecting antigen-specific T cells in a wide variety of research and clinical situations.

Studies have shown that systemic delivery of MHC class I or class II ligands can expand disease-suppressing antigen-specific regulatory T cells by using a pMHC-containing complex to selectively target and dampen a certain immune response (e.g., an autoimmune response) without also suppressing overall “normal” immune system function (e.g., Singha et al. (2017) NAT. NANOTECHNOL., 12(7):701-710). Despite the ability to trigger differentiation and expansion of specific populations of T-cells using pMHC-based agents, no clinically-translatable agents and associated treatments have emerged from these studies.

Accordingly, despite the efforts that have been made to date, there remains a need for additional agents and methods for determining peptide-MHC-TCR interactions, as well as compositions for use in treating subjects in need of specific and selective modulation of T-cell-mediated immune responses.

### SUMMARY

The present disclosure provides multivalent Major Histocompatibility Complex (MHC) multimers that can be used for determining peptide-MHC-TCR interactions and for modulating immune responses in vivo. The multimers provided herein comprise a fusion of an MHC moiety and a multimerization moiety, wherein the MHC moiety comprises an MHC molecule, optionally bound with a cognate peptide, operatively linked to a multimerization moiety from an IgM or IgA molecule. In some embodiments, the MHC molecule comprises an MHC Class I or Class II molecule. In some embodiments, where an MHC molecule comprises an MHC Class I molecule, it is optionally bound with an MHC Class I binding peptide. In some embodiments, where an MHC molecule comprises an MHC Class II molecule, it is optionally bound with an MHC II binding peptide. In some embodiments, the multimerization moiety comprises a region from an IgM or IgA molecule. For example, in some embodiments, the multimerization moiety comprises at least one C region from IgM or IgA, as well as a tailpiece region from IgM or IgA, such that the fusion molecule forms multimers. In some embodiments, the multimerization moiety comprises a tailpiece region of IgM or IgA alone. Thus, such multimers are multivalent and soluble. The combination of the peptide-MHC complex and the multi-valency of IgM or IgA results in a peptide-MHC-IgM or peptide-MHC-IgA fusion molecule with high avidity toward a T cell receptor (TCR) that recognizes the peptide-MHC complex. Still further, the multimers can comprise an effector moiety to impart the fusion molecule with effector-moiety mediated immunomodulatory functionality.

Multimers in accordance with the present disclosure can be used as therapeutic agents by administration to a subject in need thereof. For example, in some embodiments, administration of a composition comprising a multimer provided herein can modulate an immune response in a subject, such as in the treatment of autoimmune disorders with known pMHC drivers. In some embodiments, multimers can also be used as therapeutic agents by treating cells ex vivo and administering cells contacted by multimers to a subject to modulate an immune response. Thus, in some embodiments, peptide-MHC-Ig multimers provided herein offer an alternative route to autoantigen tolerization in autoimmunity while still exploiting the specificity of the TCR/pMHC interaction. Moreover, the multimers of the disclosure can be used for detecting pMHC/TCR interactions, e.g., in in vitro assays.

Accordingly, in some embodiments, the present disclosure provides a Major Histocompatibility Complex (MHC) multimers comprising an MHC moiety operatively linked to a multimerization moiety, wherein: (a) the MHC moiety comprises an MHC molecule, optionally bound with a cognate peptide; and (b) the multimerization moiety comprises (i) an IgM Cμ4 constant region and a tailpiece region; or (ii) an IgA Cα3 constant region and a tailpiece region, wherein the tailpiece region in (b)(i) or (b)(ii) is an IgM tailpiece region or an IgA tailpiece region.

In some embodiments, the Major Histocompatibility Complex (MHC) multimer comprises (i) an MHC Class I molecule, optionally bound with an MHC Class I-binding peptide; or (ii) an MHC Class II molecule, optionally bound with an MHC Class II-binding peptide; and (b) the multimerization moiety comprises (i) an IgM Cμ4 constant region and a tailpiece region; or (ii) an IgA Cα3 constant region and a tailpiece region, wherein the tailpiece region in (b)(i) or (b)(ii) is an IgM tailpiece region or an IgA tailpiece region.

In some embodiments, the present disclosure provides multimers comprising an MHC moiety operatively linked to a multimerization moiety, wherein the MHC moiety comprises an MHC molecule bound with a cognate peptide.

In some embodiments, a multimer comprises an MHC moiety operatively linked to a multimerization moiety, wherein: (a) the MHC moiety comprises an MHC molecule; and (b) the multimerization moiety comprises (i) an IgM Cμ4 constant region and a tailpiece region; or (ii) an IgA Cα3 constant region and a tailpiece region, wherein the tailpiece region in (b)(i) or (b)(ii) is an IgM tailpiece region or an IgA tailpiece region.

In some embodiments, the multimerization moiety comprises an IgM Cμ4 constant region and an IgM tailpiece region and further comprises an IgM Cμ3 constant region. In some embodiments, the multimerization moiety comprises an IgM Cμ4 constant region and an IgM tailpiece region and further comprises an IgM Cμ2 constant region. In some embodiments, the multimerization moiety comprises an IgM Cμ4 constant region and an IgM tailpiece region and further comprises an IgM Cμ1 constant region. In some embodiments, the multimerization moiety comprises an IgM Cμ4 constant region and an IgM tailpiece region and further comprises IgM Cμ2 and Cμ3 constant regions. In some embodiments, the multimerization moiety comprises an IgM Cμ4 constant region and an IgM tailpiece region and further comprises IgM Cμ1 and Cμ3 constant regions. In some embodiments, the multimerization moiety comprises an IgM Cμ4 constant region and an IgM tailpiece region and further comprises IgM Cμ1 and Cμ2 constant regions. In some embodiments, the multimerization moiety comprises an IgM Cμ4 constant region and an IgM tailpiece region and further comprises IgM Cμ1, Cμ2 and Cμ3 constant regions.

In some embodiments, the multimerization moiety comprises an IgA Cα3 constant region and an IgA tailpiece region and further comprises an IgA Cα2 constant region. In some embodiments, the multimerization moiety comprises an IgA Cα3 constant region and an IgA tailpiece region and further comprises an IgA Cal constant region. In some embodiments, the multimerization moiety comprises an IgA Cα3 constant region and an IgA tailpiece region and further comprises IgA Cal and Cα2 constant regions.

In some embodiments of the multimerization moiety, the IgM or IgA tailpiece region alone is used for multimerization, without inclusion of any immunoglobulin C regions. Accordingly, in another aspect, the disclosure pertains to a Major Histocompatibility Complex (MHC) multimer comprising an MHC moiety operatively linked to a multimerization moiety, wherein: (a) the MHC moiety comprises (i) an MHC Class I molecule, optionally bound with an MHC Class I-binding peptide; or (ii) an MHC Class II molecule, optionally bound with an MHC Class II-binding peptide; and (b) the multimerization moiety comprises an IgM tailpiece region or an IgA tailpiece region.

In certain embodiments, the multimerization moiety comprises the IgM tailpiece region. In other embodiments, the multimerization moiety comprises the IgA tailpiece region.

In some embodiments of the multimer, the MHC moiety is operatively linked to the multimerization moiety through an MHC Class I α chain. In other embodiments, the MHC moiety is operatively linked to the multimerization moiety through an MHC Class I β2-microglobulin. In still other embodiments, the MHC moiety is operatively linked to the multimerization moiety through an MHC Class II β chain. In some embodiments, the MHC moiety is operatively linked to the multimerization moiety through an MHC Class II α chain.

In some embodiments, the MHC moiety comprises an MHC Class I molecule bound with an MHC Class I-binding peptide. In some embodiments, the MHC moiety is operatively linked to the multimerization moiety through an MHC Class I α chain. In certain embodiments, the MHC moiety comprises MHC Class I α and β2-microglobulin chains.

In some embodiments, the MHC moiety is in a single polypeptide comprising, from the N-terminus to the C-terminus, the MHC Class I binding peptide, a linker, a β2M chain, a second linker, and an MHC Class I α chain, wherein the MHC Class I α chain optionally comprises a Y84A substitution. In some embodiments, the MHC Class I α chain comprises a Y84C substitution, and the first linker comprises a cysteine residue, such that a disulfide bond forms between the cysteine residue corresponding to position 84 of the MHC Class I α chain and the cysteine residue of the first linker.

In some embodiments, the MHC moiety comprises an MHC Class II molecule bound with an MHC Class II-binding peptide. In certain embodiments, the MHC moiety comprises MHC Class II α and β chains. In some embodiments, the MHC moiety is linked to the multimerization moiety through an MHC Class II β chain or an MHC Class II α chain. In some embodiments, the multimer further comprises a first heterodimerization motif operatively linked to an α chain of the MHC Class II molecule, and a second heterodimerization motif linked to a β chain of the MHC Class II molecule, wherein the first and second heterodimerization motifs form a heterodimer. In some embodiments, the first and/or second heterodimerization motifs are linked to the multimerization moiety. In some such embodiments, the first and/or second heterodimerization motifs comprise a leucine zipper.

In certain embodiments, the multimer further comprises an immunoglobulin J chain.

In certain embodiments, the multimer is soluble. In other embodiments, the multimer can be fixed to a solid support (e.g., a plate or a bead).

In certain embodiments, the MHC moiety is a single chain molecule (e.g., a single chain comprising a Class I-binding peptide, an MHC Class I α chain and a β2-microglobulin chain, with linkers interspersed, as described further herein).

In some embodiments, the multimer is a dimer, a trimer, a tetramer, a pentamer, or a hexamer. In one embodiment, the multimer is in a composition comprising a mixture of at least two multimers selected from the group consisting of dimers, trimers, tetramers, pentamers, and hexamers.

In some embodiment, the MHC moiety and the multimerization moiety are separated by a linker. In some such embodiments, the linker is a flexible linker, a rigid linker or a semi-rigid linker.

In certain embodiments, the multimer comprises an immunoglobulin J chain and the multimer further comprises an effector moiety operatively linked to the J chain. In some embodiments, the multimer comprises an effector moiety, optionally, wherein the effector moiety is operatively linked to the multimerization moiety.

In certain embodiments, the effector moiety comprises a checkpoint protein agonist, e.g., a checkpoint protein agonist antibody. In some embodiments, the checkpoint protein agonist comprises a PD-L1 extracellular domain or domain 1, an anti-PD1 agonist antibody, an anti-CTLA4 agonist antibody, an anti-Lag3 agonist antibody, an anti-TIM3 agonist antibody, an anti-TIGIT agonist antibody, an anti-LILRB1 agonist antibody, an anti-LILRB2 agonist antibody, or any portion or antigen-binding fragment thereof.

In other embodiments, the effector moiety comprises a TNFR family protein agonist. In some embodiments, the TNFR family protein agonist comprises 4-1BBL (CD137L), an anti-CD137 agonist antibody or any portion or antigen-binding fragment thereof, an anti-TNFR2 agonist antibody, or any portion or antigen-binding fragment thereof.

In some embodiments, the effector moiety comprises an anti-inflammatory agent (e.g., cytokine). In some such embodiments, the anti-inflammatory cytokine comprises IL10 or TGFβ. In some embodiments, the anti-inflammatory agent comprises an extracellular region of HLA-G, an anti-LILRB1 agonist antibody, an anti-LILRB2 agonist antibody, or any fragments or variants thereof.

In certain embodiments, the effector moiety comprises an immune cell engaging agent. In some embodiments, the immune cell engaging agent comprises an anti-CD3 antibody, an anti-NKp46 antibody, an anti-CD16a antibody, an anti-CD56 antibody, an anti-NKG2D antibody, an anti-NKp30 antibody, an anti-CD64 antibody, or any portion or antigen-binding fragments thereof. In other embodiments, the effector moiety comprises a cytokine. In some such embodiments, the cytokine comprises IL-2, IL-12, IL-15, TGFβ, or any variants or fragments thereof. In certain embodiments, where the cytokine comprises IL2, the IL-2 comprises a variant with reduced or no binding affinity for CD25/IL2Rα.

In certain embodiments, the effector moiety comprises a cell-death-inducing agent. In some embodiments, the cell-death-inducing agent comprises a tubulin inhibitor, a DNA damaging agent, FasL, anti-Fas agonist antibody or any portion or antigen-binding fragment thereof

In some embodiments, the effector moiety comprises a PD-1 binding moiety, such as a PD-1 agonist antibody or antigen binding fragment thereof. In one embodiment, the PD-1 binding moiety comprises PD-L1, such as PD-L1 ECD or domain 1.

In some embodiments, a subject suffering from an autoimmune disease or from cancer may be treated by administering a multimer comprising an effector moiety in accordance with the present disclosure.

In certain embodiments, the disclosure provides a composition comprising at least two multimers in accordance with the present disclosure, wherein the at least two multimers are in a mixture and wherein the multimers are selected from the group consisting of dimers, trimers, tetramers, pentamers, and hexamers.

In certain embodiments, the effector moiety is attached to the multimer through a linker (e.g., positioned between the J chain and the effector moiety or between the multimerization moiety and the effector moiety). In some embodiments, the linker is a flexible linker, a rigid linker, or a semi-rigid linker.

In yet another embodiment, the multimer further comprises one or more heterologous functional domains (e.g., a moiety for half-life extension, a cytokine, a cytokine receptor).

Multimers of the disclosure can be prepared by chemical conjugation of the components (as described further herein) but more typically are prepared recombinantly using expression vectors encoding the multimer components introduced into host cells. Accordingly, in another aspect, the disclosure pertains to isolated nucleic acids encoding subunits of the Major Histocompatibility Complex (MHC) multimers. In some embodiments, one or more multimers provided herein is/are encoded by one or more isolated nucleic acid molecules.

In some aspects, the present disclosure provides isolated nucleic acid molecules encoding an MHC moiety operatively linked to a multimerization moiety, wherein: (a) the MHC moiety comprises an MHC molecule and optionally a cognate peptide; and (b) the multimerization moiety comprises (i) an IgM Cμ4 constant region and a tailpiece region; or (ii) an IgA Cα3 constant region and a tailpiece region, wherein the tailpiece region in (b)(i) or (b)(ii) is an IgM tailpiece region or an IgA tailpiece region.

In some embodiments, the MHC moiety comprises (i) an MHC Class I molecule, and optionally an MHC Class I-binding peptide; or (ii) an MHC Class II molecule and optionally an MHC Class II-binding peptide.

In some aspects, the present disclosure provides isolated nucleic acid molecules encoding an MHC moiety operatively linked to a multimerization moiety, wherein: (a) the MHC moiety comprises (i) an MHC Class I molecule, optionally bound with an MHC Class I-binding peptide; or (ii) an MHC Class II molecule, optionally bound with an MHC Class II-binding peptide; and (b) the multimerization moiety comprises (i) an IgM Cμ4 constant region and a tailpiece region; or (ii) an IgA Cα3 constant region and a tailpiece region, wherein the tailpiece region in (b)(i) or (b)(ii) is an IgM tailpiece region or an IgA tailpiece region.

In some embodiments of the isolated nucleic acid, the multimerization moiety comprises an IgM Cμ4 constant region and an IgM tailpiece region and further comprises an IgM Cμ3 constant region. In some embodiments, the multimerization moiety comprises an IgM Cμ4 constant region and an IgM tailpiece region and further comprises an IgM Cμ2 constant region. In some embodiments, the multimerization moiety comprises an IgM Cμ4 constant region and an IgM tailpiece region and further comprises an IgM Cμ1 constant region. In some embodiments, the multimerization moiety comprises an IgM Cμ4 constant region and an IgM tailpiece region and further comprises IgM Cμ2 and Cμ3 constant regions. In some embodiments, the multimerization moiety comprises an IgM Cμ4 constant region and an IgM tailpiece region and further comprises IgM Cμ1 and Cμ3 constant regions. In some embodiments, the multimerization moiety comprises an IgM Cμ4 constant region and an IgM tailpiece region and further comprises IgM Cμ1 and Cμ2 constant regions. In some embodiments, the multimerization moiety comprises an IgM Cμ4 constant region and an IgM tailpiece region and further comprises IgM Cμ1, Cμ2 and Cμ3 constant regions.

In one embodiment of the isolated nucleic acid, the multimerization moiety comprises an IgA Cα3 constant region and an IgA tailpiece region and further comprises an IgA Cα2 constant region. In one embodiment, the multimerization moiety comprises an IgA Cα3 constant region and an IgA tailpiece region and further comprises an IgA Cal constant region. In one embodiment, the multimerization moiety comprises an IgA Cα3 constant region and an IgA tailpiece region and further comprises IgA Cal and Cα2 constant regions.

In another embodiment of the isolated nucleic acid encoding the MHC multimer, the multimerization moiety comprises the IgM or IgA tailpiece region alone, without inclusion of any immunoglobulin C regions. Accordingly, in one aspect, the disclosure pertains to isolated nucleic acid molecules encoding multimers comprising a MHC moiety operatively linked to a multimerization moiety, wherein: (a) the MHC moiety comprises (i) an MHC Class I molecule, optionally bound with an MHC Class I-binding peptide; or (ii) an MHC Class II molecule, optionally bound with an MHC Class II-binding peptide; and (b) the multimerization moiety comprises an IgM tailpiece region or an IgA tailpiece region.

In certain embodiments, the multimerization moiety comprises the IgM tailpiece region. In other embodiments, the multimerization moiety comprises the IgA tailpiece region.

In some embodiments, the MHC moiety is operatively linked to the multimerization moiety through an MHC Class I α chain. In some embodiments, the MHC moiety is operatively linked to the multimerization moiety through an MHC Class II β chain or an MHC Class II α chain.

In one embodiment of the isolated nucleic acid, the MHC moiety and the multimerization moiety are separated by a linker. In some embodiments, a linker is a flexible linker, a rigid linker, or a semi-rigid linker.

In yet another aspect, the disclosure pertains to expression vectors and host cells comprising a nucleic acid of the disclosure. In some embodiments, an isolated nucleic acid encoding a peptide-MHC-IgM or peptide-MHC-IgA fusion provided in accordance with the present disclosure can be incorporated into an expression vector and the expression vector can be introduced into a host cell to thereby express the fusion protein. In certain embodiments, the host cell comprises an expression vector encoding an MHC moiety comprising an MHC chain linked to the IgM or IgA C region. In other embodiments, a host cell further comprises an expression vector encoding the cognate MHC chain that pairs with its Class I or Class II counterpart (e.g., resulting in Class I α/β2microglobulin pairs or Class II α/β pairs in the host cell).

In meso embodiments, the host cell further comprises an expression vector encoding an immunoglobulin J chain and a separate expression vector encoding the multimer. In some embodiments, in the expression vector, the immunoglobulin J chain is operatively linked to an effector moiety. In one embodiment, the effector moiety comprises a PD-1 binding moiety. In one embodiment, the PD-1 binding moiety comprises PD-L1.

Methods of preparing multimers of the disclosure are also provided wherein host cells carrying expression vectors encoding the multimer components are cultured to thereby express the multimer. In some embodiments, the method further comprises isolating the multimer from the host cells or the host cell culture supernatant.

In yet another aspect, the disclosure provides pharmaceutical compositions comprising a multimer of the disclosure and a pharmaceutically acceptable carrier.

In certain embodiments, the present disclosure provides kits comprising a multimer in as provided herein, which multimer is packaged in a container with instructions for use in detecting peptide-MHC-TCR interactions and/or modulating an immune response.

In yet another aspect, the disclosure provides methods of detecting a TCR-peptide-MHC complex. In some embodiments, the method comprises contacting a TCR with a multimer of the disclosure and detecting binding of the TCR to the multimer to thereby detect binding of the TCR to the peptide-MHC complex of the multimer. In some embodiments, the multimer comprises a detectable moiety. In one embodiment, the TCR is on the surface of T cell and the multimer is contacted with the T cell.

In yet another aspect, the disclosure provides methods of modulating an immune response in a subject. In some such embodiments, the method comprises administering to the subject an effective amount of a multimer of the disclosure or a pharmaceutical composition comprising a multimer of the disclosure such that an immune response is modulated in the subject.

In certain embodiments, the subject has an autoimmune disorder and the immune response to the autoimmune disorder is modulated (e.g., down modulated). In other embodiments, the subject has an inflammatory disorder and the immune response to the inflammatory disorder is modulated (e.g., down modulated). Accordingly, in certain embodiments, the disclosure provides methods of treating an autoimmune disorder in a subject, the method comprising administering to the subject a multimer of the disclosure, wherein the MHC moiety of the multimer recognizes an autoantigen of the subject's autoimmune disorder. In some embodiments, the autoimmune disorder is Type I diabetes. In some embodiments, the autoantigen is an insulin autoantigen. In some embodiments, the subject is further treated with one or more additional therapies.

In some embodiments, the present disclosure provides methods of treating an infectious disease. In some such embodiments, the infectious disease is a virus. In some embodiments, cells infected by the virus are targeted (e.g., in cis or trans as provided and described herein) by multimers of the present disclosure.

In certain embodiments, the present disclosure provides methods of treating cancer. In some embodiments, the method of treating cancer comprises using an immunotherapy comprising administering to a subject diagnosed with cancer a multimer provided by the present disclosure or a pharmaceutical composition comprising a multimer provided by the present disclosure. In some such embodiments, cancer cells are targeted (e.g., in cis or trans as provided and described herein) by multimers of the present disclosure.

Among other things, in some aspects, the present disclosure provides use of an MHC multimer in the manufacture of a medicament for treating an autoimmune disorder, inflammatory disorder, infectious disease, or cancer. In some embodiments, the present disclosure provides a method of treating cancer using an immunotherapy comprising administering to a subject diagnosed with cancer a multimer as provided herein. In some embodiments, the subject is treated with one or more additional therapies.

Further details of the nature and advantages of the present disclosure can be found in the following detailed description taken in conjunction with the accompanying figures. The present disclosure is capable of modification in various respects without departing from the spirit and scope of the present disclosure. Accordingly, the figures and description of these embodiments are not restrictive.

### BRIEF DESCRIPTION OF THE FIGURES

A better understanding of the features and advantages of the present disclosure will be obtained by reference to the following detailed description that sets forth illustrative embodiments, in which the principles of the disclosure are utilized, and the accompanying drawings of which:

FIGS. 1A-1C are schematic representations of exemplary MHC multimers of the disclosure. In FIG. 1A, the multimer comprises an MHC moiety of an MHC Class I α chain, paired with β2-microglobulin and bound with an MHC Class I-binding peptide, and a multimerization moiety of an IgM Cμ3 and Cμ4 constant regions and IgM tailpiece region, as well as an immunoglobulin J chain. In FIG. 1B, the multimer is the same as in FIG. 1A except that the multimerization moiety includes IgM Cμ2, Cμ3, and Cμ4 constant regions and IgM tailpiece region. In FIG. 1C, the multimer comprises an MHC moiety of an MHC Class I α chain, paired with β2-microglobulin and bound with an MHC Class I-binding peptide, and a multimerization moiety of an IgA Cα2 and Cα3 constant region and IgA tailpiece region, as well as an immunoglobulin J chain. In FIG. 1D, the multimer is a single-chain pMHC comprising an MHC moiety of an MHC Class I α chain, paired with β2-microglobulin and bound with an MHC Class I-binding peptide, with at least one linker between the MHC Class I-binding peptide and the β2-microglobulin, and a multimerization moiety of an IgM Cμ3 and Cμ4 constant regions and IgM tailpiece region, as well as an immunoglobulin J chain. Disulfide bridges are indicated by dashed lines; linkers are indicated by double-dashed lines (FIG. 1D).

FIGS. 2A-2C are schematic representations of exemplary MHC multimers of the disclosure that comprise an effector/targeting moiety (labeled “effector moiety”). In FIG. 2A, the multimer comprises an MHC moiety of an MHC Class I α chain, paired with β2-microglobulin and bound with an MHC Class I-binding peptide, and a multimerization moiety of an IgM Cμ3 and Cμ4 constant regions and IgM tailpiece region, as well as an immunoglobulin J chain linked to an effector/targeting moiety (labeled “effector moiety”). In FIG. 2B, the multimer is the same as in FIG. 2A except that the multimerization moiety includes IgM Cμ2, Cμ3, and Cμ4 constant regions and IgM tailpiece region. In FIG. 2C the multimer comprises an MHC moiety of an MHC Class I α chain, paired with β2-microglobulin and bound with an MHC Class I-binding peptide, and a multimerization moiety of an IgA Cα2 and Cα3 constant region and IgA tailpiece region, as well as an immunoglobulin J chain linked to an effector/targeting moiety (labeled “effector moiety”). Disulfide bridges are indicated by dashed lines.

FIGS. 3A-3C are schematic representations of exemplary MHC multimers of the disclosure. In FIG. 3A, the multimer comprises an MHC moiety of an MHC Class II α chain, paired with an MHC Class II β chain and bound with an MHC Class II-binding peptide, and a multimerization moiety of an IgM Cμ3 and Cμ4 constant regions and IgM tailpiece region, as well as an immunoglobulin J chain. In FIG. 3B, the multimer is the same as in FIG. 3A except that the multimerization moiety includes IgM Cμ2, Cμ3 and Cμ4 constant regions and IgM tailpiece region. In FIG. 3C the multimer comprises an MHC moiety of an MHC Class II α chain, paired with an MHC Class II β chain and bound with an MHC Class II-binding peptide, and a multimerization moiety of an IgA Cα2 and Cα3 constant region and IgA tailpiece region, as well as an immunoglobulin J chain. Disulfide bridges are indicated by dashed black lines.

FIGS. 4A-4C are schematic representations of exemplary MHC multimers of the disclosure. In FIG. 4A, the multimer comprises an MHC moiety of an MHC Class II α chain, paired with an MHC Class II β chain and bound with an MHC Class II-binding peptide, and a multimerization moiety of an IgM Cμ3 and Cμ4 constant regions and IgM tailpiece region, as well as an immunoglobulin J chain linked to an effector/targeting moiety (labeled “effector moiety”). In FIG. 4B, the multimer is the same as in FIG. 4A except that the multimerization moiety includes IgM Cμ2, Cμ3, and Cμ4 constant regions and IgM tailpiece region. In FIG. 4C the multimer comprises an MHC moiety of an MHC Class II α chain, paired with an MHC Class II β chain and bound with an MHC Class II-binding peptide, and a multimerization moiety of an IgA Cα2 and Cα3 constant region and IgA tailpiece region, as well as an immunoglobulin J chain linked to an effector/targeting moiety (labeled “effector moiety”). Disulfide bridges are indicated by dashed black lines.

FIGS. 5A-5I are schematic representations of exemplary nucleic acid constructs used to recombinantly express an MHC multimer of the disclosure. Components of the multimer encoded by each nucleic acid are indicated (P=Peptide, L=Linker, tp=tailpiece, J=J chain, EM=effector moiety, HetD1=heterodimerization domain 1, HetD2=heterodimerization domain 2, T2A=T2A self-cleaving peptide). FIG. 5A is a schematic representation of exemplary constructs encoding peptide-β2m-MHC Class I-IgM and J-chain. FIG. 5B is a schematic representation of exemplary constructs encoding peptide-β2m-MHC Class I-IgM and J-chain-EM fusion. FIG. 5C is a schematic representation of exemplary constructs encoding peptide-MHC Class II β-IgM, MHC Class II α, and J-chain. FIG. 5D is a schematic representation of exemplary constructs encoding peptide-MHC Class II β-IgM, MHC Class II α, and J-chain-EM fusion. FIG. 5E is a schematic representation of exemplary constructs encoding peptide-MHC Class II β-IgM and a bicistronic construct encoding MHC Class II α and J-chain-EM fusion. FIG. 5F is a schematic representation of exemplary constructs encoding peptide-MHC Class II β-heterodimerization domain 1-IgM, MHC Class II α-heterodimerization domain 2, and J-chain. FIG. 5G is a schematic representation of exemplary constructs encoding peptide-MHC Class II β-heterodimerization domain 1-IgM and a bicistronic exemplary construct encoding MHC Class II α-heterodimerization domain 2 and J-chain. FIG. 5H is a schematic representation of exemplary constructs encoding peptide-MHC Class II β-heterodimerization domain 1-IgM, MHC Class II α-heterodimerization domain 2, and J-chain-EM fusion. FIG. 5I is a schematic representation of exemplary constructs encoding peptide-MHC Class II β-heterodimerization domain 1-IgM and a bicistronic construct encoding MHC Class II α-heterodimerization domain 2 and J-chain-EM fusion.

FIG. 6A is a schematic representation of an exemplary bispecific MHC class I multimer of the disclosure comprising two different MHC-binding peptides (Peptide 1 and Peptide 2). The use of knob-into-hole (KIH) bispecific technology to promote heterodimerization of the Cμ2, Cμ3, and/or Cμ4 regions is indicated. FIG. 6B is a schematic representation of an exemplary bispecific MHC class I multimer of the disclosure comprising one or two different MHC-binding peptides (Peptide 1 and Peptide 2). The use of knob-into-hole (KIH) bispecific technology to promote heterodimerization of the Cα2 and Cα3 regions is indicated. Disulfide bridges are indicated by dashed lines.

FIG. 7 illustrates a schematic representation of a process for assessing the ability of an exemplary pMHC-Ig-immunosuppressor to suppress the activity of target T cells, where (I) represents measurement of increased % live target cells, (II) represents monitoring reduced activation level of effector T cells by flow cytometry or by western blot, (III) represents measurement of reduced proliferation of antigen-specific T cells, and (IV) represents testing for reduced cytokine secretion (e.g., IFNγ, IL2, and/or TNFα).

FIG. 8 is a schematic representation of an approach for assessing the ability of pMHC-Ig-FasL for inducing T cell apoptosis.

FIG. 9 is a schematic representation of an approach for assessing the ability of pMHC-Ig-cytotoxin for inducing targeted T cell death.

FIG. 10 is a schematic representation of an approach for assessing the ability of pMHC-Ig-anti-CD3 for redirected T cell killing.

FIG. 11 is a schematic representation of an approach for assessing the ability of pMHC-Ig fusions for efficacy to expand regulatory T cells. Exemplary fusions include: TGFβ, TNFR2 agonist, TRAIL, TRAIL-R1, and/or TRAIL-R2 agonist.

FIGS. 12A-12C show results from expression and purification of an exemplary MHC multimer. FIG. 12A shows a western blot of a MHC multimer expression sample in EXPI293 cells probed with Anti-IgM (left panel) and Anti-Flag antibodies for the J-chain (right panel). FIG. 12B shows a western blot of MHC multimer sample fractions probed with Anti-IgM (left panel) and Anti-Flag antibodies (right panel) after immobilized metal affinity chromatography (IMAC) purification. FIG. 12C shows an SEC purification chromatogram of a MHC multimer.

FIGS. 13A-13C show results of a purity analysis from an exemplary overexpressed Class I pMHC-IgM protein. FIG. 13A shows an analytical Size Exclusion Chromatogram (SEC) of purified Class I pMHC-IgM protein. FIG. 13B shows a SDS-PAGE-Coomassie (left panel) and western blots (two right panels) with anti-IgM and anti-Flag antibodies that show presence of IgM and J-chain in a non-reduced, unboiled sample. FIG. 13C shows a SDS-PAGE-Coomassie (left panel) and western blots (right panels) with anti-IgM and anti-Flag antibodies that show presence of IgM and J-chain in a reduced, boiled sample.

FIG. 14A shows results of a flow cytometry study, which shows binding of ELA-HLA-A2-IgM to peptide expanded T cells: ELAGIGILTV-expanded T cells (upper panels), non-cognate peptide-expanded T cells (lower panels). FIG. 14B shows flow cytometry plots of pMHC-IgM to ELA/HLA-A2-specific TCR Jurkat cells at 6 nM concentration. The upper left panel shows IgM Fc control, the upper right panel shows ELA/HLA-A2-IgM, the lower left panel shows non-cognate-peptide 9-mer 2-HLA-A2-IgM, and the lower right panel shows ELA-HLA-A2-SA tetramer.

FIG. 15 shows the Mean Fluorescence Intensity (MFI) of pMHC-IgM binding to anti-MART1-Jurkat cells as measured by flow cytometry at various concentrations (0.24 nM, 1.2 nM, 6 nM, 30 nM, and 150 nM). IgM-Fc, non-cognate-peptide 9-mer 2-HLA-A2/IgM and UV-exchanged ELA-HLA-A2-SA tetramers were included as controls.

FIGS. 16A-16C show results from the production of an exemplary Class II pMHC-IgM protein. FIG. 16A shows post-expression protein samples on western blots detecting the IgM with anti-IgM antibody (third from left panel), the α chain with anti-Myc antibody (right panel), and the J-chain with anti-Flag antibody (second from left panel). FIG. 16B shows post-gravity purification with Ni Excel resin protein samples on western blots detecting IgM with anti-IgM antibody (right panel) and α chain with anti-Myc antibody (left panel). The box indicates the area of the gel where both antibody signals overlay with each other. FIG. 16C shows post-SEC purification western blots detecting J-chain with anti-Flag antibody (left panel), IgM with anti-IgM antibody (middle panel), and α chain with anti-Myc antibody (right panel).

FIGS. 17A-17B depict an exemplary single chain HLA (sc-HLA) (FIG. 17A) and an exemplary cysteine engineered disulfide stabilized sc-HLA (cys-trap) (FIG. 17B). FIG. 17C shows western blots of three sc-HLA-IgM loaded with exemplary 9-mer 5, 9-mer 4, and 9-mer 2 peptides without or with engineered disulfide probed with anti-IgM (left panel) and anti-Flag (right panel) antibodies.

FIGS. 18A-18D show results from production of an exemplary Class I pMHC-IgM with multiple immunomodulators (J-chain, anti-CD3, and PD-L1). FIGS. 18A and 18B show western blots performed on purified KLW-A2-IgM with J-chain, anti-CD3, or PD-L1 using an anti-human IgM antibody conjugated to DyLight800 and an anti-DYKDDDDK (anti-Flag) conjugated to DyLight680 to confirm the presence of both the pMHC-IgM chain and J-chain, respectively. In FIG. 18A: non-boiled, nonreduced samples, FIG. 18B: boiled, reduced samples. FIG. 18C shows SDS-PAGE with Coomassie staining for non-reduced (left panel) and reduced (right panel) samples. Lanes 1: J chain, 2: OKT3-J chain (anti-CD3), 3: PD-L1-ECD-J chain. FIG. 18D shows exemplary representative production yields in mg/L of KLW-A2-IgM with J-chain, anti-CD3, and PD-L1.

FIG. 19A-19B illustrate a schematic representation of an approach for producing a pMHC-IgM protein via sortase addition (FIG. 19A) or via click chemistry (FIG. 19B).

FIGS. 20A-20B show results from generation of an exemplary pMHC-IgM through a sortase reaction. FIG. 20A shows a western blot analysis of sortase reactions to conjugate pMHC monomer to IgM-Fc, under two different reaction conditions: Reaction 1 and Reaction 2. The antibodies used were an anti-human IgM antibody conjugated to DyLight800 and an anti-HLA A chain conjugated to DyLight680 to confirm the presence of both the IgM and HLA chains, respectively. FIG. 20B shows an analytical SEC on starting samples and Reaction 1 and 2 to confirm high valency of HLA in Reaction 1; corresponding western blot lanes are indicated in the table to the right of the chromatogram.

FIG. 21 shows exemplary flow cytometric analyses demonstrating that pMHC-IgM multimers produced via sortase reactions bind cognate engineered TCR cell lines.

### DETAILED DESCRIPTION

The present disclosure provides technologies such as compositions, methods of manufacturing, and methods of treatment comprising multivalent Major Histocompatibility Complex (MHC) multimers. As provided herein, each MHC multimer comprises a fusion of an MHC moiety and a multimerization moiety, wherein the MHC moiety comprises an MHC Class I molecule, optionally bound with an MHC Class I-binding peptide, or an MHC Class II molecule, optionally bound with an MHC Class II-binding peptide, operatively linked to a multimerization moiety that comprises at least one C region from an IgM or IgA molecule such that the fusion molecule forms multimers. The multimers of the disclosure can further comprise an immunoglobulin J chain, which facilitates formation of certain forms of multimers (e.g., pentamers). Additionally, the multimers can further comprise an effector/targeting moiety, which can facilitate cellular redirection, such as suppressing or killing, supporting, or otherwise influencing certain T cell populations to treat autoimmune diseases and cancer.

The MHC multimers can be prepared with a peptide bound to the peptide-binding groove of the MHC molecule by expression in a host cell using an expression construct that includes encoding of the peptide epitope. For example, in one embodiment, an expression construct is used that encodes the peptide and additional multimer components, wherein the peptide is fused with a flexible linker to an MHC chain of the MHC moiety of the multimer.

Alternatively, “empty” MHC multimers, comprising non-peptide-bound MHC Class I or Class II molecules linked to the IgM- or IgA-derived multimerization domain, can be prepared and then subsequently loaded with antigenic peptide, as described herein. In one embodiment, the “empty” multimers are prepared using a similar strategy to that of the peptide-loaded multimers except that the peptide moiety is omitted. Furthermore, one or more modifications can be introduced into an MHC chain to increase stability of the empty assembled MHC (see e.g., Example 4). Empty MHC molecules, and methods of preparing same, have been described in the art (see e.g., Serra et al. (2019) NATURE COMM. 10:4917).

Various components and aspects of the disclosure are described in further detail in the subsections below.

### I. Definitions

All technical and scientific terms used herein, unless otherwise defined below, are intended to have the same meaning as commonly understood by one of ordinary skill in the art. Mention of techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

In the disclosure, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method provided and described herein can be combined in a variety of ways without departing from the spirit and scope of the present disclosure and invention(s) herein, whether explicit or implicit herein. For example, where reference is made to a particular compound, that compound can be used in various embodiments of compositions of the present disclosure and/or in methods of the present disclosure, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of invention(s) provided, described, and depicted herein.

As used herein, “about” will be understood by persons of ordinary skill and will vary to some extent depending on the context in which it is used. If there are uses of the term which are not clear to persons of ordinary skill given the context in which it is used, “about” will mean up to plus or minus 10% of the particular value.

The articles “a” and “an” are used in this disclosure to refer to one or more than one (i.e., to at least one) of the grammatical object of the article, unless the context is inappropriate. By way of example, “an element” means one element or more than one element.

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

It should be understood that the order of steps or order for performing certain actions is immaterial so long as the present invention remain operable. Moreover, two or more steps or actions may be conducted simultaneously.

At various places in the present specification, variable or parameters are disclosed in groups or in ranges. It is specifically intended that the description include each and every individual subcombination of the members of such groups and ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present disclosure and does not pose a limitation on the scope of any invention(s) unless claimed. No language in the specification should be construed as indicating any non-claimed element as essential to the practice of that provided by the present disclosure.

The terms “antigenic peptide”, “antigenic determinant” or “epitope” refer to a site on an antigen to which a T-cell receptor, an MHC molecule or antibody specifically binds. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation and typically can include up to about 25 amino acids. Methods for determining what epitopes are bound by a given TCR or antibody (i.e., epitope mapping) are well known in the art and include, for example, immunoblotting and immunoprecipitation assays, wherein overlapping or contiguous peptides from the antigen are tested for reactivity with the given TCR or immunoglobulin. Methods of determining spatial conformation of epitopes include techniques in the art and those described herein, for example, x-ray crystallography, nuclear magnetic resonance, cryo-electron microscopy (cryo-EM), hydrogen deuterium exchange mass spectrometry (HDX-MS), and site-directed mutagenesis (see, e.g., EPITOPE MAPPING PROTOCOLS IN METHODS IN MOLECULAR BIOLOGY, Vol. 66, G. E. Morris, Ed. (1996)).

As used herein, the terms “antigen-presenting cell” or “APC” refer to a cell that mediate a cellular immune response by displaying an epitope on its outer surface, presented by a protein or protein complex (e.g., a major histocompatibility complex (MHC) or MHC-related protein such as CD1 that can present lipids to T cells), for recognition by an immune cell such as a T cell. APCs include professional APCs, such as dendritic cells, macrophages, Langerhans cells, and B cells, that express both class I and class II MHCs, and non-professional APCs (e.g., nucleated cells) that express only class I MHCs. APCs also include artificial APCs, such as cells (e.g., Drosophila cells) engineered to express a MHC that presents a T cell epitope.

The term “avidity” as used herein, refers to the binding strength as a function of the cooperative interactivity of multiple binding sites of a multivalent molecule (e.g., a soluble multivalent MHC multimer) with a target molecule. A number of technologies exist to characterize the avidity of molecular interactions including switchSENSE and surface plasmon resonance (Gjelstrup et al. (2012) J. IMMUNOL., 188:1292-1306; Vorup-Jensen (2012) ADV. DRUG. DELIV. REV. 64:1759-1781).

As used herein a “barcode”, also referred to as an oligonucleotide barcode, is a typically short nucleotide sequence (e.g., about 1, 2, 3, 4, 5, 6, 7, 8, 9, 10, 11, 12, 13, 14 or 15 nucleotides long or longer) that identifies a molecule to which it is conjugated. Barcodes can be used, for example, to identify molecules in a reaction mixture. Barcodes uniquely identify the molecule to which it is conjugated, for example, by performing reverse transcription using primers that each contain a “unique molecular identifier” barcode. In other embodiment, primers can be utilized that contain “molecular barcodes” unique to each molecule. The process of labeling a molecule with a barcode is referred to herein as “barcoding.” A “DNA barcode” is a DNA sequence used to identify a target molecule during DNA sequencing. In some embodiments, a library of DNA barcodes is generated randomly, for example, by assembling oligos in pools. In other embodiments, the library of DNA barcodes is rationally designed in silico and then manufactured.

“Binding affinity” generally refers to the strength of the sum total of noncovalent interactions between a single binding site of a molecule (e.g., a TCR variable region) and its binding partner. Unless indicated otherwise, as used herein, “binding affinity” refers to intrinsic binding affinity which reflects a 1:1 interaction between members of a binding pair (e.g., TCR and peptide-MHC). The affinity of a molecule X for its partner Y can generally be represented by the dissociation constant (Kd). For example, the Kd can be about 1 μM, 500 nM, 200 nM, 150 nM, 100 nM, 60 nM, 50 nM, 40 nM, 30 nM, 20 nM, 10 nM, 8 nM, 6 nM, 4 nM, 2 nM, 1 nM, or less than 1 nM. A variety of methods of measuring binding affinity are known in the art, any of which can be used for purposes of the present disclosure.

As used herein, the terms “carrier” refers to a material, composition or vehicle, such as a liquid or solid filler, diluent, excipient, solvent or encapsulating material, involved in carrying or transporting a pharmaceutical agent, or a pharmaceutically acceptable salt thereof, from one organ, or portion of the body, to another organ, or portion of the body. A “pharmaceutically acceptable carrier” includes any and all solvents, dispersion media, coatings, antibacterial and antifungal agents, isotonic and absorption delaying agents, and the like that are physiologically compatible.

As used herein, the term “cleavage site” or “cleavable moiety” refers to a site, a motif or sequence that is cleavable, such as by an enzyme (e.g., a protease) or by particular reaction conditions. In some embodiments, the cleavage moiety comprises a protein, e.g., enzymatic, cleavage site. In some embodiments, the cleavage moiety comprises a chemical cleavage site, e.g., through exposure to oxidation/reduction conditions, light/sound, temperature, pH, pressure, etc.

The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

As used herein, “effective amount” or “therapeutically-effective amount” refers to the amount of an active agent (e.g., a pMHC multimer as provided herein) sufficient to effect beneficial or desired results. An effective amount can be administered in one or more administrations, applications or dosages and is not intended to be limited to a particular formulation or administration route.

As used herein, the term “expression construct” refers to a vector designed for gene expression, e.g., in a host cell. An expression vector promotes the expression (i.e., transcription/translation) of an encoded polypeptide (e.g., fusion polypeptide). Typically, the vector is a plasmid, although other suitable vectors, including viral and non-viral vectors are also encompassed by the term “expression construct.”

As used herein, an “IgM constant region” refers to all or a portion of the four constant region domains of immunoglobulin M, referred to as Cμ1, Cμ2, Cμ3 and Cμ4 (each approximately 110 amino acids), including alleles (including the four human IgM constant region alleles), variants and modified forms that retain the functional activity of the naturally-occurring IgM constant region domain(s).

As used herein, an “IgA constant region” refers to all or a portion of the three constant region domains of immunoglobulin A (isotype A1 or A2), referred to as Cal, Cα2 and Cα3 (each approximately 110 amino acids in length), including alleles (including the three human IgA constant region alleles of A1 or A2), variants and modified forms that retain the functional activity of the naturally-occurring IgA constant region domain(s).

As used herein, the term “immunoglobulin J chain” refers to an optional accessory protein of IgM or IgA that is not essential for IgM or IgA assembly (polymerization) but that enhances IgM or IgA assembly and plays a role in mucosal secretion and complement activation. An exemplary full-length human J chain has the amino acid sequence shown in SEQ ID NO: 26.

As used herein, the terms “immunoglobulin tailpiece” or “tailpiece region” refers to a short extension region at the C-terminal end of the constant region of an immunoglobulin, e.g., IgM or IgA (including all alleles thereof). The IgM and IgA tailpieces are typically about 18 amino acid regions at the carboxy terminus, which contain a cysteine that is involved in multimerization of IgM or IgA. Exemplary human IgM and IgA tailpieces have the amino acid sequences shown in SEQ ID NOs: 129 and 130, respectively.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

As used herein, the terms “linked,” “conjugated,” “fused,” or “fusion,” are used interchangeably when referring to the joining together of two more elements or components or domains, by whatever means including recombinant or chemical means.

As used herein, the term “linker sequence” refers to a nucleotide sequence, and corresponding encoded amino acid sequence, or “linker” that serves to link or separate two polypeptides, such as two polypeptide domains of a fusion protein. For example, an intervening linker sequence can serve to provide flexibility and/or additional space between the two polypeptides that flank the linker. A linker sequence also can contain, for example, a protease cleavage site or a ribosomal skipping sequence.

As used herein, the terms “operatively linked” and “operably linked” are used interchangeably to describe configurations between sequences within an expression construct that allow for particular operations to be carried out. For example, when a regulatory sequence is “operatively linked” to a coding sequence within an expression construct, the regulatory sequence operates to regulate the expression of the coding sequence. Similarly, when a cleavage sequence (site) is “operatively linked” to a peptide sequence within an expression construct, cleavage at the cleavage sequence operates to cleave the peptide sequence away from the rest of the polypeptide encoded by the expression construct. Similarly, when two polypeptides within a fusion protein are “operatively linked”, the sequences encoding the two polypeptides are linked in-frame in an expression construct such that transcription and translation of the construct leads to a contiguous fusion protein comprised of the two polypeptides.

The term “major histocompatibility complex” or “MHC” refers to a genomic locus containing a group of genes that encode the polymorphic cell-membrane-bound glycoproteins known as MHC class I and class II molecules, which regulate the immune response by presenting peptides of fragmented proteins to CD8⁺ (e.g., cytotoxic) and CD4⁺(e.g., helper) T lymphocytes, respectively. In humans, this group of genes is also called “human leukocyte antigen” or “HLA.” A HLA can be selected from multiple serotypes (e.g., HLA-A*02, HLA-DPA1*02) which each may include multiple alleles (e.g., HLA-A*02:01, HLA-DPA1*02:02). Nucleotide sequences and a gene map of human MHC are publicly available (e.g., The MHC sequencing consortium, (1999) NATURE, 401:921-923.

The terms “major histocompatibility complex” and “MHC” also refer to the polymorphic glycoproteins encoded by the MHC class I or class II genes, where appropriate in the context, and proteins comprising variants thereof that bind T cell epitopes (e.g., class I or class II epitopes). Such proteins are also referred to as “MHC molecule” or “MHC protein” herein. The terms “MHC class I” or “MHC I” are used interchangeably to refer to protein molecules comprising an α chain composed of three domains (α1, α2 and α3), and a second, invariant β2-microglobulin. The α3 domain is linked to the transmembrane domain, anchoring the MHC class I molecule to the cell membrane. Antigen-derived peptide epitopes, which are located in the peptide-binding groove, in the central region of the α1/α2 heterodimer. MHC Class I molecules such as HLA-A are part of a process that presents short polypeptides to the immune system. These polypeptides are typically 8-11 amino acids in length and originate from proteins being expressed by the cell, which can be endogenous proteins or exogenous proteins (e.g., viral or bacterial proteins, vaccine proteins). MHC class I molecules present antigen to CD8+ cytotoxic T cells. The terms “MHC class II” and “MHC II” are used interchangeably to refer to protein molecules containing an α chain with two domains (α1 and α2) and a β chain with two domains (β1 and β2). The peptide-binding groove is formed by the α1/β1 heterodimer. MHC class II molecules present antigen to specific CD4+ T cells. Antigens delivered endogenously to APCs are processed primarily for association with MHC class I. Antigens delivered exogenously to APCs are processed primarily for association with MHC class II. As used herein, MHC proteins (MHC Class I or Class II proteins) also includes MHC variants which contain amino acid substitutions, deletions or insertions and yet which still bind MHC peptide epitopes (MHC Class I or MHC Class II peptide epitopes). The term “MHC,” “MHC molecule,” or “MHC protein” also includes an extracellular fragment of a full-length MHC protein that retains the ability to bind the cognate epitope, for example, a soluble MHC. As used herein, the term “soluble MHC” refers to an extracellular fragment of a MHC comprising corresponding α1 and α2 domains that bind a class I T cell epitope or corresponding α1 and 31 domains that bind a class II T cell epitope, where the α1 and α2 domains or the α1 and β1 domains are derived from a naturally occurring MHC or a variant thereof.

The term “MHC protein” also includes MHC proteins of non-human species of vertebrates. MHC proteins of non-human species of vertebrates play a role in the examination and healing of diseases of these species of vertebrates, for example, in veterinary medicine and in animal tests in which human diseases are examined on an animal model, for example, experimental autoimmune encephalomyelitis (EAE) in mice (Mus musculus), which is an animal model of the human disease multiple sclerosis. Non-human species of vertebrates are, for example, and more specifically mice (Mus musculus), rats (Rattus norvegicus), cows (Bos taurus), horses (Equus equus) and green monkeys (Macaca mulatta). MHC proteins of mice are, for example, referred to as H-2-proteins, wherein the MHC class I proteins are encoded by the gene loci H2K, H2L, and H2D and the MHC class II proteins are encoded by the gene loci H2I.

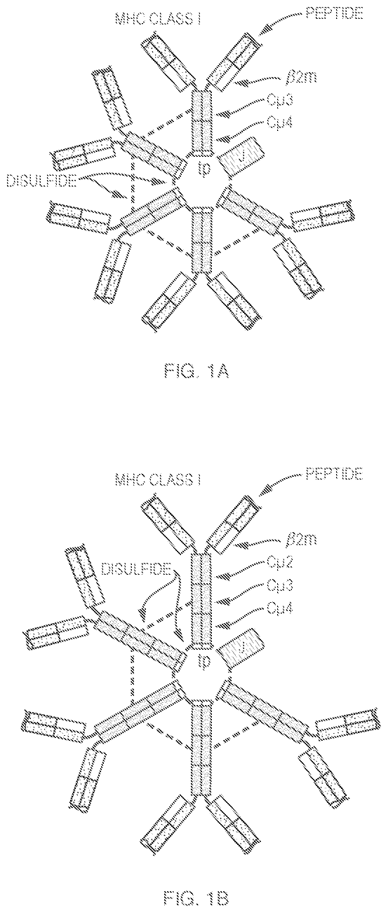

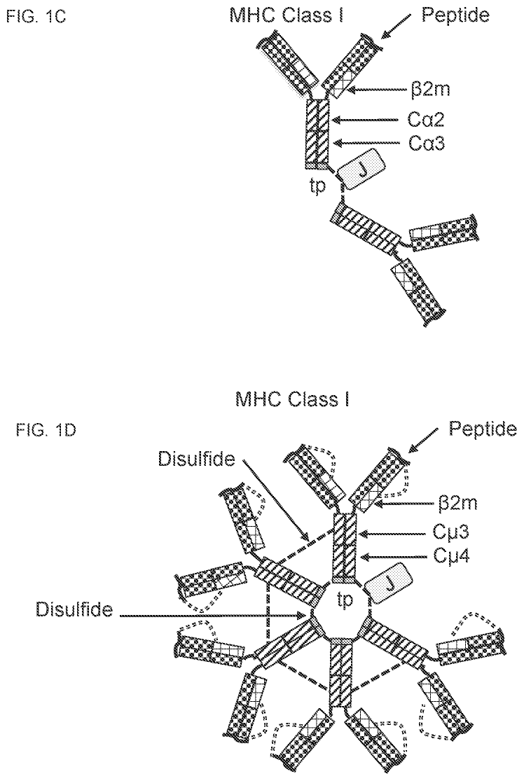

As used herein, the term “multimer” refers to a plurality (two or more) of units, e.g., a plurality of units comprising an MHC moiety-multimerization moiety fusion. The multimerization moiety is comprised of an immunoglobulin heavy chain constant region, which dimerizes to form the immunoglobulin Fc domain. As used herein, a multimer can be a dimer, a trimer, a tetramer, a pentamer, or a hexamer, with respect to the immunoglobulin Fc domain of the multimer (e.g., corresponding to the Ig Fc region within the multimer). However, since the Fc domain is a dimer and presents two MHC moieties, each unit of the multimer is bivalent (i.e., has two peptide-binding regions) and there will be 2× the number of peptide-binding sites as there are Ig subunits in each multimer. Thus, for example, a “hexamer” multimer comprises twelve peptide-binding sites provided by the MHC moiety and a “pentamer” multimer comprises ten peptide-binding sites provided by the MHC moiety. This is illustrated, for example, in FIGS. 1A-1B, and 1D-FIGS. 4A-4B, which each show exemplary IgM pentamer MHC multimers (i.e., five Ig subunits) having ten peptide-binding sites provided by the MHC moiety or FIGS. 1C, 2C, 3C, and 4C, which each show exemplary IgA dimer MHC multimers.

As used herein, “percent identity” between a polypeptide sequence and a reference sequence is defined as the percentage of amino acid residues in the polypeptide sequence that are identical to the amino acid residues in the reference sequence, after aligning the sequences and introducing gaps, if necessary, to achieve the maximum percent sequence identity. Similarly, percent “identity” between a nucleic acid sequence and a reference sequence is defined as the percentage of nucleotides in the nucleic acid sequence that are identical to the nucleotides in the reference sequence, after aligning the sequences and introducing gaps, if necessary, to achieve the maximum percent sequence identity. Alignment for purposes of determining percent sequence identity (e.g., amino acid sequence identity or nucleic acid sequence identity) can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared.

As used herein, “pharmaceutical composition” or “pharmaceutical formulation” refers to the combination of an active agent with a carrier, inert or active, making a composition especially suitable for diagnostic or therapeutic use in vivo or ex vivo.

As used herein, the phrases “pharmaceutically acceptable” and “pharmacologically acceptable,” refer to compounds, molecular entities, compositions, materials, and/or dosage forms that do not produce an adverse, allergic or other untoward reaction when administered to an animal, or a human, as appropriate. For human administration, preparations should meet sterility, pyrogenicity, and general safety and purity standards as required by FDA Office of Biologics standards or its international equivalents. “Pharmaceutically acceptable” and “pharmacologically acceptable” can mean approved or approvable by a regulatory agency of the federal or a state government or the corresponding agency in countries other than the United States, or that is listed in the U.S. Pharmacopoeia or other generally recognized pharmacopoeia for use in animals, and more particularly, in humans.

As used herein, “pharmaceutically acceptable excipient” refers to a substance that aids administration of an active agent to and/or absorption by a subject and can be included in the compositions of the present invention without causing a significant adverse toxicological effect on the patient. Non-limiting examples of pharmaceutically acceptable excipients include water, NaCl, normal saline solutions, such as a phosphate buffered saline solution, emulsions (e.g., such as an oil/water or water/oil emulsions), lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. For examples of excipients, see, e.g., REMINGTON'S PHARMACEUTICAL SCIENCES, 18th Edition (Easton, Pennsylvania: Mack Publishing Company, 1990.

As used herein the terms “polypeptide,” “peptide”, and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. The terms “isolated protein” and “isolated polypeptide” are used interchangeably to refer to a protein (e.g., a soluble, multimeric protein) which has been separated or purified from other components (e.g., proteins, cellular material) and/or chemicals. Typically, a polypeptide is purified when it constitutes at least 60 (e.g., at least 65, 70, 75, 80, 85, 90, 92, 95, 97, or 99) % by weight of the total protein in the sample.

As used herein, “subject” and “patient” are used interchangeably and refer to an organism to be treated by the methods and compositions provided herein. Such organisms are preferably a mammal (e.g., human, mouse, rat, guinea pig, dog, cat, horse, cow, pig, or non-human primate, such as a monkey, chimpanzee, baboon, and rhesus), and more preferably, a human.

The term “substantial identity” indicates that two amino acid sequences, when optimally aligned and compared, are identical, with appropriate insertions or deletions, in at least about 90% of the amino acids and usually at least about 95% of the amino acids. In various embodiments, the two sequences may be at least 90%, 91%, 92%, 93%, 94%, 95%, 96%, 97%, 98%, 99% or 99.5% identical at the amino acid sequence level.

As used herein, the term “tag” refers to an additional molecular component that is affixed to a multimer to thereby label it, for example for detection and/or purification purposes. A “tag” encompasses polypeptide or peptide molecules that can serve as detectable labels or targets used in purification e.g., by affinity chromatography (e.g., 6×His, Flag, V5 tags, described further herein). A “tag” also can be an oligonucleotide component, generally DNA, that provides a means of addressing a target molecule (e.g., a multimer) to which it is joined (described further herein).

As used herein, the term “T cell” refers to a type of white blood cell that can be distinguished from other white blood cells by the presence of a T cell receptor on the cell surface. As will be known to those of skill in the art, there are several subsets of T cells, including, but not limited to, T helper cells (a.k.a. T_{H} cells or CD4⁺ T cells) and subtypes, including T_{H}1, T_{H}2, T_{H}3, T_{H}17, T_{H}9, and T_{FH} cells, cytotoxic T cells (a.k.a. T_{C} cells, CD8⁺ T cells, cytotoxic T lymphocytes, T-killer cells, killer T cells), memory T cells and subtypes, including central memory T cells (T_{CM} cells), effector memory T cells (T_{EM} and T_{EMRA} cells), and resident memory T cells (T_{RM} cells), regulatory T cells (a.k.a. T_{reg} cells or suppressor T cells) and subtypes, including CD4⁺ FOXP3⁺ T_{reg} cells, CD4⁺ FOXP3⁻ T_{reg} cells, Tr1 cells, Th3 cells, and T_{reg}17 cells, natural killer T cells (a.k.a. NKT cells), mucosal associated invariant T cells (MAITs), and gamma delta T cells (γδ T cells), including Vγ9/Vδ2 T cells. The term “T cell cytotoxicity” includes any immune response that is mediated by CD8+ T cell activation.

As used herein, the terms “T cell receptor” and “TCR” refer to a surface protein (e.g., a heterodimeric protein) of a T cell that allows the T cell to recognize an antigen and/or an epitope thereof, typically presented by a major histocompatibility complex (MHC), or a fragment of such surface protein comprising at least its variable domains. Typically, TCRs are heterodimers comprising two different protein chains. In many T cells, the TCR comprises an alpha (α) chain and a beta (β) chain. Each chain, in its native form, typically comprises two extracellular domains, a variable (V) domain and a constant (C) domain, the latter of which is membrane-proximal. The variable domain of α-chain (Vα) and the variable domain of β-chain (VO) each comprise three hypervariable regions that are also referred to as the complementarity determining regions (CDRs) such as CDR1, CDR2, and CDR3. The CDRs, in particular CDR3, are primarily responsible for contacting epitopes and thus define the specificity of the TCR, although CDR1 of the α-chain can interact with the N-terminal part of the antigen, and CDR1 of the β-chain interacts with the C-terminal part of the antigen. All numbering of the amino acid sequences and designation of protein loops and sheets of TCRs is according to the IMGT numbering scheme (the international ImMunoGeneTics information system; Lefranc et al. (2003) DEV. COMP. IMMUNOL., 27:55 77; Lefranc et al. (2005) DEV. COMP. IMMUNOL., 29:185-203). The terms “T cell receptor” and “TCR” also include an “engineered T cell receptor” or “engineered TCR,” such as a recombinantly modified protein comprising a fragment of a naturally occurring TCR that bind a T cell epitope-MHC complex, or a variant of such fragment. For example, an engineered TCR may contain a modified binding cassette (e.g., where one or more CDR sequences or other elements is modified, for example, by introducing corresponding sequences from a different TCR). For example, the α and/or β chain CDR3 sequences of a first TCR identified herein may be introduced into a second, different TCR present in or derived from a given T cell. The TCR may also contain modification, truncation, or deletion of its constant region, hinge region, transmembrane region, and/or intracellular region. For example, at least the transmembrane region and the intracellular region can be deleted to generate a soluble TCR. According, in certain embodiments, a TCR (e.g., engineered TCR) comprises corresponding Vα and Vβ domains that bind a T cell epitope-MHC complex, where the Vα and Vβ domains are derived from a naturally occurring TCR or a variant thereof.

As used herein, a “TCR/pMHC complex” refers to a protein complex formed by binding between a T cell receptor (TCR), or a soluble portion thereof, and a peptide-loaded MHC molecule. Accordingly, a “component of a TCR/pMHC complex” refers to one or more subunits of a TCR (e.g., Vα, Vβ, Cα, Cβ), or to one or more subunits of an MHC or pMHC class I or II molecule.

As used herein, the terms “treat”, “treating” or “treatment” include any effect, for example, lessening, reducing, modulating, ameliorating, inhibiting (such as arresting development, either in a first instance or recurrence), or eliminating, that results in the prevention or improvement of the condition, disease, disorder, and the like, or ameliorating a symptom thereof. Treating can be curing, improving, or at least partially ameliorating (including relieving by regression of a disease state) the disorder. In certain embodiments, treating is curing the disease. As used herein, “reducing” or “reduction” of a symptom or symptoms (and grammatical equivalents of this phrase) means decreasing of the severity or frequency of the symptom(s), or elimination of the symptom(s). Treating includes treatment of a disease in a subject, such as a human.

As used herein, a “variant” refers to a modified form of the protein or polypeptide in which one or more modifications, such as amino acid swaps, substitutions, deletions and/or insertions, have been made such that the amino acid sequence of the variant differs from the parental amino acid sequence from which it is derived. Thus, a “variant” is derived from a parental protein or polypeptide through introduction of one or more modifications. Typically, a “variant” retains “substantial identity” to the parental protein or polypeptide from which it is derived.

### II. Multimer Components

The multimers of the disclosure comprise an MHC moiety that is operatively linked to a multimerization moiety. As provided herein, an MHC moiety comprises (i) an MHC Class I molecule, optionally bound with an MHC Class I-binding peptide; or (ii) an MHC Class II molecule, optionally bound with an MHC Class II-binding peptide. In general, the multimerization moiety comprises at least one constant (C) region and a tailpiece region of IgM or IgA. This multimerization moiety mediates polymerization of the fusion protein into multimers, which can be dimers, trimers, tetramers, pentamers, or hexamers. The resultant multimer is a soluble multivalent molecule that can bind to TCRs recognized by the peptide-MHC portion of the multimer. A composition comprising the multimers can comprise a single type of multimer (e.g., only pentamers or only hexamers). Alternatively, a composition comprising the multimers can comprise a mixture of at least two multimers selected from the group consisting of dimers, trimers, tetramers, pentamers, or hexamers (e.g., a mixture of hexamers and pentamers or a mixture of pentamers and dimers).

Accordingly, in one aspect, the disclosure provides a multimer comprising:
(a) an MHC moiety comprising (i) an MHC Class I molecule, optionally bound with an MHC Class I-binding peptide; or (ii) an MHC Class II molecule, optionally bound with an MHC Class II-binding peptide; and
(b) the multimerization moiety comprises (i) an IgM Cμ4 constant region and an IgM tailpiece region; or (ii) an IgA Cα3 constant region and an IgA tailpiece region.

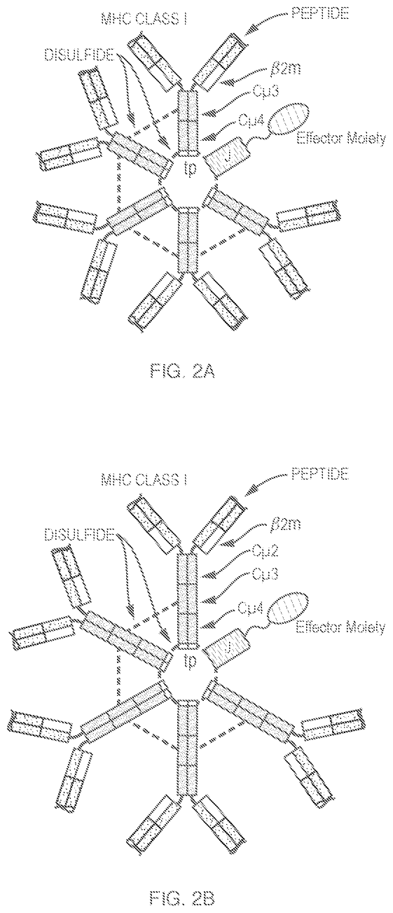

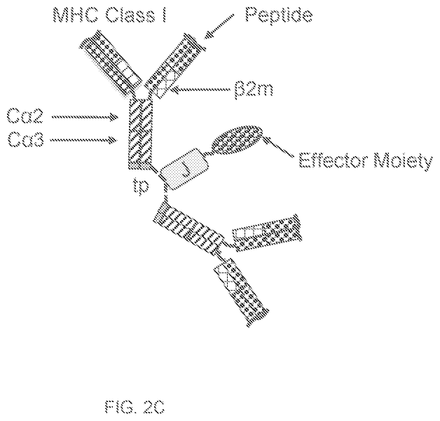

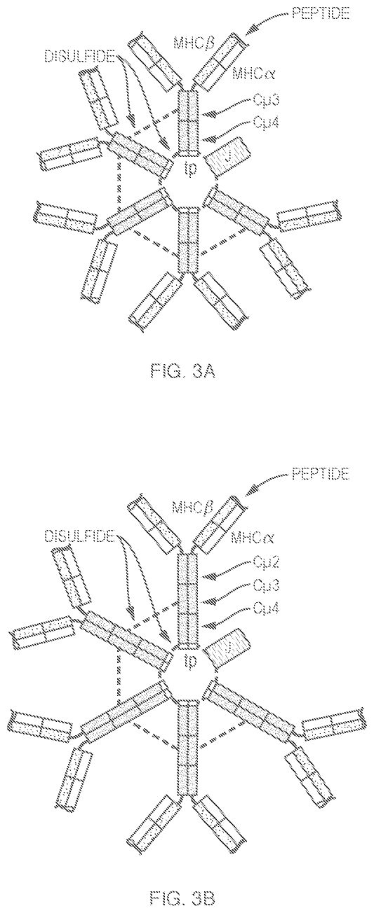

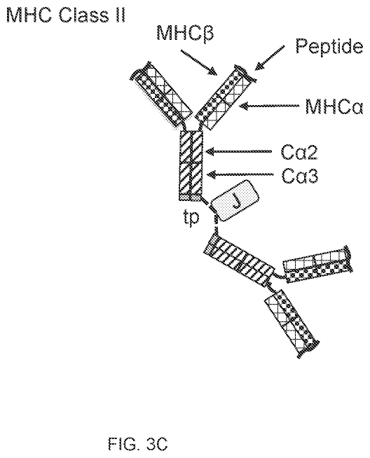

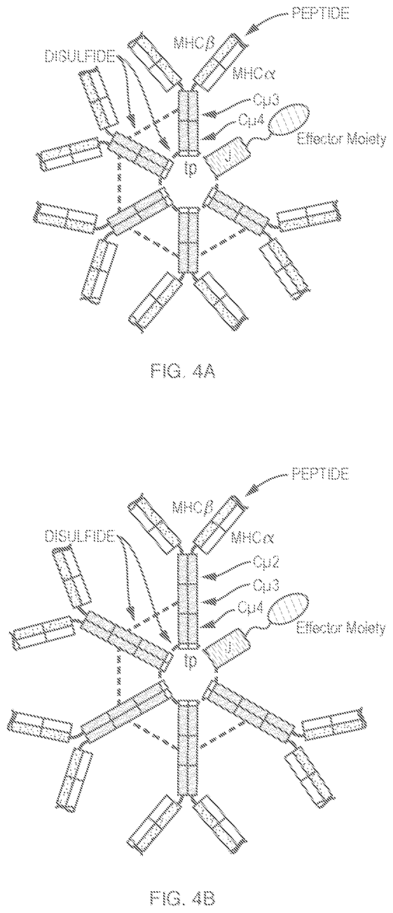

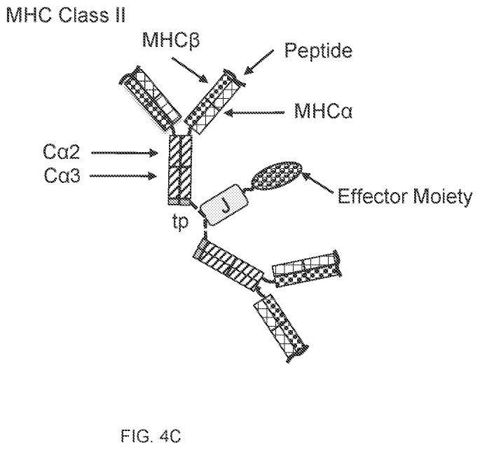

The multimers optionally can also include an immunoglobulin J chain, an effector/targeting moiety and/or a functional moiety. Non-limiting representative multimer structures are illustrated schematically in FIGS. 1A-D, FIGS. 2A-C, FIGS. 3A-C and FIGS. 4A-C. The various components of the multimers are described in further detail below.

### A. MHC Class I Polypeptides

MHC Class I molecules are defined and described herein. The Class I histocompatibility ternary complex contains three parts associated by noncovalent bonds. Human MHC class I genes encode, for example, HLA-A, HLA-B, and HLA-C proteins. Class I HLA protein is a heterodimer composed of a heavy α-chain and a smaller β-chain. The α-chain is encoded by a variant HLA gene, and the R chain is an invariant β2 microglobulin (β2m) polypeptide encoded by a separate region of the human genome. The MHCI heavy chain (also referred to herein as the MHCI α chain) is a polymorphic transmembrane glycoprotein of about 45 kDa containing three extracellular domains, each containing about 90 amino acids (α1 at the N-terminus, α2 and α3), a transmembrane domain of about 40 amino acids and a cytoplasmic tail of about 30 amino acids. The α1 and α2 domains of the MHCI heavy chain contain two segments of a helix that form a peptide-binding groove or cleft. A short peptide typically about 8-11 amino acids binds noncovalently (“fits”) into this groove between the two a helices. The α3 domain of the MHCI heavy chain is proximal to the plasma membrane. The MHCI heavy chain is non-covalently bound to a β2 microglobulin (β2m) polypeptide, forming a ternary complex. In MHCI, the binding groove is closed at both ends by conserved tyrosine residues leading to a size restriction of the bound peptides to usually about 8-11 residues with its C-terminal end docking into the F-pocket.

In one embodiment of a multimer comprising MHCI, the heavy chain and β2m are recombinantly expressed as a single chain molecule, with a flexible linker between the heavy chain and β2m. In one embodiment, a Y84A substitution is introduced into the heavy chain to facilitate formation of the complete MHCI molecule in single chain format. Non-limiting examples of amino acid sequences of single chain format MHCI molecules with the Y84A mutation are shown in SEQ ID NOs: 1, 3, 5, 6, 8, 9, 11, 199-204, 235-237, 248-249, 259-260.

In accordance with the present disclosure, a suitable MHCI molecule can be that of a vertebrate MHC molecule such as a human, a mouse, a rat, a porcine, a bovine or an avian MHC molecule.

In some embodiments, the multimeric MHCI multimers described herein comprise an MHC molecule comprising a human MHC class I protein selected from HLA-A, HLA-B or HLA-C. In some embodiments, the multimer comprises MHC Class I-like molecules (including non-classical MHC Class I molecules) including, but not limited to, CD1d, HLA E, HLA G, HLA F, HLA H, MIC A, MIC B, ULBP-1, ULBP-2, and ULBP-3. The amino acid sequences of the MHCI heavy chains, β2m polypeptides and of MHC Class I like molecules from a variety of vertebrate species are known in the art and publicly available.

In some embodiments, the MHCI heavy chain α domain is human, and comprises, for example, an MHCI heavy chain α domain(s) from a human MHC Class I molecule(s) selected from the group consisting of HLA-A*02:01, HLA-A*01:01, HLA-A*03:01, HLA-A*11:01, HLA-A*24:02, HLA-B*07:02, HLA-C*04:01, HLA-C*07:02, HLA-B*08:01, HLA-B*35:01, HLA-B*57:01, HLA-B*57:03, HLA-E, HLA-C*16:01, HLA-C*08:02, HLA-C*07:01, HLA-C*05:01, HLA-B*44:02, HLA-A*29:02, HLA-B*44:03, HLA-C*03:04, HLA-B*40:01, HLA-C*06:02, HLA-B*15:01, HLA-C*03:03, HLA-A*30:01, HLA-B*13:02, HLA-C*12:03, HLA-A*26:01, HLA-B*38:01, HLA-B*14:02, HLA-A*33:01, HLA-A*23:01, HLA-A*25:01, HLA-B*18:01, HLA-B*37:01, HLA-B*51:01, HLA-C*14:02, HLA-C*15:02, HLA-C*β2:02, HLA-B*27:05, HLA-A*31:01, HLA-A*30:02, HLA-B*42:01, HLA-C*17:01, HLA-B*35:02, HLA-B*39:06, HLA-C*03:02, HLA-B*58:01, HLA-A*33:03, HLA-A*68:02, HLA-C*01:02, HLA-C*07:04, HLA-A*68:01, HLA-A*32:01, HLA-B*49:01, HLA-B*53:01, HLA-B*50:01, HLA-A*02:05, HLA-B*55:01, HLA-B*45:01, HLA-B*52:01, HLA-C*12:02, HLA-B*35:03, HLA-B*40:02, HLA-B*15:03, HLA-A*74:01, HLA-A*02:02, HLA-A*02:06, HLA-A*68:03 and/or HLA-C*08:01. The amino acid sequences of soluble forms of these MHCI molecules (lacking signal sequence and transmembrane domain) are shown in SEQ ID NOs: 30-96, and 261-264 respectively.

In some embodiments, the MHCI multimers provided herein comprise the α1 and α2 domains of an MHCI heavy chain. In some embodiments, MHCI multimers provided herein comprise the α1, α2, and α3 domains of an MHCI heavy chain.

In some embodiments, the two or more pMHCI or pMHCI-like polypeptides in an MHCI multimer comprise a β2-microglobulin polypeptide, e.g., a human β2-microglobulin. In some embodiments, the β2-microglobulin is wild-type human β2-microglobulin. In some embodiments, the β2-microglobulin comprises an amino acid sequence that is at least 80, 85, 90, 95, or 99% identical to the amino acid sequence of the human 32 microglobulin, the full-length sequence of which is set forth in SEQ ID NO: 25 (UniProt Id. No. P61769).

In some embodiments, the multimeric protein comprises a soluble MHCI polypeptide. In some embodiments the MHC multimeric protein comprises a soluble MHCI α domain and a β2-microglobulin polypeptide. In some embodiments, the soluble MHCI protein comprises the MHCI heavy chain α1 domain and the MHCI heavy chain α2 domain.

Other embodiments of the MHC moiety are composed only of the MHCI heavy chain and β2-microglobulin, without loaded peptide, referred to herein as “empty” MHCI.

Alternatively, in some embodiments, the MHCI monomer is a fusion protein comprising a β2m polypeptide or functional fragment thereof covalently linked to the MHCI heavy chain or functional fragment thereof. In some embodiments the carboxy (—COOH) terminus of β2m is covalently linked to the amino (—NH₂) terminus of the MHCI heavy chain.

In some embodiments, the MHC monomers comprise one or more linkers between the individual components of the MHCI monomer. In some embodiments, the MHCI monomer comprises a heavy chain fused with β2m through a linker. In some embodiments, the linker between the heavy chain and β2m is a flexible linker, e.g., made of glycine and serine. In some embodiments, the flexible linker between the heavy chain and β2m is between 5-20 residues long. In other embodiments, the linker between the heavy chain and β2m is rigid with a defined structure, e.g. made of amino acids like glutamate, alanine, lysine, and leucine. In one embodiment, the linker is a (G₄S)₄ linker (SEQ ID NO: 141).

### B. MHC Class II Polypeptides

MHC class II molecules are defined and described herein. Human MHC class II genes encode, for example, HLA-DM, HLA-DO, HLA-DP, HLA-DQ, and HLA-DR proteins. Class II HLA protein is a heterodimer composed of a and β chains both encoded by variant HLA genes. The antigen binding groove is where a peptide epitope binds. Since the antigen binding groove of MHC class II molecules is open at both ends, the groove can accommodate longer peptide epitopes than MHC class I molecules. Peptide epitopes presented by MHC class II molecules typically are about 15-24 amino acid residues in length.

MHCII molecules of the present disclosure can be vertebrate MHCII molecules such as a human, a mouse, a rat, a porcine, a bovine or an avian MHCII molecule. In some embodiments, the multimeric MHCII multimers described herein comprise an MHC molecule comprising a human MHC class II protein selected from HLA-DR, HLA-DQ, HLA-DX, HLA-DO, HLA-DZ, and HLA-DP. The amino acid sequences of the MHCII α and β chains from a variety of vertebrate species, including humans, are known in the art and publicly available.

In some embodiments, the human MHCII molecule is of an allotype selected from the group consisting of DRB1*0101 (see, e.g., Cameron et al. (2002) J. IMMUNOL. METHODS, 268:51-69; Cunliffe et al. (2002) EUR. J. IMMUNOL., 32:3366-3375; Danke et al. (2003) J. IMMUNOL., 171:3163-3169), DRB1*1501 (see, e.g., Day et al. (2003) J. CLIN. INVEST, 112:831-842), DRB5*0101 (see, e.g., Day et al., ibid), DRB1*0301 (see, e.g., Bronke et al. (2005) HUM. IMMUNOL., 66:950-961), DRB1*0401 (see, e.g., Meyer et al. (2000) PROC. NATL. ACAD. SCI. USA, 97:11433-11438; Novak et al. (1999) J. CLIN. INVEST, 104:R63-R67; Kotzin et al. (2000) PROC. NATL. ACAD. SCI. USA, 97:291-296), DRB1*0402 (see, e.g., Veldman et al. (2007) CLIN. IMMUNOL., 122:330-337), DRB1*0404 (see, e.g., Gebe et al. (2001) J. IMMUNOL., 167:3250-3256), DRB1*1101 (see, e.g., Cunliffe, ibid; Moro et al. (2005) BMC IMMUNOL., 6:24), DRB1*1302 (see, e.g., Laughlin et al. (2007) INFECT. IMMUNOL. 75:1852-1860), DRB1*0701 (see, e.g., Danke, ibid), DQA1*0102 (see, e.g., Kwok et al. (2000) J. IMMUNOL., 164:4244-4249), DQB1*0602 (see, e.g., Kwok, ibid), DQA1*0501 (see, e.g., Quarsten et al. (2001) J. IMMUNOL., 167:4861-4868), DQB1*0201 (see, e.g., Quarsten, ibid), DPA1*0103 (see, e.g., Zhang et al. (2005) EUR. J. IMMUNOL., 35:1066-1075; Yang et al. (2005) J. CLIN. IMMUNOL., 25:428-436), and DPB1*0401 (see, e.g., Zhang, ibid; Yang, ibid).

In some embodiments, the MHCII molecule is human, and comprise, for example, an MHCII α and β chains selected from the group consisting of HLA-DRA*01:01, HLA-DRB1*01:01, HLA-DRB1*01:02, HLA-DRB1*03:01, HLA-DRB1*04:01, HLA-DRB1*04:04, HLA-DRB1*07:01, HLA-DRB1*08:01, HLA-DRB1*10:01, HLA-DRB1*11:01, HLA-DRB1*11:04, HLA-DRB1*13:01, HLA-DRB1*13:02, HLA-DRB1*14:01, HLA-DRB1*15:01, HLA-DRB1*15:03, HLA-DQA1*01:01, HLA-DQB1*05:01, HLA-DQA1*01:02, HLA-DQB1*06:02, HLA-DQA1*03:01, HLA-DQB1*03:02, HLA-DQA1*05:01, HLA-DQB1*02:01, HLA-DQB1*03:01, HLA-DQB1*03:03, HLA-DQB1*04:02, HLA-DQB1*05:03, HLA-DQB1*06:03, HLA-DQB1*06:04 HLA-DRB1*04:03, HLA-DRB1*04:07, HLA-DRB1*08:02, HLA-DRB1*09:01, HLA-DRB1*12:01, HLA-DRB1*13:03, HLA-DRB1*15:02, HLA-DRB5*01:01, HLA-DPA1*01:03, and/or HLA-DPB1*04:01. The amino acid sequences of soluble forms of these MHCII chains (lacking signal sequence and transmembrane domain) are shown in SEQ ID NOs: 97-126, respectively. HLA-DR sequences are shown in SEQ ID NOs: 97-112 and 265-272. HLA-DQ sequences are shown in SEQ ID NOs: 113-126 and HLA-DP sequences are shown in SEQ ID NOs: 273-274.

In certain embodiments, an additional amino acid sequence can be appended to the C-terminal sequence of the α or β chain of the MHCII molecule, for example for purposes of labeling and/or for attaching a moiety that mediates attachment (e.g., conjugation) to the multimerization domain. For example, an avitag (that mediates binding through the biotin binding site of Sav) can be appended, such as an avitag with a Myc tag (SEQ ID NO: 149), an avitag with a Myc tag and a His tag (SEQ ID NO: 150) or an avitag with a His tag and a FLAG tag (SEQ ID NO: 151).

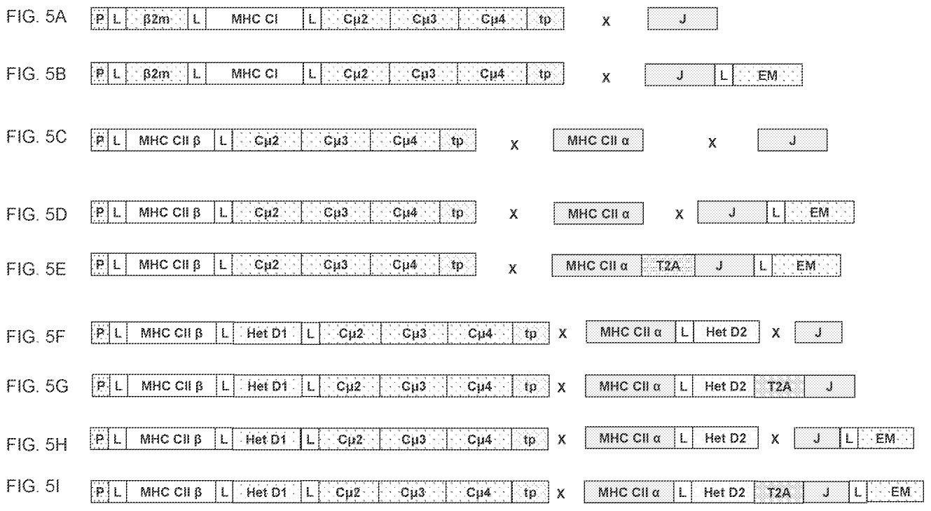

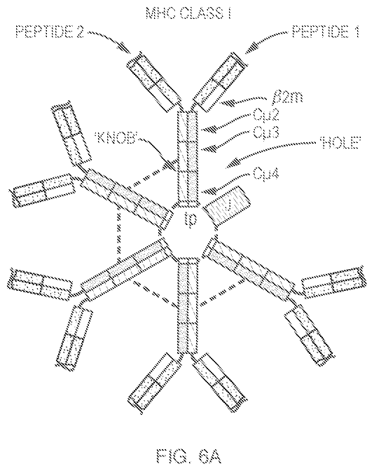

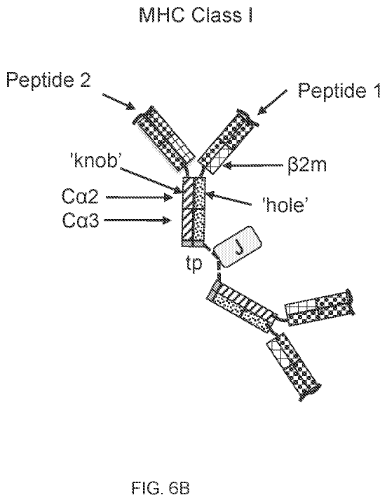

In certain embodiments, heterodimerization pairs can be appended to the C-terminal sequence of the α and/or β chains of the MHCII molecule. Non-limiting examples of such heterodimerization pair sequences include Fos and Jun (e.g., having the amino acid sequences shown in SEQ ID NOs: 152 and 153, respectively), acidic and basic leucine zippers (e.g., having the amino acid sequences shown in SEQ ID NOs: 154 and 155, respectively), knob and hole sequences (e.g., having the amino acid sequences shown in SEQ ID NOs: 156 and 157, respectively) for knobs-into-holes technology or spytag and spycatcher sequences (e.g., having the amino acid sequences shown in SEQ ID NOs: 158 and 159, respectively). The use of leucine zippers with MHC Class II is described further in Voller and Stem (2008) IMMUNOLOGY 123:305-313, and demonstrated as part of an exemplary construct herein as set forth in Example 3 using a Fos/Jun variant as described in Mason J M et al. (2006) PROC NATL ACAD SC USA, 13; 103(24):8989-94). Additional heterodimerization pairs can include designed coiled-coils with increased affinity and stability, such as FosW, which is a variants of wild type Fos, described in Mason et al. (2006) PROC. NATL. ACAD. SC. USA, 103:8989-8994. Exemplary constructs incorporating a heterodimerization motif into MHC II α and β are shown in FIGS. 5F-5I.

Typically, an MHCII-binding peptide is encoded in the expression construct adjacent to the coding sequences of the MHCII chains such that the peptide and a digestible linker are encoded in the construct (e.g., upstream of (N-terminally)) and in operative linkage with the coding sequences for the MHCII chain. In certain embodiments, an expression tag is also encoded upstream or downstream of the peptide. Non-limiting examples of such tags include a FLAG tag (e.g., having the amino acid sequence shown in SEQ ID NO: 143), a 6×His tag (e.g., having the amino acid sequence shown in SEQ ID NO: 144), a V5 tag (e.g., having the amino acid sequence shown in SEQ ID NO: 145), a Strep-Tag (e.g., having the amino acid sequence shown in SEQ ID NO: 146), a Protein C tag (e.g., having the amino acid sequence shown in SEQ ID NO: 147) and/or a Myc tag (e.g., having the amino acid sequence shown in SEQ ID NO: 148).

In some embodiments, in constructs comprising a heterodimerization moiety, a flexible linker may precede and/or follow the moiety. In some embodiments, in constructs comprising a heterodimerization moiety, there may be no linker preceding and/or following the moiety.

In some embodiments, the pMHCII multimers described herein comprise the α1 and α2 domains of an MHCII α chain and the β1 and β2 domains of an MHCII β chain. In some embodiments, the multimer described herein comprises only the α1 and β31 domains of an MHCII heavy chain. In other embodiments, the pMHCII multimers comprise an α-chain and a β-chain combined with a peptide. Other embodiments include an MHCII molecule comprised only of α-chain and β-chain (so-called “empty” MHC II without loaded peptide), a truncated α-chain (e.g. the α1 domain) combined with full-length β-chain, either empty or loaded with a peptide, a truncated β-chain (e.g. the β1 domain) combined with a full-length α-chain, either empty or loaded with a peptide, or a truncated α-chain combined with a truncated β-chain (e.g. α1 and β1 domain), either empty or loaded with a peptide.

In some embodiments, the multimeric protein comprises a soluble MHCII polypeptide, which can be a soluble MHCII lacking transmembrane and intracellular domains.

### C. Multimerization Moiety

The multimerization moiety portion of the multimer comprises at least one region (domain) from IgM or IgA sufficient such that multimerization occurs. In various embodiments, at least the most C-terminal constant region of either an IgM or IgA molecule (Cμ4 or Cα3, respectively) is used in the multimerization moiety, as well as a tailpiece region from either IgM or IgA. A constant region or a plurality of constant regions from IgM can be paired with a tailpiece region from either IgM or IgA. Likewise, a constant region(s) from IgA can be paired with a tailpiece region from either IgM or IgA. In some embodiments, the multimerization moiety can include one, two, three or four C regions from IgM or IgA (as described further below). In some embodiments, the multimerization moiety uses only the IgM or IgA tailpiece region without any constant regions.

The native IgM constant region contains four distinct constant region domains, Cμ1, Cμ2, Cμ3, and Cμ4, each approximately 110 amino acids in length. Human IgM has four alleles and the constant region sequences from any of these four alleles can be used in the multimers. The constant region also includes an 18 amino acid “tailpiece”, the amino acid sequence of which in humans is shown in SEQ ID NO: 129. The predominant native form of human IgM is a pentamer, although hexamers can also form. However, hexameric IgM does not contain a J chain (the presence of a J chain prevents hexamer formation), whereas pentameric IgM may or may not include a J chain. The Cμ2 and Cμ3 domains and the tailpiece each include a cysteine that form a disulfide bond with another β chain. Deletion of the tailpiece has been shown to prevent the formation of polymeric IgM (Davis et al. (1989) J. IMMUNOL., 43:1352-1357).

Accordingly, in one embodiment, the multimerization moiety comprises an IgM Cμ4 constant region and an IgM or IgA tailpiece region.

In one embodiment, the multimerization moiety contains one additional IgM C region. Thus, the multimerization moiety can comprise (i) IgM Cμ3 and Cμ4 constant regions and IgM or IgA tailpiece region, (ii) IgM Cμ2 and Cμ4 constant regions and IgM or IgA tailpiece region or (iii) IgM Cμ1 and Cμ4 constant regions and IgM or IgA tailpiece region.

In one embodiment, the multimerization moiety contains two additional IgM C regions. Thus, the multimerization moiety can comprise (i) IgM Cμ2, Cμ3 and Cμ4 constant regions and IgM or IgA tailpiece region, (ii) IgM Cμ1, Cμ3 and Cμ4 constant regions and IgM or IgA tailpiece region or (iii) IgM Cμ1, Cμ2 and Cμ4 constant regions and IgM or IgA tailpiece region.

In one embodiment, the multimerization moiety contains three additional IgM C regions, namely IgM Cμ1, Cμ2, Cμ3 and Cμ4 constant regions and IgM or IgA tailpiece region.

The native IgA constant region contains three distinct constant region domains, Cal, Cα2 and Cα3. Human IgA has two isotypes, A1 and A2, each of which have three alleles. The constant region sequences from any of these six alleles (three from A1 and three from A2) can be used in the multimers. The IgA constant region also includes an 18 amino acid “tailpiece”, the amino acid sequence of which in humans is shown in SEQ ID NO: 130. The predominant native form of human IgA is a dimer, facilitated by the J chain, although it can also exist naturally as a monomer.

Accordingly, in one embodiment, the multimerization moiety comprises an IgA Cα3 constant region and an IgA or IgM tailpiece region.

In one embodiment, the multimerization moiety contains one additional IgA C region. Thus, the multimerization moiety can comprise (i) IgA Cα2 and Cα3 constant regions and IgA or IgM tailpiece region or (ii) IgA Cal and Cα3 constant regions and IgA or IgM tailpiece region.

In one embodiment, the multimerization moiety contains two additional IgA C regions, namely IgA Cal, Cα2 and Cα3 constant regions and IgA or IgM tailpiece region.

As used herein, a “multimerization moiety” is intended to encompass variants, mutants and otherwise modified forms of the IgM or IgA constant region sequences that still retain the polymerization ability of the IgM or IgA constant region sequences from which the modified form is derived. Such variants include modifications to eliminate N-linked glycosylation motifs, modifications that substitute a non-cysteine residue with a cysteine residue to thereby improve stability by introducing additional disulfide bonds, modifications that substitute a cysteine residue with a non-cysteine residue to eliminate disulfide bonds, variants with improved half-life or other pharmacokinetic property, variants with increased or decreased effector function (e.g., to alter complement activation or neutrophil activation) or variants that enhance the manufacturability of the multimer.

Specific modifications of Ig constant regions that alter effector function have been described in the art and can be applied to the multimerization moiety of the multimer to, e.g., alter disulfide bridging, alter half-life or alter complement activation ability. For instance, modification such as substitutions at certain amino acid positions may be used to impact certain activities (e.g., complement dependent cytotoxicity, or FcuR binding). For example, as is known to those of skill in the art, mutations in residues 309-316, based on the sequential Fc numbering scheme present at Uniprot Ref. ID P01871, can be introduced into an IgM C region to minimize complement-dependent cytotoxicity (CDC) (see e.g., WO2018/187702A2; see also Sharp et al., (2019) PROC. NATL. ACAD. SCI. USA, 116: 11900-11905, reporting DLPSP residues 432-436 (residues 309-313 using UniProt P01871 numbering) are critical for C1 complement protein binding). An additional example, as is known to those of skill in the art, is described as a P311G mutation which can be introduced into an IgM C region to minimize CDC (see e.g., Chen et al. (2020) MABS, 12:1818436). Exemplary IgM C region sequences with mutations to minimize complement activation include SEQ ID NOS: 193-198. Exemplary peptide HLA-IgM complement-reduced sequences are set forth in SEQ ID NOs.: 199-204.

Other modifications such as mutations at Q387R can be introduced to reduce binding to FcuR receptors (Nyambora, et al. (2020) BBA PROTEINS AND PROTEOMICS, 1868: 140266). Additional constant region modifications to increase half-life and/or reduce binding to FcuR receptor are known in the art and include those described in U.S. Patent Publication 20200239572.

### D. MHC Binding Peptides

In certain embodiments of methods and constructs provided herein, MHC multimer expression constructs encode an MHC-binding peptide that binds to the MHC molecule also encoded by the construct such that upon expression in a host cell, MHC molecules loaded with peptide are expressed by the host cell. For example, in one embodiment, the peptide can be a placeholder peptide that is known to bind the MHC molecule used in the multimer. Methods for peptide exchange are known in the art, for example, including for Class I: UV-mediated cleavage (Rodenko et al., NATURE PROTOCOLS 1:1120-1132, 2006), dipeptide exchange (Saini et al., (2015) PROC. NATL. ACAD. SCI. USA, 112:202-207), TAPBPR-mediated exchange (Overall et al., (2020) NATURE COMM., 11:1909), or for Class II, exchange under acidic conditions as in Day et al., (2003) J. CLIN. INVEST., 112:831-842. In another embodiment, the peptide can be a peptide of interest or epitope peptide that is known to be involved in a disease or disorder, which can be incorporated in a multimer to be used for therapeutic purposes.

In some embodiments, a peptide may be linked (e.g., covalently linked) to β2m via a linker. For example, in some embodiments, a cognate peptide may be covalently linked to an MHC class I molecule via a flexible linker to β2m. In other embodiments, an MHC Class II molecule (e.g., α or β) may be linked to a cognate peptide via a flexible linker.

For example, in some embodiments, the present disclosure provides multimers comprising a Major Histocompatibility Complex (MHC) moiety operatively linked to a multimerization moiety, wherein the MHC moiety comprises an MHC molecule bound with a cognate peptide. To give but one example, in some embodiments, the present disclosure provides a multimer comprising an MHC moiety operatively linked to a multimerization moiety, wherein: (a) the MHC moiety comprises an MHC molecule; and (b) the multimerization moiety comprises (i) an IgM Cμ4 constant region and a tailpiece region; or (ii) an IgA Cα3 constant region and a tailpiece region, wherein the tailpiece region in (b)(i) or (b)(ii) is an IgM tailpiece region or an IgA tailpiece region.

In an alternative embodiment, “empty” MHC multimers are prepared (e.g., as provided herein, by modifying certain amino acid residues to stabilize an empty MHC) and then subsequently loaded with peptide by methods known in the art.

In some embodiments, an MHC moiety may comprise an MHC molecule optionally bound with a cognate peptide. For example, in some such embodiments, a peptide may be bound in a covalent manner, or a non-covalent manner. In some such embodiments, covalent or non-covalent binding may be accomplished via a linker. In some embodiments, no peptide is bound. For example, as described herein, the present disclosure contemplates that, in some embodiments, an “empty” stabilized MHCI peptide could be used to make an “empty” IgM multimer in which a peptide could be exogenously loaded.

In some embodiments, the peptide of the multimer is thermolabile. In some embodiments, the peptide is thermolabile at a temperature between about 30-37° C. In some embodiments, the peptide is labile at a temperature at or above 30° C., at or above 32° C., at or above 34° C., at or above 35° C., at or above 36° C., or at about 37° C. Thermal labile peptides and methods of identifying and producing thermal labile peptides have been described (e.g., WO 93/10220; WO 2005/047902; U.S. Patent Publication 2008/0206789; Luimstra et al. (2019) CURR. PROTOC. IMMUNOL., 126(1):e85; Luimstra et al. (2018) J. EXP. MED., 215(5):1493-1504).

In some embodiments the peptide is labile at an acidic pH. In some embodiments, the peptide is labile between about pH 2.5 and 6.5. In some embodiments, the peptide is labile at a pH of about 2.5-6.0, 3.0-6.0, 3.0-6.5, 3.5-6.0 3.5-6.5, 4.0-6.0, 4.0-6.5, 4.5-6.0, 4.5-6.5, 5.0-6.0, 5.0-6.5, 5.0, 5.5, 6.0 or 6.5. In some embodiments, the peptide is labile at a basic pH. In some embodiments, the peptide is labile between about pH 9-11. In some embodiments, the peptide is labile at or above pH 9, at or above pH 9.5, at or about pH 10, at or about pH 10.5, or at or about pH 11. Methods of generating and using pH sensitive peptides are described in WO 93/10220; U.S. Patent Publication 2008/0206789; and Cameron et al. (2002) J. IMMUNOL. METH., 268:51-69.

In some embodiments, the peptide comprises a cleavable moiety. Various types of cleavable moieties are known in the art and include, for example, moieties that are cleaved by photoirradiation, enzymes, nucleophilic or electrophilic agents, reducing and oxidizing reagents (e.g., reviewed in Leriche et al. (2012) BIORG. MED. CHEM., 20(2):571-582).

In some embodiments, the peptide comprises a chemoselective moiety. In some embodiments, the chemoselective moiety comprises a sodium dithionite sensitive azobenzene linker, wherein the azobenzene comprises at least one aromatic group comprising an electron-donor group and is located between two amino acid residues. Azobenzine linkers and methods for chemoselective peptide exchange are known in the art, for example, as described in U.S. Pat. No. 10,400,024.

In some embodiments, the peptide comprises a cleavable moiety that is cleaved upon exposure to an aminopeptidase. In some embodiments, the cleavage of the amino acid residue occurs via the use of a methionine aminopeptidase. The methionine aminopeptidase can cleave a methionine from a peptide when the amino acid residue at position two is, for example, glycine, alanine, serine, cysteine, or proline. In some embodiments, the cleavable moiety comprises a thrombin cleavage domain.

### 1. MHC Class I Peptides

For MHCI multimers, MHCI monomers typically are expressed such that they are loaded with a peptide to facilitate proper folding of the MHCI monomers to produce peptide-loaded MHCI (p*MHCI) within the multimers. Examples of placeholder peptides and methods of inducing folding MHCI heavy chains and β2-microglobulin in vitro in the presence of a placeholder peptide have been described in the art (e.g., Bakker et al. (2008) PROC. NATL. ACAD. SCI. USA, 105:3825-3830; Rodenko et al. (2006) NAT. PROT., 1: 1120-1132).

In some embodiments, the MHCI-binding peptide is an HLA-A, HLA-B or HLA-C peptide. In some embodiments, the peptide is an HLA-A1 peptide (e.g., A1:01 binding peptide). In some embodiments, the peptide is an HLA-A2 peptide (e.g., A02-01 binding peptide). In other embodiments, the peptide is an HLA-A3 peptide (e.g., A3:01 binding peptide), an HLA-A11 peptide (e.g., A11:01 binding peptide), an HLA-A24 peptide (e.g., A24:02 binding peptide), an HLA-B7 peptide (e.g., B7:02 binding peptide), an HLA-B8 peptide (e.g., B8:01 binding peptide), an HLA-B15 peptide (e.g., B15:01 binding peptide), an HLA-B35 peptide (e.g., B35:01 binding peptide), an HLA-B40 peptide (e.g., B40:01 binding peptide), an HLA-B58 peptide (e.g., B58:01 binding peptide), an HLA-C3 peptide (e.g., C3:04 binding peptide), an HLA-C4 peptide (e.g., C4:01 binding peptide), an HLA-C7 peptide (e.g., C7:02 binding peptide), an HLA-C8 peptide (e.g., C8:01 binding peptide), an HLA-B57 peptide (e.g., B57:01 binding peptide or B57:03 binding peptide), an HLA-E peptide (e.g., HLA-E binding peptide), an HLA-C16 peptide (e.g., C16:01 binding peptide), an HLA-C08 peptide (e.g., C08:02 binding peptide), an HLA-C07 peptide (e.g., C07:01 binding peptide), an HLA-C05 peptide (e.g., C05:01 binding peptide), an HLA-B44 peptide (e.g., B44:02 binding peptide), an HLA-A29 peptide (e.g., A29:02 binding peptide), an HLA-B44 peptide (e.g., B44:03 binding peptide), an HLA-C06 peptide (e.g., C06:02 binding peptide), an HLA-C03 peptide (e.g., C03:03 binding peptide), an HLA-A30 peptide (e.g., A30:01 binding peptide), an HLA-B13 peptide (e.g., B13:02 binding peptide), an HLA-C12 peptide (e.g., C12:03 binding peptide), an HLA-A26 peptide (e.g., A26:01 binding peptide), an HLA-B38 peptide (e.g., B38:01 binding peptide), an HLA-B14 peptide (e.g., B14:02 binding peptide), an HLA-A33 peptide (e.g., A33:01 binding peptide), an HLA-A23 peptide (e.g., A23:01 binding peptide), an HLA-A25 peptide (e.g., A25:01 binding peptide), an HLA-B18 peptide (e.g., B18:01 binding peptide), an HLA-B37 peptide (e.g., B37:01 binding peptide), an HLA-B51 peptide (e.g., B51:01 binding peptide), an HLA-C14 peptide (e.g., C14:02 binding peptide), an HLA-C15 peptide (e.g., C15:02 binding peptide), an HLA-Cβ2 peptide (e.g., Cβ2:02 binding peptide), an HLA-B27 peptide (e.g., B27:05 binding peptide), an HLA-A31 peptide (e.g., A31:01 binding peptide), an HLA-A30 peptide (e.g., A30:02 binding peptide), an HLA-B42 peptide (e.g., B42:01 binding peptide), an HLA-C17 peptide (e.g., C17:01 binding peptide), an HLA-B35 peptide (e.g., B35:02 binding peptide), an HLA-B39 peptide (e.g., B39:06 binding peptide), an HLA-C03 peptide (e.g., C03:02 binding peptide), an HLA-A33 peptide (e.g., A33:03 binding peptide), an HLA-A68 peptide (e.g., A68:02 binding peptide), an HLA-C01 peptide (e.g., C01:02 binding peptide), an HLA-C07 peptide (e.g., C07:04 binding peptide), an HLA-A68 peptide (e.g., A68:01 binding peptide), an HLA-A32 peptide (e.g., A32:01 binding peptide), an HLA-B49 peptide (e.g., B49:01 binding peptide), an HLA-B53 peptide (e.g., B53:01 binding peptide), an HLA-B50 peptide (e.g., B50:01 binding peptide), an HLA-A02 peptide (e.g., A02:05 binding peptide), an HLA-B55 peptide (e.g., B55:01 binding peptide), an HLA-B45 peptide (e.g., B45:01 binding peptide), an HLA-B52 peptide (e.g., B52:01 binding peptide), an HLA-C12 peptide (e.g., C12:02 binding peptide), an HLA-B35 peptide (e.g., B35:03 binding peptide), an HLA-B40 peptide (e.g., B40:02 binding peptide), an HLA-B15 peptide (e.g., B15:03 binding peptide), an HLA-A74 peptide (e.g., A74:01 binding peptide), an HLA-A02 peptide (e.g., A02:02 binding peptide), an HLA-A02 peptide (e.g., A02:06 binding peptide), and/or an HLA-A68 peptide (e.g., A68:03 binding peptide). In some embodiments, the peptide is a synthetic peptide.

In some embodiments, the MHCI molecule is an HLA-A*02:01 molecule and the peptide is an HLA-A*02:01-restricted peptide. In one embodiment, the HLA-A*02:01-restricted peptide is a CMV pp65 peptide epitope. In one embodiment, the CMV pp65 peptide epitope comprises the amino acid sequence NLVPMVATV (SEQ ID NO: 19). In some embodiments, the CMV pp65 peptide epitope consists of the amino acid sequence NLVPMVATV (SEQ ID NO: 19). Other HLA-A*02:01-restricted peptide sequences include the heteroclitic MART-1 sequence ELAGIGILTV (SEQ ID NO: 20), the HPV sequence YMLDLQPETT (SEQ ID NO: 160), the HSV sequence SLPITVYYA (SEQ ID NO: 161) and the WT-1 sequence RMFPNAPYL (SEQ ID NO: 162).

In other embodiments, the HLA-A2 peptide is KILGFVFTV (SEQ ID NO: 163) or GILGFVFTL (SEQ ID NO: 164). In yet other embodiments, the MHCI/peptide combination can be selected from the group consisting of A*1:01, VTEHDTLLY (SEQ ID NO: 165); A*3:01, TVRSHCVSK (SEQ ID NO: 166); A*11:01, TTFLQTMLR (SEQ ID NO: 167); A*24:02, RYPLTFGWCF (SEQ ID NO: 168); B*7:02, RPHERNGFTVL (SEQ ID NO: 169); B*35:01, IPSINVHHY (SEQ ID NO: 170); C*3:04, FVYGGSKTSL (SEQ ID NO: 171), B*8:01, FLRGRAYGL (SEQ ID NO: 172); C*7:02, RYRPGTVAL (SEQ ID NO: 173); C*4:01, QYDPVAALF (SEQ ID NO: 174); B*15:01, GQFLTPNSH (SEQ ID NO: 175); B*40:01, KEVNSQLSL (SEQ ID NO: 176); B*58:01, VSFIEFVGW (SEQ ID NO: 177); and C*8:02, IAPWYAFAL (SEQ ID NO: 178).

In some embodiments, the peptide is a dipeptide. In some embodiments, the dipeptide binds to the F pocket of the MHCI binding groove. In some embodiments, the second amino acid of the dipeptide is hydrophobic. In some embodiments, the dipeptide is selected from the group consisting of glycyl-leucine (GL), glycyl-valine (GV), glycyl-methionine (GM), glycyl-cyclohexylalanine (GCha), glycyl-homoleucine (GHle) and glycyl-phenylalanine (GF). Methods for producing and using dipeptides as placeholder peptides are publicly available, for example, as described in Saini et al. (2015) PROC. NATL. ACAD. SCI. USA, 112:202-207.

In some embodiments, the peptide further comprises a detectable label. In some embodiments, the detectable label is a fluorescent label. In some embodiments, the fluorescent label is attached to a cysteine residue in the peptide.

### 2. MHC Class II Peptides

In accordance with the present disclosure, MHCII multimers comprise MHCII monomers, which monomers are typically expressed such that they are loaded with an MHCII-binding peptide to facilitate proper folding of the MHCII monomers to produce peptide loaded MHCII (p*MHCII) within the multimers. In various embodiments, the peptide is a peptide that binds HLA-DR, HLA-DQ, HLA-DX, HLA-DO, HLA-DZ or HLA-DP. In some embodiments, the peptide is a synthetic peptide.

In one embodiment, the MHCII placeholder peptide is a CLIP peptide, such as having the amino acid sequence PVSKMRMATPLLMQA (SEQ ID NO: 22). In one embodiment, the CLIP peptide is cleavable. In one embodiment, the MHCII monomers are synthesized with the cleavable CLIP peptide covalently attached, such as by synthesis of single-chain MHC class II chain-peptide complexes, directed by engineering peptide-specific complementary DNA (cDNA) sequences proximal to the β-chain cDNA (see e.g., Day et al. (2003) J. CLIN. INVEST., 112:831-842). Cleavage of the covalent linkage between the CLIP peptide (as the placeholder peptide) and MHCII thus allows for peptide exchange with other MHCII-binding peptides.

Other MHCII binding peptides have been described in the art that can be used, based on appropriate pairing of an MHCII molecule and its known MHCII binding peptide. Non-limiting examples of known MHCII molecule/MHCII binding peptide pairs include: DRA1*0101/DRB1*0401 and the immunodominant peptide of hemagglutinin, HA₃₀₇₋₃₁₉ (see Novak et al. (1999) J. CLIN. INVEST., 104:R63-R67) and HLA-DR*1101 and tetanus-toxoid (TT)-derived β2 peptide (TT₈₃₀₋₈₄₄) (see Cecconi et al. (2008) CYTOMETRY, 73A:1010-1018).

### E. Immunoglobulin J Chain

In certain embodiments, a multimer includes an immunoglobulin J chain (also referred to herein simply as a “J chain”). As provided herein, a J chain contains eight cysteine residues, two of which link the Cμ chains of IgM or the Cα chains of IgA via disulfide bridges. The J chain is not essential for polymerization of either IgM or IgA and thus is not a required component for multimerization of a multimer to occur. However, the J chain can enhance multimerization of IgM or IgA and thus, for at least this reason, may be included in a multimer of the present disclosure. Additionally, the J chain plays a role in complement activation. For example, IgM hexamers that lack a J chain are more effective at activating complement than IgM pentamers that include a J chain. Thus, in situations in which it is desirable for a multimer to activate complement, omission of the J chain from the multimer may be desirable. Alternatively, in situations in which it is desirable to avoid or limit complement activation (e.g., to avoid damage to epithelial membranes from complement activation), inclusion of the J chain in the multimer is desirable.

To include a J chain in the multimer, an expression construct encoding the J chain can be co-transfected into a host cell along with the other multimer components (see e.g., FIGS. 1-5 and Example 5). In one embodiment, the J chain is expressed from a construct that also encodes one or more other components of the multimer (e.g., as shown in FIG. 5E, in which the MHCII α chain sequences are encoded by the same construct as the J chain, with a ribosomal skipping sequence in between). Additionally, the J chain can serve as the component to which an effector moiety (also referred to as a targeting moiety, as described and provided herein) and/or one or more additional functional moieties (as described herein) can be coupled (e.g., by attachment or other association, e.g., non-covalent association). This can be accomplished recombinantly using an expression construct in which the J chain sequences are operatively linked to the effector moiety sequence (encoding the effector/targeting moiety and comprising one or more effector domains) or functional moiety sequence (as illustrated schematically in FIGS. 2A-B and FIGS. 4A-B for PD-L1 as the effector moiety). A linker sequence can be positioned between the J chain sequence and the effector moiety sequence (encoding the effector/targeting moiety) or functional moiety sequence. Suitable linker sequences are described herein.

The full-length sequence of human J chain is shown in SEQ ID NO: 26. Example 5 describes the preparation of non-limiting examples of J chain-effector moiety fusion constructs in which at least a portion of the extracellular domain of PD-L1 (i.e., a PD-1-binding portion) is used as the effector/targeting moiety. In one embodiment, the effector/targeting moiety is Domain 1 (D1) of PD-L1, which can be linked to the C-terminus of the J chain (e.g., a fusion construct as shown in SEQ ID NO: 27). In one embodiment, the effector/targeting moiety is the Extracellular Domain (ECD) of PD-L1, which can linked to the C-terminus of the J chain (e.g., a fusion construct as shown in SEQ ID NO: 28). Additional exemplary effector/targeting moieties are described herein and may be coupled to a multimer through use of a J-chain.

### F. Effector/Targeting Moiety

In certain embodiments, a multimer of the disclosure includes an effector moiety or a targeting moiety. As used herein, the term “effector moiety” is used interchangeably with the term “targeting moiety” to refer to a region of the multimer that comprises one or more effector domains, e.g., one or more domains of a molecule that have or can impart biological activity, such as an immunoglobulin antigen binding site. For example, an effector moiety (or targeting moiety) can “effect” or “target” specific biological responses that contribute to immune modulation, for example, by preventing or driving (i.e., trigger or advance) certain downstream activities (e.g., PD-L1 binding to PD1 to drive immune exhaustion). For avoidance of doubt, the present disclosure contemplates that the effector/targeting moiety can function in a target-specific manner via spatial localization through the pMHC multimer to particular TCRs. That is, the pMHC multimer(s) is/are responsible for imparting spatial localization to a target, while the effector/targeting moiety can bind to a receptor or target (e.g., a cellular component) on or near a cell/tissue where the cognate TCR for the pMHC multimer is located.

In one embodiment, the effector/targeting moiety also imparts at least one additional functional property to the multimer, such as enhanced immune cell modulation or enhanced target cell killing (e.g., as compared to otherwise identical multimers not comprising the effector/targeting moiety). In various embodiments, the effector/targeting moiety can be or can comprise, for example, a ligand or binding fragment thereof, a cytotoxic molecule, an antibody, antibody fragment, peptide, or antibody-drug conjugate, scFV, or VHH that binds to a surface molecule of interest and/or effects one or more changes in the target cell (e.g., changes downstream activity, e.g., internalizes effector and causes cell death, etc.).

In multimers that include a J chain, an effector moiety (e.g., comprising one or more effector domains) is typically appended to either the C-terminus or N-terminus of the J chain (see, e.g., Example 5). For multimers that lack a J chain, an effector moiety can be attached to the MHC subunit that is not fused to the multimerization domain. For example, in a multimer in which the MHC Class II β chain is fused to the multimerization domain, the effector moiety can be fused to the MHC Class II α chain. Alternatively, the effector moiety can be attached to the C-terminus of the multimerization moiety.

In some embodiments, an effector/targeting moiety may be or comprise any agent that causes or prevents a downstream biological effect upon binding. For instance, in some embodiments, an effector/targeting moiety may be or comprise a binding agent or any binding portion thereof that can bind and act on a T cell receptor of a targeted T cell.

In some embodiments, an effector/targeting moiety acts in cis, binding to the T cell that it will impact directly (i.e., binding to a TCR on a targeted T cell). In some embodiments, an effector/targeting moiety acts in trans, binding to and influencing another different population of cells (e.g., T cells or NK cells) that, in turn, influence a targeted population of T cells.

In one embodiment, the effector/targeting moiety binds to a T cell surface antigen. In some such embodiments, the present disclosure contemplates that binding of the effector/targeting moiety leads to redirection of those T cells expressing the surface antigen to the multimer such that the T cells function differently from prior to the binding (e.g., an activated T cell becomes suppressed, or may be killed or otherwise removed, a suppressed T cell becomes stimulated, etc.). Additionally, in some embodiments, binding of the effector/targeting moiety to those T cells may modulate T cell activation and/or effector function. Non-limiting examples of suitable effector/targeting moieties for redirection of T cells (and possible mechanism of action) include molecules such as checkpoint protein agonists, anti-inflammatory cytokines, apoptosis-inducing/driving agents, T cell stimulators or expanders, or cytokines that operate in particular ways depending on context (e.g., cytokine-mediated context dependent suppression). For example, in some embodiments, an effector/targeting moiety is or comprises known or putative checkpoint protein agonists such as PD-L1 (Domain 1 or Full ECD) or an checkpoint protein agonist such as anti-PD1, anti-TIGIT, anti-CTLA4, anti-TIM3, anti-Lag3, anti-LILRB1, or anti-LILRB2 agonist antibodies, or a binding domain or portion (e.g., an antigen-binding fragment) of any of these antibodies. In some embodiments, an effector/targeting moiety is or comprises a TNF receptor superfamily agonist (e.g., a TNF family molecule), such as an anti-CD137 agonist antibody or an antigen-binding fragment thereof, or CD137L. In some embodiments, an effector/targeting moiety comprises HLA-G or an extracellular fragment thereof. In some embodiments, an effector/targeting moiety is or comprises an anti-inflammatory cytokine such as TGFβ or IL10 (promotion of Treg phenotype and context-dependent immunosuppression effects). In some embodiments, an effector/targeting moiety is or comprises an inducer of cell death. For example, in some embodiments, the inducer of cell death is a TNF receptor superfamily agonist, such as FasL (a driver of apoptosis by engaging with Fas on target cells), anti-Fas agonist antibody or an antigen-binding fragment (e.g., scFv) thereof (a driver of apoptosis by engaging with Fas on target cells), TRAIL (TNF related apoptosis inducing ligand that activates DR4 and DR5 to induce apoptosis), or anti-DR4 or anti-DR5 agonist antibody or an antigen-binding fragment thereof (induction of apoptosis). In some embodiments, the inducer of cell death is a cytotoxic agent such as a microtubule inhibitor or DNA damaging agent (cell death/elimination). In some embodiments, an effector/targeting moiety is or comprises an agent to enhance or induce expansion of regulatory T cells such as TRAIL, TNFR2 agonist scFv, anti-TRAIL R1, or anti-TRAIL R2 agonist. Furthermore, it is contemplated that two or more such effector moieties, either separately, or conjugated or fused together, may be included in the multimeric compositions described herein.

In one embodiment, the effector/targeting moiety acts, in trans, as a T cell or NK cell engager by binding to a T cell or an NK cell surface antigen, thereby leading to redirection of those T or NK cells expressing the surface antigen to the MHC multimer, or to MHC-multimer-bound cells. Additionally, binding of the effector/targeting moiety to those T or NK cells can modulate T or NK cell activation and/or effector function. Non-limiting examples of suitable targeting moieties for redirection of T cells include anti-CD3 and for redirection of NK cells effector/targeting moieties that bind CD56, CD16, NKp46, NKp30, CD64 or NKG2D.

In one embodiment, the effector/targeting moiety binds to a macrophage surface antigen, thereby leading to redirection of those macrophages expressing the surface antigen to the multimer, or to multimer-bound cells. Additionally, binding of the effector/targeting moiety to those macrophages can modulate macrophage activation and/or effector function. A non-limiting example of a suitable effector/targeting moiety for redirection of macrophages is a moiety that binds CD14.

In one embodiment, the effector/targeting moiety binds to a neutrophil surface antigen, thereby leading to redirection of those neutrophils expressing the surface antigen to the multimer, or to multimer-bound cells. Additionally, binding of the effector/targeting moiety to those neutrophils can modulate neutrophil activation and/or effector function. Non-limiting examples of suitable targeting moieties for redirection to neutrophils include moieties that bind CD16b and CD177.

In another embodiment, the effector/targeting moiety is an Fc receptor (FcR) binding molecule, thereby redirecting the multimer to FcR-expressing cells, which includes a variety of immune cells (e.g., B cells, follicular dendritic cells, NK cells, macrophages, neutrophils, eosinophils). For example, the effector/targeting moiety can be an FcγR-binding moiety, which can be used e.g., to drive ADCC activity by the multimer.

In another embodiment, the effector/targeting moiety is either a cytokine or is directed against (binds to) a cytokine. For example, the effector/targeting moiety can be a cytokine that retains its receptor binding capacity, thereby directing the multimer to cells expressing the cytokine receptor. Non-limiting examples of suitable cytokines as targeting moieties can bind include IL-2 or a mutein thereof, IL-12 and IL-15.

In another embodiment, the effector/targeting moiety is or comprises a checkpoint inhibitor, for example, an antagonist anti-PD1, anti-TIGIT, anti-CTLA4, anti-TIM3, anti-Lag3, anti-LILRB1, or anti-LILRB2 antibody or an antigen-binding fragment of any of these antibodies.

In addition to the foregoing, various modified J chain constructs have been described in the art that comprise a J chain fused to an effector/targeting moiety, which also are suitable for use in the multimers of the disclosure. Non-limiting examples include the modified J chains described in U.S. Pat. No. 9,951,134 and U.S. Patent Publication No. 2019/0185570.

By way of non-limiting example, as provided herein, in certain embodiments, an MHC multimer comprises an immunoglobulin J chain linked to an effector/targeting moiety. For instance, to give but one example, a J chain-effector/targeting moiety fusion comprising an effector/targeting moiety, such as PD-L1 (or a PD-1-binding portion thereof), or anti-CD3 scFv from OKT3 can be fused, recombinantly to a C- or N-terminus of a J-chain.

Alternatively or additionally, in some embodiments, an effector/targeting moiety as provided herein, can be fused at the N-terminus of a J-chain separated by a linker moiety as described herein (e.g., (G₄S)ₙ linker), and, optionally, comprise Flag and/or His-tags. To give but a few examples, effector/targeting moieties such as a PD-L1-D1, PD-L1-ECD, or anti-CD3 scFv as described herein, can be fused at the N-terminus of a J-chain separated by a (G₄S)ₙ linker, and, optionally, comprise Flag and/or His-tags.

The full-length sequence of human immunoglobulin J chain is shown in SEQ ID NO: 26. To give several non-limiting examples, fusion constructs comprising a human J chain and an effector moiety, along with a linker may be composed. For example, such fusion constructs may be designed as follows: (i) the human J chain with a PD-L1 Domain 1 (D1) at the C-terminus, separated by a (G₄S)₃ linker, such as that set forth by the amino acid sequence shown in SEQ ID NO: 27 or PD-L1-D1 at the N-terminus of the J-chain separated by a (G₄S)₅ linker, and, optionally with C-terminal Flag and His-tags as set forth in SEQ ID NO: 246; (ii) the human J chain with a PD-L1 extracellular domain (ECD) at the C-terminus, separated by a (G₄S)₃ linker, such as that set forth by the amino acid sequence shown in SEQ ID NO: 28 or PD-L1-ECD at the N-terminus of the J-chain separated by a (G₄S)₅ linker, and, optionally with C-terminal Flag and His-tags as set forth in SEQ ID NO: 245; (iii) the human J chain with an anti-CD3 scFv fused recombinantly to the N-terminus of the J-chain separated by a (G₄S)₃ linker, and, optionally with a C-terminal Flag and His tag as set forth in SEQ ID NO: 247; and (iv) the human J chain with an anti-CD3 scFv fused recombinantly to the C-terminus of the J-chain separated by a (G₄S)₃ linker, and, optionally with a C-terminal Flag and His tag as set forth in SEQ ID NO: 275.

Such J chain-effector moiety constructs as provided by the present disclosure can be transfected into a host cell and coexpressed with MHC multimer construct(s), such as those described herein, to prepare multimers comprising a J chain and an effector/targeting moiety.

### G. Additional Functional Moieties

In certain embodiments, a multimer provided by the disclosure includes one or more additional heterologous functional moieties. Such a “functional moiety” as used herein refers to one or more additional domains or regions that are incorporated into the multimer to thereby impart one or more functional properties of interest to the multimer, but, like an effector/targeting moiety, does not primarily serve to direct or redirect the location of the multimer. A “heterologous” functional moiety refers to the domain or region being from a molecule (e.g., protein, polypeptide) other than the MHC used in the MHC moiety and the Ig C region(s) used in the multimerization domain. Non-limiting examples of heterologous functional moieties include moieties that increase the half-life, or other pharmacokinetic properties, of the multimer, cytokines (e.g., IL-2, IL-12, IL-15) and cytokine receptors (e.g., IL-2R, IL-12R, IL-15R). A heterologous functional moiety can be incorporated into a multimer in a similar manner as that described above for incorporating an effector/targeting moiety.

### H. Linkers

In the multimer compositions, in certain embodiments, a linker (also referred to as a spacer) is positioned between the MHC moiety and the multimerization moiety. Linkers can also be positioned between other domains or regions of various components of the multimer. In some embodiments, a multimer does not comprise a linker. For example, in some embodiments, multimers provided by the present disclosure comprise MHC and IgM constant domains that do not comprise a linker in between.

The terms “linker” or “spacer” denotes a linear amino acid chain of natural and/or synthetic origin. The linkers described herein are designed to ensure that polypeptides conjugated to each other can perform their biological activity by allowing the polypeptides to fold correctly and to be presented properly. The linker may contain repetitive amino acid sequences or sequences of naturally occurring polypeptides. In some embodiments, a peptide linker has a length of from 2 to 50 amino acids. In some embodiments, a peptide linker is between 3 and 30 amino acids, between 5 to 25 amino acids, between 5 to 20 amino acids, or between 10 and 20 amino acids.

In certain embodiments, the sequence of the linker serves primarily to provide additional space (distance) between domains/regions of a fusion protein. In other embodiments, the sequence of the linker imparts one or more additional functionalities, such as a protease cleavage site to allow cleavage of a fusion protein containing the linker or a ribosomal skipping site to allow for translation of two distinct polypeptides from a fusion mRNA, as described further herein.

In one embodiment, the linker is a flexible linker, e.g., composed of a glycine-serine-rich sequence, such as the linker shown in SEQ ID NO: 131. In another embodiment, the spacer linker is a rigid linker, e.g., composed of a proline-rich sequence, such as the linker shown in SEQ ID NO: 132. In yet another embodiment, the spacer linker is a flexible-rigid linker, comprising both a flexible region (e.g., a glycine-serine-rich sequence) and a rigid region (e.g., a proline-rich sequence), such as the linker shown in SEQ ID NO: 133.

In various other embodiments, the peptide linker is rich in glycine, glutamine, and/or serine residues. These residues are arranged e.g. in small repetitive units of up to five amino acids. This small repetitive unit may be repeated for one to five times. At the amino- and/or carboxy-terminal ends of the multimeric unit up to six additional arbitrary, naturally occurring amino acids may be added. Other synthetic peptidic linkers are composed of a single amino acid, which is repeated between 10 to 20 times and may comprise at the amino- and/or carboxy-terminal end up to six additional arbitrary, naturally occurring amino acids. All peptidic linkers can be encoded by a nucleic acid molecule and therefore can be recombinantly expressed. As the linkers are themselves peptides, the polypeptide connected by the linker are connected to the linker via a peptide bond that is formed between two amino acids.

Suitable peptide linkers are well known in the art, and are disclosed in, e.g., U.S. Patent Publications 2010/0210511 and US2010/0179094, and U.S. Patent Publication 2012/0094909. Other linkers are provided, for example, in U.S. Pat. No. 5,525,491; Alfthan et al. (1995) PROTEIN ENG., 8:725-731; Shan et al. (1999) J. IMMUNOL., 162:6589-6595; Newton et al. (1996) BIOCHEMISTRY, 35:545-553; Megeed et al. (2006) BIOMACROMOLECULES, 7:999-1004; and Perisic et al. (1994) STRUCTURE, 12:1217-1226.

In some embodiments, the linker is synthetic. As used herein, the term “synthetic” with respect to a linker includes peptides (or polypeptides) which comprise an amino acid sequence (which may or may not be naturally occurring) that is linked in a linear sequence of amino acids to a sequence (which may or may not be naturally occurring) to which it is not naturally linked in nature. For example, the linker may comprise non-naturally occurring polypeptides which are modified forms of naturally occurring polypeptides (e.g., comprising a mutation such as an addition, substitution or deletion) or which comprise a first amino acid sequence (which may or may not be naturally occurring). Preferably, a linker will be relatively non-immunogenic and not inhibit any non-covalent association among monomer subunits of a binding protein.

In some embodiments, the linker is a Gly-Ser polypeptide linker, i.e., a peptide that consists of glycine and serine residues. One exemplary Gly-Ser polypeptide linker comprises the amino acid sequence (Gly₄Ser)ₙ, wherein n=1-6 (SEQ ID NO: 134). In certain embodiments, n=1. In certain embodiments, n=2. In certain embodiments, n=3. In certain embodiments, n=4. In certain embodiments, n=5. In certain embodiments, n=6. Another exemplary Gly-Ser polypeptide linker comprises the sequence SSSSGSSSSGSAA (SEQ ID NO: 135). Another linker comprises only glycine, e.g., G₅ linkers (GGGGG; SEQ ID NO: 136). Another exemplary Gly-Ser polypeptide linker comprises the amino acid sequence Ser(Gly₄Ser)ₙ, wherein n=1-10 (SEQ ID NO: 137). In certain embodiments, n=1. In certain embodiments, n=2. In certain embodiments, n=3, i.e., Ser(Gly₄Ser)₃. In certain embodiments, n=4, i.e., Ser(Gly₄Ser)₄. In certain embodiments, n=5. In certain embodiments, n=6. In certain embodiments, n=7. In certain embodiments, n=8. In certain embodiments, n=9. In certain embodiments, n=10.

Other exemplary linkers include GS linkers (i.e., (GS)ₙ), GGSG linkers (i.e., (GGSG)n), wherein n=1-5 (SEQ ID NO: 138), GSAT linkers (SEQ ID NO: 139), SEG linkers, GGS linkers (i.e., (GGSGGS)ₙ) (SEQ ID NO: 140), wherein n is a positive integer (e.g., 1, 2, 3, 4, or 5), (Gly₄Ser)₄ (GGGGSGGGGSGGGGSGGGGS; SEQ ID NO: 141) and (GS)₂AG₂SGSG₃S linkers (GSGSAGGSGSGGGS; SEQ ID NO: 142). Other suitable linkers for use in multimeric fusion proteins can be found using publicly available databases, such as the Linker Database (ibi.vu.nl/programs/linkerdbwww). The Linker Database is a database of inter-domain linkers in multi-functional enzymes which serve as potential linkers in novel multimeric fusion proteins (see, e.g., George et al. (2002) PROTEIN ENGINEERING, 15:871-9).

In one embodiment, the linker comprises an amino acid sequence selected from the group consisting of SEQ ID NOs: 131-142.

In certain embodiments, the sequence of the linker imparts a functionality to the linker beyond simply serving as a spacer. For example, in one embodiment, the linker comprises a protease recognition (cleavage) site such that the linker can be cleaved by a protease. In one embodiment the protease is an amino-peptidase. In another embodiment, the protease is a methionine amino-peptidase. In yet other embodiments, the protease is selected from FXa, thrombin, TEV, HRV3C, and furin.

In certain embodiments, the linker sequence comprises a ribosomal skipping site (sequence) such as a 2A peptide. In an expression construct, when such a ribosomal skipping site is included in a linker positioned between two polypeptide sequences, translation of the transcribed mRNA results in production of the two polypeptides separately, rather than as a fusion protein. The 2A peptide sequences share a core sequence motif of DXEXNPGP, wherein X is any amino acid (SEQ ID NO: 179). Non-limiting examples of suitable 2A peptide sequences include T2A (EGRGSLLTCGDVEENPGP; SEQ ID NO: 180), P2A (ATNFSLLKQAGDVEENPGP; SEQ ID NO: 181), E2A (QCTNYALLKLAGDVESNPGP; SEQ ID NO: 182) and F2A (VKQTLNFDLLKLAGDVESNPGP; SEQ ID NO: 183).

Additionally, an optional Gly-Ser-Gly (GSG) tripeptide can be added to the N-terminal end of a 2A peptide to enhance efficiency.

### I. Labels

In certain embodiments, one or more detectable labels are attached to a multimer of the disclosure. As used herein, a “detectable label” is any molecule or functional group that allows for the detection of a biological or chemical characteristic or change in a system, such as the presence of a target substance in the sample. For example, multimers can be conjugated with a fluorescent label, allowing for identification of binding the multimer to a target molecule or cell, for example via flow cytometry or microscopy. Cells can also be selected based on a fluorescence label through, e.g., fluorescence or magnetic activated cell sorting.

Non-limiting examples of detectable labels include protein tags (e.g., that can be detected with an anti-tag antibody), fluorophores, chromophores, electro chemiluminescent labels, bioluminescent labels, polymers, polymer particles, bead or other solid surfaces, gold or other metal particles or heavy atoms, spin labels, radioisotopes, enzyme substrates, haptens, antigens, Quantum Dots, aminohexyl, pyrene, nucleic acids or nucleic acid analogs, or proteins, such as receptors, peptide ligands or substrates, enzymes, and antibodies (including antibody fragments).

Numerous protein tags are known in the art that can be appended at the N- or C-terminus of a polypeptide or protein to facilitate detection (e.g., using an anti-tag antibody) and/or purification (e.g., using affinity chromatography). Non-limiting examples of protein tags include a Flag tag (e.g., SEQ ID NO: 143), a 6× histidine tag (His6) (e.g., SEQ ID NO: 144), a V5 tag (e.g., SEQ ID NO: 145), a strep tag (e.g., SEQ ID NO: 146), a protein C tag (e.g., SEQ ID NO: 147), a myc tag (e.g., SEQ ID NO: 148), an avitag-Myc sequence (e.g., SEQ ID NO: 149), an avitag-Myc-His6 sequence (e.g., SEQ ID NO: 150) and/or an avitag-His6-Flag sequence (e.g., SEQ ID NO: 151), and combinations thereof.

Additional tags suitable for use in the methods and compositions provided herein include affinity tags, including but not limited to enzymes, protein domains, or small polypeptides which bind with high specificity to a range of substrates, such as carbohydrates, small biomolecules, metal chelates, antibodies, etc., to allow rapid and efficient purification of proteins. Solubility tags enhance proper folding and solubility of a protein and are frequently used in tandem with affinity tags. Sequences encoding such a tag(s) can be incorporated into an expression construct of the disclosure, such as at the C-terminus or N-terminus of the multimer-encoding regions to thereby incorporate a detectable tag into the expressed polypeptide.

Examples of enzymes which may be used comprise horse radish peroxidase (HRP), alkaline phosphatase (AP), β-galactosidase (GAL), glucose-6-phosphate dehydrogenase, β-N-acetylglucosaminidase, β-glucuronidase, invertase, Xanthine Oxidase, firefly luciferase and glucose oxidase (GO). Examples of commonly used substrates for horse radish peroxidase (HRP) include 3,3′-diaminobenzidine (DAB), diaminobenzidine with nickel enhancement, 3-amino-9-ethylcarbazole (AEC), Benzidine dihydrochloride (BDHC), Hanker-Yates reagent (HYR), Indophane blue (IB), tetramethylbenzidine (TMB), 4-chloro-1-naphtol (CN), α-naphtol pyronin (.α.-NP), o-dianisidine (OD), 5-bromo-4-chloro-3-indolylphosphate (BCIP), Nitroblue tetrazolium (NBT), 2-(p-iodophenyl)-3-p-nitrophenyl-5-phenyltetrazolium chloride (INT), tetranitro blue tetrazolium (TNBT), delta.-bromo-chloro-S-indoxyl-β-D-galactoside/ferro-ferricyanide (BCIG/FF). Examples of commonly used substrates for Alkaline Phosphatase include Naphthol-AS-B1-phosphate/fast red TR (NABP/FR), Naphthol-AS-MX-phosphate/fast red TR (NAMP/FR), Naphthol-AS-B1-phosphate/fast red TR (NABP/FR), Naphthol-AS-MX-phosphate/fast red TR (NAMP/FR), Naphthol-AS-B1-phosphate/new fuschin (NABP/NF), bromochloroindolylphosphate/nitroblue tetrazolium (BCIP/NBT), b-Bromo-chloro-S-indolyl-β-delta-galactopyranoside (BCIG).

Examples of luminescent labels which may be used include luminol, isoluminol, acridinium esters, 1,2-dioxetanes and pyridopyridazines. Examples of electrochemiluminescent labels include ruthenium derivatives. Examples of radioactive labels which may be used include radioactive isotopes of iodide, cobalt, selenium, hydrogen, carbon, sulfur, and phosphorous.

Some “detectable labels” also include “color labels,” in which the biological change or event in the system may be assayed by the presence of a color, or a change in color. Examples of “color labels” are chromophores, fluorophores, chemiluminescent compounds, electrochemiluminescent labels, bioluminescent labels, and enzymes that catalyze a color change in a substrate.

“Fluorophores” as described herein are molecules that emit detectable electro-magnetic radiation upon excitation with electro-magnetic radiation at one or more wavelengths. A large variety of fluorophores are known in the art and are developed by chemists for use as detectable molecular labels and can be conjugated to the multimers provided herein. Examples include FLUORESCEIN™. or its derivatives, such as FLUORESCEIN®-5-isothiocyanate (FITC), 5-(and 6)-carboxyFLUORESCEIN®, 5- or 6-carboxyFLUORESCEIN®,6-(FLUORESCEIN®)-5-(and 6)-carboxamido hexanoic acid, FLUORESCEIN® isothiocyanate, rhodamine or its derivatives such as tetramethyl rhodamine and tetramethylrhodamine-5-(and -6) isothiocyanate (TRITC). Other fluorophores include: coumarin dyes such as (diethyl-amino)coumarin or 7-amino-4-methylcoumarin-3-acetic acid, succinimidyl ester (AMCA); sulforhodamine 101 sulfonyl chloride (TexasRed® or TexasRed® sulfonyl chloride; 5-(and -6)-carboxyrhodamine 101, succinimidyl ester, also known as 5-(and -6)-carboxy-X-rhodamine, succinimidyl ester (CXR); lissamine or lissamine derivatives such as lissamine rhodamine B sulfonyl Chloride (LisR); 5-(and -6)-carboxyFLUORESCEIN®, succinimidyl ester (CFI); FLUORESCEIN®5-isothiocyanate (FITC); 7-diethylaminocoumarin-3-carboxylic acid, succinimidyl ester (DECCA); 5-(and -6)-carboxytetramethyl-rhodamine, succinimidyl ester (CTMR); 7-hydroxycoumarin-3-carboxylic acid, succinimidyl ester (HCCA); 6->FLUORESCEIN®.-5-(and -6)-carboxamidolhexanoic acid (FCHA); N-(4,4-difluoro-5,7-dimethyl-4-bora-3a,4a-diaza-3-indacenepropionic acid, succinimidyl ester; also known as 5,7-dimethylBODIPY® propionic acid, succinimidyl ester (DMBP); “activated FLUORESCEIN® derivative” (FAP), available from Probes, Inc.; eosin-5-isothiocyanate (EITC); erythrosin-5-isothiocyanate (ErlTC); and Cascade® Blue acetylazide (CBAA) (the O-acetylazide derivative of 1-hydroxy-3,6,8-pyrene-trisulfonic acid). Yet other potential fluorophores useful in this disclosure include fluorescent proteins such as green fluorescent protein and its analogs or derivatives, fluorescent amino acids such as tyrosine and tryptophan and their analogs, fluorescent nucleosides, and other fluorescent molecules such as Cy2, Cy3, Cy 3.5, CY5™, CY5™5, Cy 7, IR dyes, Dyomics dyes, phycoerythrine, Oregon green 488, pacific blue, rhodamine green, and Alexa dyes. Yet other examples of fluorescent labels include conjugates of R-phycoerythrin orallophycoerythrin, inorganic fluorescent labels such as particles based on semiconductor material like coated CdSe nanocrystallites.

In yet other embodiments, a multimer of the disclosure comprises an identifier tag or label. that facilitates identification of the multimer. An “identifier” as used herein refers to a readable representation of data that provides information, such as an identity, that corresponds with the identifier. For instance, in some embodiments, an identifier is or comprises an oligonucleotide barcode. Typically, an oligonucleotide barcode is a unique oligonucleotide sequence ranging from 10 to more than 50 nucleotides. The barcode has shared amplification sequences in the 3′ and 5′ ends, and a unique sequence in the middle. This sequence can be revealed by sequencing and can serve as a specific barcode for a given molecule. The use of barcode technology is well known in the art, see for example Shiroguchi et al. (2012) PROC. NATL. ACAD. SCI. USA, 109(4):1347-52; and Smith et al. (2010) NUCLEIC ACIDS RESEARCH, 38(13)11:e142. Further methods and compositions for using barcode technology include those described in U.S. Patent Publication No. 2016/0060621. Standard methods for preparing barcode oligonucleotides and conjugating them to molecules of interest are known in the art.

### J. Modifications

In certain embodiments, the multimers of the disclosure comprise one or more modifications as compared to the wild-type version of the MHC molecule (Class I or Class II), the MHC-binding peptide and/or the Ig-derived multimerization domain. Such modifications can be introduced to impart new features to the multimer and/or to eliminate undesired features of the multimer.

For example, in one embodiment, a component of the multimer can be mutated to eliminate putative or confirmed N-linked or O-linked glycosylation sites to thereby alter the glycosylation of the component.

In another embodiment, a component of the multimer can be mutated to introduce one or more disulfide bridges to increase stability of the component itself or the multimer as a whole. Disulfide bridging can be introduced, for example, into an MHC chain or an MHC-binding peptide to enhance stability. The crystal structure of certain MHC molecules are known in the art (e.g., 1HXY.pdb; Petersson et al. (2001) EMBO J., 20:3306-3312) and can be used to design appropriate cysteine mutations. Example 4 describes introduction of two disulfide bridges into an MHC Class I heavy chain sequence to increase the stability of empty MHC Class I monomers. In another embodiment, one or more disulfide bridges can be introduced into an MHC-binding peptide to create a “disulfide-locked” peptide to enhance stability of the peptide-MHC interaction. In another embodiment, one cysteine can be introduced into the peptide and one into the HLA molecule to form a disulfide to thereby stabilize the peptide-loaded MHC conformation (e.g., as described in Serra et al. (2019) NATURE COMM., 10:4917).

Furthermore, specific modifications of Ig constant regions that alter effector function have been described in the art and can be applied to the multimerization moiety of the MHC multimer to, e.g., alter disulfide bridging, alter half-life or alter complement activation ability. For example, the P311G mutation can be introduced into IgM C region to minimize complement-dependent cytotoxicity (CDC) (see e.g., Chen et al. (2020) MABS, 12:1818436). Additional constant region modifications known in the art include those described in U.S. Pat. No. 10,351,631 and U.S. Patent Publication No. 2020/0239572.

For example, modifications to the IgM Fc region or J chain have been described in the art that increase serum half-life (see e.g., U.S. Pat. No. 10,899,835, published as U.S. Patent Publication No. 2020/0239572). Accordingly, such modification(s) can be introduced into the Fc region or J chain of a multimer of the disclosure to increase serum half-life.

Additionally, modifications to the IgM constant regions that modulate complement-dependent cytotoxicity effector functions have been described in the art (see e.g., PCT Publication WO 2018/187702). Accordingly, such modification(s) can be introduced into the Fc region of a multimer of the disclosure to modulate complement-mediated effector functions.

In another embodiment, an MHC multimer of the disclosure is prepared as a bispecific multimer, wherein the bispecific multimer presents two different MHC-binding peptides on two different MHC molecules, as illustrated schematically in FIG. 6A and FIG. 6B. Such bispecific multimers can be prepared using knobs-into-holes (KIH) technology or electrostatic steering technology, which are well-established approaches in the art for creating bispecific antibodies. KIH technology involves engineering constant domains to create either a “knob” or a “hole” in each heavy chain to promote heterodimerization (see e.g., U.S. Pat. No. 10,351,631). Electrostatic steering involves engineering salt bridges through pairs of oppositely charged amino acids to promote heterodimerization (see e.g., Gunasekaran et al., (2010) J BIOL CHEM., 285(25):19637-46). Among other things, the present disclosure contemplates that KIH technology or electrostatic steering are useful for making various multimers as provided herein.

For example, in some embodiments, KIH technology can be used to introduce a knob into the Cμ4 of one chain of the Ig-derived multimerization domain and a hole into the Cμ4 of the other chain, as illustrated in FIG. 6A, to create a bispecific MHC multimer presenting two different MHC-binding peptides. Such KIH technology can also be used, for example, to introduce a knob into a Cα3 of one chain of an IgA multimerization domain and a hole into a Cα3 of the other chain (FIG. 6B) to create a bispecific MHC multimer presenting two different MHC binding peptides (see e.g., U.S. Pat. No. 10,822,399). In some embodiments, electrostatic steering may be used, wherein amino acid changes introducing salt-bridges can be introduced into at least one of a Cμ2, Cμ3 or Cμ4 domain of an IgM to promote heterodimerization. In other embodiments, KIH technology or electrostatic steering technology may be used to make a pMHC Class II IgM multimer that includes an α-IgM (“knob”) and a peptide-β-IgM (“hole”). Such a strategy would create a pentavalent pMHC class II IgM having five pMHC II moieties.

### 1. Modification of MHC Class I Alpha (a) Chain to Enhance Stability

While MHC Class I multimers have typically been prepared as “peptide-loaded” (i.e., having a peptide bound to the peptide-binding groove) for stability, the present disclosure contemplates that, in some embodiments, one or more modifications within an MHC Class I α chain of an MHC Class I multimer can be introduced to stabilize the “empty” MHC Class I molecule such that multimers can be prepared a pre-requisite of being peptide-loaded. Rather, by using stabilization techniques provided by the present disclosure, such empty multimers, comprising one or more modifications to an MHC Class I α chain, can be prepared and then peptide addition can be performed at a later time to load the multimers.

Mutations and/or modifications (e.g., engineered disulfide bonds) that stabilize empty MHC Class I molecules (e.g., between alpha helices) have been described in the art (e.g., as described in Saini et al. (2019) SCIENCE IMMUNOL., 4:eaau9039). In particular, standard recombinant DNA technology can be used to introduce a disulfide bond to link the α1 and α2 helices of the MHC Class I α chain. For example, for HLA-A*02:01, Y84C and A139C mutations can be introduced. For instance, one example is a fusion protein having the structure: β2m-linker-A*02:01 (Y84C, A139C)-linker-IgM Cμ2-Cμ3-Cμ4-TP has the amino acid sequence shown in SEQ ID NO: 23 and comprises α-chain modifications designed to increase stability of the “empty” multimer. A construct encoding this fusion protein can be expressed in a host cell to create a stable “empty” MHCI-IgM multimer to which peptides can be added for loading.

Alternatively, in some instances, an MHCI α chain and β2-microglobulin chain can be expressed separately in host cell. For example, a fusion protein having the structure: A*02:01 (Y84C, A139C)-linker-IgM Cμ2-Cμ3-Cμ4-TP has the amino acid sequence shown in SEQ ID NO: 24. The human β2-microglobulin chain has the amino acid sequence shown in SEQ ID NO: 25. Separate expression constructs encoding the α chain-IgM fusion protein and the β2-microglobulin chain can be expressed in a host cell to create a stable “empty” MHCI-IgM multimer to which peptides can be added for loading.

### III. Preparation of Multimers

The multimers of the disclosure can be prepared by methods established in the art. In one embodiment, a multimer is prepared using recombinant DNA technology, as described further below. In another embodiment, a multimer is prepared using chemical conjugation, various approaches for which are described further below.

### A. Recombinant Expression Strategies

As provided herein, certain exemplary recombinant expression strategies may be employed in design and manufacture of a multimer. In one embodiment, a multimer composition of the disclosure is prepared by standard recombinant DNA techniques using one or more nucleic acid constructs that encode polypeptides that when expressed in a host cell self-assemble into a multimer in which an MHC moiety is operatively linked to a multimerization moiety (typically with linker sequences positioned between the sequences encoding the MHC moiety and the multimerization moiety). Non-limiting representative nucleic acid constructs for expressing a multimer composition are shown schematically in FIGS. 5A-5I. Use of such expression constructs for recombinantly preparing a multimer are described in detail in Examples 1 and 3.

Exemplary approaches for recombinantly expressing peptide-MHCI-Ig multimers of the disclosure are provided and illustrated schematically herein. For instance, representative non-limiting examples of peptide-MHCI-Ig multimer structures are illustrated schematically in FIGS. 1A-1D and FIGS. 2A-2C, wherein the multimers of FIGS. 2A-2C include an effector/targeting moiety (e.g., PD-L1).

In one approach, MHC Class I multimers (as schematically illustrated in FIG. 5A) are made using two different constructs to create the peptide-MHC-Ig multimer. The first expression construct encodes, 5′ to 3′, an MHC Class I-binding peptide, β-2-microglobulin and an MHC Class I α chain (each separated by linkers), operatively linked to IgM Cμ2, Cμ3 and Cμ4 constant regions and the tailpiece region, with a linker sequence positioned between the peptide-MHC and IgM sequences. The second expression construct encodes the J chain. To express the complete peptide-MHC-Ig multimer, the two expression constructs are transfected into a host cell and the host cell is cultured to allow for expression of the two encoded constructs.

Another approach to make MHC Class I multimers (as schematically illustrated in FIG. 5B), is similar to the first approach, as depicted in FIG. 5A, using two separate constructs, except that the second expression construct encoding the immunoglobulin J chain is operatively linked to an effector/targeting moiety with a linker sequence positioned between the J chain and the effector moiety sequences. Again, to express the complete peptide-MHC-Ig multimer, the two expression constructs are transfected into a host cell and the host cell is cultured to allow for expression of the two encoded constructs.

SEQ ID NOs: 1 and 2 set forth non-limiting examples of amino acid and nucleotide sequences, respectively, for constructs comprising: peptide-GS linker-β2microglobulin-GS linker-MHC Class I α chain-GS linker-IgM Cμ2, Cμ3 and Cμ4 constant regions and the tailpiece region. SEQ ID NOs: 3 and 4 show non-limiting examples of amino acid and nucleotide sequences, respectively, for constructs comprising: peptide-GS linker-β2microglobulin-GS linker-MHC Class I α chain-GS linker-IgM Cμ3 and Cμ4 constant regions and the tailpiece region. The MHC Class I molecule used in these constructs is HLA-A*02:01 and peptide used in these constructs is the CMV pp65 epitope having the amino acid sequence shown in SEQ ID NO: 19.

SEQ ID NOs: 5 and 6 show the amino acid sequences of exemplary peptide-MHCI-IgM constructs similar to those of SEQ ID NOs: 1 and 3 (having the structure of: peptide-GS linker-β2microglobulin-GS linker-MHC Class I α chain-GS linker-IgM domains) except that SEQ ID NOs: 5 and 6 use an HLA-A*02:01-restricted MART-1 epitope, having the amino acid sequence shown in SEQ ID NO: 20, as the peptide. The construct of SEQ ID NO: 5 uses IgM Cμ2, Cμ3 and Cμ4, as well as the tailpiece region, whereas the construct of SEQ ID NO: 6 uses IgM Cμ3 and Cμ4, as well as tailpiece region. The construct of SEQ ID NO: 6 is encoded by the nucleotide sequence shown in SEQ ID NO: 7.

In some embodiments, a multimer comprises a Cμ3-4 variant (“var2”) set forth in SEQ ID NO: 254. SEQ ID NOs: 249 and 248 show the amino acid sequences of (i) ELA- and (ii) NLV-pMHC-IgM Cu3-4var2, respectively.

SEQ ID NOs: 8, 9 and 11 show the amino acid sequences of exemplary peptide-MHCI-IgA constructs having the structure of: peptide-GS linker-β2microglobulin-GS linker-MHC Class I α chain-GS linker-IgA regions. All three constructs use HLA-A*02:01 as the MHC Class I molecule and the CMV pp65 epitope as the peptide. The construct of SEQ ID NO: 8 includes the IgA1.1 regions. The construct of SEQ ID NO: 9 includes the IgA2m2.1 regions. The construct of SEQ ID NO: 11 includes the IgA2m2.2 regions. The construct of SEQ ID NO: 9 is encoded by the nucleotide sequence of SEQ ID NO: 10. The construct of SEQ ID NO: 11 is encoded by the nucleotide sequence of SEQ ID NO: 12.

In some embodiments, exemplary schematics for recombinantly expressing peptide-MHCII-Ig multimer structures are provided in FIGS. 3A-3C and FIGS. 4A-4C, wherein the multimers of FIGS. 4A-C include an effector/targeting moiety (e.g., PD-L1).

Three expression approaches for MHC Class II multimers are schematically illustrated in FIGS. 5C-5E. In the approach schematically illustrated in FIG. 5C, three different constructs are used to create the peptide-MHC-Ig multimer. The first expression construct encodes, in 5′ to 3′ order, an MHC Class II-binding peptide and an MHC Class II R chain (separated by a linker), operatively linked to IgM Cμ2, Cμ3 and Cμ4 constant regions and the tailpiece region, with a linker sequence positioned between the peptide-MHC and IgM sequences. The second expression construct encodes an MHC Class II α chain and the third expression construct encodes the J chain. To express the complete peptide-MHC-Ig multimer, the three expression constructs are transfected into a host cell and the host cell is cultured to allow for expression of the three encoded constructs.

The approach schematically illustrated in FIG. 5D is similar to the approach of FIG. 5C, using three separate constructs, except that the third expression construct encoding the immunoglobulin J chain is operatively linked to an effector moiety sequence encoding a PD-L1 effector/targeting moiety, with a linker sequence positioned between the J chain and the PD-L1 sequences. Again, to express the complete peptide-MHC-Ig multimer, the three expression constructs are transfected into a host cell and the host cell is cultured to allow for expression of the two encoded constructs.

In the approach schematically illustrated in FIG. 5E, only two expression constructs are used for the MHC Class II multimers, wherein the first expression construct (encoding peptide-MHCII β chain-IgM) is the same as in FIG. 5C and FIG. 5D and the second expression construct encodes, in 5′ to 3′ order, an MHC Class II α chain, a skipping peptide (T2A), the J chain and a PD-L1 effector/targeting moiety (with a linker between the J chain and PD-L1).

For all of the approaches shown in FIGS. 5C-E, after transfection of the expression constructs into a host cell, the peptide-MHC-Ig multimer self-assembles and can be purified from the host cells, or culture supernatant thereof, by standard methods.

Non-limiting representative examples of peptide-MHCII-Ig expression constructs can be prepared to achieve the MHCII α chain/P chain pairings shown below in Table 1 in the assembled multimers. In some embodiments, any of these pairings may also comprise a construct encoding a J-chain (as set forth in SEQ ID NO: 251).

TABLE 1
MHCII Pairings in peptide-MHCII-Ig Multimers

| Pairing | Protein name | MHCII Alpha chain | MHCII Beta chain |
|---|---|---|---|
| #1 | HA-DR1-IgM(Cμ2-4) | DRA1*01 | HA-DRB1*01-Cμ2-4 |
| #2 | HA-Xa-DR1-IgM(Cμ2-4) | DRA1*01 | HA-Xa-DRB1*01-Cμ2-4 |
| #3 | HA-DR1-DS-IgM(Cμ2-4) | DRA1*01(S95C) | HA-DRB1*01(S120C)-Cμ2-4 |
| #4 | CLIP-Xa-DR1-IgM(Cμ2-4) | DRA1*01 | CLIP-Xa-DRB1*01-Cμ2-4 |

For pairing #1, the amino acid sequence of the DRA1*01 α chain is shown in SEQ ID NO: 13 and the amino acid sequence for the peptide-MHCII β chain-IgM construct is shown in SEQ ID NO: 14. The latter construct has the structure: peptide-GS linker-DRB1*01 MHCII-GS linker-IgM Cμ2, Cμ3 and Cμ4, as well as the tailpiece region. The peptide used is hemagglutinin 306-318, having the amino acid sequence shown in SEQ ID NO: 21.

For pairing #2, SEQ ID NO: 13 was again used for the α chain construct. The peptide-MHCII β chain-IgM construct is shown in SEQ ID NO: 15. The latter construct is the same as that used in pairing #1, except that an Xa linker is used between the peptide and the MHCII β chain, rather than a GS linker.

For pairing #3, a mutant DRA1*01 α chain, having S95C mutation, was used. The amino acid sequence of this α chain is shown in SEQ ID NO: 16. The peptide-MHCII β chain-IgM construct is shown in SEQ ID NO: 17. The latter construct is the same as that used in pairing #1, except that there is an S120C mutation introduced into the MHCII β chain.

For pairing #4, SEQ ID NO: 13 was again used for the α chain construct. The peptide-MHCII β chain-IgM construct is shown in SEQ ID NO: 18. The latter construct is the same as that used in pairing #2 (using an Xa linker between the peptide and the R chain), except that a CLIP peptide having the amino acid sequence shown in SEQ ID NO: 22 is used instead of the HA peptide.

The use of an engineered disulfide bond, a cys-trap, to improve expression of Class I pMHC-IgM, described in Example 4, can be applied to Class II pMHC-IgM multimers to improve expression. For instance, certain exemplary constructs made using such techniques include any of those set forth in SEQ ID NOs: 216-220. Representative pairings of peptide-MHC II β chain and MHC II α chains to make Cys-trapped Class II pMHC-IgM multimers are listed in Table 2.

TABLE 2
MHCII Pairings for Cys-trapped Class II pMHC-IgM Multimers

| | | | SEQ | | | | |
|---|---|---|---|---|---|---|---|
| | | MHCII Beta | ID | MHCII | SEQ | | SEQ ID |
| Pairing | Protein Name | Chain Name | NO: | Alpha Chain | ID NO: | J chain | NO: |
| #1 | MTB(C15)- | MTB(C15)- | 218 | DRA1*01- | 216 | J chain- | 251 |
| | DR15- | DRB1*15:01- | | I92C-cJun | | (G4S)2- | |
| | FosW/cJun-IgM | FosW-Cu2-4 | | | | Flag-His | |
| #2 | RQ13(C14)- | RQ13(C14)- | 219 | DRA1*01- | 217 | J chain- | 251 |
| | DR1-IgM | DRB1*01:01- | | I92C | | (G4S)2- | |
| | | Cu2-4 | | | | Flag-His | |
| #3 | MBP(C14)- | MBP(C14)- | 220 | DRA1*01- | 216 | J chain- | 251 |
| | DR15- | DRB1*15:01- | | I92C-cJun | | (G4S)2- | |
| | FosW/cJun-IgM | FosW/cJun-Cu2-4 | | | | Flag-His | |

For pairing #1, the amino acid sequence for the peptide-MHCII β chain-FosW-IgM construct is shown in SEQ ID NO: 218. The amino acid sequence for the DRA1*01 α chain-cJun is shown in SEQ ID NO: 216. The J-chain amino acid sequence is shown in SEQ ID NO: 251.

For pairing #2, the amino acid sequence for the peptide-MHCII β chain-IgM construct is shown in SEQ ID NO: 219. The amino acid sequence for the DRA1*01 α chain is shown in SEQ ID NO: 217. The J-chain amino acid sequence is shown in SEQ ID NO: 251.

For pairing #3, the amino acid sequence for the peptide-MHCII β chain-IgM is show in SEQ ID NO: 220. The amino acid sequence for the DRA1*01 α chain-cJun is shown in SEQ ID NO: 216. The J-chain amino acid sequence is shown in SEQ ID NO: 251.

General techniques for nucleic acid manipulation are well established in the art, such as described in, for example, Sambrook et al. (1989) MOLECULAR CLONING: A LABORATORY MANUAL, 2ND EDITION, Vols. 1-3, Cold Spring Harbor Laboratory Press, or Ausubel, F. et al. (1987) CURRENT PROTOCOLS IN MOLECULAR BIOLOGY, Green Publishing and Wiley-Interscience, New York. Generally, the DNA encoding the polypeptide is operably linked to suitable transcriptional or translational regulatory elements derived from mammalian, viral, or insect genes. Such regulatory elements include a transcriptional promoter, an optional operator sequence to control transcription, a sequence encoding suitable mRNA ribosomal binding site, and sequences that control the termination of transcription and translation. The ability to replicate in a host, usually conferred by an origin of replication, and a selection gene to facilitate recognition of transformants is additionally incorporated.

In one embodiment, the nucleic acid construct is designed to be suitable for in vitro transcription/translation (IVTT). In one embodiment, the nucleic acid is designed to be suitable for recombinant expression in a host cell. Appropriate cloning and expression vectors for use with bacterial, fungal, yeast, and mammalian cellular hosts can be found in CLONING VECTORS: A LABORATORY MANUAL (Elsevier, New York (1985)), the relevant disclosure of which is hereby incorporated by reference.

In some embodiments, the multimer composition is synthesized utilizing an in vitro transcription/translation (IVTT) system that can both transcribe, for example, a DNA construct into RNA, and then translate the RNA into a protein. IVTT can allow for protein production in a cell-free environment directly from a DNA or RNA template. An IVTT method used herein can be performed using, for example, a PCR product, a linear DNA plasmid, a circular DNA plasmid, or an mRNA template with a ribosome-binding site (RBS) sequence. After the appropriate template has been isolated, transcription components can be added to the template including, for example, ribonucleotide triphosphates, and RNA polymerase. After transcription has been completed, translation components can be added, which can be found in, for example, rabbit reticulocyte lysate, or wheat germ extract. In some methods, the transcription and translation can occur during a single step, in which purified translation components found in, for example, rabbit reticulocyte lysate or wheat germ extract are added at the same time as adding the transcription components to the nucleic acid template.

In one embodiment, the nucleic acid sequence is incorporated into a vector, such as a plasmid vector, a viral vector or a non-viral vector. The vector is selected to be suitable for use in the intended host cell (i.e., the vector incudes all necessary transcriptional regulatory elements to allow for expression of the encoded multimer composition in the host cell). Suitable vectors, including transcriptional regulatory elements for use in various host cells, including mammalian host cells, are well established in the art.

As appreciated by those skilled in the art, because of third base degeneracy, almost every amino acid can be represented by more than one triplet codon in a coding nucleotide sequence. In addition, minor base pair changes may result in a conservative substitution in the amino acid sequence encoded but are not expected to substantially alter the biological activity of the gene product. Therefore, a nucleic acid sequence encoding a protein described herein may be modified slightly in sequence and yet still encode its respective gene product.

Nucleic acids encoding any of the various proteins or polypeptides described herein may be synthesized chemically or prepared through standard recombinant DNA techniques. Codon usage may be selected so as to improve expression in a cell. Such codon usage will depend on the cell type selected. Specialized codon usage patterns have been developed for E. coli and other bacteria, as well as mammalian cells, plant cells, yeast cells and insect cells. See for example: Mayfield et al. (2003) PROC. NATL. ACAD. SCI. USA, 100(2):438-442; Sinclair et al. (2002) PROTEIN EXPR. PURIF., 26(I):96-105; Connell, N. D. (2001) CURR. OPIN. BIOTECHNOL., 12(5):446-449; Makrides et al. (1996) MICROBIOL. REV., 60(3):512-538; and Sharp et al. (1991) YEAST, 7(7):657-678.

In one embodiment, the vector is designed for expression in a prokaryotic host cell (e.g., E. coli). In one embodiment, the vector is designed for expression in a eukaryotic host cell (e.g., yeast). In one embodiment, the vector is designed for expression in a mammalian host cell. In one embodiment, the mammalian host cells are human host cells. In one embodiment, the human host cells are human embryonic kidney (HEK) cells. In one embodiment, the HEK cells are 293 cells or are a 293-derived HEK strain. Such HEK cells are commercially available in the art, a non-limiting example of which is the Expi293F™ cell line (Fisher ThermoScientific). In yet another embodiment, the mammalian host cell is a CHO cell line.

When mammalian host cells are used, typically the signal sequence used in the expression construct is derived from a mammalian protein. A signal sequence used to express a secreted protein can be a homologous signal sequence (derived from the same protein being expressed) or a heterologous signal sequence (derived from a different protein than the one being expressed). In one embodiment, a heterologous signal sequence is from an Ig supergroup member. In one embodiment, the signal sequence is an immunoglobulin chain signal sequence. In one embodiment, the signal sequence is an Ig Kappa chain V-III region CLL signal peptide, e.g., having the sequence MEAPAQLLFLLLLWLPDTTG (SEQ ID NO: 184). Other suitable signal sequences include a human CD4 signal peptide, e.g., having the sequence MNRGVPFRHLLLVLQLALLPAAT (SEQ ID NO: 185), a mouse Ig kappa chain V-III region signal peptide, e.g., having the sequence METDTLLLWVLLLWVPGSTG (SEQ ID NO: 186), a mouse H-2Kb signal peptide, e.g., having the sequence MVPCTLLLLLAAALAPTQTRA (SEQ ID NO: 187), a human serum albumin signal peptide, e.g., having the sequence MKWVTFISLLFLFSSAYS (SEQ ID NO: 188), a human IL-2 signal peptide, e.g., having the sequence MYRMQLLSCIALSLALVTNS (SEQ ID NO: 189), a human HLA-A*02:01 signal peptide, e.g., having the sequence MAVMAPRTLLLLLSGALALTQTWA (SEQ ID NO: 190) and a human β2m signal peptide, e.g., having the sequence MSRSVALAVLALLSLSGLEA (SEQ ID NO: 191).

Furthermore, the transcriptional regulatory sequences used in the vector are selected for their effectiveness in mammalian host cell expression.

Other expression systems include stable Drosophila cell transfectants and baculovirus infected insect-cells suitable for expression of proteins.

For prokaryotic host cells that do not recognize and process a native signal sequence, the signal sequence is substituted by a prokaryotic signal sequence selected, for example, from the group of the alkaline phosphatase, penicillinase, 1 pp, or heat-stable enterotoxin II leaders.

For yeast secretion the native signal sequence may be substituted by, e.g., a yeast invertase leader, a factor leader (including Saccharomyces and Kluyveromyces α-factor leaders), or acid phosphatase leader, the C. albicans glucoamylase leader, or the signal sequence described in U.S. Pat. No. 5,631,144. In mammalian cell expression, mammalian signal sequences as well as viral secretory leaders, for example, the herpes simplex gD signal, are available. The DNA for such precursor regions may be ligated in reading frame to DNA encoding the protein.

Both expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2 micron plasmid origin is suitable for yeast, and various viral origins (SV40, polyoma, adenovirus, VSV or BPV) are useful for cloning vectors in mammalian cells. Generally, the origin of replication component is not needed for mammalian expression vectors (the SV40 origin may typically be used only because it contains the early promoter).

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

Expression and cloning vectors usually contain a promoter that is recognized by the host organism and is operably linked to the coding sequences. Promoters suitable for use with prokaryotic hosts include the phoA promoter, β-lactamase and lactose promoter systems, alkaline phosphatase, a tryptophan (trp) promoter system, and hybrid promoters such as the tan promoter. However, other known bacterial promoters are suitable. Promoters for use in bacterial systems also will contain a Shine-Dalgarno (S.D.) sequence operably linked to the DNA encoding the protein described herein. Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Examples of suitable promoting sequences for use with yeast hosts include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Transcription from vectors in mammalian host cells can be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and most preferably Simian Virus 40 (SV40), from heterologous mammalian promoters, e.g., the actin promoter or an immunoglobulin promoter, from heat-shock promoters, provided such promoters are compatible with the host cell systems.

Transcription of a DNA encoding protein described herein by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv (1982) NATURE, 297:17-18 on enhancing elements for activation of eukaryotic promoters. The enhancer may be spliced into the vector at a position 5′ or 3′ to the peptide-encoding sequence, but is preferably located at a site 5′ from the promoter.

Expression vectors used in eukaryotic host cells (e.g., yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of mRNA encoding the protein described herein. One useful transcription termination component is the bovine growth hormone polyadenylation region.

In one embodiment, the expression construct comprises a signal sequence operatively linked upstream from the sequences encoding the multimer composition to thereby facilitate secretion of the multimer composition from a host cell.

The expression construct is introduced into the host cell using a method appropriate to the host cell, as will be apparent to one of skill in the art. A variety of methods for introducing nucleic acids into host cells are known in the art, including, but not limited to, electroporation; transfection employing calcium chloride, rubidium chloride, calcium phosphate, DEAE-dextran, or other substances; microprojectile bombardment; lipofection; and infection (where the vector is an infectious agent).

Suitable host cells include prokaryotes, yeast, mammalian cells, or bacterial cells. Suitable bacteria include gram negative or gram positive organisms, for example, E. coli or Bacillus spp. Yeast, preferably from the Saccharomyces species, such as S. cerevisiae, may also be used for production of polypeptides. Various mammalian or insect cell culture systems can also be employed to express recombinant proteins. Baculovirus systems for production of heterologous proteins in insect cells are reviewed by Luckow et al. (1988) BIO/TECHNOLOGY, 6:47. Examples of suitable mammalian host cell lines include endothelial cells, COS-7 monkey kidney cells, CV-1, L cells, C127, 3T3, Chinese hamster ovary (CHO), human embryonic kidney cells, HeLa, 293, 293T, and BHK cell lines. Purified polypeptides are prepared by culturing suitable host/vector systems to express the recombinant proteins. For many applications, the small size of many of the polypeptides described herein would make expression in E. coli as the preferred method for expression. The protein is then purified from culture media or cell extracts.

The host cells used to produce the proteins of this disclosure may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma)) are suitable for culturing the host cells. In addition, many of the media described in Ham et al. (1979) METH. ENZYMOL., 58:44, Barites et al. (1980) ANAL. BIOCHEM., 102:255, U.S. Pat. Nos. 4,767,704, 4,657,866, 4,927,762, 4,560,655, 5,122,469, 6,048,728, 5,672,502, or U.S. Pat. No. RE 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Proteins described herein can also be produced using cell-free translation systems. For such purposes the nucleic acids encoding the polypeptide must be modified to allow in vitro transcription to produce mRNA and to allow cell-free translation of the mRNA in the particular cell-free system being utilized (eukaryotic such as a mammalian or yeast cell-free translation system or prokaryotic such as a bacterial cell-free translation system).

Proteins described herein can also be produced by chemical synthesis (e.g., by the methods described in SOLID PHASE PEPTIDE SYNTHESIS, 2ND EDITION, The Pierce Chemical Co., Rockford, Ill. (1984)). Modifications to the protein can also be produced by chemical synthesis.

The proteins of the present disclosure can be purified by isolation/purification methods for proteins generally known in the field of protein chemistry. For example, an affinity tag (such as His6) can be incorporated into a component of the MHC multimer and the multimer can be purified by affinity chromatography (e.g., immobilized metal affinity chromatography for the His6 tag). Other non-limiting examples of suitable purification methods include immunoaffinity chromatography, extraction, recrystallization, salting out (e.g., with ammonium sulfate or sodium sulfate), centrifugation, dialysis, ultrafiltration, adsorption chromatography, ion exchange chromatography, hydrophobic chromatography, normal phase chromatography, reversed-phase chromatography, get filtration, gel permeation chromatography, electrophoresis, countercurrent distribution or any combinations of these. After purification, polypeptides may be exchanged into different buffers and/or concentrated by any of a variety of methods known to the art, including, but not limited to, filtration and dialysis.

The purified polypeptide is preferably at least 85% pure, or preferably at least 95% pure, and most preferably at least 98% pure. Regardless of the exact numerical value of the purity, the polypeptide is sufficiently pure for its intended use.

In certain embodiments, the expression construct includes at least one tag sequence, most typically as at the C-terminal end of the coding region, although inclusion of a tag at the N-terminal end (alternative to or in addition to the C-terminal end) is also encompassed. Suitable tag sequences are known in the art and described further herein.

Additional tags suitable for use in the methods and compositions provided herein include affinity tags, including but not limited to enzymes, protein domains, or small polypeptides which bind with high specificity to a range of substrates, such as carbohydrates, small biomolecules, metal chelates, antibodies, etc., to allow rapid and efficient purification of proteins. Solubility tags enhance proper folding and solubility of a protein and are frequently used in tandem with affinity tags. Sequences encoding such a tag(s) can be incorporated into an expression construct of the disclosure, such as at the C-terminus or N-terminus of the peptide-multimer-encoding regions to thereby incorporate a detectable tag into the expressed polypeptide.

### B. Chemical Conjugation Preparation of Multimers

In another embodiment, a multimer composition of the disclosure is produced by covalent conjugation of the MHC moiety (MHCM) to the multimerization moiety. In certain embodiments, a multimer composition is produced by covalent conjugation of the multimerization moiety to the C-terminus of one subunit of an MHC Class I α/β2-microglobulin chain pair or MHC Class II α/β chain pair functioning as the MHCM.

For chemical conjugation approaches, the MHCM and multimerization moiety polypeptide components can be prepared separately, either recombinantly or chemically. For example, MHC chain pairings (Class I α/β2-microglobulin or Class II α/β), can be expressed using recombinant DNA technology by introducing an expression construct into bacterial cells, insect cells, or mammalian cells, and purifying the recombinant protein from cell extracts, e.g., as described above. The multimerization moiety polypeptide similarly can be prepared recombinantly, prior to conjugation to the MHCM.

A number of suitable methods for forming covalent bonds between the MHC moiety and the multimerization moiety are provided herein.

### 1. Chemical Bioconjugation

In some embodiments, the chemical conjugation can be mediated by, for example, by cysteine bioconjugation. In some embodiments, the cysteine bioconjugation is mediated by cysteine alkylation. In some embodiments, the cysteine bioconjugation is mediated by cysteine oxidation. In other embodiments, the cysteine bioconjugation is mediated by a desulfurization reaction. In some embodiments, cysteine bioconjugation is mediated by iodoacetamide. In some embodiments, the cysteine bioconjugation is mediated by maleimide. Methods for utilizing cysteine mediated linkage of two moieties which can be used to produce the multimer composition disclosed herein have been described, for example, see Chalker et al. (2009) CHEM ASIAN J., 4(5):630-40; Spicer et al. (2015) NAT. COMMUN., 5:4740.

In some embodiments, the multimer compositions are produced by chemical modification of amino acids other than cysteine, including but not limited to lysine, tyrosine, arginine, glutamate, aspartate, serine, threonine, methionine, histidine and tryptophan side-chains, as well as N-terminal amines or C-terminal carboxyls, as previously described (Baslé et al. (2010) M. CHEM. BIOL., 17(3):213-27; Hu et al. (2016) CHEM. SOC. REV., 45(6):1691-719; Lin et al. (2017) SCIENCE, 355(6325):597-602).

### 2. Native Chemical Ligation

In some embodiments, the multimer compositions are produced by native chemical ligation (NCL), wherein one polypeptide comprises a C-terminal thioester and the other polypeptide comprises an N-terminal cysteine residue, or functional equivalent thereof, wherein the reaction between the cysteine side-chain and the thioester irreversibly forms a native peptide bond, thus ligating the polypeptides together. Methods for NCL have been described in, for example, Hejjaoui et al. (2015) M. PROTEIN SCI., 24(7):1087-99.) Mandal et al. (2012) PROC. NATL. ACAD. SCI. USA., 109(37):14779-84; Torbeev et al. (2013) PROC. NATL. ACAD. SCI. USA., 110(50):20051-6.

In some embodiments, β- and/or γ-thio amino acids are incorporated into the polypeptides. Desulfurization protocols can then produce the desired native side-chain. In some embodiments, non chemical ligation is performed at an alanine residue. In other embodiments, non chemical ligation is performed at phenylalanine, valine, leucine, threonine, lysine, proline, glutamine, arginine, tryptophan, aspartate, glutamate, and asparagine. Ligation/desulfurization approaches that remove purification steps and increase the yield of ligated products have been described.

### 3. Click Chemistry Mediated Bioorthogonal Conjugation

In some embodiments, the multimer compositions are produced by bioorthogonal conjugation between a conjugation moiety at the C-terminus of one polypeptide and a conjugation moiety at the N-terminus of the other polypeptide. As used herein, bioorthogonal conjugation refers to any chemical reaction that can occur inside of living systems without interfering with native biochemical processes, including chemical reactions that are chemical reactions that occur in vitro at physiological pH in, or in the presence of water. To be considered bioorthogonal, reactions must be selective and avoid side-reactions with other functional groups found in a given starting compound. In addition, any resulting covalent bond(s) between the reaction partners should be strong and chemically inert to biological reactions and should not affect the biological activity of the desired molecule.

In some embodiments, the bioorthogonal conjugation is mediated by “click chemistry” (see, e.g., Kolb et al. (2001) ANGEWANDTE CHEMIE INTERNATIONAL EDITION, 40: 2004-2021). The term “click chemistry”, as used herein, refers to a set of reliable and selective bioorthogonal reactions for the rapid synthesis of new compounds and combinatorial libraries. Properties of click reactions include modularity, wideness in scope, high yielding, stereospecificity and simple product isolation (separation from inert by-products by non-chromatographic methods) to produce compounds that are stable under physiological conditions. A “click reaction” can be with copper, or it can be a copper-free click reaction.

For conjugation of two polypeptides via click chemistry, click chemistry moieties of components have to be reactive with each other, for example, in that the reactive group of a click chemistry moiety on one polypeptide reacts with the reactive group of the second click chemistry moiety on the other polypeptide to form a covalent bond.

Conjugation moieties suitable for click chemistry, reaction conditions, and associated methods are available in the art (e.g., Kolb et al. (2001) ANGEWANDTE CHEMIE INTERNATIONAL EDITION, 40:2004-2021; Evans (2007) AUSTRALIAN JOURNAL OF CHEMISTRY, 60: 384-395; Lahann (2009) CLICK CHEMISTRY FOR BIOTECHNOLOGY AND MATERIALS SCIENCE, John Wiley & Sons Ltd, ISBN 978-0-470-69970-6). In some embodiments, a click chemistry moiety may comprise or consist of a terminal alkyne, azide, strained alkyne, diene, dieneophile, alkoxyamine, carbonyl, phosphine, hydrazide, thiol, or alkene moiety. In certain embodiments, the azide is a copper-chelating azide. In one embodiment, the copper-chelating azide is a picolyl azide. Reagents for use in click chemistry reactions are commercially available, such as from Click Chemistry Tools (Scottsdale, AZ) or GenScript (Piscataway, NJ).

In some embodiments, sortase-mediated conjugation is used to install a first click chemistry moiety at one terminus of one polypeptide and a second click chemistry moiety at one terminus of the other polypeptide. Methods of attaching click chemistry moieties utilizing sortase are described, for example, in WO2013/00355. In some embodiments, intein-mediated conjugation is used to install a first click chemistry moiety at one terminus of one polypeptide and a second click chemistry moiety at one terminus of the other polypeptide. Sortase-mediated and intein-mediated conjugation is described further below.

In some embodiments, the methods of click chemistry mediated covalent conjugation of two polypeptides provided herein comprise native chemical ligation of C-terminal thioesters with β-amino thiols (Xiao J. et al. (2009) ORG. LETT., 11(18):4144-7).

In some embodiments, the click chemistry used to produce the multimer composition comprises 1,3-dipolar cycloaddition (e.g., the Cu(I)-catalyzed stepwise variant, often referred to simply as the “click reaction”; see, e.g., Tornoe et al. (2002) JOURNAL OF ORGANIC CHEMISTRY, 67: 3057-3064). Copper and ruthenium are the commonly used catalysts in the reaction. The use of copper as a catalyst results in the formation of 1,4-regioisomer whereas ruthenium results in formation of the 1,5-regioisomer.

In some embodiments, the click chemistry conjugation comprises a cycloaddition reaction, such as the Diels-Alder reaction. In some embodiments, the polypeptides are conjugated by azide-alkyne 1,3-dipolar cycloaddition (“click chemistry). In some embodiments, the cycloaddition is promoted by the presence of Cu(I)-catalyzed cycloaddition (CuAAC).

In some embodiments, the click chemistry conjugation comprises nucleophilic addition to small strained rings like epoxides and aziridines. In some embodiments, the cycloaddition is promoted by strained cyclooctyne systems, for example, as described in Agard et al. (2004) J. AM. CHEM. SOC., 126(46):15046-7.

In some embodiments, the click chemistry conjugation comprises nucleophilic addition to activated carbonyl groups.

In some embodiments, the conjugation of the polypeptides occurs by a bioorthogonal reaction. In some embodiments, the polypeptides are conjugated by inverse-electron demand Diels-Alder reactions between strained dienophiles and tetrazine dienes, for example, as described in Blackman et al. (2008) J. AM. CHEM. SOC., 130(41):13518-9; and Devaraj et al. (2008) BIOCONJUG. CHEM., 19(12):2297-9). In some embodiments, the dienophile is a trans-cyclooctene. In some embodiments, the dienophile is a norbornene.

### 4. Sortase Mediated Conjugation

In some embodiments, conjugation between two polypeptides is mediated by a cysteine transpeptidase. In some embodiments, the cysteine transpeptidase is a sortase, or enzymatically active fragment thereof. A variety of sortase enzymes have been described and are commercially available (e.g., Antos et al. (2016) CURR. OPIN. STRUCT. BIOL., 38:111-118). Sortases recognize and cleave an amino acid motif, referred to as a “sortag”, to produce a peptide bond between the acyl donor and acceptor site on two polypeptides, resulting in the ligation of different polypeptides which contain N- or C-terminal sortags. In particular, sortases join a C-terminal LPETGG recognition motif (SEQ ID NO: 192) to an N-terminal GGG (oligoglycine) motif.

Accordingly, in some embodiments, the recognition motif is added to the C-terminus of the MHC moiety and an oligo-glycine motif is added to the N-terminus of the multimerization moiety. Upon addition of sortase to the mixture, the two polypeptides are covalently linked through a native peptide bond.

Methods of conjugation of sortags into proteins have also been described. (Matsumoto et al. (2016) ACS SYNTH BIOL., 5(11):1284-1289; Williams et al. (2016) PLOS ONE, 11(4):e0154607; and Witte et al. (2012) PROC. NATL. ACAD. SCI. USA., 109(30):11993-8; Mao et al. (2004) J. AM. CHEM. SOC., 126(9):2670-1; Guimaraes et al. (2013) NAT. PROTOC., 8(9):1787-99 and Theile et al. (2013) NAT. PROTOC., 8(9):1800-7).

In some embodiments, the aminoglycine peptide fragment generated by the sortase reaction, is removed by dialysis or centrifugation, e.g., while the reaction is proceeding (Freiburger et al. (2015) J. BIOMOL. NMR., 63(1):1-8). In some embodiments, affinity immobilization strategies or flow-based platforms are used for the selective removal of reaction components (Policarpo et al. (2014) ANGEW. CHEM. INT. ED. ENGL., 53(35):9203-8).

In some embodiments, the equilibrium of the reaction can be controlled by ligation product or by-product deactivation, e.g., as described in Yamamura et al. (2011) CHEM. COMMUN. (CAMB)., 47(16):4742-4. In other embodiments, by-products are deactivated by chemical modification of the acyl donor glycine as described, for example, in Liu et al. (2014) J. ORG. CHEM., 79(2):487-92; and Williamson et al. (2014) NAT. PROTOC., 9(2):253-62.

Exemplary MHCII constructs made using a sortag technique are set forth in SEQ ID NOs.: 221-224 with DRA1 (SEQ ID NO: 205), SEQ ID NOs: 227-228 with DRA1 (SEQ ID NO: 216), and SEQ ID NOs: 230-231 with DRA1 (SEQ ID NO: 216 or 217, respectively).

### 5. Intein-Mediated Conjugation

Inteins are naturally occurring, self-splicing protein subdomains that are capable of excising out their own protein subdomain from a larger protein structure while simultaneously joining the two formerly flanking peptide regions (“exteins”) together to form a mature host protein. Intein-based methods of protein modification and ligation have been developed. An intein is an internal protein sequence capable of catalyzing a protein splicing reaction that excises the intein sequence from a precursor protein and joins the flanking sequences (N- and C-exteins) with a peptide bond.

As used herein, the term “split intein” refers to any intein in which one or more peptide bond breaks exists between the N-terminal intein segment and the C-terminal intein segment such that the N-terminal and C-terminal intein segments become separate molecules that can non-covalently reassociate, or reconstitute, into an intein that is functional for splicing or cleaving reactions. Any catalytically active intein, or fragment thereof, may be used to derive a split intein for use in the systems and methods disclosed herein. For example, in one aspect the split intein may be derived from a eukaryotic intein. In another aspect, the split intein may be derived from a bacterial intein. In another aspect, the split intein may be derived from an archaeal intein. Preferably, the split intein so-derived will possess only the amino acid sequences essential for catalyzing splicing reactions.

As used herein, the “N-terminal intein segment” refers to any intein sequence that comprises an N-terminal amino acid sequence that is functional for splicing and/or cleaving reactions when combined with a corresponding C-terminal intein segment. An N-terminal intein segment thus also comprises a sequence that is spliced out when splicing occurs. An N-terminal intein segment can comprise a sequence that is a modification of the N-terminal portion of a naturally occurring (native) intein sequence. For example, an N-terminal intein segment can comprise additional amino acid residues and/or mutated residues so long as the inclusion of such additional and/or mutated residues does not render the intein non-functional for splicing or cleaving. Preferably, the inclusion of the additional and/or mutated residues improves or enhances the splicing activity and/or controllability of the intein. Non-intein residues can also be genetically fused to intein segments to provide additional functionality, such as the ability to be affinity purified or to be covalently immobilized.

As used herein, the “C-terminal intein segment” refers to any intein sequence that comprises a C-terminal amino acid sequence that is functional for splicing or cleaving reactions when combined with a corresponding N-terminal intein segment. In one aspect, the C-terminal intein segment comprises a sequence that is spliced out when splicing occurs. In another aspect, the C-terminal intein segment is cleaved from a peptide sequence fused to its C-terminus. A C-terminal intein segment can comprise a sequence that is a modification of the C-terminal portion of a naturally occurring (native) intein sequence. For example, a C terminal intein segment can comprise additional amino acid residues and/or mutated residues so long as the inclusion of such additional and/or mutated residues does not render the C-terminal intein segment non-functional for splicing or cleaving.

Expressed protein ligation (EPL) refers to a native chemical ligation between a recombinant protein with a C-terminal thioester and a second agent with an N-terminal cysteine. The C-terminal thioester can readily be introduced onto any recombinant protein (i.e., the targeting ligand) through the use of auto-processing, also known as protein-splicing, mediated by an intein (intervening protein). Inteins are proteins that can excise themselves from a larger precursor polypeptide chain, utilizing a process that results in the formation of a native peptide bond between the flanking extein (external protein) fragments. When an auto-processing protein is cloned downstream of the targeting ligand, thiols (e.g., 2-mercaptoethanesulfonic acid, MESNA) can be used to induce the site-specific cleavage of the auto-processing protein, resulting in the formation of a reactive thioester. The thioester will then react with any agent that has an N-terminal cysteine. EPL operates in a site-specific manner, and the reaction is known to be very efficient if both functional groups are in high concentrations. (reviewed in Elias et al. (2010) SMALL, 6:2460-2468).

Accordingly, in some embodiments, one polypeptide component of the multimer is ligated to an alkynated peptide by expressed protein ligation (EPL) and then conjugated to an azide-labeled second polypeptide of the multimer by Cu(I)-catalyzed terminal azide-alkyne cycloaddition (CuAAC).

A number of inteins have now been described including, but not limited to MxeGyrA (Frutos et al. (2010); Southworth et al. (1999)); SspDnaE (Shah et al. (2012); Wu et al. (1998)); NpuDnaE (Shah et al. (2012); Vila-Perello et al. (2013)); AvaDnaE (David et al. (2015); Shah et al. (2012)); Cfa (consensus DnaE split intein) (Stevens et al. (2016); gp41-1 and gp41-8 (Carvajal-Vallejos et al. (2012)); NrdJ-1 (Carvajal-Vallejos et al. (2012)); IMPDH-1 (Carvajal-Vallejos et al. (2012)) and AceL-TerL (Thiel et al. (2014)).

Suitable intein sequences and protocols for use in protein conjugation have been described in the art, such as in Stevens et al. (2016) J. AM. CHEM. SOC., 138, 2162-2165; Shah et al. (2012) J. AM. CHEM. SOC., 134, 11338-11341; and Vila-Perello et al. (2013) J. AM. CHEM. SOC., 135, 286-292; Batjargal et al. (2015) J. AM. CHEM. SOC., 137(5):1734-7; and Guan et al. (2013) BIOTECHNOL. BIOENG., 110(9):2471-81.

### 6. Additional Bioconjugation Methods

In some embodiments, the conjugation of the polypeptides of the multimer is mediated enzymatically. In some embodiments, the enzyme is formylglycine generating enzyme (FGE) that recognizes the CXPXR amino acid sequence motif and converts the cysteine residue to formylglycine, thus introducing an aldehyde functional group (Wu et al. (2009) PROC. NATL. ACAD. SCI. USA., 106(9):3000-5), which is subjected to bio-orthogonal transformations such as oximation and Hydrazino-Pictet-Spengler reactions (Agarwal et al. (2013) BIOCONJUG. CHEM., 24(6):846-51; Dirksen et al. (2008) BIOCONJUG. CHEM., 19(12):2543-8).

Site-specific bioconjugation strategies offer many possibilities for directed protein modifications. Among the various enzyme-based conjugation protocols, formylglycine-generating enzymes allow to post-translationally introduce the amino acid Cα-formylglycine (FGly) into recombinant proteins, starting from cysteine or serine residues within distinct consensus motifs. The aldehyde-bearing FGly-residue displays orthogonal reactivity to all other natural amino acids and can, therefore, be used for site-specific labeling reactions on protein scaffolds. (Reviewed in Kruger et al. (2019) BIOL. CHEM., 400(3):289-297).

Formylglycine generating enzyme (FGE) recognizes a pentapeptide consensus sequence, C×P×R, and it specifically oxidizes the cysteine in this sequence to an unusual aldehyde-bearing formylglycine. The FGE recognition sequence, or aldehyde tag, can be inserted into heterologous recombinant proteins produced in either prokaryotic or eukaryotic expression systems. The conversion of cysteine to formylglycine is accomplished by co-overexpression of FGE, either transiently or as a stable cell line, and the resulting aldehyde can be selectively reacted with α-nucleophiles to generate a site-selectively modified bioconjugate (Rabuka et al. (2012) NAT. PROTOC., 7(6): 1052-1067).

In some embodiments, the enzyme is lipoic acid ligase, an enzyme that modifies a lysine side-chain within the 13-residue target sequence (Uttamapinant et al. (2010) PROC. NATL. ACAD. SCI. USA, 107(24):10914-9) to introduce bio-orthogonal groups, including azides, aryl aldehydes and hydrazines, p-iodophenyl derivatives, norbornenes, and trans-cyclooctenes (reviewed in Debelouchina et al. (2017) Q. REV. BIOPHYS., 50 e7).

In other embodiments, the enzyme is biotin ligase, farnesyltransferase, transglutaminase or N-myristoyltransferase (reviewed in Rashidian et al. (2013) BIOCONJUG. CHEM., 24(8):1277-94).

### IV. Pharmaceutical Compositions and Kits

The multimers can be incorporated into a pharmaceutical composition, formulated together with a pharmaceutically acceptable carrier. Such compositions may include one or a combination of (e.g., two or more different) multimers. The multimer pharmaceutical composition also can be used in combination with other therapeutics agents, or other therapeutic modalities, in a subject (so-called combination therapy that combines two or more treatment approaches).

Preferably, in some embodiments, the carrier is suitable for intravenous, intramuscular, subcutaneous, parenteral, spinal or epidermal administration (e.g., by injection or infusion).

The pharmaceutical composition also may include one or more pharmaceutically acceptable salts. A “pharmaceutically acceptable salt” refers to a salt that retains the desired biological activity of the parent compound and does not impart any undesired toxicological effects (see e.g., Berge et al. (1977) J. PHARM. SCI., 66:1-19). Non-limiting examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

The pharmaceutical composition also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, α-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

The pharmaceutical compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms are dictated by and directly dependent on (a) the unique characteristics of the active compound and the particular therapeutic effect to be achieved, and (b) the limitations inherent in the art of compounding such an active compound for the treatment of sensitivity in individuals.

Pharmaceutical compositions can be administered via one or more routes of administration using one or more of a variety of methods known in the art. As will be appreciated by the skilled artisan, the route and/or mode of administration will vary depending upon the desired results.

Preferred routes of administration for multimers of the disclosure include intratumoral, intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intratumoral, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, and intrasternal injection and infusion.

In another aspect, the disclosure provides kits comprising a multimer, e.g., for use in the methods described herein. Any of the compositions of the disclosure can be formulated into a kit, including the components, typically packaged in one or more containers, along with instructions for use of the components for the desired use. For example, in one embodiment, the disclosure provides a kit comprising at least one multimer composition packaged in a container, along with instructions for use of the multimer, e.g., for detecting pMHC-TCR interactions or for immunomodulation. In another embodiment, the kit comprises at least one expression construct for expressing a multimer of the disclosure, typically packaged in a container, along with instructions for use of the construct(s), e.g., for expression of a multimer in a host cell. In some embodiments, a kit comprises components for constructing a multimer, such as, e.g., one or more plasmids encoding one or more constructs as provided herein, that can be transfected into a host to generate a multimer.

### V. Methods of Use

Multimers of the disclosure can be used in a variety of in vitro and in vivo methods, as research reagents, for diagnostic purposes and for therapeutic uses, based on the binding specificity of the multimers and on the effector functions of the multimers.

Since the MHC moiety of a provided multimer is designed to be recognized by T cell receptors, in some embodiments, the multimers can be used to detect TCR-peptide-MHC complex interactions. In another aspect, the present disclosure provides methods of detecting a TCR-peptide-MHC interaction, the method comprising contacting a TCR with a multimer of the disclosure and detecting binding of the TCR to the peptide-MHC complex of the MHC moiety of the multimer to thereby detect the TCR-peptide-MHC interaction. In some embodiments, detection is used as a diagnostic for one or more diseases. In addition to use in detection, compositions comprising MHC multimers may be used for therapeutic purposes such as for treatment of one or more diseases or disorders in a subject in need thereof.

### A. TCR Localization and Specificity

In one embodiment, the TCR is on the surface of a cell, e.g., a T cell, and the multimer is contacted with the T cell to thereby contact the multimer with the TCR on the T cell. In another embodiment, the TCR is fixed to a solid support, such as a plate or a bead. In another embodiment, the peptide-MHC complex is fixed to a solid support, such as a plate, bead, tips, or other solid surface. In some such embodiments, the complex is fixed to the solid support indirectly, such as, for example, through another agent such as, e.g., streptavidin. In yet another embodiment, the TCR is soluble. Binding of the multimer to the TCR can be detected by standard methods known in the art, e.g., by labeling the multimer with a detectable label and then detecting the amount of label associated with the TCR. Alternatively, the TCR can be labeled with a detectable label and then the amount of label associated with the multimer can be detected.

In one embodiment, the MHC moiety of the multimer has a known TCR specificity and the method can be used to detect that particular TCR in a sample, e.g., for research purposes or on the surface of patient cells for the purposes of characterizing a patient's T cell population or diagnosing a patient as having an autoimmune disorder with a particular TCR reactivity.

In another embodiment, the detection method can be used to analyze the binding specificity of the MHC moiety using in the multimer. For example, an MHC molecule whose antigenic specificity is unknown can be used in the MHC moiety to determine the binding specificity of the MHC moiety using the detection method.

### B. Targeting Specific T Cell Populations

The present disclosure provides the insight that pMHC-multimers can be coupled to certain effector/targeting moieties to target changes to specific populations of T cells.

T cells as disclosed herein can be from a T cell subpopulation defined by, e.g., function, activation state, maturity, potential for differentiation, expansion, recirculation, localization, and/or persistence capacities, antigen specificity, type of antigen receptor, presence in a particular organ or compartment, marker or cytokine secretion profile, and/or degree of differentiation. For example, in some embodiments subpopulations of T cells are naive T (Tn) cells, effector T cells (Teff), memory T cells and sub-types thereof, such as stem cell memory T (Tscm), central memory T (Tcm), effector memory T (Tem), or terminally differentiated effector memory T cells, tumor-infiltrating lymphocytes (TIL), immature T cells, mature T cells, helper T cells, cytotoxic T cells, mucosa-associated invariant T (MALT) cells, naturally occurring and adaptive regulatory T (Treg) cells, helper T cells (e.g., TH1 cells, TH2 cells, TH3 cells, TH17 cells, TH9 cells, TH22 cells, follicular helper T cells), natural killer T (NKT) cells, alpha(α)/beta(β) T cells, and gamma(γ)/delta(δ) T cells. T cells can be characterized by the expression of certain cell surface molecules such as the T cell receptor (TCR), a multiprotein complex that is involved in MHC binding.

In some embodiments, T cells will be targeted by using pMHC multimers coupled to effector/targeting moieties that act either in cis (i.e., directly on target T cells) or in trans (i.e., to engage or impact a population of cells that then acts on T cells). Particular tissues or T cell populations to be targeted and effector/targeting moieties chosen will depend on context and desired outcome. For instance, in accordance with technologies provided by the present disclosure, a population of T cells (e.g., epitope-specific T cells) may be targeted for inhibition, exhaustion, anergization, and/or depletion. Without wishing to be bound by theory, in some embodiments, such suppressive signals may be useful in modulating T cells (e.g., effector T cells, helper T cells) that attack a self tissue, thereby to treat an autoimmune disease. In another embodiment, a population of T cells (e.g., epitope-specific T cells) may be targeted for support (e.g., survival), expansion, activation, and/or memory formation. The present disclosure also contemplates that in some such embodiments, such activation or activating signals are useful in stimulating T cells (e.g., effector T cells, helper T cells) that attack undesired cells or tissues, such as cancer cells, thereby treating cancer. Alternatively or additionally, where target T cells include regulatory T cells, such activating signals may be used to induce tolerance to selected epitopes, such as autoimmunity-associated epitopes, thereby to treat autoimmune diseases.

### 1. Therapeutic Multimers for Treating Autoimmune Diseases

i. Inhibiting and/or Exhausting T Effector Cells by Cis pMHC-Mediated Targeting

In some embodiments, the present disclosure provides technologies that can be used to treat autoimmune disease.

For example, in a context of autoimmunity, specific self-reactive T effector cells may be targeted using a pMHC multimer coupled to an effector/targeting moiety. In some such embodiments, the effector/targeting moiety provides or acts to induce a signal that inhibits activity or, first activates the T effector, but with chronic activation, induces self-reactive T cells (i.e., those attacking self-tissue) into an exhausted state. For example, without being bound by any particular theory, the present disclosure contemplates that even if a population of T cells experiences an initial activation upon binding of an effector/targeting moiety, upon continued exposure, undesirable T cells will be exhausted and no longer able to attack the tissue of a subject suffering from an autoimmune disease.

In some embodiments, specific populations of T cells (e.g., effector T cells) are targeted for inhibition or exhaustion in a cis approach using an pMHC multimer coupled to an effector/targeting moiety as provided herein. In some such embodiments, the effector/targeting moiety comprises one or more of a checkpoint protein agonist, a TNFR superfamily agonist (e.g., a TNF superfamily member), or an anti-inflammatory cytokine, or binding fragment (e.g., an antigen-binding fragment) thereof. For example, in some embodiments, an effector/targeting moiety may be or comprise a PD-L1 extracellular domain or domain 1, anti-PD1 agonist antibody, anti-CTLA4 agonist antibody, anti-Lag3 agonist antibody, anti-TIM3 agonist antibody, anti-TIGIT agonist, 4-1BBL (CD137L), anti-CD137 agonist antibody, HLA-G, anti-LILRB1 agonist antibody, anti-LILRB2 agonist antibody, IL10, TGFβ or an antigen-binding fragment thereof (e.g., that can bind and/or act on a targeted T cell, e.g., agonistic binding).

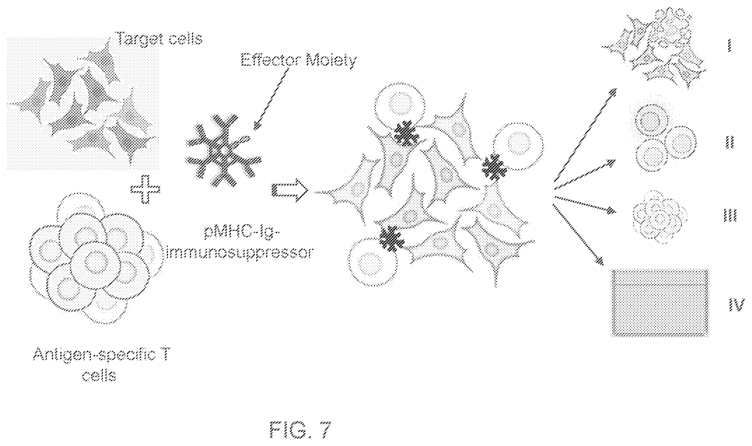

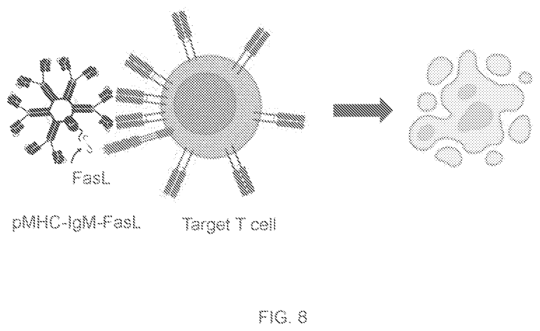

Immunosuppression or exhaustion may be assessed by, among other things, evaluating ability to chronically reduce TCR signaling, proliferation, cytokine secretion, or cell killing by pMHC-multimer targeted, antigen-specific effector T cells, as illustrated in an exemplary schematic (see FIG. 7) and further described in Example 7.

ii. Killing or Inducing Apoptosis of T Effector Cells by Cis pMHC-Mediated Targeting

In some embodiments, self-antigen-specific T cells may be targeted to induce cell death, whether by apoptosis or other mechanisms. For example, by using a pMHC multimer comprising an effector/targeting moiety where the effector/targeting moiety triggers or causes cell toxicity, damage (e.g., inducing programmed cell death), or causes death by another pathway, T effector cells that are attacking self-tissue can be eliminated. For example, in some embodiments, a pMHC multimer may comprise an effector/targeting moiety comprising FasL, an anti-Fas agonist antibody domain, a tubulin inhibitor, a DNA damaging agent, or any or any portion or component thereof that can bind or be delivered to act on a targeted T cell.

To give but one example, pMHC multimers comprising an effector/targeting moiety comprising FasL or anti-Fas agonist peptide can be used to trigger apoptosis in effector T cells. In such an example, a pMHC multimer coupled to a FasL or anti-Fas agonist peptide can be co-incubated with a mix of antigen- and non-antigen-specific T cells. Antigen-specific T cells should be particularly targeted for delivery of the effector/targeting moiety on the pMHC multimer, thus, specifically eliminated from the T cell population, relieving the autoimmune attack. One way to evaluate impact of such a strategy is to use differential labeling techniques (such as using a discriminating anti-HLA label) can be employed to determine relative numbers of each cell type (e.g., using flow cytometry). In addition, markers such as Annexin V or propidium iodide, and/or counts/relative amounts of certain antigen specific T cells can determine if apoptosis has selectively and/or successfully been induced (see, e.g., schematic at FIG. 8 for an exemplary approach).

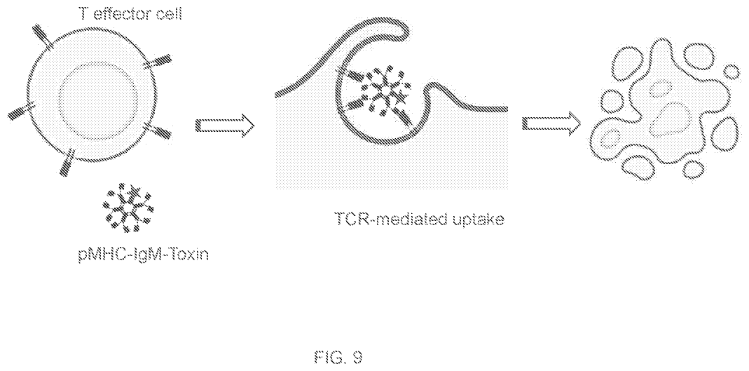

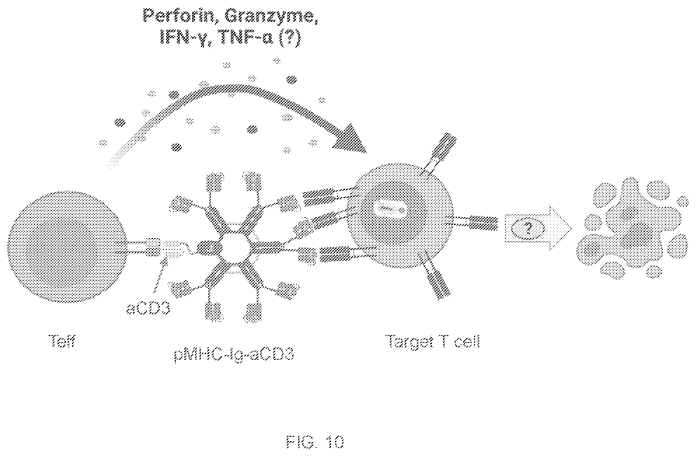

Another approach for eliminating targeting specific T cell populations includes using a pMHC multimer coupled to a cytotoxic drug. For example, a pMHC multimer can be coupled to a cytotoxic agent such as a tubulin inhibitor or DNA damaging agent. The pMHC multimer comprising a cytotoxic effector moiety can be co-incubated with a mix of antigen-specific and antigen-non-specific T cells. The present disclosure contemplates that when such a multimer is incubated with a mixture of T-cells, the pMHC multimer will be taken up by the antigen-specific T cells and the cytotoxic effector moiety will only impact that population, leaving the non-antigen-specific T cells. Cells can be evaluated using exemplary strategies such as shown in the schematic of FIG. 9, where, for instance, differential labeling techniques (such as using a discriminating anti-HLA label) can be employed to determine relative numbers of each cell type (e.g., using flow cytometry) present and/or markers such as Annexin V or propidium iodide, and/or counts/relative amounts of certain populations of T cells (antigen-specific and antigen non-specific) can be used to determine if apoptosis has selectively and/or successfully been induced in a targeted population.

iii. Killing T Effector Cells by Trans pMHC-Mediated Targeting to Recruit Other Immune Cell Populations

In some embodiments, self-antigen-specific T effector cells may be depleted by using a pMHC multimer comprising an effector/targeting moiety that binds an immune effector cell (e.g., T cell, NK cell). This is a trans approach, which indirectly acts on a target T cell by facilitating the formation of an immunological synapse between an immune effector cell and a T cell targeted by the pMHC, thereby to kill the target T cell. For example, in some embodiments, the effector/targeting moiety comprises a binding domain (e.g., an antibody or an antigen-binding fragment thereof) that binds a T cell surface protein, such as CD3, CD2, or CD8. In a specific embodiment, the effector/targeting moiety comprises an anti-CD3 antibody or an antigen-binding fragment thereof. Such peptide-MHC multimer (e.g., peptide-MHC-Ig multimer) fusion protein can engage effector T cells to antigen-specific T cells, where the pMHC multimer is targeted to the antigen-specific T cells, and the anti-CD3 domain engages the other T cells (e.g., effector T cells), which kill the antigen-specific T cell via secretion of perforin, granzyme and other mechanisms (FIG. 10). Differential labeling of the different cells (CFSE or, for instance, a discriminating anti-HLA antibody) can be used to determine relative number of each cell type by flow, and the loss of the antigen-specific T cells caused by the peptide-MHC-αCD3 multimer can be assessed. In some embodiments, the effector/targeting moiety comprises a binding domain (e.g., an antibody or an antigen-binding fragment thereof) that binds an NK cell surface protein, such as NKp46, CD16a, CD56, NKG2D, NKp30, or CD64. Such peptide-MHC multimer (e.g., peptide-MHC-Ig multimer) fusion protein can engage NK cells to antigen-specific T cells, thereby to kill the antigen-specific T cell via secretion of perforin, granzyme and other mechanisms.

iv. Reprogramming, Amplifying, or Supporting T Regulatory Cells by Targeting in Cis

In some embodiments, specific populations of T cells may be targeted for support, amplification of response, or for reprogramming. For instance, in some embodiments, regulatory T cells are targeted and provided support using an pMHC multimer coupled to an effector/targeting moiety comprising an IL-2 mutein (e.g. that has reduced or no binding affinity for CD25/ILR2α) or TGF-β, or a variant of any of these cytokines, or an anti-TNFR2 agonist (e.g., an antigen-binding antibody fragment). Stimulation of regulatory T cells can modulate immunity against a self tissue, thereby to treat an autoimmune disease.

Among other things, the present disclosure also contemplates that, in some embodiments, MHC multimers can downmodulate an autoimmune response by reprogramming cognate antigen-experienced CD4+ cells into disease-suppressing T-regulatory type 1 (TR1) cells. In some embodiments, the MHC multimer comprises an effector/targeting moiety that promotes immunosuppression and/or induction of TR1 cells. Non-limiting examples of such targeting moieties are described herein and include TGFβ, FasL, TNFR2 and IL-10. In some embodiments, Treg or TR1-promoting cytokines include IL-10 or Treg-focused IL-2.

v. Combining Expansion of T Regulatory Cells and Killing of T Effector Cells

Depending on circumstances, in some embodiments, autoimmune disease may be treated by amplifying activity or expanding a particular population of T cells, such as T regulatory cells. In some embodiments, for instance, a pMHC multimer coupled to an effector/targeting moiety to provide a specific tolerizing or stimulatory signal. For example, in some embodiments, T regulatory cells can be targeted for expansion and/or effector T cells targeted for cell death simultaneously or sequentially. To give but one example, in some embodiments, a pMHC multimer coupled to an effector/targeting moiety can act in one way on one type of cells (e.g., T regs) and another way on a different type of cells (e.g., T effs). In some embodiments, an effector/targeting moiety may comprise TRAIL, anti-TRAILR1 agonist antibody domain, anti-TRAILR2 agonist antibody domain, TGRβ, TNFR2 agonists, or any portion or fragment thereof that can bind and act on a T cell receptor of a targeted T cell. In certain embodiments, if two different peptides are expressed on, e.g., a single IgM, it may be possible to eliminate/deplete/anergize T cells that recognize either epitope (i.e. of the two expressed peptides). Cell populations may be assessed to determine relative abundance of certain cell types (e.g., T regulatory cells, T effectors) by using cell markers (e.g., Foxp3, CD25) depending on context and cell type targeted and/or desired. (FIG. 11).

### 2. Therapeutic Multimers for Treating Cancer

i. pMHC Multimers to Enhance T Effector Cell Activity in Cancer

The present disclosure provides the insight that pMHC multimers may also be used to treat cancer. For example, in some embodiments, the present disclosure contemplates that amplification (e.g., activation, proliferation, etc.) of effector T cells (e.g., effector T cells that recognize a cancer cell surface peptide-MHC are useful in treatment of cancer. Accordingly, in some embodiments, a multimer comprises an effector/targeting moiety, which effector/targeting moiety is used to enhance T effector cell activity. Without wishing to be bound by theory, in some such embodiments, enhancement of a specific population of T effector cell activity causes cells to attack a population of cancer cells (e.g., a tumor). In some embodiments, by way of non-limiting example, an effector/targeting moiety is or comprises IL-12, IL-15, or a variant of any of these cytokines (e.g., an IL-2 mutein with reduced or no binding affinity for CD25/IL2Rα), an anti-CD28 antibody, an anti-CD40L antibody, an anti-4-1BB antibody, or an antigen-binding fragment of any of these antibodies, thereby to enhance the activity of the T effector cell. In some embodiments, an effector/targeting moiety is or comprises an antagonist anti-PD1, anti-TIGIT, anti-CTLA4, anti-TIM3, anti-Lag3, anti-LILRB1, or anti-LILRB2 antibody or an antigen-binding fragment of any of these antibodies, thereby to enhance the activity of the T effector cell.

### 3. Therapeutic Proteins for Treating Infection

i. pMHC Multimers to Enhance T Effector Cell Activity in Infectious Diseases

The present disclosure provides the insight that pMHC multimers may also be used to treat infection (e.g., a viral infection). For example, in some embodiments, the present disclosure contemplates that amplification (e.g., activation, proliferation, etc.) of effector T cells (e.g., effector T cells that recognize an infectious epitope presented by an infected cell such as, for example, a viral epitope), can be used to target infected cells. Accordingly, in some embodiments, a multimer comprises an effector/targeting moiety, which effector/targeting moiety is used to enhance T effector cell activity. Without wishing to be bound by theory, in some such embodiments, enhancement of a specific population of T effector cell activity causes cells to attack a population of infected cells (e.g., virally-infected cells)._In some embodiments, by way of non-limiting example, an effector/targeting moiety is or comprises IL-2, IL-12, IL-15, or a variant of any of these cytokines, an anti-CD28 antibody, an anti-CD40L antibody, an anti-4-1BB antibody, or an antigen-binding fragment of any of these antibodies, thereby to enhance the activity of the T effector cell.

In some embodiments, an effector/targeting moiety is or comprises an antagonist anti-PD1, anti-TIGIT, anti-CTLA4, anti-TIM3, anti-Lag3, anti-LILRB1, or anti-LILRB2 antibody or an antigen-binding fragment of any of these antibodies, thereby to enhance the activity of the T effector cell.

### 4. Therapeutic Proteins for Treating Autoimmune Diseases or Cancer

i. Combinations for Targeting Multiple Populations

The present disclosure also contemplates that targeting one or more populations of T cells using distinct mechanisms with distinct effector/targeting moieties can be accomplished using multiple compositions, each composition comprising a distinct pMHC multimer formulation. For example, in some embodiments, one population of T cells may be targeted for expansion and one targeted for cell death and each targeted by a distinct pMHC multimer coupled to an effector capable of supporting or killing particular T cells. In some embodiments, such an approach may be used in treatment of autoimmune diseases or in cancers.

### C. Administration

When used therapeutically, the MHC multimers can be administered to a subject (e.g., intravenously or intramuscularly) to modulate an immune response in the subject. Accordingly, in another aspect, the disclosure pertains to a method of modulating an immune response in a subject, the method comprising administering to the subject an MHC multimer of the disclosure such that an immune response is modulated in the subject. Modulation of an immune response in the subject results from the combination of the binding of the peptide-MHC moiety to its target TCRs in a multivalent manner (due to the multivalency of the multimer) and the effector functions mediated by the IgM or IgA C regions within the multimerization domain, such as complement-mediated cytotoxicity, as well as any effector functions mediated by an effector/targeting moiety and/or additional functional moiety included in the MHC multimer. For example, inclusion of a PD-L1-binding effector/targeting moiety can redirect (e.g., exhaust) endogenous T cells to the MHC multimers or MHC-multimer-bound target cell in vivo; the endogenous T cells are then modulated in the subject (e.g., endogenous antigen-specific T cells that recognize the MHC multimers are exhausted), thereby modulating an immune response.

In some embodiments, compositions provided by the present disclosure are administered in vivo or ex vivo. In some embodiments, compositions are administered subcutaneously, intramuscularly, or intravenously. In some embodiments, compositions are administered by targeted injection into a particular site (e.g., an inflamed tissue or organ, e.g., a tumor).

In certain embodiments, a method or composition provided herein, is administered in combination with one or more additional therapies, e.g., surgery, radiation therapy, anti-inflammatory therapy, biologic therapy, etc. In some embodiments, the additional therapy may include chemotherapy, e.g., a cytotoxic agent. In some embodiments the additional therapy may include a targeted therapy, e.g., a tyrosine kinase inhibitor, a proteasome inhibitor, or a protease inhibitor. In some embodiments, the additional therapy may include an anti-inflammatory, anti-angiogenic, anti-fibrotic, or anti-proliferative compound, e.g., a steroid, a biologic immunomodulator, a disease modifying anti-rheumatic drug, a monoclonal antibody, an antibody fragment, an aptamer, an siRNA, an antisense molecule, a fusion protein, a cytokine, a cytokine receptor, a bronchodilator, a statin, an anti-inflammatory agent (e.g., methotrexate), or an NSAID. In certain embodiments, the additional therapy may include a combination of therapeutics of different classes.

In some embodiments, one or more agents administered in combination with one another may be administered in one or more doses. In some embodiments, administration of a combination of two or more agents can be sequential or concomitant.

### D. Diseases

The present disclosure provides the insight that one or more immune-mediated disease, such as autoimmune disease, inflammatory disease, and/or cancer, may be detected, diagnosed, and/or treated using a composition comprising an MHC multimer as provided herein.

In one embodiment, an MHC multimer of the disclosure is administered to a subject with an autoimmune disease or disorder to downmodulate the autoimmune response in the subject. Accordingly, in one embodiment, the method comprises administering to the subject an MHC multimer of the disclosure wherein the peptide-MHC moiety of the multimer is recognized by autoimmune T cells of the subject.

In various embodiments, the autoimmune disease is one of the following (with known disease-associated HLA alleles shown in parentheses): Type I diabetes (HLA-DQ2, HLA-DQ8), celiac disease (HLA-DQ2, HLA-DQ8), primary sclerosing cholangitis (HLA-DR8, HLA-DRB1), vitiligo and autoimmune skin diseases (HLA-B27), atopic dermatitis (HLA-DRB1), rheumatoid arthritis, psoriatic arthritis, and ulcerative colitis, ankylosing spondylitis, Crohn's disease, inflammatory bowel disease, Alzheimer's disease, uveitis, psoriasis, scleroderma, lupus erythematosus, Parkinson's disease, amyotrophic lateral sclerosis, autoimmune hepatitis, primary biliary cholangitis, Sjogren's syndrome, narcolepsy, multiple sclerosis, alopecia areata, Grave's disease, Addison's disease, Hashimoto's disease, Myasthenia gravis, neuromyelitis optica, pemphigus vulgaris, bullous pemphigoid, myelin oligodendrocyte glycoprotein antibody-associated disease and acquired factor VIII deficiency. In one embodiment, the autoimmune disease is Type I diabetes. In one embodiment, the peptide used in the MHC multimer is a Type I diabetes-associated peptide, such as an insulin peptide.

In some embodiments, a pMHC multimer of the disclosure is administered to a subject with an infectious disease. Accordingly, in some such embodiments, the method comprises administering to the subject a pMHC multimer of the disclosure wherein the MHC moiety of the multimer is recognized by immune cells of the subject and, in some embodiments, can act either in cis or trans to directly bind to a target cell, or to recruit another population of cells to impact a target cell population and treat the infection. In some embodiments, the infectious disease is bacterial. In some embodiments, the infectious disease is viral.

In another embodiment, an MHC multimer is administered to a subject with cancer and modulates the immune response to the cancer in the subject. In some embodiments, the present disclosure provides a method of treating cancer using an immunotherapy comprising a pMHC multimer comprising an effector/targeting moiety. In some such embodiments, the effector/targeting moiety modulates one or more immune cell populations to treat the cancer, such as by amplifying an anti-tumor response or depleting or suppressing tumor cells. Accordingly, in one embodiment, the method comprises administering to the subject an MHC multimer of the disclosure wherein the peptide-MHC moiety of the MHC multimer is recognized by T cells (e.g., cancer-specific T cells) of the subject. In one embodiment, the MHC multimer includes an effector/targeting moiety. In one embodiment, the effector/targeting moiety is an activating agent, e.g., an agent that expands tumor-reactive T cells. Non-limiting examples of such activating targeting moieties include IL-2 or muteins thereof (e.g., an IL2 mutein with reduced or no binding affinity for CD25/IL2Rα), IL12, IL15, anti-CD28 antibody domain, anti-CD40L antibody domain, anti-4-1BB antibody domain, CD80, CD86, and 4-1BBL. In another embodiment, the effector/targeting moiety is an inhibitory agent, e.g., an immunosuppressive agent and/or an agent that selectively inhibits Tregs. Non-limiting examples of such inhibitory targeting moieties include PD-L1, FasL and TGFβ and others provided herein. Additionally or alternatively, the subject can be treated with additional anti-cancer agents, including chemotherapeutic agents, immunotherapy agents and immune checkpoint inhibitors.

In some embodiments, the cancer is or comprises a solid tumor. In some embodiments, the cancer is a hematological cancer.

For example, in certain embodiments, the cancer is brain cancer, bladder cancer, breast cancer, cervical cancer, colon cancer, colorectal cancer, endometrial cancer, esophageal cancer, leukemia, lung cancer, liver cancer, melanoma, ovarian cancer, pancreatic cancer, prostate cancer, rectal cancer, renal cancer, stomach cancer, testicular cancer, or uterine cancer, squamous cell carcinoma, adenocarcinoma, small cell carcinoma, melanoma, glioma, neuroblastoma, sarcoma (e.g., an angiosarcoma or chondrosarcoma), larynx cancer, parotid cancer, biliary tract cancer, thyroid cancer, acral lentiginous melanoma, actinic keratoses, acute lymphocytic leukemia, acute myeloid leukemia, adenoid cystic carcinoma, adenomas, adenosarcoma, adenosquamous carcinoma, anal canal cancer, anal cancer, anorectum cancer, astrocytic tumor, bartholin gland carcinoma, basal cell carcinoma, biliary cancer, bone cancer, bone marrow cancer, bronchial cancer, bronchial gland carcinoma, carcinoid, cholangiocarcinoma, chondosarcoma, choroid plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangiblastomas, hemangioendothelioma, hemangiomas, hepatic adenoma, hepatic adenomatosis, hepatobiliary cancer, hepatocellular carcinoma, Hodgkin's disease, ileum cancer, insulinoma, intaepithelial neoplasia, interepithelial squamous cell neoplasia, intrahepatic bile duct cancer, invasive squamous cell carcinoma, jejunum cancer, joint cancer, Kaposi's sarcoma, pelvic cancer, large cell carcinoma, large intestine cancer, leiomyosarcoma, lentigo maligna melanomas, lymphoma, male genital cancer, malignant melanoma, malignant mesothelial tumors, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma nodular melanoma, non-epithelial skin cancer, non-Hodgkin's lymphoma, oat cell carcinoma, oligodendroglial cancer, oral cavity cancer, osteosarcoma, papillary serous adenocarcinoma, penile cancer, pharynx cancer, vascularized tumor, pituitary tumor, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, striated muscle cancer, submesothelial cancer, superficial spreading melanoma, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well differentiated carcinoma, or Wilms tumor, T cell leukemia, or non-Hodgkin's lymphoma (such as a B-cell lymphoma or a T-cell lymphoma). In certain embodiments, the non-Hodgkin's lymphoma is a B-cell lymphoma, such as a diffuse large B-cell lymphoma, primary mediastinal B-cell lymphoma, follicular lymphoma, small lymphocytic lymphoma, mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, hairy cell leukemia, or primary central nervous system (CNS) lymphoma. In certain other embodiments, the non-Hodgkin's lymphoma is a T-cell lymphoma, such as a precursor T-lymphoblastic lymphoma, peripheral T-cell lymphoma, cutaneous T-cell lymphoma, angioimmunoblastic T-cell lymphoma, extranodal natural killer/T-cell lymphoma, enteropathy type T-cell lymphoma, subcutaneous panniculitis-like T-cell lymphoma, anaplastic large cell lymphoma, or peripheral T-cell lymphoma.

### EXAMPLES

Below are examples of specific embodiments for carrying out what is disclosed herein. The examples are offered for illustrative purposes only and are not intended to limit scope.

The practice of the present disclosure will employ, unless otherwise indicated, conventional methods of protein chemistry, biochemistry, recombinant DNA techniques and pharmacology, within the skill of the art. Such techniques are explained fully in the literature. See, e.g., T. E. Creighton, PROTEINS: STRUCTURES AND MOLECULAR PROPERTIES (W.H. Freeman and Company, 1993); A. L. Lehninger, BIOCHEMISTRY (Worth Publishers, Inc., current addition); Sambrook, et al. MOLECULAR CLONING: A LABORATORY MANUAL (2nd Edition, 1989); METHODS IN ENZYMOLOGY (S. Colowick and N. Kaplan eds., Academic Press, Inc.); REMINGTON'S PHARMACEUTICAL SCIENCES, 18th Edition (Easton, Pennsylvania: Mack Publishing Company, 1990); Carey and Sundberg ADVANCED ORGANIC CHEMISTRY 3^{rd} Ed. (Plenum Press) Vols A and B (1992).

Unless otherwise stated, all reagents and chemicals were obtained from commercial sources and used without further purification.

### Example 1—Recombinant Expression of MHC Class I Multimers

This example describes the expression, purification, and analysis of exemplary single-chain pMHC-IgM/J-chain Class I multimers.

To produce single chain-pMHC-IgM/J-chain complex loaded with ELA peptide (ELA-A2-IgM/J-chain complex) DNA constructs encoding: 1) the MHC Class I binding peptide ELAGIGILTV (SEQ ID NO:20), a flexible linker, β2m, a flexible linker, HLA-A2 (Y84A) soluble domain (SEQ ID NO: 276), a flexible linker, and IgM Cμ2, Cμ3, Cμ4 and the tailpiece regions and 2) J-chain were produced and cloned into expression plasmids. Other exemplary single-chain sequences comprising a substitution corresponding to Y84A are set forth in SEQ ID NOs.: 1-12, 199-204, 235-237, 243, 248-249, 259-260. An exemplary schematic of the constructs is shown in FIG. 5A. A first plasmid encoding a single chain-pMHC-IgM (ELA-A2-IgM, SEQ ID NO: 5, 88.2 kDa) and a second plasmid encoding the J-chain with Flag-tag and His-tag (SEQ ID NO: 251, 18.3 kDa) were transiently transfected into Expi293F cells using Expifectamine (ThermoFisher) for expression and secretion of the ELA-A2-IgM multimer. Cells were cultured for 5 days and harvested by centrifugation to separate the supernatant from the cells. Samples from the cell supernatant were subjected to SDS-PAGE and further analyzed by western blotting. The samples were split and analyzed under non-reducing/non-boiled conditions with NuPAGE™ Tris-Acetate 3-8% Gels (ThermoFisher) at 4° C. and reduced/boiled conditions with Bolt 4-12% Bis-Tris Plus Gels at room temperature. After the SDS-PAGE separation, western blots were probed with Dylight800-labeled anti-human IgM (ThermoFisher) and Dylight680-labeled anti-Flag (ThermoFisher) to assess expression levels (FIG. 12A for ELA-A2-IgM).

Both anti-IgM and anti-Flag antibodies detected a high molecular weight band in the samples from the non-reducing/non-boiling conditions (migrating higher than the highest molecular weight marker at 260 kDa) indicating co-migration of pMHC-IgM and J-chain-Flag and formation of an intact multimeric complex of pMHC-IgM/J-chain.

The pMHC-IgM/J-chain complex was purified by immobilized metal affinity chromatography (IMAC) using Nickel Sepharose excel resin (Ni Excel, Cytiva) utilizing the His-tag on the J-chain. The Ni Excel resin was pre-equilibrated in binding buffer (23 mM sodium phosphate, 500 mM NaCl, pH 7.4). Supernatant containing protein of interest was incubated with resin overnight and gravity flow purification was performed the next day. The Ni Excel resin was washed with wash buffer (23 mM sodium phosphate, 500 mM NaCl, 20 mM imidazole, pH 7.4) to remove unbound protein contaminants. Protein of interest was eluted using a gradient of 100 mM-500 mM imidazole in 23 mM sodium phosphate, 500 mM NaCl, pH 7.4. IMAC elution fractions were analyzed by western blot and fractions corresponding to intact complex of pMHC-IgM/J-chain were identified. IMAC elution fractions were pooled based on total amount and purity of pMHC-IgM/J-chain complex determined by western blot and concentrated using ultrafiltration (FIG. 12B). The concentrated pMHC-IgM/J-chain complex was loaded onto a Superdex 200 pg size exclusion chromatography (SEC) column pre-equilibrated with SEC buffer (20 mM HEPES, 150 mM NaCl, pH 7.2) for further purification (FIG. 12C). Elution fractions from the SEC were pooled based on total amount and purity of pMHC-IgM/J-chain complex determined by western blot and concentrated using ultrafiltration. To determine the protein concentration samples were measured by absorbance at 280 nm and the concentration calculated using the protein extinction coefficient.

Analytical size exclusion chromatography was performed to assess homogeneity and size using an AdvanceBio SEC 300 column, 4.6×50 mm, 2.7 μm (Agilent). FIG. 13A shows the analytical SEC for ELA-A2-IgM. The chromatogram shows a single, uniform peak indicative of a homogeneous sample. The purified pMHC-IgM/J-chain complex was characterized by SDS-PAGE stained with Coomassie (InstantBlue, Sigma-Aldrich) and western blot under non-reducing and reducing conditions. Analysis of the non-reduced/unboiled Coomassie-stained sample on the SDS-PAGE showed a single band at high molecular weight indicating an intact, pure ELA-A2-IgM/J-chain complex and western blot showed co-migrating bands with anti-IgM and anti-Flag blots indicating the formation of an intact complex of pMHC-IgM/J-chain (FIG. 13B). Analysis of the reduced/boiled Coomassie-stained sample showed an intense band at approximately 105 kDa corresponding to ELA-A2-IgM and a band at approximately 28 kDa corresponding to J-chain. The calculated molecular weights for ELA-A2-IgM and J-chain are 88.2 kDa and 18.3 kDa, respectively. Size differences on the SDS-PAGE from observed and calculated molecular weight are believed to result from the presence of glycosylation on both proteins. Western blots of reduced/boiled samples were probed with anti-IgM and anti-Flag antibodies and showed bands at approximately 105 kDa, corresponding to the ELA-A2-IgM and approximately 28 kDa corresponding to the J-chain (FIG. 13C).

Additional pMHC-IgM/J-chain complexes were cloned, expressed, and purified as described above. Details for additional complexes are shown in Table 3.

TABLE 3

| | | Pep- | | |
|---|---|---|---|---|
| | | tide | pMHC- | J |
| pMHC- | | SEQ | IgM | Chain |
| IgM | Peptide | ID | SEQ | SEQ |
| complex | Sequence | NO: | ID NO: | ID NO: |
| NLV-A2- | NLVPMVATV | 19 | SEQ ID | SEQ ID |
| pMHC-IgM | | | NO: 1 | NO: 251 |
| ELA-A2- | ELAGIGILTV | 20 | SEQ ID | SEQ ID |
| pMHC-IgM | | | NO: 5 | NO: 251 |
| ALW-A2- | ALWEIQQVV* | 257 | SEQ ID | SEQ ID |
| pMHC-IgM | | | NO: 235 | NO: 251 |
| KLW-A2- | KLWAQCVQL* | 258 | SEQ ID | SEQ ID |
| pMHC-IgM | | | NO: 236 | NO: 251 |
| YML-A2- | YMLDLQPETT | 160 | SEQ ID | SEQ ID |
| pMHC-IgM | | | NO: 237 | NO: 251 |

*Francis et al. (2022) SCI. IMMUNOL.

Additional pMHC-IgM/J-chain multimers comprising linker variants were cloned, expressed, and purified. 9-mer 1-pMHC-IgM (9-mer 1), 9-mer 3-pMHC-IgM (9-mer 3), 9-mer 4-pMHC-IgM (9-mer 4), and 9-mer 5-pMHC-IgM (9-mer 5), differed in the amino acid sequence of the 9-mer peptide, but had a common linker-b2m-linker-HLA-A2-linker-IgM sequence (SEQ ID NO: 259) and J-chain (SEQ ID NO: 251). 9-mer 2-pMHC-IgM (9-mer 2) had linker-b2m-linker-HLA-A2-IgM sequence (SEQ ID NO: 260) and J-chain (SEQ ID NO: 251).

Expression levels and purification yields of pMHC-IgM complexes varied depending on the bound peptide with respect to HLA-A*02:01. Final purification yields ranged between 0.4-40 mg of protein to expression below the limit of detection depending on the N-terminal peptide as shown in Table 4.

TABLE 4
pMHC-IgM production yields based on loading peptide.

| pMHC-IgM complex | Yield (mg/L of culture) |
|---|---|
| NLV-A2-pMHC-IgM | 0.498 |
| ELA-A2-pMHC-IgM | 1.495 |
| ALW-A2-pMHC-IgM* | 23.5 |
| KLW-A2-pMHC-IgM* | 28.5 |
| YML-A2-pMHC-IgM | 9.09 |
| 9-mer 1-pMHC-IgM | 2.82 |
| 9-mer 2-pMHC-IgM | 39 |
| 9-mer 3-pMHC-IgM | 7.35 |
| 9-mer 4-pMHC-IgM | BLOD |
| 9-mer 5-pMHC-IgM | BLOD |

BLOD = below limit of detection

### Example 2—Binding of MHC Class I Multimers to T Cells

This example describes the binding of pMHC-IgM complexes to T cells as measured by flow cytometry.

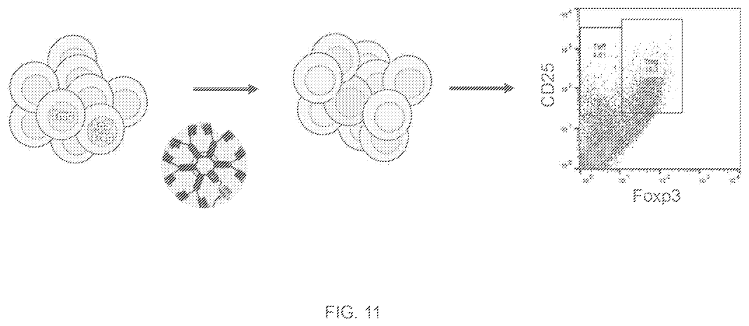

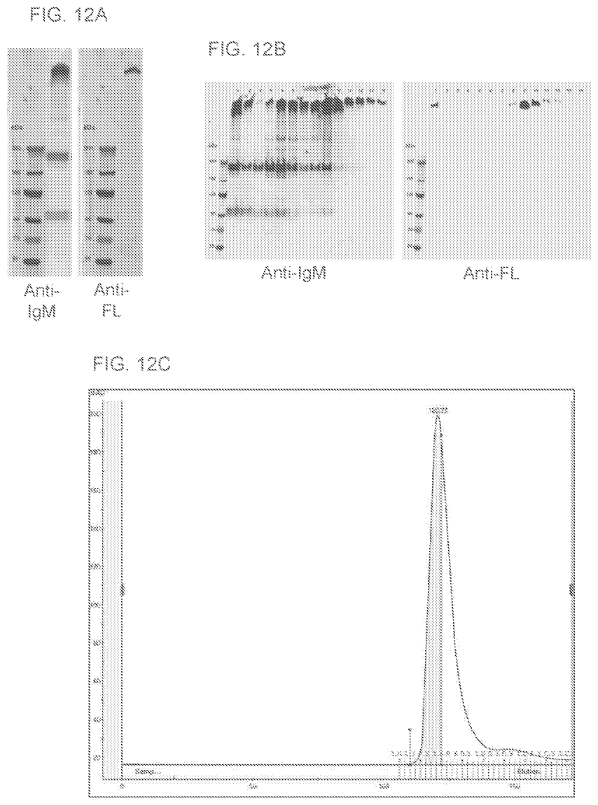

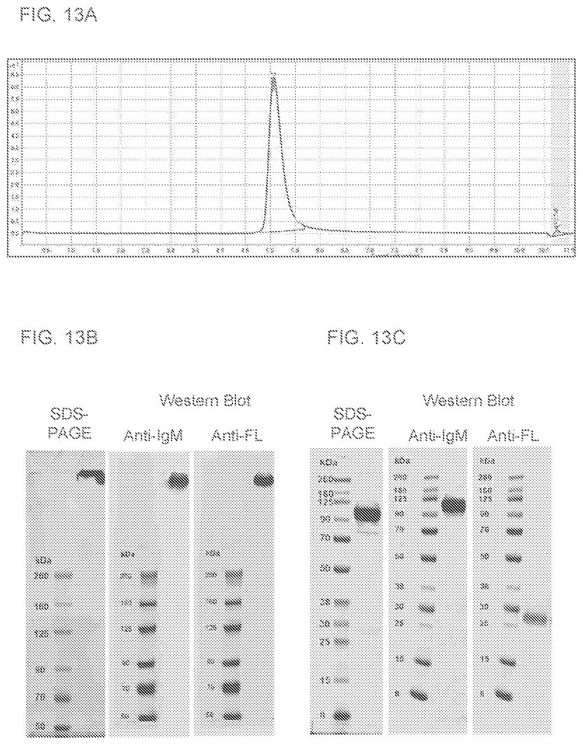

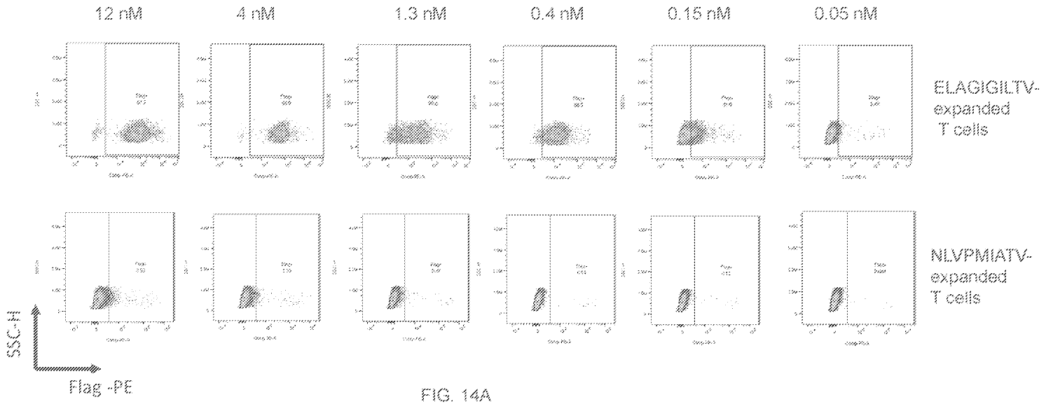

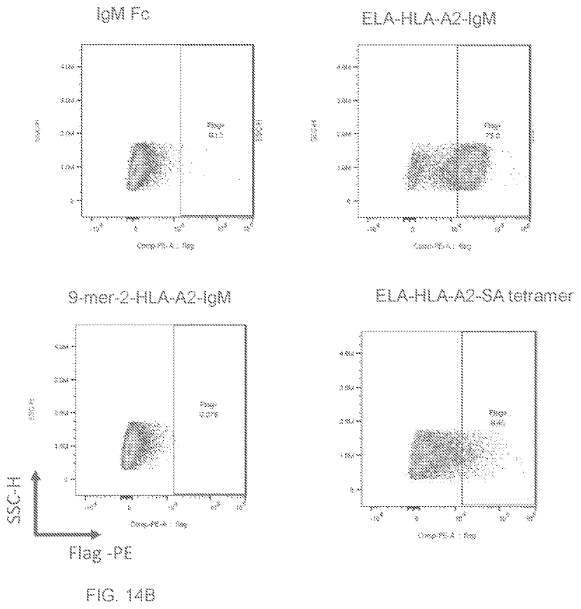

Flow cytometry was used to test binding of pMHC-IgM/J-chain complexes to T cells. An ELA-A2-pMHC-IgM/J-chain multimer complex containing a Flag tag at the J-chain C-terminus was produced essentially as described in Example 1. Anti-MART1 donor T cells specific for ELAGIGILTV (SEQ ID NO: 20)/HLA-A2 (Cellero, Northridge, CA) or non-cognate peptide NLVPMIATV (SEQ ID NO: 233)-expanded donor T cells were incubated with varying concentrations of ELA-A2-IgM/J-chain (12 nM, 4 nM, 1.3 nM, 0.4 nM, 0.15 nM, and 0.05 nM). Bound ELA-A2-IgM/J-chain was detected with PE-labeled anti-Flag antibody (FIG. 14A). ELA-A2-IgM/J-chain showed specific binding to cognate peptide-expanded T cells at all concentrations tested, and only minimal apparent binding to non-cognate-expanded T cells at the highest concentration. Exemplary binding of ELA-A2-pMHC-IgM/J-chain to T cells is shown in Table 5.

TABLE 5

| | percent | | | |
|---|---|---|---|---|
| ELA-A2-pMHC- | positive anti- | | percent positive | |
| IgM/J-chain | Flag | MFI anti-Flag | anti-Flag | MFI anti-Flag |
| concentration | ELAGIGILTV- | ELAGIGILTV- | NLVPMIATV- | NLVPMIATV- |
| (nM) | T cells | T cells | T cells | T cells |
| 12 | 97.7 | 62402 | 3.99 | 1371 |
| 4 | 99 | 51941 | 1.33 | 1107 |
| 1.3 | 86 | 18288 | 0.37 | 903 |
| 0.4 | 96.3 | 13649 | 0.14 | 801 |
| 0.15 | 31 | 3056 | 0.12 | 743 |
| 0.05 | 3.48 | 1682 | 0.084 | 743 |

ELA-A2-pMHC-IgM/J-chain complex was also tested for binding to J76-CD8 cell line expressing recombinant ELAGIGILTV-HLA-A2 specific TCR by flow cytometry at varying concentrations of pMHC-IgM/J-chain. J76-CD8-NFAT-GFP cells are a Jurkat subline that is devoid of endogenous TCR α and TCR β chains, thereby circumventing the problem of TCR mis-pairing and unexpected specificities.

ELA-A2-pMHC-IgM, 9-mer 2-HLA-A2-IgM, IgM-Fc, and ELA/HLA-A2-SA were tested for binding to ELAGIGILTV-HLA-A2 TCR on J76-CD8 cells at several concentrations as shown in Table 6.

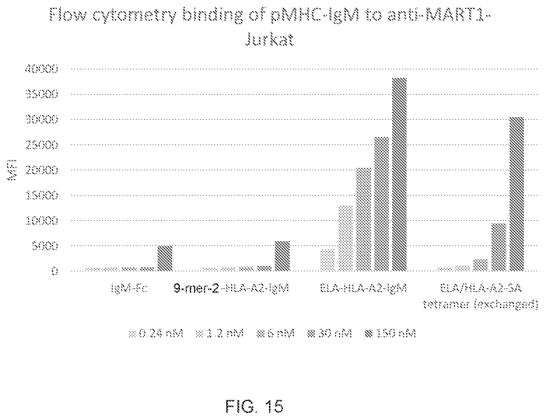

Exemplary flow cytometry plots for ELA-A2-pMHC-IgM, 9-mer 2-HLA-A2-IgM, IgM-Fc, and ELA/HLA-A2-SA at 6 nM concentration for binding to ELAGIGILTV-HLA-A2 TCR on J76-CD8 cells are shown in FIG. 14B. A graph of ELA-A2-pMHC-IgM, 9-mer 2-HLA-A2-IgM, IgM-Fc, and ELA/HLA-A2-SA binding to ELAGIGILTV-HLA-A2 TCR on J76-CD8 cells at several concentrations is shown in FIG. 15. ELA-A2-IgM/J-chain showed specific binding to its corresponding cognate ELAGIGILTV-HLA-A2 TCR on J76-CD8 cells at all concentrations tested, whereas no binding was detected with negative controls IgM-Fc or non-cognate 9-mer 2-HLA-A2-IgM/J-chain at and below 30 nM of ELA-A2-pMHC-IgM/J-chain (FIG. 15). ELA-A2-pMHC-IgM/J-chain showed increased binding to cognate TCR compared to ELAGIGILTV-peptide exchanged HLA-A2-streptavidin tetramer (ELA/HLA-A2-SA).

Exemplary binding of ELA-A2-pMHC-IgM/J-chain to T cells is shown in Table 6 using Mean Fluorescent Intensity at various concentrations of multimer components.

TABLE 6

| | | | | 9-mer 2-HLA- | | ELA/HLA-A2- | | ELA-A2-pMHC- | |
|---|---|---|---|---|---|---|---|---|---|
| | | | | A2-IgM | | SA | | IgM | |
| Protein | | IgM Fc | | % | | % | | % | |
| Concentration | | % positive | MFI | positive | MFI | positive | MFI | positive | MFI |
| 150 | nM | 1.94 | 4936 | 3.66 | 5899 | 77.2 | 30531 | 87.5 | 38247 |
| 30 | nM | 0.27 | 794 | 0.2 | 1048 | 41.4 | 9438 | 79.7 | 26545 |
| 6 | nM | 0.13 | 714 | 0.076 | 823 | 6.45 | 2326 | 75 | 20442 |
| 1.2 | nM | 0.14 | 765 | 0.17 | 743 | 0.61 | 1099 | 51.2 | 12999 |
| 0.24 | nM | 0.13 | 779 | 0.19 | 714 | 0.17 | 845 | 2.5 | 4377 |

### Example 3—Recombinant Expression of MHC Class II Multimers

This example describes the expression, purification, and analysis of exemplary pMHC-IgM/J-chain Class II multimers.

To produce single chain-pMHC-IgM/J-chain complex loaded with MTB peptide MTB-DR15-FosW/cJun-IgM(Cμ2-4) complex) DNA constructs encoding: 1) the MHC Class II binding peptide (SEQ ID NO: 234), a flexible linker, MHC Class II β chain, Fos heterodimerization motif (Mason et al. (2006) PROC. NATL. ACAD. SCI. USA, 103:8989-94), and IgM Cμ2, Cμ3, Cμ4, and the tailpiece regions, 2) MHC Class II α chain and Jun heterodimerization motif (Mason et al. (2006) PROC. NATL. ACAD. SCI. USA, 103:8989-94), and 3) J-chain were produced and cloned into three separate expression vectors. The Class II pMHC-IgM multimer MTB-DR15-FosW/cJun-IgM(Cu2-4) was expressed by transiently transfecting ExpiCHO cells with three expression constructs. The first expression construct encoded the MTB peptide-Linker-DRB1*15:01-Linker-FosW-Linker-IgM(Cμ2-4), SEQ ID NO: 207. The MTB peptide comprises residues 1-15 of ESAT-6-like protein EsxO from Mycobacterium tuberculosis (UniProt P9WNI7; SEQ ID NO: 234, MTINYQFGDVDAHGA). The second expression construct encoded DRA1*01-Linker-cJun-Myc, SEQ ID NO: 205. The third expression construct encoded the J-chain with C-terminal Flag and His tags, SEQ ID NO: 251.

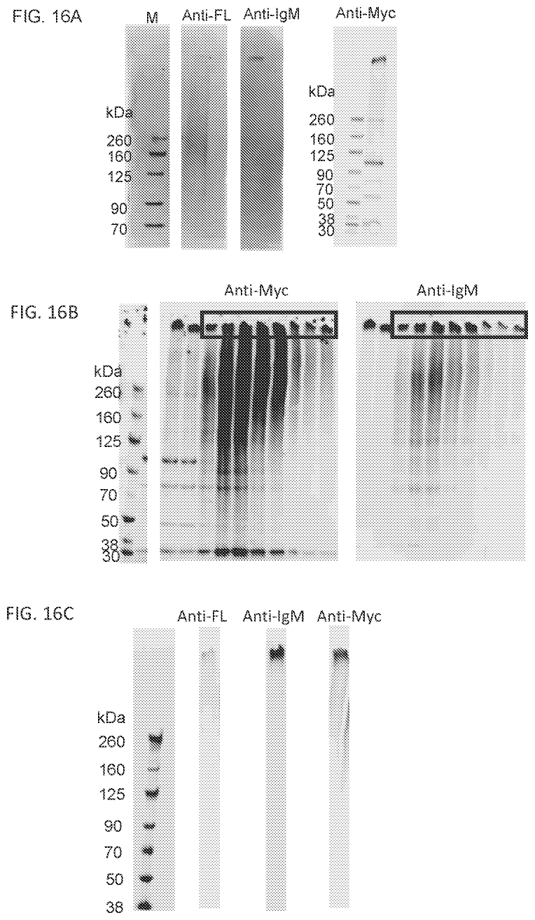

Transfection cultures were harvested 11 days after transfection, and expression of the pMHC-IgM was verified by western blot, shown in FIG. 16A. Samples from supernatant were separated by SDS-PAGE and analyzed by western blotting. Samples were analyzed under non-reducing/non-boiled conditions with NuPAGE™ Tris-Acetate 3-8% Gels (ThermoFisher) at 4° C. After SDS-PAGE separation, a western blot was probed with Dylight800-labeled anti-human IgM (ThermoFisher) antibody, Dylight680-labeled anti-Flag (ThermoFisher) antibody, rabbit polyclonal anti-Myc antibody, and goat anti-rabbit secondary antibody to assess expression levels. The anti-Myc antibody was used to detect the α chain, the anti-IgM antibody was used to detect the peptide-β chain-IgM, and the anti-Flag antibody was used to detect the J chain. A Western blot performed under non-reducing/non-boiled conditions detected a band at very high molecular weight (migrating higher than the highest molecular weight marker at 260 kDa). The anti-IgM, anti-Flag and anti-Myc antibodies detected a band in the same position on the blot, indicating co-migration of pMHC-β-IgM, MHC-α, and J-chain-Flag, and therefore, the formation of an intact complex of Class II pMHC-IgM/J-chain. The transfection cultures were centrifuged to pellet cells and the pMHC-IgM-containing supernatant was filtered before performing batch binding and gravity purification using Ni Excel resin according to the manufacturer's instructions. The Ni Excel resin was equilibrated in binding buffer (23 mM sodium phosphate, 500 mM NaCl, pH 7.4). The wash buffer used for gravity purification was 20 mM imidazole, 23 mM sodium phosphate, 500 mM NaCl, pH 7.4 and the elution buffer used was 500 mM imidazole, 23 mM sodium phosphate, 500 mM NaCl, pH 7.4. The presence of the pMHC-IgM in elution fractions was determined by western blot, shown in FIG. 16B. Elution fractions containing pMHC-IgM were pooled and concentrated before loading onto a Superdex 200 column for size exclusion chromatography to further purify the pMHC-IgM. The SEC buffer used was 20 mM HEPES, 150 mM NaCl, pH 7.2. Western blots were used to determine the presence of the α chain, peptide-β chain-IgM and J chains in the pMHC-IgM in the SEC fractions collected by size exclusion chromatography, shown in FIG. 16C. Western blot of a non-reduced sample shows co-migrating bands with anti-IgM, anti-Myc, and anti-Flag blots confirming the formation of an intact complex of pMHC-Class II-IgM/J-chain.

### Example 4—Modification of MHC Class I Alpha (a) Chain to Enhance Stability

This example describes the introduction of cysteines into the MHC Class I α chain to create stability enhancing cysteine traps.

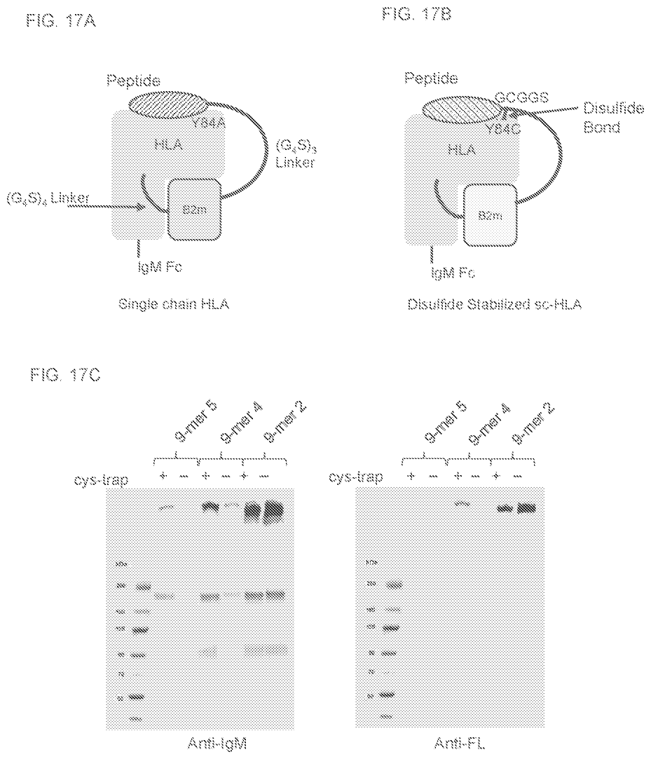

Cys-traps (Disulfide traps) have been reported to increase the expression levels of single-chain pMHC (Truscott, et al. (2007) J. IMMUNOL., 178: 6280-6289) (see, e.g., FIGS. 17A and 17B). To increase stability and expression due to the possible formation of a disulfide bonds, a pair of cysteines was introduced into the pMHC-IgM at the second G position (e.g., GGGGS to GCGGS) in the first G₄S portion of the (G₄S linker between the peptide and β2m and at position Y84 (Y84C) in the HLA chain. Constructs with the peptides 9-mer 2, 9-mer 4, and 9-mer 5, as described in Example 1, were produced as cys-9-mer 2-pMHC-IgM (cys-9-mer 2), cys-9-mer 4-pMHC-IgM (cys-9-mer 4), and cys-9-mer 5-pMHC-IgM (cys 9-mer 5).

Proteins were expressed essentially as described in Example 1. After expression, the supernatant was recovered and analyzed by western blotting with anti-IgM and anti-Flag antibodies to assess relative expression levels of the Cys-trap pMHC-IgM compared to the unaltered pMHC-IgM construct. FIG. 17C shows western blots loaded with purified cys-9-mer 2-pMHC-IgM, cys-9-mer 4-pMHC-IgM, cys-9-mer 5-pMHC-IgM and control 9-mer 2-pMHC-IgM, 9-mer 4-pMHC-IgM, 9-mer 5-pMHC-IgM and probed with anti-IgM antibody (left panel) and anti-FLAG tag antibody (right panel). Improvements in expression were observed for cys-trap 9-mer 5-A2-pMHC-IgM and cys-trap 9-mer 4-A2-pMHC-IgM compared to 9-mer 5-A2-pMHC-IgM and 9-mer 4-A2-pMHC-IgM.

### Example 5—Preparation of J Chain Effector/Targeting Moiety Constructs

This example describes expression, purification, and analysis of pMHC-IgM/J-chain fusions with different effector/targeting moieties.

Expression, production, and characterization of pMHC-IgM with anti-CD3 and PD-L1 effector/targeting moieties was demonstrated using a peptide derived from Orf1ab of SARS-CoV2 fused to HLA-A2 and IgM: KLWAQCVQL (SEQ ID NO:265). Plasmids encoding KLWAQCVQL-HLA-A2-pMHC-IgM, referred to as KLW-A2-pMHC-IgM (SEQ ID NO: 236), and anti-CD3-scFv-J-chain-Flag-His (SEQ ID NO: 254) or PD-L1-ECD-J-chain-Flag-His (SEQ ID NO: 245) or J-chain-Flag-His (SEQ ID NO: 251) were used for transfection of mammalian cells. Constructs were expressed, purified, and analyzed essentially as described in Example 1.

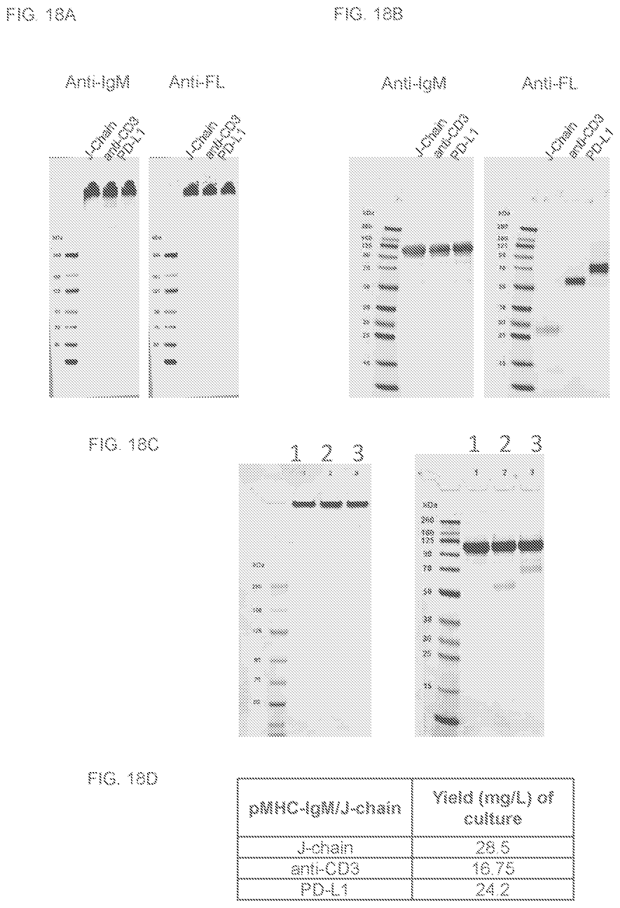

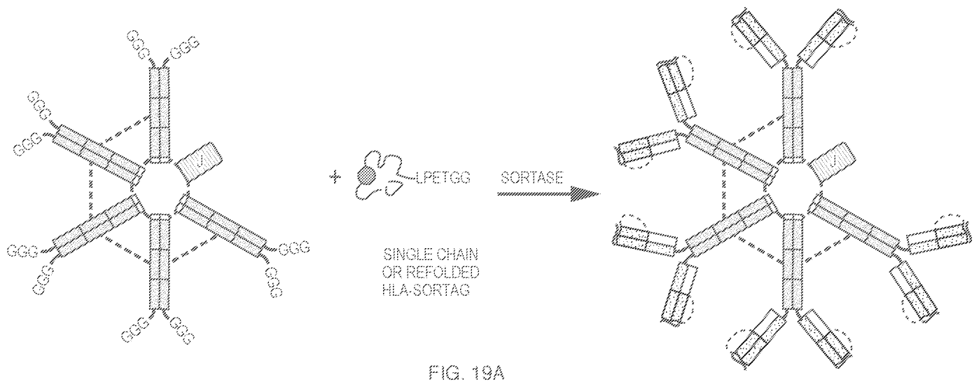

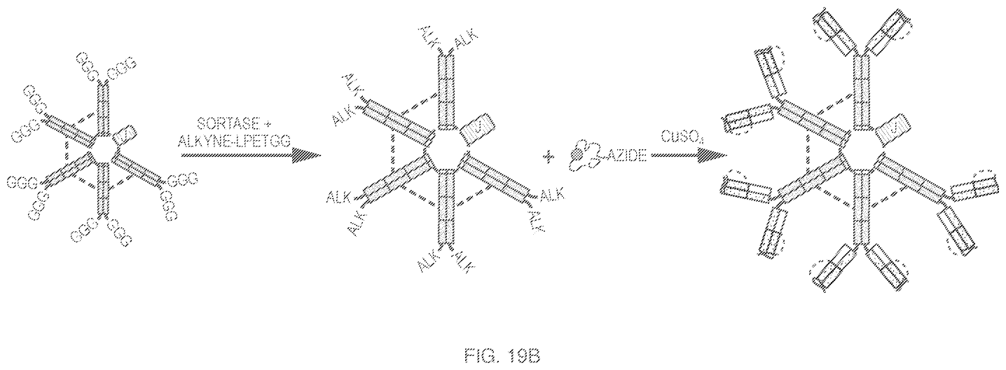

A western blot performed under non-reducing/non-boiling sample conditions detected a band at very high molecular weight (migrating higher than the highest molecular weight marker at 260 kDa) for all three proteins. FIG. 18A shows a western blot of non-reduced samples probed with anti-IgM antibody (left panel) and anti-FLAG tag antibody (right panel) (Lanes 1: J chain, 2: OKT3-J chain (anti-CD3), 3: PD-L1-ECD-J chain). For all three proteins, the anti-IgM and anti-Flag antibodies detected a band in the same position on the blot, indicating co-migration of pMHC-IgM and Flag-containing species and the formation of an intact complex of KLW-A2-pMHC-IgM/anti-CD3-J-chain, KLW-A2-pMHC-IgM/PD-L1-ECD-J-chain, and KLW-A2-pMHC-IgM/J-chain. A western blot performed under reducing conditions confirmed the identity and size of the individual chains. FIG. 18B shows a western blot of reduced samples probed with anti-IgM antibody (left panel) and anti-FLAG tag antibody (right panel) (Lanes 1: J chain, 2: OKT3-J chain (anti-CD3), 3: PD-L1-ECD-J chain). For each of the three proteins, the reduced/boiled samples on the western blot (FIG. 18B) and Coomassie-stained SDS-PAGE gel (FIG. 18C) show a band at approximately 105 kDa corresponding to KLW-A2-pMHC-IgM. The calculated molecular weight of KLW-A2-pMHC-IgM is 86.2 kDa and the difference in SDS-PAGE-observed and calculated molecular weight is believed to result from glycosylation. As observed in Example 1, the reduced sample of KLW-A2-pMHC-IgM/J-chain also has a band migrating at approximately 28 kDa in the SDS-PAGE. The reduced SDS-PAGE analysis of KLW-A2-pMHC-IgM/anti-CD3-J-chain shows a band migrating at approximately 58 kDa, which differs from the calculated molecular weight (45.1 kDa), and the difference is believed to result from glycosylation. The reduced SDS-PAGE analysis of KLW-A2-pMHC-IgM/PD-L1-ECD-J-chain also shows a band migrating at approximately 70 kDa, which differs from the calculated molecular weight (45.1 kDa), and the difference is likely due to glycosylation. PD-L1-ECD-J-chain has 5 predicted N-linked glycosylation sites whereas anti-CD3-J-chain and J-chain have only 1 predicted N-linked glycosylation site.

FIG. 18C shows non-reduced (left panel) and reduced (right panel) samples on SDS-PAGE (in both left and right panels, lane 1 corresponds to J-chain, lane 2 corresponds to anti-CD3, and lane 3 corresponds to PD-L1). The final material has a high purity determined by western blot and SDS-PAGE analysis. The yield of final protein varied based on the effector moieties from 16 to 28 mg/L culture (FIG. 18D). These results demonstrate generation of pure, homogeneous pMHC-IgM molecules with different effector-moieties fused to the J-chain.

### Example 6—Sortase Mediated Production of pMHC-IgM

This example describes pMHC-IgM generation via Sortase-mediated protein-to-protein conjugation.

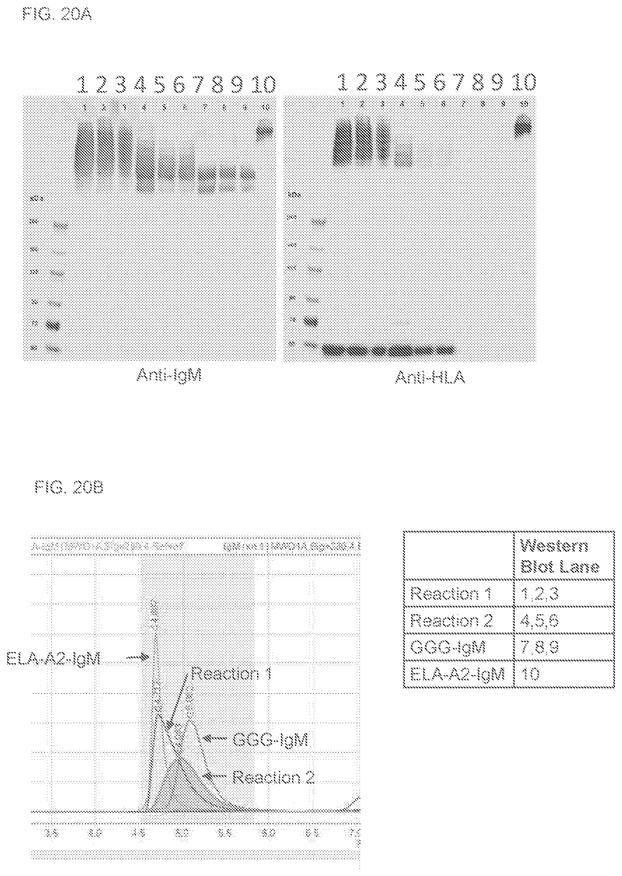

ELA/HLA-A2 monomer with a C-terminal Sortase recognition motif (LPETGG SEQ ID NO: 192) and C-terminal Flag and His-tag encoded by the sequence (SEQ ID NO: 243) was transiently expressed in Expi293F cells and purified by IMAC essentially as described in Example 1. IgM-Cμ2-4 Fc/J-chain with an N-terminal GGG Sortase conjugation motif (GGG-IgM) consisting of protein chains GGG-IgM Fc and J-chain (SEQ ID NO: 244 and 251, respectively) was transiently expressed in Expi293F cells and purified by IMAC essentially as described in Example 1. Purified single-chain ELA/HLA-A2 monomer and purified GGG-IgM/J-chain were mixed with Sortase and calcium chloride and incubated for 1 hour at 4° C. (Reaction 1). A second reaction condition was at room temperature with excess sortase (Reaction 2). Exemplary Sortase-mediated protein-to-protein conjugations are shown in FIGS. 19A and 19B. After incubation, the reaction sample was loaded onto a Superdex 200 Increase 10/300 size exclusion chromatography column. Elution fractions associated with high valency ELA-IgM multimers were pooled and concentrated using Amicon 100 kDa MWCO to at least 1 mg/mL and filtered with a 0.22 μm spin filter.

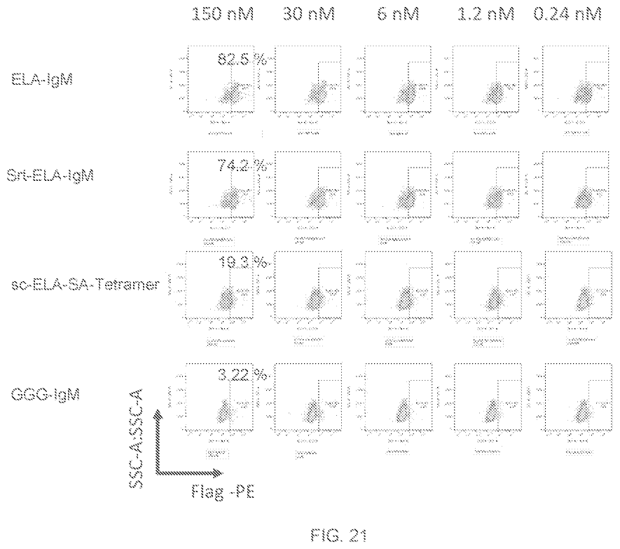

The valency and purity of sortase-mediated ELA-IgM multimers (Srt-ELA-IgM) were determined by western blot and analytical SEC. FIG. 20A shows western blots probed with anti-IgM antibody (left panel) and anti-HLA antibody (right panel). Lanes 1, 2, and 3 correspond to samples of Srt-ELA-IgM from Reaction 1; lanes 4, 5, and 6 correspond to samples of Srt-ELA-IgM from Reaction 2; lanes 7, 8, and 9 correspond to samples of unconjugated GGG-IgM, and lane 10 corresponds to ELA-A2-IgM control. The gel shows a characteristic “ladder” effect for lanes 1, 2, and 3 corresponding to the individual Srt-ELA-IgM Cμ2-4 Fc chains that were coupled to the individual ELA/HLA-A2 monomers, thus representing a mixed valency of 1, 2, 3, 4, 5, 6, 7, 8, 9, or 10 for HLA. Samples of Reaction 2 show lower HLA valency, and the GGG-IgM control shows zero valency for HLA. The recombinantly produced single-chain pMHC ELA-A2-IgM corresponds to a valency of 10 for HLA. The corresponding size exclusion chromatogram shows that the retention time of Srt-ELA-IgM (Reaction 1) corresponds to a high valency when compared to the control proteins ELA-A2-IgM and GGG-IgM-Fc which have a valency of 10 and 0 HLA's, respectively (FIG. 20B). The product of Reaction 2 showed a lower valency of HLA's indicating Srt-ELA-IgM reacted efficiently and was of high valency.

Srt-ELA-IgM binding of JurkatJ76-CD8-NFAT-GFP cells expressing recombinant ELAGIGILTV-HLA-A2 specific TCR by flow cytometry was tested as described in Example 2. Binding was tested at 5 different concentrations of IgM: 150 nM, 30 nM, 6 nM, 1.2 nM, and 0.24 nM. Srt-ELA-IgM showed specific binding to its cognate TCR on Jurkat cells (FIG. 21), with 74.5% cells positive at 150 nM Srt-ELA-IgM concentration, whereas no binding was detected with negative control GGG-IgM-Fc (3.22% positive cells). Srt-ELA-IgM showed comparable binding to cognate TCR with recombinantly produced ELA-A2-IgM (ELA-IgM) (82.5% positive cells). Sc-ELA-SA-tetramer showed 19.3% positive cells.

### Example 7—Inhibition of Effector T Cells In Vitro by pMHC-Ig Multimers Coupled to an Immunosuppressive Agent

This example describes the inhibition of effector T cells by pMHC-Ig multimers coupled to an effector/targeting moiety that acts as an immunosuppressive agent.

pMHC-Igs coupled to one or more of TGF-b, IL-10, HLA-G, an LILRB1 or LILRB2 agonist, or an exhaustion-inducing agent like PD-L1 or a PD1 agonist are assessed for their ability to reduce TCR signaling, proliferation, cytokine secretion, or cell killing by antigen-specific effector T cells, as shown in the schematic in FIG. 7.

To assess TCR signaling inhibition, J76-CD8-NFAT-GFP cell lines expressing recombinant TCRs specific to the peptide-MHC (or T cells expanded from PBMCs with cognate peptide) are incubated with an allele-matched APC cell line loaded with the cognate peptide, plus or minus the pMHC-Ig-immunosuppressor multimers. Analysis of NFAT-GFP expression over time at 37° C. is expected to show a reduction in Nuclear factor of activated T-cells (NFAT) signaling in the presence of the pMHC-Ig multimers. Antigen-specific T cells co-cultured with allele-matched APC cells loaded with the cognate peptide can also be stained with anti-CD69-APC (Clone FN50) antibodies and analyzed by flow to measure a reduction in the percentage of CD69+ cells in the presence of the peptide-MHC-Ig multimers. Another measure of effects on TCR signaling is to monitor inhibition of phosphorylation of SLP-76, PLCgamma and ZAP-70 by western blot when pMHC-Ig multimers are incubated with antigen-specific T cells co-cultured with APCs loaded with cognate peptide.

To assess inhibition of T cell proliferation by pMHC-Ig-immunosuppressor multimers, antigen-specific T cells are loaded with a dye like CFSE, then co-cultured with allele-matched APCs loaded with the cognate peptide plus and minus the multimers. Every generation of cells appears as a new, lower mean fluorescent intensity (MFI) peak on a flow cytometry histogram. Reduced proliferation in the presence of the pMHC-Ig multimers is evidenced by a shift to a higher percentage of T cells with high MFI.

To assess inhibition of cytokine secretion by pMHC-Ig-immunosuppressor multimers, multimers are added to co-cultures of antigen-specific T cells and allele-matched APCs loaded with cognate peptide, and the culture is monitored for IL-2, IFN-γ or TNFα by ELISA/ELISPOT/Mesoscale. A reduction in secretion indicates immunosuppression by the pMHC-Ig multimer.

Inhibition of T cell cytotoxicity by pMHC-Ig-immunosuppressor multimers is measured by coculturing antigen-specific T cells with target allele-matched APCs loaded with the cognate peptide. By differential labeling of the T cells vs the target APCs, the loss of viability (live/dead stain) and overall reduction of APC cell counts can be monitored by flow. Co-incubation of pMHC-Ig-immunosuppressor multimers inhibits targeted killing of APCs, resulting in higher numbers of live cells.

### INCORPORATION BY REFERENCE

The entire disclosure of each of the patent and scientific documents referred to herein is incorporated by reference for all purposes.

### EQUIVALENTS

An invention may be embodied in other specific forms without departing from the spirit or essential characteristics thereof. The foregoing embodiments are therefore to be considered in all respects illustrative rather than limiting on any invention disclosed herein. Scope of an invention is thus indicated by the appended claims rather than by the foregoing description, and all changes that come within the meaning and range of equivalency of the claims are intended to be embraced therein.

SEQUENCE LISTING SUMMARY

| SEQ | |
|---|---|
| ID | |
| NO: | DESCRIPTION |
| 1 | NLVPMVATVGGGGSGGGGSGGGGSIQRTPKIQVYSRHPAENGKSNFLNCYVSGFHPSDIEVDLLKNGE |
| | RIEKVEHSDLSFSKDWSFYLLYYTEFTPTEKDEYACRVNHVTLSQPKIVKWDRDMGGGGSGGGGSGGG |
| | GSGGGGSGSHSMRYFFTSVSRPGRGEPRFIAVGYVDDTQFVRFDSDAASQRMEPRAPWIEQEGPEYWD |
| | GETRKVKAHSQTHRVDLGTLRGAYNQSEAGSHTVQRMYGCDVGSDWRFLRGYHQYAYDGKDYIALKED |
| | LRSWTAADMAAQTTKHKWEAAHVAEQLRAYLEGTCVEWLRRYLENGKETLQRTDAPKTHMTHHAVSDH |
| | EATLRCWALSFYPAEITLTWQRDGEDQTQDTELVETRPAGDGTFQKWAAVVVPSGQEQRYTCHVQHEG |
| | LPKPLTLRWEPSSGGGGSGGGGSGGGGSIAELPPKVSVFVPPRDGFFGNPRKSKLICQATGESPRQIQ |
| | VSWLREGKQVGSGVTTDQVQAEAKESGPTTYKVTSTLTIKESDWLGQSMFTCRVDHRGLTFQQNASSM |
| | CVPDQDTAIRVFAIPPSFASIFLTKSTKLTCLVTDLTTYDSVTISWTRQNGEAVKTHTNISESHPNAT |
| | FSAVGEASICEDDWNSGERFTCTVTHTDLPSPLKQTISRPKGVALHRPDVYLLPPAREQLNLRESATI |
| | TCLVTGFSPADVFVQWMQRGQPLSPEKYVTSAPMPEPQAPGRYFAHSILTVSEEEWNTGETYTCVVAH |
| | EALPNRVTERTVDKSTGKPTLYNVSLVMSDTAGTCY |
| | (NLV_B2M_HLAA2_IgM2-4 amino acid sequence) |
| 2 | AACCTTGTGCCTATGGTCGCCACAGTGGGTGGTGGCGGCTCCGGAGGCGGTGGCTCCGGCGGCGGCGG |
| | CAGTATTCAAAGAACCCCCAAGATCCAGGTTTATTCTCGCCATCCCGCTGAGAACGGTAAATCTAATT |
| | TCCTTAACTGCTACGTTTCAGGCTTCCATCCAAGCGACATCGAAGTAGATCTGCTCAAAAATGGGGAG |
| | AGAATCGAGAAGGTAGAGCACAGTGACCTAAGTTTCAGTAAAGACTGGTCCTTCTACCTGCTCTACTA |
| | CACTGAGTTCACCCCCACAGAGAAGGACGAATATGCATGTCGGGTCAATCACGTCACCTTGTCTCAAC |
| | CTAAGATCGTGAAATGGGATCGGGATATGGGGGGTGGCGGCTCAGGCGGTGGAGGCTCAGGCGGGGGA |
| | GGATCTGGAGGCGGCGGTAGCGGCTCACACTCTATGAGGTATTTTTTTACCTCAGTGAGCAGGCCAGG |
| | CAGAGGTGAACCCAGATTCATTGCAGTTGGTTACGTTGATGATACCCAGTTCGTCCGCTTTGATTCTG |
| | ACGCCGCATCACAGAGGATGGAACCTAGGGCACCTTGGATAGAACAGGAAGGCCCAGAGTATTGGGAT |
| | GGCGAGACTAGAAAAGTGAAGGCTCATAGCCAGACACATCGAGTAGACCTGGGAACTCTCCGTGGAGC |
| | ATATAACCAGTCCGAGGCTGGCTCCCATACAGTGCAGAGGATGTACGGATGCGACGTGGGCAGCGATT |
| | GGCGTTTTCTACGAGGTTACCATCAGTACGCATACGATGGAAAAGACTATATTGCACTCAAGGAGGAC |
| | CTCCGAAGCTGGACCGCAGCTGATATGGCTGCACAGACTACCAAACATAAGTGGGAGGCAGCTCATGT |
| | TGCAGAGCAACTACGGGCATACCTCGAAGGGACTTGTGTGGAGTGGCTGAGAAGGTACCTAGAGAACG |
| | GGAAGGAGACCCTGCAGCGGACAGACGCCCCAAAGACCCATATGACCCACCACGCTGTCAGTGATCAT |
| | GAAGCCACATTGCGCTGCTGGGCACTTAGTTTCTATCCCGCAGAAATCACCTTGACCTGGCAGCGTGA |
| | CGGCGAAGATCAGACCCAGGACACCGAGCTTGTGGAAACAAGACCTGCCGGAGATGGAACCTTCCAGA |
| | AGTGGGCCGCTGTGGTCGTTCCCAGCGGCCAGGAGCAGCGGTATACTTGTCATGTCCAACATGAGGGA |
| | CTGCCAAAACCCCTCACCCTTAGGTGGGAGCCTAGTTCCGGTGGCGGAGGCTCCGGAGGGGGCGGAAG |
| | CGGAGGTGGTGGATCCATCGCAGAACTGCCACCTAAAGTCAGCGTCTTTGTGCCTCCCCGTGATGGCT |
| | TTTTTGGCAACCCCCGGAAGAGTAAACTAATATGCCAGGCCACAGGATTTAGTCCGCGGCAGATTCAG |
| | GTGTCTTGGCTCCGGGAGGGAAAACAGGTCGGAAGTGGAGTGACCACAGACCAAGTGCAGGCCGAGGC |
| | TAAGGAGAGTGGTCCTACAACTTACAAGGTTACAAGTACCTTGACAATTAAAGAGTCCGATTGGCTGG |
| | GACAGAGTATGTTTACATGTAGAGTGGACCACCGTGGTCTCACTTTCCAGCAGAATGCTAGTTCAATG |
| | TGCGTTCCGGATCAGGATACCGCAATTAGAGTGTTCGCCATCCCTCCCTCTTTTGCTTCCATCTTCCT |
| | GACCAAGAGCACCAAGCTTACATGCTTGGTTACCGATCTTACAACCTATGATTCTGTCACTATCTCCT |
| | GGACACGCCAGAACGGGGAGGCCGTGAAGACACATACTAATATCAGCGAGAGCCATCCCAACGCTACA |
| | TTCTCAGCAGTGGGCGAGGCCTCTATCTGCGAAGACGACTGGAATTCAGGGGAACGGTTCACGTGTAC |
| | TGTCACCCATACAGATCTGCCTTCCCCTCTTAAGCAGACCATTTCACGACCGAAAGGGGTGGCTCTTC |
| | ACCGGCCCGACGTGTACCTACTCCCACCTGCCCGTGAACAACTGAATCTGCGCGAGAGCGCTACCATT |
| | ACCTGCCTGGTAACCGGATTTAGTCCTGCAGACGTCTTTGTTCAGTGGATGCAACGCGGCCAGCCCCT |
| | CTCCCCTGAGAAGTATGTGACTAGTGCCCCTATGCCCGAACCCCAGGCTCCTGGCAGATATTTTGCCC |
| | ATAGCATCCTGACAGTATCAGAAGAGGAGTGGAACACTGGTGAAACCTATACTTGCGTGGTGGCACAC |
| | GAGGCTTTGCCGAACCGCGTCACAGAACGAACCGTTGACAAAAGCACAGGTAAGCCCACCCTGTACAA |
| | TGTCAGCCTCGTAATGTCTGATACAGCAGGTACCTGTTAT |
| | (NLV_B2M_HLAA2_gM2-4 nucleotide sequence) |
| 3 | NLVPMVATVGGGGSGGGGSGGGGSIQRTPKIQVYSRHPAENGKSNFLNCYVSGFHPSDIEVDLLKNGE |
| | RIEKVEHSDLSFSKDWSFYLLYYTEFTPTEKDEYACRVNHVTLSQPKIVKWDRDMGGGGSGGGGSGGG |
| | GSGGGGSGSHSMRYFFTSVSRPGRGEPRFIAVGYVDDTQFVRFDSDAASQRMEPRAPWIEQEGPEYWD |
| | GETRKVKAHSQTHRVDLGTLRGAYNQSEAGSHTVQRMYGCDVGSDWRFLRGYHQYAYDGKDYIALKED |
| | LRSWTAADMAAQTTKHKWEAAHVAEQLRAYLEGTCVEWLRRYLENGKETLQRTDAPKTHMTHHAVSDH |
| | EATLRCWALSFYPAEITLTWQRDGEDQTQDTELVETRPAGDGTFQKWAAVVVPSGQEQRYTCHVQHEG |
| | LPKPLTLRWEPSSGGGGSGGGGSGGGGSIRVFAIPPSFASIFLTKSTKLTCLVTDLTTYDSVTISWTR |
| | QNGEAVKTHTNISESHPNATFSAVGEASICEDDWNSGERFTCTVTHTDLPSPLKQTISRPKGVALHRP |
| | DVYLLPPAREQLNLRESATITCLVTGFSPADVFVQWMQRGQPLSPEKYVTSAPMPEPQAPGRYFAHSI |
| | LTVSEEEWNTGETYTCVVAHEALPNRVTERTVDKSTGKPTLYNVSLVMSDTAGTCY |
| | (NLV_B2M_HLAA2_gM3-4 amino acid sequence) |
| 4 | AACCTTGTGCCTATGGTCGCCACAGTGGGTGGTGGCGGCTCCGGAGGCGGTGGCTCCGGCGGCGGCGG |
| | CAGTATTCAAAGAACCCCCAAGATCCAGGTTTATTCTCGCCATCCCGCTGAGAACGGTAAATCTAATT |
| | TCCTTAACTGCTACGTTTCAGGCTTCCATCCAAGCGACATCGAAGTAGATCTGCTCAAAAATGGGGAG |
| | AGAATCGAGAAGGTAGAGCACAGTGACCTAAGTTTCAGTAAAGACTGGTCCTTCTACCTGCTCTACTA |
| | CACTGAGTTCACCCCCACAGAGAAGGACGAATATGCATGTCGGGTCAATCACGTCACCTTGTCTCAAC |
| | CTAAGATCGTGAAATGGGATCGGGATATGGGGGGTGGCGGCTCAGGCGGTGGAGGCTCAGGCGGGGGA |
| | GGATCTGGAGGCGGCGGTAGCGGCTCACACTCTATGAGGTATTTTTTTACCTCAGTGAGCAGGCCAGG |
| | CAGAGGTGAACCCAGATTCATTGCAGTTGGTTACGTTGATGATACCCAGTTCGTCCGCTTTGATTCTG |
| | ACGCCGCATCACAGAGGATGGAACCTAGGGCACCTTGGATAGAACAGGAAGGCCCAGAGTATTGGGAT |
| | GGCGAGACTAGAAAAGTGAAGGCTCATAGCCAGACACATCGAGTAGACCTGGGAACTCTCCGTGGAGC |
| | ATATAACCAGTCCGAGGCTGGCTCCCATACAGTGCAGAGGATGTACGGATGCGACGTGGGCAGCGATT |
| | GGCGTTTTCTACGAGGTTACCATCAGTACGCATACGATGGAAAAGACTATATTGCACTCAAGGAGGAC |
| | CTCCGAAGCTGGACCGCAGCTGATATGGCTGCACAGACTACCAAACATAAGTGGGAGGCAGCTCATGT |
| | TGCAGAGCAACTACGGGCATACCTCGAAGGGACTTGTGTGGAGTGGCTGAGAAGGTACCTAGAGAACG |
| | GGAAGGAGACCCTGCAGCGGACAGACGCCCCAAAGACCCATATGACCCACCACGCTGTCAGTGATCAT |
| | GAAGCCACATTGCGCTGCTGGGCACTTAGTTTCTATCCCGCAGAAATCACCTTGACCTGGCAGCGTGA |
| | CGGCGAAGATCAGACCCAGGACACCGAGCTTGTGGAAACAAGACCTGCCGGAGATGGAACCTTCCAGA |
| | AGTGGGCCGCTGTGGTCGTTCCCAGCGGCCAGGAGCAGCGGTATACTTGTCATGTCCAACATGAGGGA |
| | CTGCCAAAACCCCTCACCCTTAGGTGGGAGCCTAGTTCCGGTGGCGGAGGCTCCGGAGGGGGCGGAAG |
| | CGGAGGTGGTGGATCCATCCGAGTATTCGCTATCCCACCCAGTTTCGCCTCTATCTTCCTTACCAAAT |
| | CTACTAAACTGACATGCTTGGTCACTGATCTAACCACTTACGATTCTGTGACAATTTCCTGGACAAGG |
| | CAGAACGGAGAGGCAGTAAAGACTCATACGAATATTTCAGAATCCCATCCCAACGCCACGTTTTCCGC |
| | CGTTGGCGAGGCCTCCATTTGTGAGGACGATTGGAATAGCGGAGAGCGCTTTACCTGCACAGTGACGC |
| | ATACAGATCTGCCTAGCCCACTCAAGCAGACTATCTCTCGCCCAAAGGGGGTGGCTCTGCATAGGCCT |
| | GACGTATATCTCCTTCCACCCGCTCGAGAGCAGCTGAACTTGAGAGAATCCGCAACTATTACTTGCTT |
| | GGTGACGGGCTTTTCTCCCGCCGATGTCTTCGTGCAGTGGATGCAGCGTGGACAGCCTCTCTCTCCTG |
| | AGAAATACGTAACATCTGCCCCCATGCCCGAGCCTCAGGCTCCTGGTCGTTATTTTGCTCACAGTATC |
| | CTGACCGTCAGCGAAGAGGAATGGAACACTGGAGAGACCTACACCTGTGTCGTGGCTCATGAAGCTCT |
| | GCCTAACAGGGTCACCGAGCGCACCGTGGACAAAAGCACTGGCAAACCGACCCTGTATAACGTGTCAC |
| | TCGTGATGAGCGACACAGCCGGCACTTGCTAT |
| | (NLV_B2M_HLAA2_gM3-4 nucleotide sequence) |
| 5 | ELAGIGILTVGGGGSGGGGSGGGGSIQRTPKIQVYSRHPAENGKSNFLNCYVSGFHPSDIEVDLLKNG |
| | ERIEKVEHSDLSFSKDWSFYLLYYTEFTPTEKDEYACRVNHVTLSQPKIVKWDRDMGGGGSGGGGSGG |
| | GGSGGGGSGSHSMRYFFTSVSRPGRGEPRFIAVGYVDDTQFVRFDSDAASQRMEPRAPWIEQEGPEYW |
| | DGETRKVKAHSQTHRVDLGTLRGAYNQSEAGSHTVQRMYGCDVGSDWRFLRGYHQYAYDGKDYIALKE |
| | DLRSWTAADMAAQTTKHKWEAAHVAEQLRAYLEGTCVEWLRRYLENGKETLQRTDAPKTHMTHHAVSD |
| | HEATLRCWALSFYPAEITLTWQRDGEDQTQDTELVETRPAGDGTFQKWAAVVVPSGQEQRYTCHVQHE |
| | GLPKPLTLRWEPSSGGGGSGGGGSGGGGSIAELPPKVSVFVPPRDGFFGNPRKSKLICQATGESPRQI |
| | QVSWLREGKQVGSGVTTDQVQAEAKESGPTTYKVTSTLTIKESDWLGQSMFTCRVDHRGLTFQQNASS |
| | MCVPDQDTAIRVFAIPPSFASIFLTKSTKLTCLVTDLTTYDSVTISWTRQNGEAVKTHTNISESHPNA |
| | TFSAVGEASICEDDWNSGERFTCTVTHTDLPSPLKQTISRPKGVALHRPDVYLLPPAREQLNLRESAT |
| | ITCLVTGFSPADVFVQWMQRGQPLSPEKYVTSAPMPEPQAPGRYFAHSILTVSEEEWNTGETYTCVVA |
| | HEALPNRVTERTVDKSTGKPTLYNVSLVMSDTAGTCY |
| | (ELA_B2M_HLAA2_gM2-4 amino acid sequence) |
| 6 | ELAGIGILTVGGGGSGGGGSGGGGSIQRTPKIQVYSRHPAENGKSNFLNCYVSGFHPSDIEVDLLKNG |
| | ERIEKVEHSDLSFSKDWSFYLLYYTEFTPTEKDEYACRVNHVTLSQPKIVKWDRDMGGGGSGGGGSGG |
| | GGSGGGGSGSHSMRYFFTSVSRPGRGEPRFIAVGYVDDTQFVRFDSDAASQRMEPRAPWIEQEGPEYW |
| | DGETRKVKAHSQTHRVDLGTLRGAYNQSEAGSHTVQRMYGCDVGSDWRFLRGYHQYAYDGKDYIALKE |
| | DLRSWTAADMAAQTTKHKWEAAHVAEQLRAYLEGTCVEWLRRYLENGKETLQRTDAPKTHMTHHAVSD |
| | HEATLRCWALSFYPAEITLTWQRDGEDQTQDTELVETRPAGDGTFQKWAAVVVPSGQEQRYTCHVQHE |
| | GLPKPLTLRWEPSSGGGGSGGGGSGGGGSIRVFAIPPSFASIFLTKSTKLTCLVTDLTTYDSVTISWT |
| | RQNGEAVKTHTNISESHPNATFSAVGEASICEDDWNSGERFTCTVTHTDLPSPLKQTISRPKGVALHR |
| | PDVYLLPPAREQLNLRESATITCLVTGFSPADVFVQWMQRGQPLSPEKYVTSAPMPEPQAPGRYFAHS |
| | ILTVSEEEWNTGETYTCVVAHEALPNRVTERTVDKSTGKPTLYNVSLVMSDTAGTCY |
| | (ELA_B2M_HLAA2_gM3-4 amino acid sequence) |
| 7 | AACCTTGTGCCTATGGTCGCCACAGTGGGTGGTGGCGGCTCCGGAGGCGGTGGCTCCGGCGGCGGCGG |
| | CAGTATTCAAAGAACCCCCAAGATCCAGGTTTATTCTCGCCATCCCGCTGAGAACGGTAAATCTAATT |
| | TCCTTAACTGCTACGTTTCAGGCTTCCATCCAAGCGACATCGAAGTAGATCTGCTCAAAAATGGGGAG |
| | AGAATCGAGAAGGTAGAGCACAGTGACCTAAGTTTCAGTAAAGACTGGTCCTTCTACCTGCTCTACTA |
| | CACTGAGTTCACCCCCACAGAGAAGGACGAATATGCATGTCGGGTCAATCACGTCACCTTGTCTCAAC |
| | CTAAGATCGTGAAATGGGATCGGGATATGGGGGGTGGCGGCTCAGGCGGTGGAGGCTCAGGCGGGGGA |
| | GGATCTGGAGGCGGCGGTAGCGGCTCACACTCTATGAGGTATTTTTTTACCTCAGTGAGCAGGCCAGG |
| | CAGAGGTGAACCCAGATTCATTGCAGTTGGTTACGTTGATGATACCCAGTTCGTCCGCTTTGATTCTG |
| | ACGCCGCATCACAGAGGATGGAACCTAGGGCACCTTGGATAGAACAGGAAGGCCCAGAGTATTGGGAT |
| | GGCGAGACTAGAAAAGTGAAGGCTCATAGCCAGACACATCGAGTAGACCTGGGAACTCTCCGTGGAGC |
| | ATATAACCAGTCCGAGGCTGGCTCCCATACAGTGCAGAGGATGTACGGATGCGACGTGGGCAGCGATT |
| | GGCGTTTTCTACGAGGTTACCATCAGTACGCATACGATGGAAAAGACTATATTGCACTCAAGGAGGAC |
| | CTCCGAAGCTGGACCGCAGCTGATATGGCTGCACAGACTACCAAACATAAGTGGGAGGCAGCTCATGT |
| | TGCAGAGCAACTACGGGCATACCTCGAAGGGACTTGTGTGGAGTGGCTGAGAAGGTACCTAGAGAACG |
| | GGAAGGAGACCCTGCAGCGGACAGACGCCCCAAAGACCCATATGACCCACCACGCTGTCAGTGATCAT |
| | GAAGCCACATTGCGCTGCTGGGCACTTAGTTTCTATCCCGCAGAAATCACCTTGACCTGGCAGCGTGA |
| | CGGCGAAGATCAGACCCAGGACACCGAGCTTGTGGAAACAAGACCTGCCGGAGATGGAACCTTCCAGA |
| | AGTGGGCCGCTGTGGTCGTTCCCAGCGGCCAGGAGCAGCGGTATACTTGTCATGTCCAACATGAGGGA |
| | CTGCCAAAACCCCTCACCCTTAGGTGGGAGCCTAGTTCCGGTGGCGGAGGCTCCGGAGGGGGCGGAAG |
| | CGGAGGTGGTGGATCCTGCCATCCGAGGCTCAGCCTTCACAGGCCTGCATTGGAAGACCTGCTACTTG |
| | GTTCCGAAGCCAATCTGACCTGTACTCTTACAGGCTTGCGCGACGCTTCTGGGGTCACGTTCACCTGG |
| | ACACCTTCTAGCGGAAAATCAGCCGTACAAGGGCCCCCTGAGAGAGACTTGTGTGGCTGTTATAGTGT |
| | CTCCAGCGTTTTGCCTGGTTGTGCAGAGCCTTGGAATCATGGGAAAACCTTCACTTGCACAGCTGCAT |
| | ATCCCGAGAGTAAGACTCCACTCACGGCAACCCTGAGTAAATCCGGCAACACATTCCGCCCAGAGGTA |
| | CATCTTCTGCCACCCCCTTCTGAAGAATTGGCATTGAACGAACTCGTCACCCTCACCTGTCTGGCTCG |
| | TGGCTTTAGTCCAAAGGACGTACTGGTGCGATGGCTGCAAGGGTCCCAAGAGCTCCCAAGGGAGAAGT |
| | ACCTCACTTGGGCTAGTAGACAGGAACCATCCCAAGGTACGACAACATTCGCCGTCACATCCATCCTG |
| | CGCGTCGCTGCCGAAGACTGGAAGAAGGGAGACACCTTTTCATGCATGGTCGGACATGAGGCACTCCC |
| | ACTGGCTTTCACTCAAAAGACCATTGATAGACTCGCCGGTAAACCAACTCACGTAAACGTGTCCGTCG |
| | TGATGGCAGAGGTGGATGGTACCTGCTAT |
| | (ELA_B2M_HLAA2_gM3-4 nucleotide sequence) |
| 8 | NLVPMVATVGGGGSGGGGSGGGGSIQRTPKIQVYSRHPAENGKSNFLNCYVSGFHPSDIEVDLLKNGE |
| | RIEKVEHSDLSFSKDWSFYLLYYTEFTPTEKDEYACRVNHVTLSQPKIVKWDRDMGGGGSGGGGSGGG |
| | GSGGGGSGSHSMRYFFTSVSRPGRGEPRFIAVGYVDDTQFVRFDSDAASQRMEPRAPWIEQEGPEYWD |
| | GETRKVKAHSQTHRVDLGTLRGAYNQSEAGSHTVQRMYGCDVGSDWRFLRGYHQYAYDGKDYIALKED |
| | LRSWTAADMAAQTTKHKWEAAHVAEQLRAYLEGTCVEWLRRYLENGKETLQRTDAPKTHMTHHAVSDH |
| | EATLRCWALSFYPAEITLTWQRDGEDQTQDTELVETRPAGDGTFQKWAAVVVPSGQEQRYTCHVQHEG |
| | LPKPLTLRWEPSSGGGGSGGGGSGGGGSCHPRLSLHRPALEDLLLGSEANLTCTLTGLRDASGVTFTW |
| | TPSSGKSAVQGPPERDLCGCYSVSSVLPGCAEPWNHGKTFTCTAAYPESKTPLTATLSKSGNTFRPEV |
| | HLLPPPSEELALNELVTLTCLARGFSPKDVLVRWLQGSQELPREKYLTWASRQEPSQGTTTFAVTSIL |
| | RVAAEDWKKGDTFSCMVGHEALPLAFTQKTIDRLAGKPTHVNVSVVMAEVDGTCY |
| | (NLV_B2M_HLAA2_IgA1.1 amino acid sequence) |
| 9 | NLVPMVATVGGGGSGGGGSGGGGSIQRTPKIQVYSRHPAENGKSNFLNCYVSGFHPSDIEVDLLKNGE |
| | RIEKVEHSDLSFSKDWSFYLLYYTEFTPTEKDEYACRVNHVTLSQPKIVKWDRDMGGGGSGGGGSGGG |
| | GSGGGGSGSHSMRYFFTSVSRPGRGEPRFIAVGYVDDTQFVRFDSDAASQRMEPRAPWIEQEGPEYWD |
| | GETRKVKAHSQTHRVDLGTLRGAYNQSEAGSHTVQRMYGCDVGSDWRFLRGYHQYAYDGKDYIALKED |
| | LRSWTAADMAAQTTKHKWEAAHVAEQLRAYLEGTCVEWLRRYLENGKETLQRTDAPKTHMTHHAVSDH |
| | EATLRCWALSFYPAEITLTWQRDGEDQTQDTELVETRPAGDGTFQKWAAVVVPSGQEQRYTCHVQHEG |
| | LPKPLTLRWEPSSGGGGSGGGGSGGGGSCHPRLSLHRPALEDLLLGSEANLTCTLTGLRDASGATFTW |
| | TPSSGKSAVQGPPERDLCGCYSVSSVLPGCAQPWNHGETFTCTAAHPELKTPLTANITKSGNTFRPEV |
| | HLLPPPSEELALNELVTLTCLARGFSPKDVLVRWLQGSQELPREKYLTWASRQEPSQGTTTYAVTSIL |
| | RVAAEDWKKGETFSCMVGHEALPLAFTQKTIDRMAGKPTHINVSVVMAEADGTCY |
| | (NLV_B2M_HLAA2_IgA2m2.1 amino acid sequence) |
| 10 | AACCTTGTGCCTATGGTCGCCACAGTGGGTGGTGGCGGCTCCGGAGGCGGTGGCTCCGGCGGCGGCGG |
| | CAGTATTCAAAGAACCCCCAAGATCCAGGTTTATTCTCGCCATCCCGCTGAGAACGGTAAATCTAATT |
| | TCCTTAACTGCTACGTTTCAGGCTTCCATCCAAGCGACATCGAAGTAGATCTGCTCAAAAATGGGGAG |
| | AGAATCGAGAAGGTAGAGCACAGTGACCTAAGTTTCAGTAAAGACTGGTCCTTCTACCTGCTCTACTA |
| | CACTGAGTTCACCCCCACAGAGAAGGACGAATATGCATGTCGGGTCAATCACGTCACCTTGTCTCAAC |
| | CTAAGATCGTGAAATGGGATCGGGATATGGGGGGTGGCGGCTCAGGCGGTGGAGGCTCAGGCGGGGGA |
| | GGATCTGGAGGCGGCGGTAGCGGCTCACACTCTATGAGGTATTTTTTTACCTCAGTGAGCAGGCCAGG |
| | CAGAGGTGAACCCAGATTCATTGCAGTTGGTTACGTTGATGATACCCAGTTCGTCCGCTTTGATTCTG |
| | ACGCCGCATCACAGAGGATGGAACCTAGGGCACCTTGGATAGAACAGGAAGGCCCAGAGTATTGGGAT |
| | GGCGAGACTAGAAAAGTGAAGGCTCATAGCCAGACACATCGAGTAGACCTGGGAACTCTCCGTGGAGC |
| | ATATAACCAGTCCGAGGCTGGCTCCCATACAGTGCAGAGGATGTACGGATGCGACGTGGGCAGCGATT |
| | GGCGTTTTCTACGAGGTTACCATCAGTACGCATACGATGGAAAAGACTATATTGCACTCAAGGAGGAC |
| | CTCCGAAGCTGGACCGCAGCTGATATGGCTGCACAGACTACCAAACATAAGTGGGAGGCAGCTCATGT |
| | TGCAGAGCAACTACGGGCATACCTCGAAGGGACTTGTGTGGAGTGGCTGAGAAGGTACCTAGAGAACG |
| | GGAAGGAGACCCTGCAGCGGACAGACGCCCCAAAGACCCATATGACCCACCACGCTGTCAGTGATCAT |
| | GAAGCCACATTGCGCTGCTGGGCACTTAGTTTCTATCCCGCAGAAATCACCTTGACCTGGCAGCGTGA |
| | CGGCGAAGATCAGACCCAGGACACCGAGCTTGTGGAAACAAGACCTGCCGGAGATGGAACCTTCCAGA |
| | AGTGGGCCGCTGTGGTCGTTCCCAGCGGCCAGGAGCAGCGGTATACTTGTCATGTCCAACATGAGGGA |
| | CTGCCAAAACCCCTCACCCTTAGGTGGGAGCCTAGTTCCGGTGGCGGAGGCTCCGGAGGGGGCGGAAG |
| | CGGAGGTGGTGGATCCTGTCACCCTCGCCTGTCTCTGCATCGGCCAGCCCTCGAAGATCTACTACTGG |
| | GATCAGAAGCCAATTTGACCTGTACCCTGACCGGCCTGAGAGATGCCAGCGGCGCAACATTTACTTGG |
| | ACCCCATCTTCTGGTAAATCTGCCGTCCAGGGCCCGCCCGAGCGTGACCTCTGTGGCTGTTACAGCGT |
| | GAGCTCTGTCTTGCCGGGATGTGCTCAACCCTGGAATCATGGAGAGACGTTTACCTGCACCGCCGCAC |
| | ACCCAGAACTCAAAACACCCCTGACCGCCAATATCACCAAATCCGGTAACACCTTTCGACCGGAAGTG |
| | CACCTACTGCCACCACCTTCTGAAGAGCTTGCTTTGAATGAACTCGTGACTTTGACATGCCTAGCTAG |
| | GGGTTTCTCCCCTAAAGACGTTCTGGTAAGGTGGCTCCAAGGCAGCCAAGAGCTACCTCGAGAAAAGT |
| | ATCTAACTTGGGCTTCTAGGCAAGAGCCTTCCCAAGGGACAACCACGTATGCTGTGACCTCTATCCTG |
| | CGAGTCGCAGCAGAAGATTGGAAGAAGGGAGAGACATTTTCATGTATGGTCGGTCATGAAGCTCTCCC |
| | CCTTGCATTTACACAAAAGACTATTGATAGAATGGCAGGTAAGCCAACTCACATAAATGTCTCTGTCG |
| | TGATGGCTGAGGCCGACGGAACCTGTTAC |
| | (NLV_B2M_HLAA2_IgA2m2.1 nucleotide sequence) |
| 11 | NLVPMVATVGGGGSGGGGSGGGGSIQRTPKIQVYSRHPAENGKSNFLNCYVSGFHPSDIEVDLLKNGE |
| | RIEKVEHSDLSFSKDWSFYLLYYTEFTPTEKDEYACRVNHVTLSQPKIVKWDRDMGGGGSGGGGSGGG |
| | GSGGGGSGSHSMRYFFTSVSRPGRGEPRFIAVGYVDDTQFVRFDSDAASQRMEPRAPWIEQEGPEYWD |
| | GETRKVKAHSQTHRVDLGTLRGAYNQSEAGSHTVQRMYGCDVGSDWRFLRGYHQYAYDGKDYIALKED |
| | LRSWTAADMAAQTTKHKWEAAHVAEQLRAYLEGTCVEWLRRYLENGKETLQRTDAPKTHMTHHAVSDH |
| | EATLRCWALSFYPAEITLTWQRDGEDQTQDTELVETRPAGDGTFQKWAAVVVPSGQEQRYTCHVQHEG |
| | LPKPLTLRWEPSSPCRVPPPPPCCHPRLSLHRPALEDLLLGSEANLTCTLTGLRDASGATFTWTPSSG |
| | KSAVQGPPERDLCGCYSVSSVLPGCAQPWNHGETFTCTAAHPELKTPLTANITKSGNTFRPEVHLLPP |
| | PSEELALNELVTLTCLARGFSPKDVLVRWLQGSQELPREKYLTWASRQEPSQGTTTYAVTSILRVAAE |
| | DWKKGETFSCMVGHEALPLAFTQKTIDRMAGKPTHINVSVVMAEADGTCY |
| | (NLV_B2M_HLAA2_IgA2m2.2 amino acid sequence) |
| 12 | AACCTTGTGCCTATGGTCGCCACAGTGGGTGGTGGCGGCTCCGGAGGCGGTGGCTCCGGCGGCGGCGG |
| | CAGTATTCAAAGAACCCCCAAGATCCAGGTTTATTCTCGCCATCCCGCTGAGAACGGTAAATCTAATT |
| | TCCTTAACTGCTACGTTTCAGGCTTCCATCCAAGCGACATCGAAGTAGATCTGCTCAAAAATGGGGAG |
| | AGAATCGAGAAGGTAGAGCACAGTGACCTAAGTTTCAGTAAAGACTGGTCCTTCTACCTGCTCTACTA |
| | CACTGAGTTCACCCCCACAGAGAAGGACGAATATGCATGTCGGGTCAATCACGTCACCTTGTCTCAAC |
| | CTAAGATCGTGAAATGGGATCGGGATATGGGGGGTGGCGGCTCAGGCGGTGGAGGCTCAGGCGGGGGA |
| | GGATCTGGAGGCGGCGGTAGCGGCTCACACTCTATGAGGTATTTTTTTACCTCAGTGAGCAGGCCAGG |
| | CAGAGGTGAACCCAGATTCATTGCAGTTGGTTACGTTGATGATACCCAGTTCGTCCGCTTTGATTCTG |
| | ACGCCGCATCACAGAGGATGGAACCTAGGGCACCTTGGATAGAACAGGAAGGCCCAGAGTATTGGGAT |
| | GGCGAGACTAGAAAAGTGAAGGCTCATAGCCAGACACATCGAGTAGACCTGGGAACTCTCCGTGGAGC |
| | ATATAACCAGTCCGAGGCTGGCTCCCATACAGTGCAGAGGATGTACGGATGCGACGTGGGCAGCGATT |
| | GGCGTTTTCTACGAGGTTACCATCAGTACGCATACGATGGAAAAGACTATATTGCACTCAAGGAGGAC |
| | CTCCGAAGCTGGACCGCAGCTGATATGGCTGCACAGACTACCAAACATAAGTGGGAGGCAGCTCATGT |
| | TGCAGAGCAACTACGGGCATACCTCGAAGGGACTTGTGTGGAGTGGCTGAGAAGGTACCTAGAGAACG |
| | GGAAGGAGACCCTGCAGCGGACAGACGCCCCAAAGACCCATATGACCCACCACGCTGTCAGTGATCAT |
| | GAAGCCACATTGCGCTGCTGGGCACTTAGTTTCTATCCCGCAGAAATCACCTTGACCTGGCAGCGTGA |
| | CGGCGAAGATCAGACCCAGGACACCGAGCTTGTGGAAACAAGACCTGCCGGAGATGGAACCTTCCAGA |
| | AGTGGGCCGCTGTGGTCGTTCCCAGCGGCCAGGAGCAGCGGTATACTTGTCATGTCCAACATGAGGGA |
| | CTGCCAAAACCCCTCACCCTTAGGTGGGAGCCTAGTTCCCCATGTCGAGTTCCACCACCACCACCCTG |
| | CTGTCACCCTCGCCTGTCTCTGCATCGGCCAGCCCTCGAAGATCTACTACTGGGATCAGAAGCCAATT |
| | TGACCTGTACCCTGACCGGCCTGAGAGATGCCAGCGGCGCAACATTTACTTGGACCCCATCTTCTGGT |
| | AAATCTGCCGTCCAGGGCCCGCCCGAGCGTGACCTCTGTGGCTGTTACAGCGTGAGCTCTGTCTTGCC |
| | GGGATGTGCTCAACCCTGGAATCATGGAGAGACGTTTACCTGCACCGCCGCACACCCAGAACTCAAAA |
| | CACCCCTGACCGCCAATATCACCAAATCCGGTAACACCTTTCGACCGGAAGTGCACCTACTGCCACCA |
| | CCTTCTGAAGAGCTTGCTTTGAATGAACTCGTGACTTTGACATGCCTAGCTAGGGGTTTCTCCCCTAA |
| | AGACGTTCTGGTAAGGTGGCTCCAAGGCAGCCAAGAGCTACCTCGAGAAAAGTATCTAACTTGGGCTT |
| | CTAGGCAAGAGCCTTCCCAAGGGACAACCACGTATGCTGTGACCTCTATCCTGCGAGTCGCAGCAGAA |
| | GATTGGAAGAAGGGAGAGACATTTTCATGTATGGTCGGTCATGAAGCTCTCCCCCTTGCATTTACACA |
| | AAAGACTATTGATAGAATGGCAGGTAAGCCAACTCACATAAATGTCTCTGTCGTGATGGCTGAGGCCG |
| | ACGGAACCTGTTAC |
| | (NLV_B2M_HLAA2_IgA2m2.2 nucleotide sequence) |
| 13 | IKEEHVIIQAEFYLNPDQSGEFMFDFDGDEIFHVDMAKKETVWRLEEFGRFASFEAQGALANIAVDKA |
| | NLEIMTKRSNYTPITNVPPEVTVLTNSPVELREPNVLICFIDKFTPPVVNVTWLRNGKPVTTGVSETV |
| | FLPRFDHLFRKFHYLPFLPSTEDVYDCRVEHWGLDEPLLKHWEFDAPSPLPETTE |
| | (DRA1*01 soluble) |
| 14 | PKYVKQNTLKLATGGGGSGGGGSGGGGSGDTRPRFLWQLKFECHFFNGTERVRLLERCIYNQEESVRF |
| | DSDVGEYRAVTELGRPDAEYWNSQKDLLEQRRAAVDTYCRHNYGVGESFTVQRRVEPKVTVYPSKTQP |
| | LQHHNLLVCSVSGFYPGSIEVRWFRNGQEEKAGVVSTGLIQNGDWTFQTLVMLETVPRSGEVYTCQVE |
| | HPSVTSPLTVEWRARSESAQSKGGGGSGGGGSGGGGSIAELPPKVSVFVPPRDGFFGNPRKSKLICQA |
| | TGFSPRQIQVSWLREGKQVGSGVTTDQVQAEAKESGPTTYKVTSTLTIKESDWLGQDMFTCRVDHRGL |
| | TFQQNASSMCVPDQDTAIRVFAIPPSFASIFLTKSTKLTCLVTDLTTYDSVTISWTRQNGEAVKTHTN |
| | ISESHPNATFSAVGEASICEDDWNSGERFTCTVTHTDLPSPLKQTISRPKGVALHRPDVYLLPPAREQ |
| | LNLRESATITCLVTGFSPADVFVQWMQRGQPLSPEKYVTSAPMPEPQAPGRYFAHSILTVSEEEWNTG |
| | ETYTCVVAHEALPNRVTERTVDKSTGKPTLYNVSLVMSDTAGTCY |
| | (HA 306-318-Linker-DRB1*01-linker-gM2-4 amino acid sequence) |
| 15 | PKYVKQNTLKLATGGGGGSIEGRGSGGGSGDTRPRFLWQLKFECHFFNGTERVRLLERCIYNQEESVR |
| | FDSDVGEYRAVTELGRPDAEYWNSQKDLLEQRRAAVDTYCRHNYGVGESFTVQRRVEPKVTVYPSKTQ |
| | PLQHHNLLVCSVSGFYPGSIEVRWFRNGQEEKAGVVSTGLIQNGDWTFQTLVMLETVPRSGEVYTCQV |
| | EHPSVTSPLTVEWRARSESAQSKGGGGSGGGGSGGGGSIAELPPKVSVFVPPRDGFFGNPRKSKLICQ |
| | ATGFSPRQIQVSWLREGKQVGSGVTTDQVQAEAKESGPTTYKVTSTLTIKESDWLGQSMFTCRVDHRG |
| | LTFQQNASSMCVPDQDTAIRVFAIPPSFASIFLTKSTKLTCLVTDLTTYDSVTISWTRQNGEAVKTHT |
| | NISESHPNATFSAVGEASICEDDWNSGERFTCTVTHTDLPSPLKQTISRPKGVALHRPDVYLLPPARE |
| | QLNLRESATITCLVTGFSPADVFVQWMQRGQPLSPEKYVTSAPMPEPQAPGRYFAHSILTVSEEEWNT |
| | GETYTCVVAHEALPNRVTERTVDKSTGKPTLYNVSLVMSDTAGTCY |
| | (HA 306-318-LinkerXa-DRB1*01-linker-gM2-4 amino acid sequence) |
| 16 | IKEEHVIIQAEFYLNPDQSGEFMFDFDGDEIFHVDMAKKETVWRLEEFGRFASFEAQGALANIAVDKA |
| | NLEIMTKRSNYTPITNVPPEVTVLTNCPVELREPNVLICFIDKFTPPVVNVTWLRNGKPVTTGVSETV |
| | FLPRFDHLFRKFHYLPFLPSTEDVYDCRVEHWGLDEPLLKHWEFDAPSPLPETTE |
| | (DRA1*01 (S95C) amino acid sequence) |
| 17 | PKYVKQNTLKLATGGGGSGGGGSGGGGSGDTRPRFLWQLKFECHFFNGTERVRLLERCIYNQEESVRF |
| | DSDVGEYRAVTELGRPDAEYWNSQKDLLEQRRAAVDTYCRHNYGVGESFTVQRRVEPKVTVYPSKTQP |
| | LQHHNLLVCSVCGFYPGSIEVRWFRNGQEEKAGVVSTGLIQNGDWTFQTLVMLETVPRSGEVYTCQVE |
| | HPSVTSPLTVEWRARSESAQSKGGGGSGGGGSGGGGSIAELPPKVSVFVPPRDGFFGNPRKSKLICQA |
| | TGFSPRQIQVSWLREGKQVGSGVTTDQVQAEAKESGPTTYKVTSTLTIKESDWLGQDMFTCRVDHRGL |
| | TFQQNASSMCVPDQDTAIRVFAIPPSFASIFLTKSTKLTCLVTDLTTYDSVTISWTRQNGEAVKTHTN |
| | ISESHPNATFSAVGEASICEDDWNSGERFTCTVTHTDLPSPLKQTISRPKGVALHRPDVYLLPPAREQ |
| | LNLRESATITCLVTGFSPADVFVQWMQRGQPLSPEKYVTSAPMPEPQAPGRYFAHSILTVSEEEWNTG |
| | ETYTCVVAHEALPNRVTERTVDKSTGKPTLYNVSLVMSDTAGTCY |
| | (HA 306-318-Linker-DRB1*01 (S120C)-linker-gM2-4 amino acid |
| | sequence) |
| 18 | PVSKMRMATPLLMQAGGGGGSIEGRGSGGGSGDTRPRFLWQLKFECHFFNGTERVRLLERCIYNQEES |
| | VRFDSDVGEYRAVTELGRPDAEYWNSQKDLLEQRRAAVDTYCRHNYGVGESFTVQRRVEPKVTVYPSK |
| | TQPLQHHNLLVCSVSGFYPGSIEVRWFRNGQEEKAGVVSTGLIQNGDWTFQTLVMLETVPRSGEVYTC |
| | QVEHPSVTSPLTVEWRARSESAQSKGGGGSGGGGSGGGGSIAELPPKVSVFVPPRDGFFGNPRKSKLI |
| | CQATGFSPRQIQVSWLREGKQVGSGVTTDQVQAEAKESGPTTYKVTSTLTIKESDWLGQSMFTCRVDH |
| | RGLTFQQNASSMCVPDQDTAIRVFAIPPSFASIFLTKSTKLTCLVTDLTTYDSVTISWTRQNGEAVKT |
| | HTNISESHPNATFSAVGEASICEDDWNSGERFTCTVTHTDLPSPLKQTISRPKGVALHRPDVYLLPPA |
| | REQLNLRESATITCLVTGFSPADVFVQWMQRGQPLSPEKYVTSAPMPEPQAPGRYFAHSILTVSEEEW |
| | NTGETYTCVVAHEALPNRVTERTVDKSTGKPTLYNVSLVMSDTAGTCY |
| | (CLIP-LinkerXa-DRB1*01-linker-gM2-4 amino acid sequence) |
| 19 | NLVPMVATV |
| | (HLA-A*02:01 restricted CMV pp65 epitope) |
| 20 | ELAGIGILTV |
| | (HLA-A*02:01 restricted MART-1 epitope) |
| 21 | PKYVKQNTLKLAT |
| | (HA 306-318 peptide) |
| 22 | PVSKMRMATPLLMQA |
| | (CLIP peptide) |
| 23 | IQRTPKIQVYSRHPAENGKSNFLNCYVSGFHPSDIEVDLLKNGERIEKVEHSDLSFSKDWSFYLLYYT |
| | EFTPTEKDEYACRVNHVTLSQPKIVKWDRDMGGGGSGGGGSGGGGSGGGGSGSHSMRYFFTSVSRPGR |
| | GEPRFIAVGYVDDTQFVRFDSDAASQRMEPRAPWIEQEGPEYWDGETRKVKAHSQTHRVDLGTLRGCY |
| | NQSEAGSHTVQRMYGCDVGSDWRFLRGYHQYAYDGKDYIALKEDLRSWTAADMCAQTTKHKWEAAHVA |
| | EQLRAYLEGTCVEWLRRYLENGKETLQRTDAPKTHMTHHAVSDHEATLRCWALSFYPAEITLTWQRDG |
| | EDQTQDTELVETRPAGDGTFQKWAAVVVPSGQEQRYTCHVQHEGLPKPLTLRWEPSSGGGGSGGGGSG |
| | GGGSIAELPPKVSVFVPPRDGFFGNPRKSKLICQATGFSPRQIQVSWLREGKQVGSGVTTDQVQAEAK |
| | ESGPTTYKVTSTLTIKESDWLGQSMFTCRVDHRGLTFQQNASSMCVPDQDTAIRVFAIPPSFASIFLT |
| | KSTKLTCLVTDLTTYDSVTISWTRQNGEAVKTHTNISESHPNATFSAVGEASICEDDWNSGERFTCTV |
| | THTDLPSPLKQTISRPKGVALHRPDVYLLPPAREQLNLRESATITCLVTGFSPADVFVQWMQRGQPLS |
| | PEKYVTSAPMPEPQAPGRYFAHSILTVSEEEWNTGETYTCVVAHEALPNRVTERTVDKSTGKPTLYNV |
| | SLVMSDTAGTCY |
| | (beta2m-HLA-A2 (Y84C, A139C)-gM2-4 amino acid sequence) |
| 24 | GSHSMRYFFTSVSRPGRGEPRFIAVGYVDDTQFVRFDSDAASQRMEPRAPWIEQEGPEYWDGETRKVK |
| | AHSQTHRVDLGTLRGCYNQSEAGSHTVQRMYGCDVGSDWRFLRGYHQYAYDGKDYIALKEDLRSWTAA |
| | DMCAQTTKHKWEAAHVAEQLRAYLEGTCVEWLRRYLENGKETLQRTDAPKTHMTHHAVSDHEATLRCW |
| | ALSFYPAEITLTWQRDGEDQTQDTELVETRPAGDGTFQKWAAVVVPSGQEQRYTCHVQHEGLPKPLTL |
| | RWEPSSGGGGSGGGGSGGGGSIAELPPKVSVFVPPRDGFFGNPRKSKLICQATGFSPRQIQVSWLREG |
| | KQVGSGVTTDQVQAEAKESGPTTYKVTSTLTIKESDWLGQSMFTCRVDHRGLTFQQNASSMCVPDQDT |
| | AIRVFAIPPSFASIFLTKSTKLTCLVTDLTTYDSVTISWTRQNGEAVKTHTNISESHPNATFSAVGEA |
| | SICEDDWNSGERFTCTVTHTDLPSPLKQTISRPKGVALHRPDVYLLPPAREQLNLRESATITCLVTGF |
| | SPADVFVQWMQRGQPLSPEKYVTSAPMPEPQAPGRYFAHSILTVSEEEWNTGETYTCVVAHEALPNRV |
| | TERTVDKSTGKPTLYNVSLVMSDTAGTCY |
| | (HLA-A2 (Y84C, A139C)-gM2-4 amino acid sequence) |
| 25 | msrsvalavlallslsgleaiqrtpkiqvysrhpaengksnflncyvsgfhpsdievdll |
| | kngeriekvehsdlsfskdwsfyllyyteftptekdeyacrvnhvtlsqpkivkwdrdm |
| | (full length human beta-2-microglobulin) |
| 26 | QEDERIVLVDNKCKCARITSRIIRSSEDPNEDIVERNIRIIVPLNNRENISDPTSPLRTRFVYHLSDL |
| | CKKCDPTEVELDNQIVTATQSNICDEDSATETCYTYDRNKCYTAVVPLVYGGETKMVETALTPDACYP |
| | D |
| | (Full length mature Human Immunoglobulin J chain (without signal |
| | sequence)) |
| 27 | QEDERIVLVDNKCKCARITSRIIRSSEDPNEDIVERNIRIIVPLNNRENISDPTSPLRTRFVYHLSDL |
| | CKKCDPTEVELDNQIVTATQSNICDEDSATETCYTYDRNKCYTAVVPLVYGGETKMVETALTPDACYP |
| | DGGGGSGGGGSGGGGSFTVTVPKDLYVVEYGSNMTIECKFPVEKQLDLAALIVYWEMEDKNIIQFVHG |
| | EEDLKVQHSSYRQRARLLKDQLSLGNAALQITDVKLQDAGVYRCMISYGGADYKRITVKVNA |
| | (Jch-(G4S)3-PDL1D1 amino acid sequence) |
| 28 | QEDERIVLVDNKCKCARITSRIIRSSEDPNEDIVERNIRIIVPLNNRENISDPTSPLRTRFVYHLSDL |
| | CKKCDPTEVELDNQIVTATQSNICDEDSATETCYTYDRNKCYTAVVPLVYGGETKMVETALTPDACYP |
| | DGGGGSGGGGSGGGGSFTVTVPKDLYVVEYGSNMTIECKFPVEKQLDLAALIVYWEMEDKNIIQFVHG |
| | EEDLKVQHSSYRQRARLLKDQLSLGNAALQITDVKLQDAGVYRCMISYGGADYKRITVKVNAPYNKIN |
| | QRILVVDPVTSEHELTCQAEGYPKAEVIWTSSDHQVLSGKTTTTNSKREEKLFNVTSTLRINTTTNEI |
| | FYCTFRRLDPEENHTAELVIPELPLAHPPNER |
| | Jch-(G4S)3-PDL1ECD amino acid sequence) |
| 29 | MAVMAPRTLVLLLSGALALTQTWAGSHSMRYFFTSVSRPGRGEPRFIAVGYVDDTQFVRFDSDAASQR |
| | MEPRAPWIEQEGPEYWDGETRKVKAHSQTHRVDLGTLRGYYNQSEAGSHTVQRMYGCDVGSDWRFLRG |
| | YHQYAYDGKDYIALKEDLRSWTAADMAAQTTKHKWEAAHVAEQLRAYLEGTCVEWLRRYLENGKETLQ |
| | RTDAPKTHMTHHAVSDHEATLRCWALSFYPAEITLTWQRDGEDQTQDTELVETRPAGDGTFQKWAAVV |
| | VPSGQEQRYTCHVQHEGLPKPLTLRWEPSSQPTISIVGIIAGLVLFGAVITGAVVAAVMWRRKSSDRK |
| | GGSYSQAASSDSAQGSDVSLTACKV |
| | (HLA-A*02:01 full-length) |
| 30 | GSHSMRYFFTSVSRPGRGEPRFIAVGYVDDTQFVRFDSDAASQRMEPRAPWIEQEGPEYWDGETRKVK |
| | AHSQTHRVDLGTLRGYYNQSEAGSHTVQRMYGCDVGSDWRFLRGYHQYAYDGKDYIALKEDLRSWTAA |
| | DMAAQTTKHKWEAAHVAEQLRAYLEGTCVEWLRRYLENGKETLQRTDAPKTHMTHHAVSDHEATLRCW |
| | ALSFYPAEITLTWQRDGEDQTQDTELVETRPAGDGTFQKWAAVVVPSGQEQRYTCHVQHEGLPKPLTL |
| | RWEPSS |
| | (HLA-A*02:01; soluble domain residues 25-302) |
| 31 | GSHSMRYFFTSVSRPGRGEPRFIAVGYVDDTQFVRFDSDAASQKMEPRAPWIEQEGPEYWDQETRNMK |
| | AHSQTDRANLGTLRGYYNQSEDGSHTIQIMYGCDVGPDGRFLRGYRQDAYDGKDYIALNEDLRSWTAA |
| | DMAAQITKRKWEAVHAAEQRRVYLEGRCVDGLRRYLENGKETLQRTDPPKTHMTHHPISDHEATLRCW |
| | ALGFYPAEITLTWQRDGEDQTQDTELVETRPAGDGTFQKWAAVVVPSGEEQRYTCHVQHEGLPKPLTL |
| | RWELSS |
| | (HLA-A*01:01 soluble) |
| 32 | GSHSMRYFFTSVSRPGRGEPRFIAVGYVDDTQFVRFDSDAASQRMEPRAPWIEQEGPEYWDQETRNVK |
| | AQSQTDRVDLGTLRGYYNQSEAGSHTIQIMYGCDVGSDGRFLRGYRQDAYDGKDYIALNEDLRSWTAA |
| | DMAAQITKRKWEAAHEAEQLRAYLDGTCVEWLRRYLENGKETLQRTDPPKTHMTHHPISDHEATLRCW |
| | ALGFYPAEITLTWQRDGEDQTQDTELVETRPAGDGTFQKWAAVVVPSGEEQRYTCHVQHEGLPKPLTL |
| | RWELSS |
| | (HLA-A*03:01 soluble) |
| 33 | GSHSMRYFYTSVSRPGRGEPRFIAVGYVDDTQFVRFDSDAASQRMEPRAPWIEQEGPEYWDQETRNVK |
| | AQSQTDRVDLGTLRGYYNQSEDGSHTIQIMYGCDVGPDGRFLRGYRQDAYDGKDYIALNEDLRSWTAA |
| | DMAAQITKRKWEAAHAAEQQRAYLEGRCVEWLRRYLENGKETLQRTDPPKTHMTHHPISDHEATLRCW |
| | ALGFYPAEITLTWQRDGEDQTQDTELVETRPAGDGTFQKWAAVVVPSGEEQRYTCHVQHEGLPKPLTL |
| | RWELSS |
| | (HLA-A*11:01 soluble) |
| 34 | GSHSMRYFSTSVSRPGRGEPRFIAVGYVDDTQFVRFDSDAASQRMEPRAPWIEQEGPEYWDEETGKVK |
| | AHSQTDRENLRIALRYYNQSEAGSHTLQMMFGCDVGSDGRFLRGYHQYAYDGKDYIALKEDLRSWTAA |
| | DMAAQITKRKWEAAHVAEQQRAYLEGTCVDGLRRYLENGKETLQRTDPPKTHMTHHPISDHEATLRCW |
| | ALGFYPAEITLTWQRDGEDQTQDTELVETRPAGDGTFQKWAAVVVPSGEEQRYTCHVQHEGLPKPLTL |
| | RWEPSS |
| | (HLA-A*24:02 soluble) |
| 35 | GSHSMRYFYTSVSRPGRGEPRFISVGYVDDTQFVRFDSDAASPREEPRAPWIEQEGPEYWDRNTQIYK |
| | AQAQTDRESLRNLRGYYNQSEAGSHTLQSMYGCDVGPDGRLLRGHDQYAYDGKDYIALNEDLRSWTAA |
| | DTAAQITQRKWEAAREAEQRRAYLEGECVEWLRRYLENGKDKLERADPPKTHVTHHPISDHEATLRCW |
| | ALGFYPAEITLTWQRDGEDQTQDTELVETRPAGDRTFQKWAAVVVPSGEEQRYTCHVQHEGLPKPLTL |
| | RWEPSS |
| | (HLA-B*07:02 soluble) |
| 36 | GSHSMRYFSTSVSWPGRGEPRFIAVGYVDDTQFVRFDSDAASPRGEPREPWVEQEGPEYWDRETQKYK |
| | RQAQADRVNLRKLRGYYNQSEDGSHTLQRMFGCDLGPDGRLLRGYNQFAYDGKDYIALNEDLRSWTAA |
| | DTAAQITQRKWEAAREAEQRRAYLEGTCVEWLRRYLENGKETLQRAEHPKTHVTHHPVSDHEATLRCW |
| | ALGFYPAEITLTWQWDGEDQTQDTELVETRPAGDGTFQKWAAVVVPSGEEQRYTCHVQHEGLPEPLTL |
| | RWKPSS |
| | (HLA-C*04:01 soluble) |
| 37 | CSHSMRYFDTAVSRPGRGEPRFISVGYVDDTQFVRFDSDAASPRGEPRAPWVEQEGPEYWDRETQKYK |
| | RQAQADRVSLRNLRGYYNQSEDGSHTLQRMSGCDLGPDGRLLRGYDQSAYDGKDYIALNEDLRSWTAA |
| | DTAAQITQRKLEAARAAEQLRAYLEGTCVEWLRRYLENGKETLQRAEPPKTHVTHHPLSDHEATLRCW |
| | ALGFYPAEITLTWQRDGEDQTQDTELVETRPAGDGTFQKWAAVVVPSGQEQRYTCHMQHEGLQEPLTL |
| | SWEPSS |
| | (HLA-C*07:02 soluble) |
| 38 | GSHSMRYFDTAMSRPGRGEPRFISVGYVDDTQFVRFDSDAASPREEPRAPWIEQEGPEYWDRNTQIFK |
| | TNTQTDRESLRNLRGYYNQSEAGSHTLQSMYGCDVGPDGRLLRGHNQYAYDGKDYIALNEDLRSWTAA |
| | DTAAQITQRKWEAARVAEQDRAYLEGTCVEWLRRYLENGKDTLERADPPKTHVTHHPISDHEATLRCW |
| | ALGFYPAEITLTWQRDGEDQTQDTELVETRPAGDRTFQKWAAVVVPSGEEQRYTCHVQHEGLPKPLTL |
| | RWEPSS |
| | (HLA-B*08:01 soluble) |
| 39 | GSHSMRYFYTAMSRPGRGEPRFIAVGYVDDTQFVRFDSDAASPRTEPRAPWIEQEGPEYWDRNTQIFK |
| | TNTQTYRESLRNLRGYYNQSEAGSHIIQRMYGCDLGPDGRLLRGHDQSAYDGKDYIALNEDLSSWTAA |
| | DTAAQITQRKWEAARVAEQLRAYLEGLCVEWLRRYLENGKETLQRADPPKTHVTHHPVSDHEATLRCW |
| | ALGFYPAEITLTWQRDGEDQTQDTELVETRPAGDRTFQKWAAVVVPSGEEQRYTCHVQHEGLPKPLTL |
| | RWEPSS |
| | (HLA-B*35:01 soluble) |
| 40 | GSHSMRYFYTAMSRPGRGEPRFIAVGYVDDTQFVRFDSDAASPRMAPRAPWIEQEGPEYWDGETRNMK |
| | ASAQTYRENLRIALRYYNQSEAGSHIIQVMYGCDVGPDGRLLRGHDQSAYDGKDYIALNEDLSSWTAA |
| | DTAAQITQRKWEAARVAEQLRAYLEGLCVEWLRRYLENGKETLQRADPPKTHVTHHPISDHEATLRCW |
| | ALGFYPAEITLTWQRDGEDQTQDTELVETRPAGDRTFQKWAAVVVPSGEEQRYTCHVQHEGLPKPLTL |
| | RWEPSS |
| | (HLA-B*57:01 soluble) |
| 41 | GSHSMRYFYTAMSRPGRGEPRFIAVGYVDDTQFVRFDSDAASPRMAPRAPWIEQEGPEYWDGETRNMK |
| | ASAQTYRENLRIALRYYNQSEAGSHIIQVMYGCDVGPDGRLLRGHNQYAYDGKDYIALNEDLSSWTAA |
| | DTAAQITQRKWEAARVAEQLRAYLEGLCVEWLRRYLENGKETLQRADPPKTHVTHHPISDHEATLRCW |
| | ALGFYPAEITLTWQRDGEDQTQDTELVETRPAGDRTFQKWAAVVVPSGEEQRYTCHVQHEGLPKPLTL |
| | RWEPSS |
| | (HLA-B*57:03 soluble) |
| 42 | GSHSLKYFHTSVSRPGRGEPRFISVGYVDDTQFVRFDNDAASPRMVPRAPWMEQEGSEYWDRETRSAR |
| | DTAQIFRVNLRTLRGYYNQSEAGSHTLQWMHGCELGPDGRFLRGYEQFAYDGKDYLTLNEDLRSWTAV |
| | DTAAQISEQKSNDASEAEHQRAYLEDTCVEWLHKYLEKGKETLLHLEPPKTHVTHHPISDHEATLRCW |
| | ALGFYPAEITLTWQQDGEGHTQDTELVETRPAGDGTFQKWAAVVVPSGEEQRYTCHVQHEGLPEPVTL |
| | RWKPAS |
| | (HLA-E soluble) |
| 43 | CSHSMRYFYTAVSRPGRGEPRFIAVGYVDDTQFVRFDSDAASPRGEPRAPWVEQEGPEYWDRETQKYK |
| | RQAQTDRVSLRNLRGYYNQSEAGSHTLQWMYGCDLGPDGRLLRGYDQSAYDGKDYIALNEDLRSWTAA |
| | DTAAQITQRKWEAARAAEQQRAYLEGTCVEWLRRYLENGKETLQRAEHPKTHVTHHLVSDHEATLRCW |
| | ALGFYPAEITLTWQRDGEDQTQDTELVETRPAGDGTFQKWAAVVVPSGEEQRYTCHVQHEGLPEPLTL |
| | RWEPSS |
| | (HLA-C*16:01 soluble) |
| 44 | CSHSMRYFYTAVSRPGRGEPRFIAVGYVDDTQFVQFDSDAASPRGEPRAPWVEQEGPEYWDRETQKYK |
| | RQAQTDRVSLRNLRGYYNQSEAGSHTLQRMYGCDLGPDGRLLRGYNQFAYDGKDYIALNEDLRSWTAA |
| | DKAAQITQRKWEAAREAEQRRAYLEGTCVEWLRRYLENGKKTLQRAEHPKTHVTHHPVSDHEATLRCW |
| | ALGFYPAEITLTWQRDGEDQTQDTELVETRPAGDGTFQKWAAVVVPSGEEQRYTCHVQHEGLPEPLTL |
| | RWGPSS |
| | (HLA-C*08:02 soluble) |
| 45 | CSHSMRYFDTAVSRPGRGEPRFISVGYVDDTQFVRFDSDAASPRGEPRAPWVEQEGPEYWDRETQNYK |
| | RQAQADRVSLRNLRGYYNQSEDGSHTLQRMYGCDLGPDGRLLRGYDQSAYDGKDYIALNEDLRSWTAA |
| | DTAAQITQRKLEAARAAEQLRAYLEGTCVEWLRRYLENGKETLQRAEPPKTHVTHHPLSDHEATLRCW |
| | ALGFYPAEITLTWQRDGEDQTQDTELVETRPAGDGTFQKWAAVVVPSGQEQRYTCHMQHEGLQEPLTL |
| | SWEPSS |
| | (HLA-C*07:01 soluble) |
| 46 | CSHSMRYFYTAVSRPGRGEPRFIAVGYVDDTQFVQFDSDAASPRGEPRAPWVEQEGPEYWDRETQKYK |
| | RQAQTDRVNLRKLRGYYNQSEAGSHTLQRMYGCDLGPDGRLLRGYNQFAYDGKDYIALNEDLRSWTAA |
| | DKAAQITQRKWEAAREAEQRRAYLEGTCVEWLRRYLENGKKTLQRAEHPKTHVTHHPVSDHEATLRCW |
| | ALGFYPAEITLTWQRDGEDQTQDTELVETRPAGDGTFQKWAAVVVPSGEEQRYTCHVQHEGLPEPLTL |
| | RWGPSS |
| | (HLA-C*05:01 soluble) |
| 47 | GSHSMRYFYTAMSRPGRGEPRFITVGYVDDTLFVRFDSDATSPRKEPRAPWIEQEGPEYWDRETQISK |
| | TNTQTYRENLRTALRYYNQSEAGSHIIQRMYGCDVGPDGRLLRGYDQDAYDGKDYIALNEDLSSWTAA |
| | DTAAQITQRKWEAARVAEQDRAYLEGLCVESLRRYLENGKETLQRADPPKTHVTHHPISDHEVTLRCW |
| | ALGFYPAEITLTWQRDGEDQTQDTELVETRPAGDRTFQKWAAVVVPSGEEQRYTCHVQHEGLPKPLTL |
| | RWEPSS |
| | (HLA-B*44:02 soluble) |
| 48 | GSHSMRYFTTSVSRPGRGEPRFIAVGYVDDTQFVRFDSDAASQRMEPRAPWIEQEGPEYWDLQTRNVK |
| | AQSQTDRANLGTLRGYYNQSEAGSHTIQMMYGCDVGSDGRFLRGYRQDAYDGKDYIALNEDLRSWTAA |
| | DMAAQITQRKWEAARVAEQLRAYLEGTCVEWLRRYLENGKETLQRTDAPKTHMTHHAVSDHEATLRCW |
| | ALSFYPAEITLTWQRDGEDQTQDTELVETRPAGDGTFQKWASVVVPSGQEQRYTCHVQHEGLPKPLTL |
| | RWEPSS |
| | (HLA-A*29:02 soluble) |
| 49 | GSHSMRYFYTAMSRPGRGEPRFITVGYVDDTLFVRFDSDATSPRKEPRAPWIEQEGPEYWDRETQISK |
| | TNTQTYRENLRTALRYYNQSEAGSHIIQRMYGCDVGPDGRLLRGYDQDAYDGKDYIALNEDLSSWTAA |
| | DTAAQITQRKWEAARVAEQLRAYLEGLCVESLRRYLENGKETLQRADPPKTHVTHHPISDHEVTLRCW |
| | ALGFYPAEITLTWQRDGEDQTQDTELVETRPAGDRTFQKWAAVVVPSGEEQRYTCHVQHEGLPKPLTL |
| | RWEPSS |
| | (HLA-B*44:03 soluble) |
| 50 | GSHSMRYFYTAVSRPGRGEPHFIAVGYVDDTQFVRFDSDAASPRGEPRAPWVEQEGPEYWDRETQKYK |
| | RQAQTDRVSLRNLRGYYNQSEAGSHIIQRMYGCDVGPDGRLLRGYDQYAYDGKDYIALNEDLRSWTAA |
| | DTAAQITQRKWEAAREAEQLRAYLEGLCVEWLRRYLKNGKETLQRAEHPKTHVTHHPVSDHEATLRCW |
| | ALGFYPAEITLTWQWDGEDQTQDTELVETRPAGDGTFQKWAAVVVPSGEEQRYTCHVQHEGLPEPLTL |
| | RWEPSS |
| | (HLA-C*03:04 soluble) |
| 51 | GSHSMRYFHTAMSRPGRGEPRFITVGYVDDTLFVRFDSDATSPRKEPRAPWIEQEGPEYWDRETQISK |
| | TNTQTYRESLRNLRGYYNQSEAGSHTLQRMYGCDVGPDGRLLRGHNQYAYDGKDYIALNEDLRSWTAA |
| | DTAAQISQRKLEAARVAEQLRAYLEGECVEWLRRYLENGKDKLERADPPKTHVTHHPISDHEATLRCW |
| | ALGFYPAEITLTWQRDGEDQTQDTELVETRPAGDRTFQKWAAVVVPSGEEQRYTCHVQHEGLPKPLTL |
| | RWEPSS |
| | (HLA-B*40:01 soluble) |
| 52 | CSHSMRYFDTAVSRPGRGEPRFISVGYVDDTQFVRFDSDAASPRGEPRAPWVEQEGPEYWDRETQKYK |
| | RQAQADRVNLRKLRGYYNQSEDGSHTLQWMYGCDLGPDGRLLRGYDQSAYDGKDYIALNEDLRSWTAA |
| | DTAAQITQRKWEAAREAEQWRAYLEGTCVEWLRRYLENGKETLQRAEHPKTHVTHHPVSDHEATLRCW |
| | ALGFYPAEITLTWQRDGEDQTQDTELVETRPAGDGTFQKWAAVVVPSGEEQRYTCHVQHEGLPEPLTL |
| | RWEPSS |
| | (HLA-C*06:02 soluble) |
| 53 | GSHSMRYFYTAMSRPGRGEPRFIAVGYVDDTQFVRFDSDAASPRMAPRAPWIEQEGPEYWDRETQISK |
| | TNTQTYRESLRNLRGYYNQSEAGSHTLQRMYGCDVGPDGRLLRGHDQSAYDGKDYIALNEDLSSWTAA |
| | DTAAQITQRKWEAAREAEQWRAYLEGLCVEWLRRYLENGKETLQRADPPKTHVTHHPISDHEATLRCW |
| | ALGFYPAEITLTWQRDGEDQTQDTELVETRPAGDRTFQKWAAVVVPSGEEQRYTCHVQHEGLPKPLTL |
| | RWEPSS |
| | (HLA-B*15:01 soluble) |
| 54 | GSHSMRYFYTAVSRPGRGEPHFIAVGYVDDTQFVRFDSDAASPRGEPRAPWVEQEGPEYWDRETQKYK |
| | RQAQTDRVSLRNLRGYYNQSEARSHIIQRMYGCDVGPDGRLLRGYDQYAYDGKDYIALNEDLRSWTAA |
| | DTAAQITQRKWEAAREAEQLRAYLEGLCVEWLRRYLKNGKETLQRAEHPKTHVTHHPVSDHEATLRCW |
| | ALGFYPAEITLTWQWDGEDQTQDTELVETRPAGDGTFQKWAAVVVPSGEEQRYTCHVQHEGLPEPLTL |
| | RWEPSS |
| | (HLA-C*03:03 soluble) |
| 55 | GSHSMRYFSTSVSRPGSGEPRFIAVGYVDDTQFVRFDSDAASQRMEPRAPWIEQERPEYWDQETRNVK |
| | AQSQTDRVDLGTLRGYYNQSEAGSHTIQIMYGCDVGSDGRFLRGYEQHAYDGKDYIALNEDLRSWTAA |
| | DMAAQITQRKWEAARWAEQLRAYLEGTCVEWLRRYLENGKETLQRTDPPKTHMTHHPISDHEATLRCW |
| | ALGFYPAEITLTWQRDGEDQTQDTELVETRPAGDGTFQKWAAVVVPSGEEQRYTCHVQHEGLPKPLTL |
| | RWELSS |
| | (HLA-A*30:01 soluble) |
| 56 | GSHSMRYFYTAMSRPGRGEPRFITVGYVDDTQFVRFDSDATSPRMAPRAPWIEQEGPEYWDRETQISK |
| | TNTQTYRENLRTALRYYNQSEAGSHTWQTMYGCDLGPDGRLLRGHNQLAYDGKDYIALNEDLSSWTAA |
| | DTAAQITQLKWEAARVAEQLRAYLEGECVEWLRRYLENGKETLQRADPPKTHVTHHPISDHEATLRCW |
| | ALGFYPAEITLTWQRDGEDQTQDTELVETRPAGDRTFQKWAAVVVPSGEEQRYTCHVQHEGLPKPLTL |
| | RWEPSS |
| | (HLA-B*13:02 soluble) |
| 57 | CSHSMRYFYTAVSRPGRGEPRFIAVGYVDDTQFVRFDSDAASPRGEPRAPWVEQEGPEYWDRETQKYK |
| | RQAQADRVSLRNLRGYYNQSEAGSHTLQWMYGCDLGPDGRLLRGYDQSAYDGKDYIALNEDLRSWTAA |
| | DTAAQITQRKWEAAREAEQWRAYLEGTCVEWLRRYLENGKETLQRAEHPKTHVTHHPVSDHEATLRCW |
| | ALGFYPAEITLTWQRDGEDQTQDTELVETRPAGDGTFQKWAAVVVPSGEEQRYTCHVQHEGLPEPLTL |
| | RWEPSS |
| | (HLA-C*12:03 soluble) |
| 58 | GSHSMRYFYTSVSRPGRGEPRFIAVGYVDDTQFVRFDSDAASQRMEPRAPWIEQEGPEYWDRNTRNVK |
| | AHSQTDRANLGTLRGYYNQSEDGSHTIQRMYGCDVGPDGRFLRGYQQDAYDGKDYIALNEDLRSWTAA |
| | DMAAQITQRKWETAHEAEQWRAYLEGRCVEWLRRYLENGKETLQRTDAPKTHMTHHAVSDHEATLRCW |
| | ALSFYPAEITLTWQRDGEDQTQDTELVETRPAGDGTFQKWASVVVPSGQEQRYTCHVQHEGLPKPLTL |
| | RWEPSS |
| | (HLA-A*26:01 soluble) |
| 59 | GSHSMRYFYTSVSRPGRGEPRFISVGYVDDTQFVRFDSDAASPREEPRAPWIEQEGPEYWDRNTQICK |
| | TNTQTYRENLRIALRYYNQSEAGSHTLQRMYGCDVGPDGRLLRGHNQFAYDGKDYIALNEDLSSWTAA |
| | DTAAQITQRKWEAARVAEQLRTYLEGTCVEWLRRYLENGKETLQRADPPKTHVTHHPISDHEATLRCW |
| | ALGFYPAEITLTWQRDGEDQTQDTELVETRPAGDRTFQKWAAVVVPSGEEQRYTCHVQHEGLPKPLTL |
| | RWEPSS |
| | (HLA-B*38:01 soluble) |
| 60 | GSHSMRYFYTAVSRPGRGEPRFISVGYVDDTQFVRFDSDAASPREEPRAPWIEQEGPEYWDRNTQICK |
| | TNTQTDRESLRNLRGYYNQSEAGSHTLQWMYGCDVGPDGRLLRGYNQFAYDGKDYIALNEDLSSWTAA |
| | DTAAQITQRKWEAAREAEQLRAYLEGTCVEWLRRHLENGKETLQRADPPKTHVTHHPISDHEATLRCW |
| | ALGFYPAEITLTWQRDGEDQTQDTELVETRPAGDRTFQKWAAVVVPSGEEQRYTCHVQHEGLPKPLTL |
| | RWEPSS |
| | (HLA-B*14:02 soluble) |
| 61 | GSHSMRYFTTSVSRPGRGEPRFIAVGYVDDTQFVRFDSDAASQRMEPRAPWIEQEGPEYWDRNTRNVK |
| | AHSQIDRVDLGTLRGYYNQSEAGSHTIQMMYGCDVGSDGRFLRGYQQDAYDGKDYIALNEDLRSWTAA |
| | DMAAQITQRKWEAARVAEQLRAYLEGTCVEWLRRHLENGKETLQRTDPPRTHMTHHAVSDHEATLRCW |
| | ALSFYPAEITLTWQRDGEDQTQDTELVETRPAGDGTFQKWASVVVPSGQEQRYTCHVQHEGLPKPLTL |
| | RWEPSS |
| | (HLA-A*33:01 soluble) |
| 62 | GSHSMRYFSTSVSRPGRGEPRFIAVGYVDDTQFVRFDSDAASQRMEPRAPWIEQEGPEYWDEETGKVK |
| | AHSQTDRENLRIALRYYNQSEAGSHTLQMMFGCDVGSDGRFLRGYHQYAYDGKDYIALKEDLRSWTAA |
| | DMAAQITQRKWEAARVAEQLRAYLEGTCVDGLRRYLENGKETLQRTDPPKTHMTHHPISDHEATLRCW |
| | ALGFYPAEITLTWQRDGEDQTQDTELVETRPAGDGTFQKWAAVVVPSGEEQRYTCHVQHEGLPKPLTL |
| | RWEPSS |
| | (HLA-A*23:01 soluble) |
| 63 | GSHSMRYFYTSVSRPGRGEPRFIAVGYVDDTQFVRFDSDAASQRMEPRAPWIEQEGPEYWDRNTRNVK |
| | AHSQTDRESLRIALRYYNQSEDGSHTIQRMYGCDVGPDGRFLRGYQQDAYDGKDYIALNEDLRSWTAA |
| | DMAAQITQRKWETAHEAEQWRAYLEGRCVEWLRRYLENGKETLQRTDAPKTHMTHHAVSDHEATLRCW |
| | ALSFYPAEITLTWQRDGEDQTQDTELVETRPAGDGTFQKWASVVVPSGQEQRYTCHVQHEGLPKPLTL |
| | RWEPSS |
| | (HLA-A*25:01 soluble) |
| 64 | GSHSMRYFHTSVSRPGRGEPRFISVGYVDGTQFVRFDSDAASPRTEPRAPWIEQEGPEYWDRNTQISK |
| | TNTQTYRESLRNLRGYYNQSEAGSHTLQRMYGCDVGPDGRLLRGHDQSAYDGKDYIALNEDLSSWTAA |
| | DTAAQITQRKWEAARVAEQLRAYLEGTCVEWLRRHLENGKETLQRADPPKTHVTHHPISDHEATLRCW |
| | ALGFYPAEITLTWQRDGEDQTQDTELVETRPAGDRTFQKWAAVVVPSGEEQRYTCHVQHEGLPKPLTL |
| | RWEPSS |
| | (HLA-B*18:01 soluble) |
| 65 | GSHSMRYFHTSVSRPGRGEPRFISVGYVDDTQFVRFDSDAASPRTEPRAPWIEQEGPEYWDRETQISK |
| | TNTQTYRFDLRTLLRYYNQSEAGSHTIQRMSGCDVGPDGRLLRGYNQFAYDGKDYIALNEDLSSWTAA |
| | DTAAQITQRKWEAARVAEQDRAYLEGTCVEWLRRYLENGKETLQRADPPKTHVTHHPISDHEATLRCW |
| | ALGFYPAEITLTWQRDGEDQTQDTELVETRPAGDRTFQKWAAVVVPSGEEQRYTCHVQHEGLPKPLTL |
| | RWEPSS |
| | (HLA-B*37:01 soluble) |
| 66 | GSHSMRYFYTAMSRPGRGEPRFIAVGYVDDTQFVRFDSDAASPRTEPRAPWIEQEGPEYWDRNTQIFK |
| | TNTQTYRENLRIALRYYNQSEAGSHTWQTMYGCDVGPDGRLLRGHNQYAYDGKDYIALNEDLSSWTAA |
| | DTAAQITQRKWEAAREAEQLRAYLEGLCVEWLRRHLENGKETLQRADPPKTHVTHHPVSDHEATLRCW |
| | ALGFYPAEITLTWQRDGEDQTQDTELVETRPAGDRTFQKWAAVVVPSGEEQRYTCHVQHEGLPKPLTL |
| | RWEPSS |
| | (HLA-B*51:01 soluble) |
| 67 | CSHSMRYFSTSVSRPGRGEPRFIAVGYVDDTQFVRFDSDAASPRGEPRAPWVEQEGPEYWDRETQKYK |
| | RQAQTDRVSLRNLRGYYNQSEAGSHTLQWMFGCDLGPDGRLLRGYDQSAYDGKDYIALNEDLRSWTAA |
| | DTAAQITQRKWEAAREAEQRRAYLEGTCVEWLRRYLENGKETLQRAEHPKTHVTHHPVSDHEATLRCW |
| | ALGFYPAEITLTWQWDGEDQTQDTELVETRPAGDGTFQKWAAVVVPSGEEQRYTCHVQHEGLPEPLTL |
| | RWEPSS |
| | (HLA-C*14:02 soluble) |
| 68 | CSHSMRYFYTAVSRPGRGEPHFIAVGYVDDTQFVRFDSDAASPRGEPRAPWVEQEGPEYWDRETQNYK |
| | RQAQTDRVNLRKLRGYYNQSEAGSHIIQRMYGCDLGPDGRLLRGHDQLAYDGKDYIALNEDLRSWTAA |
| | DTAAQITQRKWEAAREAEQLRAYLEGTCVEWLRRYLENGKETLQRAEHPKTHVTHHPVSDHEATLRCW |
| | ALGFYPAEITLTWQRDGEDQTQDTELVETRPAGDGTFQKWAAVVVPSGEEQRYTCHVQHEGLPEPLTL |
| | RWEPSS |
| | (HLA-C*15:02 soluble) |
| 69 | CSHSMRYFYTAVSRPSRGEPHFIAVGYVDDTQFVRFDSDAASPRGEPRAPWVEQEGPEYWDRETQKYK |
| | RQAQTDRVNLRKLRGYYNQSEAGSHTLQRMYGCDLGPDGRLLRGYDQSAYDGKDYIALNEDLRSWTAA |
| | DTAAQITQRKWEAAREAEQWRAYLEGECVEWLRRYLENGKETLQRAEHPKTHVTHHPVSDHEATLRCW |
| | ALGFYPTEITLTWQRDGEDQTQDTELVETRPAGDGTFQKWAAVVVPSGEEQRYTCHVQHEGLPEPLTL |
| | RWEPSS |
| | (HLA-C*02:02 soluble) |
| 70 | GSHSMRYFHTSVSRPGRGEPRFITVGYVDDTLFVRFDSDAASPREEPRAPWIEQEGPEYWDRETQICK |
| | AKAQTDRFDLRTLLRYYNQSEAGSHTLQNMYGCDVGPDGRLLRGYHQDAYDGKDYIALNEDLSSWTAA |
| | DTAAQITQRKWEAARVAEQLRAYLEGECVEWLRRYLENGKETLQRADPPKTHVTHHPISDHEATLRCW |
| | ALGFYPAEITLTWQRDGEDQTQDTELVETRPAGDRTFQKWAAVVVPSGEEQRYTCHVQHEGLPKPLTL |
| | RWEPSS |
| | (HLA-B*27:05 soluble) |
| 71 | GSHSMRYFTTSVSRPGRGEPRFIAVGYVDDTQFVRFDSDAASQRMEPRAPWIEQERPEYWDQETRNVK |
| | AHSQIDRVDLGTLRGYYNQSEAGSHTIQMMYGCDVGSDGRFLRGYQQDAYDGKDYIALNEDLRSWTAA |
| | DMAAQITQRKWEAARVAEQLRAYLEGTCVEWLRRYLENGKETLQRTDPPKTHMTHHAVSDHEATLRCW |
| | ALSFYPAEITLTWQRDGEDQTQDTELVETRPAGDGTFQKWASVVVPSGQEQRYTCHVQHEGLPKPLTL |
| | RWEPSS |
| | (HLA-A*31:01 soluble) |
| 72 | GSHSMRYFSTSVSRPGSGEPRFIAVGYVDDTQFVRFDSDAASQRMEPRAPWIEQERPEYWDQETRNVK |
| | AHSQTDRENLGTLRGYYNQSEAGSHTIQIMYGCDVGSDGRFLRGYEQHAYDGKDYIALNEDLRSWTAA |
| | DMAAQITQRKWEAARRAEQLRAYLEGTCVEWLRRYLENGKETLQRTDPPKTHMTHHPISDHEATLRCW |
| | ALGFYPAEITLTWQRDGEDQTQDTELVETRPAGDGTFQKWAAVVVPSGEEQRYTCHVQHEGLPKPLTL |
| | RWELSS |
| | (HLA-A*30:02 soluble) |
| 73 | GSHSMRYFYTSVSRPGRGEPRFISVGYVDDTQFVRFDSDAASPREEPRAPWIEQEGPEYWDRNTQIYK |
| | AQAQTDRESLRNLRGYYNQSEAGSHTLQSMYGCDVGPDGRLLRGHNQYAYDGKDYIALNEDLRSWTAA |
| | DTAAQITQRKWEAARVAEQDRAYLEGTCVEWLRRYLENGKDTLERADPPKTHVTHHPISDHEATLRCW |
| | ALGFYPAEITLTWQRDGEDQTQDTELVETRPAGDRTFQKWAAVVVPSGEEQRYTCHVQHEGLPKPLTL |
| | RWEPSS |
| | (HLA-B*42:01 soluble) |
| 74 | GSHSMRYFYTAVSRPGRGEPRFIAVGYVDDTQFVRFDSDAASPRGEPRAPWVEQEGPEYWDRETQKYK |
| | RQAQADRVNLRKLRGYYNQSEAGSHTIQRMYGCDLGPDGRLLRGYNQFAYDGKDYIALNEDLRSWTAA |
| | DTAAQISQRKLEAAREAEQLRAYLEGECVEWLRGYLENGKETLQRAERPKTHVTHHPVSDHEATLRCW |
| | ALGFYPAEITLTWQRDGEDQTQDTELVETRPAGDGTFQKWAAVVVPSGQEQRYTCHVQHEGLQEPCTL |
| | RWKPSS |
| | (HLA-C*17:01 soluble) |
| 75 | GSHSMRYFYTAMSRPGRGEPRFIAVGYVDDTQFVRFDSDAASPRTEPRAPWIEQEGPEYWDRNTQIFK |
| | TNTQTYRESLRNLRGYYNQSEAGSHIIQRMYGCDLGPDGRFLRGHNQYAYDGKDYIALNEDLSSWTAA |
| | DTAAQITQRKWEAARVAEQLRAYLEGLCVEWLRRYLENGKETLQRADPPKTHVTHHPVSDHEATLRCW |
| | ALGFYPAEITLTWQRDGEDQTQDTELVETRPAGDRTFQKWAAVVVPSGEEQRYTCHVQHEGLPKPLTL |
| | RWEPSS |
| | (HLA-B*35:02 soluble) |
| 76 | GSHSMRYFYTSVSRPGRGEPRFISVGYVDDTQFVRFDSDAASPREEPRAPWIEQEGPEYWDRNTQICK |
| | TNTQTDRESLRNLRGYYNQSEAGSHTWQTMYGCDVGPDGRLLRGHNQFAYDGKDYIALNEDLSSWTAA |
| | DTAAQITQRKWEAARVAEQLRTYLEGTCVEWLRRYLENGKETLQRADPPKTHVTHHPISDHEATLRCW |
| | ALGFYPAEITLTWQRDGEDQTQDTELVETRPAGDRTFQKWAAVVVPSGEEQRYTCHVQHEGLPKPLTL |
| | RWEPSS |
| | (HLA-B*39:06 soluble) |
| 77 | GSHSMRYFYTAVSRPGRGEPHFIAVGYVDDTQFVRFDSDAASPRGEPRAPWVEQEGPEYWDRETQKYK |
| | RQAQTDRVSLRNLRGYYNQSEAGSHILQRMYGCDVGPDGRLLRGYDQSAYDGKDYIALNEDLRSWTAA |
| | DTAAQITQRKWEAAREAEQLRAYLEGLCVEWLRRYLKNGKETLQRAEHPKTHVTHHPVSDHEATLRCW |
| | ALGFYPAEITLTWQWDGEDQTQDTELVETRPAGDGTFQKWAAVVVPSGEEQRYTCHVQHEGLPEPLTL |
| | RWEPSS |
| | (HLA-C*03:02 soluble) |
| 78 | GSHSMRYFYTAMSRPGRGEPRFIAVGYVDDTQFVRFDSDAASPRTEPRAPWIEQEGPEYWDGETRNMK |
| | ASAQTYRENLRIALRYYNQSEAGSHIIQRMYGCDLGPDGRLLRGHDQSAYDGKDYIALNEDLSSWTAA |
| | DTAAQITQRKWEAARVAEQLRAYLEGLCVEWLRRYLENGKETLQRADPPKTHVTHHPVSDHEATLRCW |
| | ALGFYPAEITLTWQRDGEDQTQDTELVETRPAGDRTFQKWAAVVVPSGEEQRYTCHVQHEGLPKPLTL |
| | RWEPSS |
| | (HLA-B*58:01 soluble) |
| 79 | GSHSMRYFTTSVSRPGRGEPRFIAVGYVDDTQFVRFDSDAASQRMEPRAPWIEQEGPEYWDRNTRNVK |
| | AHSQIDRVDLGTLRGYYNQSEAGSHTIQMMYGCDVGSDGRFLRGYQQDAYDGKDYIALNEDLRSWTAA |
| | DMAAQITQRKWEAARVAEQLRAYLEGTCVEWLRRYLENGKETLQRTDPPKTHMTHHAVSDHEATLRCW |
| | ALSFYPAEITLTWQRDGEDQTQDTELVETRPAGDGTFQKWASVVVPSGQEQRYTCHVQHEGLPKPLTL |
| | RWEPSS |
| | (HLA-A*33:03 soluble) |
| 80 | GSHSMRYFYTSMSRPGRGEPRFIAVGYVDDTQFVRFDSDAASQRMEPRAPWIEQEGPEYWDRNTRNVK |
| | AQSQTDRVDLGTLRGYYNQSEAGSHTIQRMYGCDVGPDGRFLRGYHQYAYDGKDYIALKEDLRSWTAA |
| | DMAAQTTKHKWEAAHVAEQWRAYLEGTCVEWLRRYLENGKETLQRTDAPKTHMTHHAVSDHEATLRCW |
| | ALSFYPAEITLTWQRDGEDQTQDTELVETRPAGDGTFQKWVAVVVPSGQEQRYTCHVQHEGLPKPLTL |
| | RWEPSS |
| | (HLA-A*68:02 soluble) |
| 81 | CSHSMKYFFTSVSRPGRGEPRFISVGYVDDTQFVRFDSDAASPRGEPRAPWVEQEGPEYWDRETQKYK |
| | RQAQTDRVSLRNLRGYYNQSEAGSHTLQWMCGCDLGPDGRLLRGYDQYAYDGKDYIALNEDLRSWTAA |
| | DTAAQITQRKWEAAREAEQRRAYLEGTCVEWLRRYLENGKETLQRAEHPKTHVTHHPVSDHEATLRCW |
| | ALGFYPAEITLTWQWDGEDQTQDTELVETRPAGDGTFQKWAAVMVPSGEEQRYTCHVQHEGLPEPLTL |
| | RWEPSS |
| | (HLA-C*01:02 soluble) |
| 82 | CSHSMRYFDTAVSRPGRGEPRFISVGYVDDTQFVRFDSDAASPRGEPRAPWVEQEGPEYWDRETQKYK |
| | RQAQADRVSLRNLRGYYNQSEDGSHTFQRMYGCDLGPDGRLLRGYDQFAYDGKDYIALNEDLRSWTAA |
| | DTAAQITQRKLEAARAAEQDRAYLEGTCVEWLRRYLENGKKTLQRAEPPKTHVTHHPLSDHEATLRCW |
| | ALGFYPAEITLTWQRDGEDQTQDTELVETRPAGDGTFQKWAAVVVPSGQEQRYTCHMQHEGLQEPLTL |
| | SWEPSS |
| | (HLA-C*07:04 soluble) |
| 83 | GSHSMRYFYTSVSRPGRGEPRFIAVGYVDDTQFVRFDSDAASQRMEPRAPWIEQEGPEYWDRNTRNVK |
| | AQSQTDRVDLGTLRGYYNQSEAGSHTIQMMYGCDVGSDGRFLRGYRQDAYDGKDYIALKEDLRSWTAA |
| | DMAAQTTKHKWEAAHVAEQWRAYLEGTCVEWLRRYLENGKETLQRTDAPKTHMTHHAVSDHEATLRCW |
| | ALSFYPAEITLTWQRDGEDQTQDTELVETRPAGDGTFQKWVAVVVPSGQEQRYTCHVQHEGLPKPLTL |
| | RWEPSS |
| | (HLA-A*68:01 soluble) |
| 84 | GSHSMRYFFTSVSRPGRGEPRFIAVGYVDDTQFVRFDSDAASQRMEPRAPWIEQEGPEYWDQETRNVK |
| | AHSQTDRESLRIALRYYNQSEAGSHTIQMMYGCDVGPDGRLLRGYQQDAYDGKDYIALNEDLRSWTAA |
| | DMAAQITQRKWEAARVAEQLRAYLEGTCVEWLRRYLENGKETLQRTDAPKTHMTHHAVSDHEATLRCW |
| | ALSFYPAEITLTWQRDGEDQTQDTELVETRPAGDGTFQKWASVVVPSGQEQRYTCHVQHEGLPKPLTL |
| | RWEPSS |
| | (HLA-A*32:01 soluble) |
| 85 | GSHSMRYFHTAMSRPGRGEPRFITVGYVDDTLFVRFDSDATSPRKEPRAPWIEQEGPEYWDRETQISK |
| | TNTQTYRENLRIALRYYNQSEAGSHTWQRMYGCDLGPDGRLLRGYNQLAYDGKDYIALNEDLSSWTAA |
| | DTAAQITQRKWEAAREAEQLRAYLEGLCVEWLRRYLENGKETLQRADPPKTHVTHHPISDHEATLRCW |
| | ALGFYPAEITLTWQRDGEDQTQDTELVETRPAGDRTFQKWAAVVVPSGEEQRYTCHVQHEGLPKPLTL |
| | RWEPSS |
| | (HLA-B*49:01 soluble) |
| 86 | GSHSMRYFYTAMSRPGRGEPRFIAVGYVDDTQFVRFDSDAASPRTEPRAPWIEQEGPEYWDRNTQIFK |
| | TNTQTYRENLRIALRYYNQSEAGSHIIQRMYGCDLGPDGRLLRGHDQSAYDGKDYIALNEDLSSWTAA |
| | DTAAQITQRKWEAARVAEQLRAYLEGLCVEWLRRYLENGKETLQRADPPKTHVTHHPVSDHEATLRCW |
| | ALGFYPAEITLTWQRDGEDQTQDTELVETRPAGDRTFQKWAAVVVPSGEEQRYTCHVQHEGLPKPLTL |
| | RWEPSS |
| | (HLA-B*53:01 soluble) |
| 87 | GSHSMRYFHTAMSRPGRGEPRFITVGYVDDTLFVRFDSDATSPRKEPRAPWIEQEGPEYWDRETQISK |
| | TNTQTYRESLRNLRGYYNQSEAGSHTWQRMYGCDLGPDGRLLRGYNQLAYDGKDYIALNEDLSSWTAA |
| | DTAAQITQRKWEAAREAEQLRAYLEGLCVEWLRRYLENGKETLQRADPPKTHVTHHPISDHEATLRCW |
| | ALGFYPAEITLTWQRDGEDQTQDTELVETRPAGDRTFQKWAAVVVPSGEEQRYTCHVQHEGLPKPLTL |
| | RWEPSS |
| | (HLA-B*50:01 soluble) |
| 88 | GSHSMRYFYTSVSRPGRGEPRFIAVGYVDDTQFVRFDSDAASRRMEPRAPWIEQEGPEYWDGETRKVK |
| | AHSQTHRVDLGTLRGYYNQSEAGSHTLQRMYGCDVGSDWRFLRGYHQYAYDGKDYIALKEDLRSWTAA |
| | DMAAQTTKHKWEAAHVAEQWRAYLEGTCVEWLRRYLENGKETLQRTDAPKTHMTHHAVSDHEATLRCW |
| | ALSFYPAEITLTWQRDGEDQTQDTELVETRPAGDGTFQKWAAVVVPSGQEQRYTCHVQHEGLPKPLTL |
| | RWEPSS |
| | (HLA-A*02:05 soluble) |
| 89 | GSHSMRYFYTAMSRPGRGEPRFIAVGYVDDTQFVRFDSDAASPREEPRAPWIEQEGPEYWDRNTQIYK |
| | AQAQTDRESLRNLRGYYNQSEAGSHTWQTMYGCDLGPDGRLLRGHNQLAYDGKDYIALNEDLSSWTAA |
| | DTAAQITQRKWEAAREAEQLRAYLEGTCVEWLRRYLENGKETLQRADPPKTHVTHHPISDHEATLRCW |
| | ALGFYPAEITLTWQRDGEDQTQDTELVETRPAGDRTFQKWAAVVVPSGEEQRYTCHVQHEGLPKPLTL |
| | RWEPSS |
| | (HLA-B*55:01 soluble) |
| 90 | GSHSMRYFHTAMSRPGRGEPRFITVGYVDDTLFVRFDSDATSPRKEPRAPWIEQEGPEYWDRETQISK |
| | TNTQTYRESLRNLRGYYNQSEAGSHTWQRMYGCDLGPDGRLLRGYNQLAYDGKDYIALNEDLSSWTAA |
| | DTAAQITQRKWEAARVAEQDRAYLEGLCVESLRRYLENGKETLQRADPPKTHVTHHPISDHEATLRCW |
| | ALGFYPAEITLTWQRDGEDQTQDTELVETRPAGDRTFQKWAAVVVPSGEEQRYTCHVQHEGLPKPLTL |
| | RWEPSS |
| | (HLA-B*45:01 soluble) |
| 91 | GSHSMRYFYTAMSRPGRGEPRFIAVGYVDDTQFVRFDSDAASPRTEPRAPWIEQEGPEYWDRETQISK |
| | TNTQTYRENLRIALRYYNQSEAGSHTWQTMYGCDVGPDGRLLRGHNQYAYDGKDYIALNEDLSSWTAA |
| | DTAAQITQRKWEAAREAEQLRAYLEGLCVEWLRRHLENGKETLQRADPPKTHVTHHPVSDHEATLRCW |
| | ALGFYPAEITLTWQRDGEDQTQDTELVETRPAGDRTFQKWAAVVVPSGEEQRYTCHVQHEGLPKPLTL |
| | RWEPSS |
| | (HLA-B*52:01 soluble) |
| 92 | CSHSMRYFYTAVSRPGRGEPRFIAVGYVDDTQFVRFDSDAASPRGEPRAPWVEQEGPEYWDRETQKYK |
| | RQAQADRVSLRNLRGYYNQSEAGSHTLQRMYGCDLGPDGRLLRGYDQSAYDGKDYIALNEDLRSWTAA |
| | DTAAQITQRKWEAAREAEQWRAYLEGTCVEWLRRYLENGKETLQRAEHPKTHVTHHPVSDHEATLRCW |
| | ALGFYPAEITLTWQRDGEDQTQDTELVETRPAGDGTFQKWAAVVVPSGEEQRYTCHVQHEGLPEPLTL |
| | RWEPSS |
| | (HLA-C*12:02 soluble) |
| 93 | GSHSMRYFYTAMSRPGRGEPRFIAVGYVDDTQFVRFDSDAASPRTEPRAPWIEQEGPEYWDRNTQIFK |
| | TNTQTYRESLRNLRGYYNQSEAGSHIIQRMYGCDLGPDGRLLRGHDQFAYDGKDYIALNEDLSSWTAA |
| | DTAAQITQRKWEAARVAEQLRAYLEGLCVEWLRRYLENGKETLQRADPPKTHVTHHPVSDHEATLRCW |
| | ALGFYPAEITLTWQRDGEDQTQDTELVETRPAGDRTFQKWAAVVVPSGEEQRYTCHVQHEGLPKPLTL |
| | RWEPSS |
| | (HLA-B*35:03 soluble) |
| 94 | GSHSMRYFHTSVSRPGRGEPRFITVGYVDDTLFVRFDSDATSPRKEPRAPWIEQEGPEYWDRETQISK |
| | TNTQTYRESLRNLRGYYNQSEAGSHTLQSMYGCDVGPDGRLLRGHNQYAYDGKDYIALNEDLRSWTAA |
| | DTAAQITQRKWEAARVAEQLRAYLEGECVEWLRRYLENGKETLQRADPPKTHVTHHPISDHEATLRCW |
| | ALGFYPAEITLTWQRDGEDQTQDTELVETRPAGDRTFQKWAAVVVPSGEEQRYTCHVQHEGLPKPLTL |
| | RWEPSS |
| | (HLA-B*40:02 soluble) |
| 95 | GSHSMRYFYTAMSRPGRGEPRFISVGYVDDTQFVRFDSDAASPREEPRAPWIEQEGPEYWDRETQISK |
| | TNTQTYRESLRNLRGYYNQSEAGSHTLQRMYGCDVGPDGRLLRGHDQSAYDGKDYIALNEDLSSWTAA |
| | DTAAQITQRKWEAAREAEQLRAYLEGLCVEWLRRYLENGKETLQRADPPKTHVTHHPISDHEATLRCW |
| | ALGFYPAEITLTWQRDGEDQTQDTELVETRPAGDRTFQKWAAVVVPSGEEQRYTCHVQHEGLPKPLTL |
| | RWEPSS |
| | (HLA-B*15:03 soluble) |
| 96 | GSHSMRYFFTSVSRPGRGEPRFIAVGYVDDTQFVRFDSDAASQRMEPRAPWIEQEGPEYWDQETRNVK |
| | AHSQTDRVDLGTLRGYYNQSEAGSHTIQMMYGCDVGPDGRLLRGYQQDAYDGKDYIALNEDLRSWTAA |
| | DMAAQITQRKWEAARVAEQLRAYLEGTCVEWLRRYLENGKETLQRTDAPKTHMTHHAVSDHEATLRCW |
| | ALSFYPAEITLTWQRDGEDQTQDTELVETRPAGDGTFQKWASVVVPSGQEQRYTCHVQHEGLPKPLTL |
| | RWEPSS |
| | (HLA-A*74:01 soluble) |
| 97 | IKEEHVIIQAEFYLNPDQSGEFMFDFDGDEIFHVDMAKKETVWRLEEFGRFASFEAQGALANIAVDKA |
| | NLEIMTKRSNYTPITNVPPEVTVLTNSPVELREPNVLICFIDKFTPPVVNVTWLRNGKPVTTGVSETV |
| | FLPRFDHLFRKFHYLPFLPSTEDVYDCRVEHWGLDEPLLKHWEFDAPSPLPETTE |
| | (HLA-DRA*01:01 soluble) |
| 98 | GDTRPRFLWQLKFECHFFNGTERVRLLERCIYNQEESVRFDSDVGEYRAVTELGRPDAEYWNSQKDLL |
| | EQRRAAVDTYCRHNYGVGESFTVQRRVEPKVTVYPSKTQPLQHHNLLVCSVSGFYPGSIEVRWFRNGQ |
| | EEKAGVVSTGLIQNGDWTFQTLVMLETVPRSGEVYTCQVEHPSVTSPLTVEWRARSESAQSK |
| | (HLA-DRB1*01:01 soluble) |
| 99 | GDTRPRFLWQLKFECHFFNGTERVRLLERCIYNQEESVRFDSDVGEYRAVTELGRPDAEYWNSQKDLL |
| | EQRRAAVDTYCRHNYGAVESFTVQRRVEPKVTVYPSKTQPLQHHNLLVCSVSGFYPGSIEVRWFRNGQ |
| | EEKAGVVSTGLIQNGDWTFQTLVMLETVPRSGEVYTCQVEHPSVTSPLTVEWRARSESAQSK |
| | (HLA-DRB1*01:02 soluble) |
| 100 | GDTRPRFLEYSTSECHFFNGTERVRYLDRYFHNQEENVRFDSDVGEFRAVTELGRPDAEYWNSQKDLL |
| | EQKRGRVDNYCRHNYGVVESFTVQRRVHPKVTVYPSKTQPLQHHNLLVCSVSGFYPGSIEVRWFRNGQ |
| | EEKTGVVSTGLIHNGDWTFQTLVMLETVPRSGEVYTCQVEHPSVTSPLTVEWRARSESAQSK |
| | (HLA-DRB1*03:01 soluble) |
| 101 | GDTRPRFLEQVKHECHFFNGTERVRFLDRYFYHQEEYVRFDSDVGEYRAVTELGRPDAEYWNSQKDLL |
| | EQKRAAVDTYCRHNYGVGESFTVQRRVYPEVTVYPAKTQPLQHHNLLVCSVNGFYPGSIEVRWFRNGQ |
| | EEKTGVVSTGLIQNGDWTFQTLVMLETVPRSGEVYTCQVEHPSLTSPLTVEWRARSESAQSK |
| | (HLA-DRB1*04:01 soluble) |
| 102 | GDTRPRFLEQVKHECHFFNGTERVRFLDRYFYHQEEYVRFDSDVGEYRAVTELGRPDAEYWNSQKDLL |
| | EQRRAAVDTYCRHNYGVVESFTVQRRVYPEVTVYPAKTQPLQHHNLLVCSVNGFYPGSIEVRWFRNGQ |
| | EEKTGVVSTGLIQNGDWTFQTLVMLETVPRSGEVYTCQVEHPSLTSPLTVEWRARSESAQSK |
| | (HLA-DRB1*04:04 soluble) |
| 103 | GDTQPRFLWQGKYKCHFFNGTERVQFLERLFYNQEEFVRFDSDVGEYRAVTELGRPVAESWNSQKDIL |
| | EDRRGQVDTVCRHNYGVGESFTVQRRVHPEVTVYPAKTQPLQHHNLLVCSVSGFYPGSIEVRWFRNGQ |
| | EEKAGVVSTGLIQNGDWTFQTLVMLETVPRSGEVYTCQVEHPSVMSPLTVEWRARSESAQSK |
| | (HLA-DRB1*07:01 soluble) |
| 104 | GDTRPRFLEYSTGECYFFNGTERVRFLDRYFYNQEEYVRFDSDVGEYRAVTELGRPSAEYWNSQKDFL |
| | EDRRALVDTYCRHNYGVGESFTVQRRVHPKVTVYPSKTQPLQHHNLLVCSVSGFYPGSIEVRWFRNGQ |
| | EEKTGVVSTGLIHNGDWTFQTLVMLETVPRSGEVYTCQVEHPSVTSPLTVEWSARSESAQSK |
| | (HLA-DRB1*08:01 soluble) |
| 105 | GDTRPRFLEEVKFECHFFNGTERVRLLERRVHNQEEYARYDSDVGEYRAVTELGRPDAEYWNSQKDLL |
| | ERRRAAVDTYCRHNYGVGESFTVQRRVQPKVTVYPSKTQPLQHHNLLVCSVNGFYPGSIEVRWFRNGQ |
| | EEKTGVVSTGLIQNGDWTFQTLVMLETVPQSGEVYTCQVEHPSVMSPLTVEWRARSESAQSK |
| | (HLA-DRB1*10:01 soluble) |
| 106 | GDTRPRFLEYSTSECHFFNGTERVRFLDRYFYNQEEYVRFDSDVGEFRAVTELGRPDEEYWNSQKDFL |
| | EDRRAAVDTYCRHNYGVGESFTVQRRVHPKVTVYPSKTQPLQHHNLLVCSVSGFYPGSIEVRWFRNGQ |
| | EEKTGVVSTGLIHNGDWTFQTLVMLETVPRSGEVYTCQVEHPSVTSPLTVEWRARSESAQSK |
| | (HLA-DRB1*11:01 soluble) |
| 107 | GDTRPRFLEYSTSECHFFNGTERVRFLDRYFYNQEEYVRFDSDVGEFRAVTELGRPDEEYWNSQKDEL |
| | EDRRAAVDTYCRHNYGVVESFTVQRRVHPKVTVYPSKTQPLQHHNLLVCSVSGFYPGSIEVRWFRNGQ |
| | EEKTGVVSTGLIHNGDWTFQTLVMLETVPRSGEVYTCQVEHPSVTSPLTVEWRARSESAQSK |
| | (HLA-DRB1*11:04 soluble) |
| 108 | GDTRPRFLEYSTSECHFENGTERVRFLDRYFHNQEENVRFDSDVGEFRAVTELGRPDAEYWNSQKDIL |
| | EDERAAVDTYCRHNYGVVESFTVQRRVHPKVTVYPSKTQPLQHHNLLVCSVSGFYPGSIEVRWFRNGQ |
| | EEKTGVVSTGLIHNGDWTFQTLVMLETVPRSGEVYTCQVEHPSVTSPLTVEWRARSESAQSK |
| | (HLA-DRB1*13:01 soluble) |
| 109 | GDTRPRFLEYSTSECHFFNGTERVRFLDRYFHNQEENVRFDSDVGEFRAVTELGRPDAEYWNSQKDIL |
| | EDERAAVDTYCRHNYGVGESFTVQRRVHPKVTVYPSKTQPLQHHNLLVCSVSGFYPGSIEVRWFRNGQ |
| | EEKTGVVSTGLIHNGDWTFQTLVMLETVPRSGEVYTCQVEHPSVTSPLTVEWRARSESAQSK |
| | (HLA-DRB1*13:02 soluble) |
| 110 | GDTRPRFLEYSTSECHFFNGTERVRFLDRYFHNQEEFVRFDSDVGEYRAVTELGRPAAEHWNSQKDLL |
| | ERRRAEVDTYCRHNYGVVESFTVQRRVHPKVTVYPSKTQPLQHYNLLVCSVSGFYPGSIEVRWFRNGQ |
| | EEKTGVVSTGLIHNGDWTFQTLVMLETVPRSGEVYTCQVEHPSVTSPLTVEWRARSESAQSK |
| | (HLA-DRB1*14:01 soluble) |
| 111 | GDTRPRFLWQPKRECHFFNGTERVRFLDRYFYNQEESVRFDSDVGEFRAVTELGRPDAEYWNSQKDIL |
| | EQARAAVDTYCRHNYGVVESFTVQRRVQPKVTVYPSKTQPLQHHNLLVCSVSGFYPGSIEVRWFLNGQ |
| | EEKAGMVSTGLIQNGDWTFQTLVMLETVPRSGEVYTCQVEHPSVTSPLTVEWRARSESAQSK |
| | (HLA-DRB1*15:01 soluble) |
| 112 | GDTRPRFLWQPKRECHFFNGTERVRFLDRHFYNQEESVRFDSDVGEFRAVTELGRPDAEYWNSQKDIL |
| | EQARAAVDTYCRHNYGVVESFTVQRRVQPKVTVYPSKTQPLQHHNLLVCSVSGFYPGSIEVRWFLNGQ |
| | EEKAGMVSTGLIQNGDWTFQTLVMLETVPRSGEVYTCQVEHPSVTSPLTVEWRARSESAQSK |
| | (HLA-DRB1*15:03 soluble) |
| 113 | EDIVADHVASCGVNLYQFYGPSGQYTHEFDGDEEFYVDLERKETAWRWPEFSKFGGFDPQGALRNMAV |
| | AKHNLNIMIKRYNSTAATNEVPEVTVFSKSPVTLGQPNTLICLVDNIFPPVVNITWLSNGQSVTEGVS |
| | ETSFLSKSDHSFFKISYLTFLPSADEIYDCKVEHWGLDQPLLKHWEPEIPAPMSELTET |
| | (HLA-DQA1*01:01 soluble) |
| 114 | GRDSPEDFVYQFKGLCYFTNGTERVRGVTRHIYNREEYVRFDSDVGVYRAVTPQGRPVAEYWNSQKEV |
| | LEGARASVDRVCRHNYEVAYRGILQRRVEPTVTISPSRTEALNHHNLLICSVTDFYPSQIKVRWFRND |
| | QEETAGVVSTPLIRNGDWTFQILVMLEMTPQRGDVYTCHVEHPSLQSPITVEWRAQSESAQSK |
| | (HLA-DQB1*05:01 soluble) |
| 115 | EDIVADHVASCGVNLYQFYGPSGQYTHEFDGDEQFYVDLERKETAWRWPEFSKFGGFDPQGALRNMAV |
| | AKHNLNIMIKRYNSTAATNEVPEVTVFSKSPVTLGQPNTLICLVDNIFPPVVNITWLSNGQSVTEGVS |
| | ETSFLSKSDHSFFKISYLTFLPSADEIYDCKVEHWGLDQPLLKHWEPEIPAPMSELTET |
| | (HLA-DQA1*01:02 soluble) |
| 116 | GRDSPEDFVFQFKGMCYFTNGTERVRLVTRYIYNREEYARFDSDVGVYRAVTPQGRPDAEYWNSQKEV |
| | LEGTRAELDTVCRHNYEVAFRGILQRRVEPTVTISPSRTEALNHHNLLVCSVTDFYPGQIKVRWFRND |
| | QEETAGVVSTPLIRNGDWTFQILVMLEMTPQRGDVYTCHVEHPSLQSPITVEWRAQSESAQSK |
| | (HLA-DQB1*06:02 soluble) |
| 117 | EDIVADHVASYGVNLYQSYGPSGQYSHEFDGDEEFYVDLERKETVWQLPLFRRFRRFDPQFALTNIAV |
| | LKHNLNIVIKRSNSTAATNEVPEVTVFSKSPVTLGQPNTLICLVDNIFPPVVNITWLSNGHSVTEGVS |
| | ETSFLSKSDHSFFKISYLTFLPSADEIYDCKVEHWGLDEPLLKHWEPEIPTPMSELTET |
| | (HLA-DQA1*03:01 soluble) |
| 118 | GRDSPEDFVYQFKGMCYFTNGTERVRLVTRYIYNREEYARFDSDVGVYRAVTPLGPPAAEYWNSQKEV |
| | LERTRAELDTVCRHNYQLELRTTLQRRVEPTVTISPSRTEALNHHNLLVCSVTDFYPAQIKVRWFRND |
| | QEETTGVVSTPLIRNGDWTFQILVMLEMTPQRGDVYTCHVEHPSLQNPIIVEWRAQSESAQSK |
| | (HLA-DQB1*03:02 soluble) |
| 119 | EDIVADHVASYGVNLYQSYGPSGQYTHEFDGDEQFYVDLGRKETVWCLPVLRQFRFDPQFALTNIAVL |
| | KHNLNSLIKRSNSTAATNEVPEVTVFSKSPVTLGQPNILICLVDNIFPPVVNITWLSNGHSVTEGVSE |
| | TSFLSKSDHSFFKISYLTLLPSAEESYDCKVEHWGLDKPLLKHWEPEIPAPMSELTET |
| | (HLA-DQA1*05:01 soluble) |
| 120 | GRDSPEDFVYQFKGMCYFTNGTERVRLVSRSIYNREEIVRFDSDVGEFRAVTLLGLPAAEYWNSQKDI |
| | LERKRAAVDRVCRHNYQLELRTTLQRRVEPTVTISPSRTEALNHHNLLVCSVTDFYPAQIKVRWFRND |
| | QEETAGVVSTPLIRNGDWTFQILVMLEMTPQRGDVYTCHVEHPSLQSPITVEWRAQSESAQSK |
| | (HLA-DQB1*02:01 soluble) |
| 121 | GRDSPEDFVYQFKAMCYFTNGTERVRYVTRYIYNREEYARFDSDVEVYRAVTPLGPPDAEYWNSQKEV |
| | LERTRAELDTVCRHNYQLELRTTLQRRVEPTVTISPSRTEALNHHNLLVCSVTDFYPAQIKVRWFRND |
| | QEETTGVVSTPLIRNGDWTFQILVMLEMTPQHGDVYTCHVEHPSLQNPITVEWRAQSESAQSK |
| | (HLA-DQB1*03:01 soluble) |
| 122 | GRDSPEDFVYQFKGMCYFTNGTERVRLVTRYIYNREEYARFDSDVGVYRAVTPLGPPDAEYWNSQKEV |
| | LERTRAELDTVCRHNYQLELRTTLQRRVEPTVTISPSRTEALNHHNLLVCSVTDFYPAQIKVRWFRND |
| | QEETTGVVSTPLIRNGDWTFQILVMLEMTPQRGDVYTCHVEHPSLQNPIIVEWRAQSESAQSK |
| | (HLA-DQB1*03:03 soluble) |
| 123 | GRDSPEDFVFQFKGMCYFTNGTERVRGVTRYIYNREEYARFDSDVGVYRAVTPLGRLDAEYWNSQKDI |
| | LEEDRASVDTVCRHNYQLELRTTLQRRVEPTVTISPSRTEALNHHNLLVCSVTDFYPAQIKVRWFRND |
| | QEETTGVVSTPLIRNGDWTFQILVMLEMTPQRGDVYTCHVEHPSLQNPIIVEWRAQSESAQSK |
| | (HLA-DQB1*04:02 soluble) |
| 124 | GRDSPEDFVYQFKGLCYFTNGTERVRGVTRHIYNREEYVRFDSDVGVYRAVTPQGRPDAEYWNSQKEV |
| | LEGARASVDRVCRHNYEVAYRGILQRRVEPTVTISPSRTEALNHHNLLICSVTDFYPSQIKVRWFRND |
| | QEETAGVVSTPLIRNGDWTFQILVMLEMTPQRGDVYTCHVEHPSLQSPITVEWRAQSESAQSK |
| | (HLA-DQB1*05:03 soluble) |
| 125 | GRDSPEDFVYQFKGMCYFTNGTERVRLVTRHIYNREEYARFDSDVGVYRAVTPQGRPDAEYWNSQKEV |
| | LEGTRAELDTVCRHNYEVAFRGILQRRVEPTVTISPSRTEALNHHNLLVCSVTDFYPGQIKVRWFRND |
| | QEETAGVVSTPLIRNGDWTFQILVMLEMTPQRGDVYTCHVEHPSLQSPITVEWRAQSESAQSK |
| | (HLA-DQB1*06:03 soluble) |
| 126 | GRDSPEDFVYQFKGMCYFTNGTERVRLVTRHIYNREEYARFDSDVGVYRAVTPQGRPVAEYWNSQKEV |
| | LERTRAELDTVCRHNYEVGYRGILQRRVEPTVTISPSRTEALNHHNLLVCSVTDFYPGQIKVQWFRND |
| | QEETAGVVSTPLIRNGDWTFQILVMLEMTPQRGDVYTCHVEHPSLQSPITVEWRAQSESAQSK |
| | (HLA-DQB1*06:04 soluble) |
| 127 | IAELPPKVSVFVPPRDGFFGNPRKSKLICQATGFSPRQIQVSWLREGKQVGSGVTTDQVQAEAKESGP |
| | TTYKVTSTLTIKESDWLGQSMFTCRVDHRGLTFQQNASSMCVPDQDTAIRVFAIPPSFASIFLTKSTK |
| | LTCLVTDLTTYDSVTISWTRQNGEAVKTHTNISESHPNATFSAVGEASICEDDWNSGERFTCTVTHTD |
| | LPSPLKQTISRPKGVALHRPDVYLLPPAREQLNLRESATITCLVTGFSPADVFVQWMQRGQPLSPEKY |
| | VTSAPMPEPQAPGRYFAHSILTVSEEEWNTGETYTCVVAHEALPNRVTERTVDKSTGKPTLYNVSLVM |
| | SDTAGTCY |
| | (IgM Cm2-Cm3-Cm4-TP) |
| 128 | IRVFAIPPSFASIFLTKSTKLTCLVTDLTTYDSVTISWTRQNGEAVKTHTNISESHPNATFSAVGEAS |
| | ICEDDWNSGERFTCTVTHTDLPSPLKQTISRPKGVALHRPDVYLLPPAREQLNLRESATITCLVTGFS |
| | PADVFVQWMQRGQPLSPEKYVTSAPMPEPQAPGRYFAHSILTVSEEEWNTGETYTCVVAHEALPNRVT |
| | ERTVDKSTGKPTLYNVSLVMSDTAGTCY |
| | (IgM Cm3-Cm4-TP) |
| 129 | PTLYNVSLVMSDTAGTCY |
| | (human IgM tailpiece) |
| 130 | PTHVNVSVVMAEVDGTCY |
| | (human IgA tailpiece) |
| 131 | GGGGSGGGGSGGGGS |
| | (Linker) |
| 132 | GSAPKPAPKPAPAPKPAPKPAP |
| | (linker) |
| 133 | GGGGSGGGGSGGGGSGGGGSAPKPAPKPAPAPKPAPKPAP |
| | (linker) |
| 134 | (GGGGS)n, wherein n = 1-6 |
| | (linker) |
| 135 | SSSSGSSSSGSAA |
| | (linker) |
| 136 | GGGGG |
| | (linker) |
| 137 | S(GGGGS)n, wherein n = 1-10 |
| | (linker) |
| 138 | (GGSG)n, wherein n = 1-5 |
| | (linker) |
| 139 | GSAT |
| | (linker) |
| 140 | (GGSGGS)n, wherein n = 1-5 |
| | (linker) |
| 141 | GGGGGGGGSGGGGSGGGGS |
| | ((G4S) 4 linker) |
| 142 | GSGSAGGSGSGGGS |
| | ((GS) 2AG2SGSG3S linker) |
| 143 | DYKDDDDK |
| | (FLAG Tag) |
| 144 | HHHHHH |
| | (His6 Tag) |
| 145 | GKPIPNPLLGLDST |
| | (V5 Tag) |
| 146 | WSHPQFEK |
| | (strep-tag) |
| 147 | EDQVDPRLIDGK |
| | (protein C tag) |
| 148 | EQKLISEEDL |
| | (Myc tag) |
| 149 | GLNDIFEAQKIEWHEGSGEQKLISEEDL |
| | (avitag-Myc (biotin-mediated)) |
| 150 | GLNDIFEAQKIEWHEGSGEQKLISEEDLHHHHHH |
| | (avitag-Myc-His (biotin-mediated)) |
| 151 | GSGSAGGGLNDIFEAQKIEWHEGSTGHHHHHHDYKDDDDK |
| | (Avitag sequence with His6 tag and Flag tag) |
| 152 | LTDTLQAETDQLEDEKSALQTEIANLLKEKEKLEFILAA |
| | (Fos) |
| 153 | RIARLEEKVKTLKAQNSELASTANMLREQVAQLKQKVMNH |
| | (Jun) |
| 154 | TTAPSAQLEKELQALQKENAQLEWELQALEKELAQ |
| | (acidic leucine zipper) |
| 155 | TTAPSAQLKKKLQALKKKNAQLKWKLQALKKKLAQ |
| | (basic leucine zipper) |
| 156 | EPKSADKTHTCPPCPAPEAAGGPSVFLFPPKPKDTLMISRTPEVTCVVVDVSHEDPEVKFNWYVDGVE |
| | VHNAKTKPREEQYNSTYRVVSVLTVLHQDWLNGKEYKCKVSNKALGAPIEKTISKAKGQPREPQVYTL |
| | PPCRDELTKNQVSLWCLVKGFYPSDIAVEWESNGQPENNYKTTPPVLDSDGSFFLYSKLTVDKSRWQQ |
| | GNVFSCSVMHEALHNHYTQKSLSLSPGK |
| | (knob (knob-in-hole)) |
| 157 | EPKSADKTHTCPPCPAPEAAGGPSVFLFPPKPKDTLMISRTPEVTCVVVDVSHEDPEVKFNWYVDGVE |
| | VHNAKTKPREEQYNSTYRVVSVLTVLHQDWLNGKEYKCKVSNKALGAPIEKTISKAKGQPREPQVCTL |
| | PPSRDELTKNQVSLSCAVKGFYPSDIAVEWESNGQPENNYKTTPPVLDSDGSFFLVSKLTVDKSRWQQ |
| | GNVFSCSVMHEALHNHYTQKSLSLSPGK |
| | (hole (knob-in-hole)) |
| 158 | RGVPHIVMVDAYKRYK |
| | (spytag) |
| 159 | VTTLSGLSGEQGPSGDMTTEEDSATHIKFSKRDEDGRELAGATMELRDSSGKTISTWISDGHVKDFYL |
| | YPGKYTFVETAAPDGYEVATPIEFTVNEDGQVTVDGEATEGDAHT |
| | (spycatcher) |
| 160 | YMLDLQPETT |
| | (HPV sequence) |
| 161 | SLPITVYYA |
| | (HSV sequence) |
| 162 | RMFPNAPYL |
| | (WT-1 sequence) |
| 163 | KILGFVFTV |
| | (HLA-A2-binding peptide) |
| 164 | GILGFVFTL |
| | (HLA-A2-binding peptide) |
| 165 | VTEHDTLLY |
| | (A1:01-binding peptide) |
| 166 | TVRSHCVSK |
| | (A3:01-binding peptide) |
| 167 | TTFLQTMLR |
| | (A11:01-binding peptide) |
| 168 | RYPLTFGWCF |
| | (A24:02-binding peptide) |
| 169 | RPHERNGFTVL |
| | (B7:02-binding peptide) |
| 170 | IPSINVHHY |
| | (B35:01-binding peptide) |
| 171 | FVYGGSKTSL |
| | (C3:04-binding peptide) |
| 172 | FLRGRAYGL |
| | (B8:01-binding peptide) |
| 173 | RYRPGTVAL |
| | (C7:02-binding peptide) |
| 174 | QYDPVAALF |
| | (C4:01-binding peptide) |
| 175 | GQFLTPNSH |
| | (B15:01-binding peptide) |
| 176 | KEVNSQLSL |
| | (B40:01-binding peptide) |
| 177 | VSFIEFVGW |
| | (B58:01-binding peptide) |
| 178 | IAPWYAFAL |
| | (C8:02-binding peptide) |
| 179 | DXEXNPGP, where X = any amino acid |
| | (2A core sequence motif) |
| 180 | EGRGSLLTCGDVEENPGP |
| | (T2A sequence) |
| 181 | ATNFSLLKQAGDVEENPGP |
| | (P2A sequence) |
| 182 | QCTNYALLKLAGDVESNPGP |
| | (E2A sequence) |
| 183 | VKQTLNFDLLKLAGDVESNPGP |
| | (F2A sequence) |
| 184 | MEAPAQLLFLLLLWLPDTTG |
| | (Ig Kappa chain V-III region CLL signal peptide) |
| 185 | MNRGVPFRHLLLVLQLALLPAAT |
| | (signal peptide of human CD4 signal peptide) |
| 186 | METDTLLLWVLLLWVPGSTG |
| | (mouse Ig kappa chain V-III region signal peptide) |
| 187 | MVPCTLLLLLAAALAPTQTRA |
| | (mouse H-2Kb signal peptide) |
| 188 | MKWVTFISLLFLFSSAYS |
| | (human serum albumin signal peptide) |
| 189 | MYRMQLLSCIALSLALVINS |
| | (human IL-2 signal peptide) |
| 190 | MAVMAPRTLLLLLSGALALTQTWA |
| | (human HLA-A*02:01 signal peptide) |
| 191 | MSRSVALAVLALLSLSGLEA |
| | (Human b2m signal peptide) |
| 192 | LPETGG |
| | (sortase recognition site) |
| 193 | IAELPPKVSVFVPPRDGFFGNPRKSKLICQATGFSPRQIQVSWLREGKQVGSGVTTDQVQAEAKESGP |
| | TTYKVTSTLTIKESDWLGQSMFTCRVDHRGLTFQQNASSMCVPDQDTAIRVFAIPPSFASIFLTKSTK |
| | LTCLVTDLTTYDSVTISWTRQNGEAVKTHTNISESHPNATFSAVGEASICEDDWNSGERFTCTVTHTD |
| | DPSPLKQTISRPKGVALHRPDVYLLPPAREQLNLRESATITCLVTGFSPADVFVQWMQRGQPLSPEKY |
| | VTSAPMPEPQAPGRYFAHSILTVSEEEWNTGETYTCVVAHEALPNRVTERTVDKSTGKPTLYNVSLVM |
| | SDTAGTCY |
| | (IgM Cm2-Cm3-Cm4-TP L310D) |
| 194 | IRVFAIPPSFASIFLTKSTKLTCLVTDLTTYDSVTISWTRQNGEAVKTHTNISESHPNATFSAVGEAS |
| | ICEDDWNSGERFTCTVTHTDDPSPLKQTISRPKGVALHRPDVYLLPPAREQLNLRESATITCLVTGFS |
| | PADVFVQWMQRGQPLSPEKYVTSAPMPEPQAPGRYFAHSILTVSEEEWNTGETYTCVVAHEALPNRVT |
| | ERTVDKSTGKPTLYNVSLVMSDTAGTCY |
| | (IgM Cm3-Cm4-TP L310D) |
| 195 | IAELPPKVSVFVPPRDGFFGNPRKSKLICQATGFSPRQIQVSWLREGKQVGSGVTTDQVQAEAKESGP |
| | TTYKVTSTLTIKESDWLGQSMFTCRVDHRGLTFQQNASSMCVPDQDTAIRVFAIPPSFASIFLTKSTK |
| | LTCLVTDLTTYDSVTISWTRQNGEAVKTHTNISESHPNATFSAVGEASICEDDWNSGERFTCTVTHTD |
| | LASSLKQTISRPKGVALHRPDVYLLPPAREQLNLRESATITCLVTGFSPADVFVQWMQRGQPLSPEKY |
| | VTSAPMPEPQAPGRYFAHSILTVSEEEWNTGETYTCVVAHEALPNRVTERTVDKSTGKPTLYNVSLVM |
| | SDTAGTCY |
| | (IgM Cm2-Cm3-Cm4-TP P311A, P313S) |
| 196 | IRVFAIPPSFASIFLTKSTKLTCLVTDLTTYDSVTISWTRQNGEAVKTHTNISESHPNATFSAVGEAS |
| | ICEDDWNSGERFTCTVTHTDLASSLKQTISRPKGVALHRPDVYLLPPAREQLNLRESATITCLVTGFS |
| | PADVFVQWMQRGQPLSPEKYVTSAPMPEPQAPGRYFAHSILTVSEEEWNTGETYTCVVAHEALPNRVT |
| | ERTVDKSTGKPTLYNVSLVMSDTAGTCY |
| | (IgM Cm3-Cm4-TP P311A, P313S) |
| 197 | IAELPPKVSVFVPPRDGFFGNPRKSKLICQATGFSPRQIQVSWLREGKQVGSGVTTDQVQAEAKESGP |
| | TTYKVTSTLTIKESDWLGQSMFTCRVDHRGLTFQQNASSMCVPDQDTAIRVFAIPPSFASIFLTKSTK |
| | LTCLVTDLTTYDSVTISWTRQNGEAVKTHTNISESHPNATFSAVGEASICEDDWNSGERFTCTVTHTD |
| | LGSPLKQTISRPKGVALHRPDVYLLPPAREQLNLRESATITCLVTGFSPADVFVQWMQRGQPLSPEKY |
| | VTSAPMPEPQAPGRYFAHSILTVSEEEWNTGETYTCVVAHEALPNRVTERTVDKSTGKPTLYNVSLVM |
| | SDTAGTCY |
| | (IgM Cm2-Cm3-Cm4-TP P311G) |
| 198 | IRVFAIPPSFASIFLTKSTKLTCLVTDLTTYDSVTISWTRQNGEAVKTHTNISESHPNATFSAVGEAS |
| | ICEDDWNSGERFTCTVTHTDLGSPLKQTISRPKGVALHRPDVYLLPPAREQLNLRESATITCLVTGFS |
| | PADVFVQWMQRGQPLSPEKYVTSAPMPEPQAPGRYFAHSILTVSEEEWNTGETYTCVVAHEALPNRVT |
| | ERTVDKSTGKPTLYNVSLVMSDTAGTCY |
| | (IgM Cm3-Cm4-TP P311G) |
| 199 | ELAGIGILTVGGGGSGGGGSGGGGSIQRTPKIQVYSRHPAENGKSNFLNCYVSGFHPSDIEVDLLKNG |
| | ERIEKVEHSDLSFSKDWSFYLLYYTEFTPTEKDEYACRVNHVTLSQPKIVKWDRDMGGGGSGGGGSGG |
| | GGSGGGGSGSHSMRYFFTSVSRPGRGEPRFIAVGYVDDTQFVRFDSDAASQRMEPRAPWIEQEGPEYW |
| | DGETRKVKAHSQTHRVDLGTLRGAYNQSEAGSHTVQRMYGCDVGSDWRFLRGYHQYAYDGKDYIALKE |
| | DLRSWTAADMAAQTTKHKWEAAHVAEQLRAYLEGTCVEWLRRYLENGKETLQRTDAPKTHMTHHAVSD |
| | HEATLRCWALSFYPAEITLTWQRDGEDQTQDTELVETRPAGDGTFQKWAAVVVPSGQEQRYTCHVQHE |
| | GLPKPLTLRWEPSSGGGGSGGGGSGGGGSIAELPPKVSVFVPPRDGFFGNPRKSKLICQATGFSPRQI |
| | QVSWLREGKQVGSGVTTDQVQAEAKESGPTTYKVTSTLTIKESDWLGQSMFTCRVDHRGLTFQQNASS |
| | MCVPDQDTAIRVFAIPPSFASIFLTKSTKLTCLVTDLTTYDSVTISWTRQNGEAVKTHTNISESHPNA |
| | TFSAVGEASICEDDWNSGERFTCTVTHTDDPSPLKQTISRPKGVALHRPDVYLLPPAREQLNLRESAT |
| | ITCLVTGFSPADVFVQWMQRGQPLSPEKYVTSAPMPEPQAPGRYFAHSILTVSEEEWNTGETYTCVVA |
| | HEALPNRVTERTVDKSTGKPTLYNVSLVMSDTAGTCY |
| | (ELA_B2M_HLAA2_gM2-4 L310D) |
| 200 | ELAGIGILTVGGGGSGGGGSGGGGSIQRTPKIQVYSRHPAENGKSNFLNCYVSGFHPSDIEVDLLKNG |
| | ERIEKVEHSDLSFSKDWSFYLLYYTEFTPTEKDEYACRVNHVTLSQPKIVKWDRDMGGGGSGGGGSGG |
| | GGSGGGGSGSHSMRYFFTSVSRPGRGEPRFIAVGYVDDTQFVRFDSDAASQRMEPRAPWIEQEGPEYW |
| | DGETRKVKAHSQTHRVDLGTLRGAYNQSEAGSHTVQRMYGCDVGSDWRFLRGYHQYAYDGKDYIALKE |
| | DLRSWTAADMAAQTTKHKWEAAHVAEQLRAYLEGTCVEWLRRYLENGKETLQRTDAPKTHMTHHAVSD |
| | HEATLRCWALSFYPAEITLTWQRDGEDQTQDTELVETRPAGDGTFQKWAAVVVPSGQEQRYTCHVQHE |
| | GLPKPLTLRWEPSSGGGGSGGGGSGGGGSIRVFAIPPSFASIFLTKSTKLTCLVTDLTTYDSVTISWT |
| | RQNGEAVKTHTNISESHPNATFSAVGEASICEDDWNSGERFTCTVTHTDDPSPLKQTISRPKGVALHR |
| | PDVYLLPPAREQLNLRESATITCLVTGFSPADVFVQWMQRGQPLSPEKYVTSAPMPEPQAPGRYFAHS |
| | ILTVSEEEWNTGETYTCVVAHEALPNRVTERTVDKSTGKPTLYNVSLVMSDTAGTCY |
| | (ELA_B2M_HLAA2_IgM Cm3-Cm4-TP L310D) |
| 201 | ELAGIGILTVGGGGSGGGGSGGGGSIQRTPKIQVYSRHPAENGKSNFLNCYVSGFHPSDIEVDLLKNG |
| | ERIEKVEHSDLSFSKDWSFYLLYYTEFTPTEKDEYACRVNHVTLSQPKIVKWDRDMGGGGSGGGGSGG |
| | GGSGGGGSGSHSMRYFFTSVSRPGRGEPRFIAVGYVDDTQFVRFDSDAASQRMEPRAPWIEQEGPEYW |
| | DGETRKVKAHSQTHRVDLGTLRGAYNQSEAGSHTVQRMYGCDVGSDWRFLRGYHQYAYDGKDYIALKE |
| | DLRSWTAADMAAQTTKHKWEAAHVAEQLRAYLEGTCVEWLRRYLENGKETLQRTDAPKTHMTHHAVSD |
| | HEATLRCWALSFYPAEITLTWQRDGEDQTQDTELVETRPAGDGTFQKWAAVVVPSGQEQRYTCHVQHE |
| | GLPKPLTLRWEPSSGGGGSGGGGSGGGGSIAELPPKVSVFVPPRDGFFGNPRKSKLICQATGFSPRQI |
| | QVSWLREGKQVGSGVTTDQVQAEAKESGPTTYKVTSTLTIKESDWLGQDMFTCRVDHRGLTFQQNASS |
| | MCVPDQDTAIRVFAIPPSFASIFLTKSTKLTCLVTDLTTYDSVTISWTRQNGEAVKTHTNISESHPNA |
| | TFSAVGEASICEDDWNSGERFTCTVTHTDLASSLKQTISRPKGVALHRPDVYLLPPAREQLNLRESAT |
| | ITCLVTGFSPADVFVQWMQRGQPLSPEKYVTSAPMPEPQAPGRYFAHSILTVSEEEWNTGETYTCVVA |
| | HEALPNRVTERTVDKSTGKPTLYNVSLVMSDTAGTCY |
| | (ELA_B2M_HLAA2_gM2-4 Cm2-Cm3-Cm4-TP P311A, P313S) |
| 202 | ELAGIGILTVGGGGSGGGGSGGGGSIQRTPKIQVYSRHPAENGKSNFLNCYVSGFHPSDIEVDLLKNG |
| | ERIEKVEHSDLSFSKDWSFYLLYYTEFTPTEKDEYACRVNHVTLSQPKIVKWDRDMGGGGSGGGGSGG |
| | GGSGGGGSGSHSMRYFFTSVSRPGRGEPRFIAVGYVDDTQFVRFDSDAASQRMEPRAPWIEQEGPEYW |
| | DGETRKVKAHSQTHRVDLGTLRGAYNQSEAGSHTVQRMYGCDVGSDWRFLRGYHQYAYDGKDYIALKE |
| | DLRSWTAADMAAQTTKHKWEAAHVAEQLRAYLEGTCVEWLRRYLENGKETLQRTDAPKTHMTHHAVSD |
| | HEATLRCWALSFYPAEITLTWQRDGEDQTQDTELVETRPAGDGTFQKWAAVVVPSGQEQRYTCHVQHE |
| | GLPKPLTLRWEPSSGGGGSGGGGSGGGGSIRVFAIPPSFASIFLTKSTKLTCLVTDLTTYDSVTISWT |
| | RQNGEAVKTHTNISESHPNATFSAVGEASICEDDWNSGERFTCTVTHTDLASSLKQTISRPKGVALHR |
| | PDVYLLPPAREQLNLRESATITCLVTGFSPADVFVQWMQRGQPLSPEKYVTSAPMPEPQAPGRYFAHS |
| | ILTVSEEEWNTGETYTCVVAHEALPNRVTERTVDKSTGKPTLYNVSLVMSDTAGTCY |
| | (ELA_B2M_HLAA2_gM2-4 Cm3-Cm4-TP P311A, P313S) |
| 203 | ELAGIGILTVGGGGSGGGGSGGGGSIQRTPKIQVYSRHPAENGKSNFLNCYVSGFHPSDIEVDLLKNG |
| | ERIEKVEHSDLSFSKDWSFYLLYYTEFTPTEKDEYACRVNHVTLSQPKIVKWDRDMGGGGSGGGGSGG |
| | GGSGGGGSGSHSMRYFFTSVSRPGRGEPRFIAVGYVDDTQFVRFDSDAASQRMEPRAPWIEQEGPEYW |
| | DGETRKVKAHSQTHRVDLGTLRGAYNQSEAGSHTVQRMYGCDVGSDWRFLRGYHQYAYDGKDYIALKE |
| | DLRSWTAADMAAQTTKHKWEAAHVAEQLRAYLEGTCVEWLRRYLENGKETLQRTDAPKTHMTHHAVSD |
| | HEATLRCWALSFYPAEITLTWQRDGEDQTQDTELVETRPAGDGTFQKWAAVVVPSGQEQRYTCHVQHE |
| | GLPKPLTLRWEPSSGGGGSGGGGSGGGGSIAELPPKVSVFVPPRDGFFGNPRKSKLICQATGFSPRQI |
| | QVSWLREGKQVGSGVTTDQVQAEAKESGPTTYKVTSTLTIKESDWLGQSMFTCRVDHRGLTFQQNASS |
| | MCVPDQDTAIRVFAIPPSFASIFLTKSTKLTCLVTDLTTYDSVTISWTRQNGEAVKTHTNISESHPNA |
| | TFSAVGEASICEDDWNSGERFTCTVTHTDLGSPLKQTISRPKGVALHRPDVYLLPPAREQLNLRESAT |
| | ITCLVTGFSPADVFVQWMQRGQPLSPEKYVTSAPMPEPQAPGRYFAHSILTVSEEEWNTGETYTCVVA |
| | HEALPNRVTERTVDKSTGKPTLYNVSLVMSDTAGTCY |
| | (ELA_B2M_HLAA2 IgM Cm2-Cm3-Cm4-TP P311G) |
| 204 | ELAGIGILTVGGGGSGGGGSGGGGSIQRTPKIQVYSRHPAENGKSNFLNCYVSGFHPSDIEVDLLKNG |
| | ERIEKVEHSDLSFSKDWSFYLLYYTEFTPTEKDEYACRVNHVTLSQPKIVKWDRDMGGGGSGGGGSGG |
| | GGSGGGGSGSHSMRYFFTSVSRPGRGEPRFIAVGYVDDTQFVRFDSDAASQRMEPRAPWIEQEGPEYW |
| | DGETRKVKAHSQTHRVDLGTLRGAYNQSEAGSHTVQRMYGCDVGSDWRFLRGYHQYAYDGKDYIALKE |
| | DLRSWTAADMAAQTTKHKWEAAHVAEQLRAYLEGTCVEWLRRYLENGKETLQRTDAPKTHMTHHAVSD |
| | HEATLRCWALSFYPAEITLTWQRDGEDQTQDTELVETRPAGDGTFQKWAAVVVPSGQEQRYTCHVQHE |
| | GLPKPLTLRWEPSSGGGGSGGGGSGGGGSIRVFAIPPSFASIFLTKSTKLTCLVTDLTTYDSVTISWT |
| | RQNGEAVKTHTNISESHPNATFSAVGEASICEDDWNSGERFTCTVTHTDLGSPLKQTISRPKGVALHR |
| | PDVYLLPPAREQLNLRESATITCLVTGFSPADVFVQWMQRGQPLSPEKYVTSAPMPEPQAPGRYFAHS |
| | ILTVSEEEWNTGETYTCVVAHEALPNRVTERTVDKSTGKPTLYNVSLVMSDTAGTCY |
| | (ELA_B2M_HLAA2 IgM Cm3-Cm4-TP P311G) |
| 205 | IKEEHVIIQAEFYLNPDQSGEFMFDFDGDEIFHVDMAKKETVWRLEEFGRFASFEAQGALANIAVDKA |
| | NLEIMTKRSNYTPITNVPPEVTVLTNSPVELREPNVLICFIDKFTPPVVNVTWLRNGKPVTTGVSETV |
| | FLPRFDHLFRKFHYLPFLPSTEDVYDCRVEHWGLDEPLLKHWEFDAPSPLPETTEGGGGSGGGGSIAR |
| | LEEKVKTLKAQNYELASTANMLREQVAQLGAPGSGSGSEQKLISEEDL |
| | (DRA1*01-Linker-Jun-Linker-Myc) |
| 206 | IKEEHVIIQAEFYLNPDQSGEFMFDFDGDEIFHVDMAKKETVWRLEEFGRFASFEAQGALANIAVDKA |
| | NLEIMTKRSNYTPITNVPPEVTVLTNSPVELREPNVLICFIDKFTPPVVNVTWLRNGKPVTTGVSETV |
| | FLPRFDHLFRKFHYLPFLPSTEDVYDCRVEHWGLDEPLLKHWEFDAPSPLPETTEGSEQKLISEEDL |
| | (DRA1*01-Linker-Myc) |
| 207 | MSQIMYNYPAMMAHAGGGGSGGGGSGSGDTRPRFLWQPKRECHFFNGTERVRFLDRYFYNQEESVRFD |
| | SDVGEFRAVTELGRPDAEYWNSQKDILEQARAAVDTYCRHNYGVVESFTVQRRVQPKVTVYPSKTQPL |
| | QHHNLLVCSVSGFYPGSIEVRWFLNGQEEKAGMVSTGLIQNGDWTFQTLVMLETVPRSGEVYTCQVEH |
| | PSVTSPLTVEWRARSESAQSKGGGGSGGGGSLDELQAEIEQLEERNYALRKEIEDLQKQLEKLGAPGG |
| | GGSGIAELPPKVSVFVPPRDGFFGNPRKSKLICQATGFSPRQIQVSWLREGKQVGSGVTTDQVQAEAK |
| | ESGPTTYKVTSTLTIKESDWLGQSMFTCRVDHRGLTFQQNASSMCVPDQDTAIRVFAIPPSFASIFLT |
| | KSTKLTCLVTDLTTYDSVTISWTRQNGEAVKTHTNISESHPNATFSAVGEASICEDDWNSGERFTCTV |
| | THTDLPSPLKQTISRPKGVALHRPDVYLLPPAREQLNLRESATITCLVTGFSPADVFVQWMQRGQPLS |
| | PEKYVTSAPMPEPQAPGRYFAHSILTVSEEEWNTGETYTCVVAHEALPNRVTERTVDKSTGKPTLYNV |
| | SLVMSDTAGTCY |
| | (MTB-linker-DRB1*15:01-Linker-FosW-Linker-IgMCm2-4) |
| 208 | ENPVVHFFKNIVTPGGGGSGGGGSGSGDTRPRFLWQPKRECHFFNGTERVRFLDRYFYNQEESVRFDS |
| | DVGEFRAVTELGRPDAEYWNSQKDILEQARAAVDTYCRHNYGVVESFTVQRRVQPKVTVYPSKTQPLQ |
| | HHNLLVCSVSGFYPGSIEVRWFLNGQEEKAGMVSTGLIQNGDWTFQTLVMLETVPRSGEVYTCQVEHP |
| | SVTSPLTVEWRARSESAQSKGGGGSGGGGSLDELQAEIEQLEERNYALRKEIEDLQKQLEKLGAPGGG |
| | GSGIAELPPKVSVFVPPRDGFFGNPRKSKLICQATGFSPRQIQVSWLREGKQVGSGVTTDQVQAEAKE |
| | SGPTTYKVTSTLTIKESDWLGQSMFTCRVDHRGLTFQQNASSMCVPDQDTAIRVFAIPPSFASIFLTK |
| | STKLTCLVTDLTTYDSVTISWTRQNGEAVKTHTNISESHPNATFSAVGEASICEDDWNSGERFTCTVT |
| | HTDLPSPLKQTISRPKGVALHRPDVYLLPPAREQLNLRESATITCLVTGFSPADVFVQWMQRGQPLSP |
| | EKYVTSAPMPEPQAPGRYFAHSILTVSEEEWNTGETYTCVVAHEALPNRVTERTVDKSTGKPTLYNVS |
| | LVMSDTAGTCY |
| | (MBP-Linker-DRB1*15:01-Linker-FosW-Linker-IgMCm2-4) |
| 209 | RFYKTLRAEQASQGSGGGGSGGGGSGDTRPRFLWQPKRECHFFNGTERVRFLDRYFYNQEESVRFDSD |
| | VGEFRAVTELGRPDAEYWNSQKDILEQARAAVDTYCRHNYGVGESFTVQRRVQPKVTVYPSKTQPLQH |
| | HNLLVCSVSGFYPGSIEVRWFLNGQEEKAGMVSTGLIQNGDWTFQTLVMLETVPRSGEVYTCQVEHPS |
| | VTSPLTVEWRARSESAQSKGGGGSGGGGSLDELQAEIEQLEERNYALRKEIEDLQKQLEKLGAPGSGS |
| | IAELPPKVSVFVPPRDGFFGNPRKSKLICQATGFSPRQIQVSWLREGKQVGSGVTTDQVQAEAKESGP |
| | TTYKVTSTLTIKESDWLGQSMFTCRVDHRGLTFQQNASSMCVPDQDTAIRVFAIPPSFASIFLTKSTK |
| | LTCLVTDLTTYDSVTISWTRQNGEAVKTHTNISESHPNATFSAVGEASICEDDWNSGERFTCTVTHTD |
| | LPSPLKQTISRPKGVALHRPDVYLLPPAREQLNLRESATITCLVTGFSPADVFVQWMQRGQPLSPEKY |
| | VTSAPMPEPQAPGRYFAHSILTVSEEEWNTGETYTCVVAHEALPNRVTERTVDKSTGKPTLYNVSLVM |
| | SDTAGTCY |
| | (HIV GAG RQ13-Linker-DRB1*15:02-Linker-FosW-Linker- IgMCm2-4) |
| 210 | FRDYVDRFYKTLRAEQASQEGGGGSIEGRGSGGGSGDTRPRFLWQPKRECHFFNGTERVRFLDRYFYN |
| | QEESVRFDSDVGEFRAVTELGRPDAEYWNSQKDILEQARAAVDTYCRHNYGVGESFTVQRRVQPKVTV |
| | YPSKTQPLQHHNLLVCSVSGFYPGSIEVRWFLNGQEEKAGMVSTGLIQNGDWTFQTLVMLETVPRSGE |
| | VYTCQVEHPSVTSPLTVEWRARSESAQSKGGGGSGGGGSGSIAELPPKVSVFVPPRDGFFGNPRKSKL |
| | ICQATGFSPRQIQVSWLREGKQVGSGVTTDQVQAEAKESGPTTYKVTSTLTIKESDWLGQSMFTCRVD |
| | HRGLTFQQNASSMCVPDQDTAIRVFAIPPSFASIFLTKSTKLTCLVTDLTTYDSVTISWTRQNGEAVK |
| | THTNISESHPNATFSAVGEASICEDDWNSGERFTCTVTHTDLPSPLKQTISRPKGVALHRPDVYLLPP |
| | AREQLNLRESATITCLVTGFSPADVFVQWMQRGQPLSPEKYVTSAPMPEPQAPGRYFAHSILTVSEEE |
| | WNTGETYTCVVAHEALPNRVTERTVDKSTGKPTLYNVSLVMSDTAGTCY |
| | (HIV GAG-LinkerXa-DRB1*15:02-Linker-IgMCm2-4) |
| 211 | FRDYVDRFYKTLRAEQASQEGSGGGGSGGGGSGDTRPRFLWQPKRECHFFNGTERVRFLDRYFYNQEE |
| | SVRFDSDVGEFRAVTELGRPDAEYWNSQKDILEQARAAVDTYCRHNYGVGESFTVQRRVQPKVTVYPS |
| | KTQPLQHHNLLVCSVSGFYPGSIEVRWFLNGQEEKAGMVSTGLIQNGDWTFQTLVMLETVPRSGEVYT |
| | CQVEHPSVTSPLTVEWRARSESAQSKGGGGSGGGGSGSIAELPPKVSVFVPPRDGFFGNPRKSKLICQ |
| | ATGFSPRQIQVSWLREGKQVGSGVTTDQVQAEAKESGPTTYKVTSTLTIKESDWLGQSMFTCRVDHRG |
| | LTFQQNASSMCVPDQDTAIRVFAIPPSFASIFLTKSTKLTCLVTDLTTYDSVTISWTRQNGEAVKTHT |
| | NISESHPNATFSAVGEASICEDDWNSGERFTCTVTHTDLPSPLKQTISRPKGVALHRPDVYLLPPARE |
| | QLNLRESATITCLVTGFSPADVFVQWMQRGQPLSPEKYVTSAPMPEPQAPGRYFAHSILTVSEEEWNT |
| | GETYTCVVAHEALPNRVTERTVDKSTGKPTLYNVSLVMSDTAGTCY |
| | (HIV GAG-Linker-DRB1*15:02-Linker-IgMCm2-4) |
| 212 | FRDYVDRFYKTLRAEQASQEGSGGGGSGGGGSGDTRPRFLWQPKRECHFFNGTERVRFLDRYFYNQEE |
| | SVRFDSDVGEFRAVTELGRPDAEYWNSQKDILEQARAAVDTYCRHNYGVGESFTVQRRVQPKVTVYPS |
| | KTQPLQHHNLLVCSVSGFYPGSIEVRWFLNGQEEKAGMVSTGLIQNCDWTFQTLVMLETVPRSGEVYT |
| | CQVEHPSVTSPLTVEWRARSESAQSKGGGGSGGGGSGGGGSCVPDQDTAIRVFAIPPSFASIFLTKST |
| | KLTCLVTDLTTYDSVTISWTRQNGEAVKTHTNISESHPNATFSAVGEASICEDDWNSGERFTCTVTHT |
| | DLPSPLKQTISRPKGVALHRPDVYLLPPAREQLNLRESATITCLVTGFSPADVFVQWMQRGQPLSPEK |
| | YVTSAPMPEPQAPGRYFAHSILTVSEEEWNTGETYTCVVAHEALPNRVTERTVDKSTGKPTLYNVSLV |
| | MSDTAGTCY |
| | (HIV GAG-Linker-DRB1*15:02-Linker-IgMCm3-4) |
| 213 | FRDYVDRFYKTLRAEQASQEGSGGGGSGGGGSGDTRPRFLWQPKRECHFFNGTERVRFLDRYFYNQEE |
| | SVRFDSDVGEFRAVTELGRPDAEYWNSQKDILEQARAAVDTYCRHNYGVGESFTVQRRVQPKVTVYPS |
| | KTQPLQHHNLLVCSVSGFYPGSIEVRWFLNGQEEKAGMVSTGLIQNGDWTFQTLVMLETVPRSGEVYT |
| | CQVEHPSVTSPLTVEWRARSESAQSKGGGGSGGGGSLDELQAEIEQLEERNYALRKEIEDLQKQLEKL |
| | GAPGSGSIAELPPKVSVFVPPRDGFFGNPRKSKLICQATGFSPRQIQVSWLREGKQVGSGVTTDQVQA |
| | EAKESGPTTYKVTSTLTIKESDWLGQSMFTCRVDHRGLTFQQNASSMCVPDQDTAIRVFAIPPSFASI |
| | FLTKSTKLTCLVTDLTTYDSVTISWTRQNGEAVKTHTNISESHPNATFSAVGEASICEDDWNSGERFT |
| | CTVTHTDLPSPLKQTISRPKGVALHRPDVYLLPPAREQLNLRESATITCLVTGFSPADVFVQWMQRGQ |
| | PLSPEKYVTSAPMPEPQAPGRYFAHSILTVSEEEWNTGETYTCVVAHEALPNRVTERTVDKSTGKPTL |
| | YNVSLVMSDTAGTCY |
| | (HIV GAG-Linker-DRB1*15:02-Linker-FosW-Linker-IgMCm2-4) |
| 214 | RFYKTLRAEQASQGGGGSGGGGSGSGDTRPRFLWQLKFECHFFNGTERVRLLERCIYNQEESVRFDSD |
| | VGEYRAVTELGRPDAEYWNSQKDLLEQRRAAVDTYCRHNYGVGESFTVQRRVEPKVTVYPSKTQPLQH |
| | HNLLVCSVSGFYPGSIEVRWFRNGQEEKAGVVSTGLIQNGDWTFQTLVMLETVPRSGEVYTCQVEHPS |
| | VTSPLTVEWRARSESAQSKGGGGSGGGGSLDELQAEIEQLEERNYALRKEIEDLQKQLEKLGAPGGGG |
| | SGIAELPPKVSVFVPPRDGFFGNPRKSKLICQATGFSPRQIQVSWLREGKQVGSGVTTDQVQAEAKES |
| | GPTTYKVTSTLTIKESDWLGQSMFTCRVDHRGLTFQQNASSMCVPDQDTAIRVFAIPPSFASIFLTKS |
| | TKLTCLVTDLTTYDSVTISWTRQNGEAVKTHTNISESHPNATFSAVGEASICEDDWNSGERFTCTVTH |
| | TDLPSPLKQTISRPKGVALHRPDVYLLPPAREQLNLRESATITCLVTGFSPADVFVQWMQRGQPLSPE |
| | KYVTSAPMPEPQAPGRYFAHSILTVSEEEWNTGETYTCVVAHEALPNRVTERTVDKSTGKPTLYNVSL |
| | VMSDTAGTCY |
| | (HIV RQ13-Linker-DRB1*01:01-Linker-FosW-Linker-IgMCm2-4) |
| 215 | RFYKTLRAEQASQGGGGSGGGGSGSGDTRPRFLWQLKFECHFFNGTERVRLLERCIYNQEESVRFDSD |
| | VGEYRAVTELGRPDAEYWNSQKDLLEQRRAAVDTYCRHNYGVGESFTVQRRVEPKVTVYPSKTQPLQH |
| | HNLLVCSVSGFYPGSIEVRWFRNGQEEKAGVVSTGLIQNGDWTFQTLVMLETVPRSGEVYTCQVEHPS |
| | VTSPLTVEWRARSESAQSKGGGGSGGGGSGGGGSIAELPPKVSVFVPPRDGFFGNPRKSKLICQATGF |
| | SPRQIQVSWLREGKQVGSGVTTDQVQAEAKESGPTTYKVTSTLTIKESDWLGQSMFTCRVDHRGLTFQ |
| | QNASSMCVPDQDTAIRVFAIPPSFASIFLTKSTKLTCLVTDLTTYDSVTISWTRQNGEAVKTHTNISE |
| | SHPNATFSAVGEASICEDDWNSGERFTCTVTHTDLPSPLKQTISRPKGVALHRPDVYLLPPAREQLNL |
| | RESATITCLVTGFSPADVFVQWMQRGQPLSPEKYVTSAPMPEPQAPGRYFAHSILTVSEEEWNTGETY |
| | TCVVAHEALPNRVTERTVDKSTGKPTLYNVSLVMSDTAGTCY |
| | (HIV RQ13-Linker-DRB1*01:01-Linker-IgMCm2-4) |
| 216 | IKEEHVIIQAEFYLNPDQSGEFMFDFDGDEIFHVDMAKKETVWRLEEFGRFASFEAQGALANIAVDKA |
| | NLECMTKRSNYTPITNVPPEVTVLTNSPVELREPNVLICFIDKFTPPVVNVTWLRNGKPVTTGVSETV |
| | FLPRFDHLFRKFHYLPFLPSTEDVYDCRVEHWGLDEPLLKHWEFDAPSPLPETTEGGGGSGGGGSIAR |
| | LEEKVKTLKAQNYELASTANMLREQVAQLGAPGSEQKLISEEDL |
| | (Cys-trap DRA1*01 (I92C)-Linker-cJun-Myc) |
| 217 | IKEEHVIIQAEFYLNPDQSGEFMFDFDGDEIFHVDMAKKETVWRLEEFGRFASFEAQGALANIAVDKA |
| | NLECMTKRSNYTPITNVPPEVTVLTNSPVELREPNVLICFIDKFTPPVVNVTWLRNGKPVTTGVSETV |
| | FLPRFDHLFRKFHYLPFLPSTEDVYDCRVEHWGLDEPLLKHWEFDAPSPLPETTEGSEQKLISEEDL |
| | (Cys-trap DRA1*01 (I92C)-Linker-Myc) |
| 218 | MSQIMYNYPAMMAHCGGGGSGGGGSGSGDTRPRFLWQPKRECHFFNGTERVRFLDRYFYNQEESVRFD |
| | SDVGEFRAVTELGRPDAEYWNSQKDILEQARAAVDTYCRHNYGVVESFTVQRRVQPKVTVYPSKTQPL |
| | QHHNLLVCSVSGFYPGSIEVRWFLNGQEEKAGMVSTGLIQNGDWTFQTLVMLETVPRSGEVYTCQVEH |
| | PSVTSPLTVEWRARSESAQSKGGGGSGGGGSLDELQAEIEQLEERNYALRKEIEDLQKQLEKLGAPGG |
| | GGSGIAELPPKVSVFVPPRDGFFGNPRKSKLICQATGFSPRQIQVSWLREGKQVGSGVTTDQVQAEAK |
| | ESGPTTYKVTSTLTIKESDWLGQSMFTCRVDHRGLTFQQNASSMCVPDQDTAIRVFAIPPSFASIFLT |
| | KSTKLTCLVTDLTTYDSVTISWTRQNGEAVKTHTNISESHPNATFSAVGEASICEDDWNSGERFTCTV |
| | THTDLPSPLKQTISRPKGVALHRPDVYLLPPAREQLNLRESATITCLVTGFSPADVFVQWMQRGQPLS |
| | PEKYVTSAPMPEPQAPGRYFAHSILTVSEEEWNTGETYTCVVAHEALPNRVTERTVDKSTGKPTLYNV |
| | SLVMSDTAGTCY |
| | (Cys-trap MTB(C15)-Linker-DRB1*15:01-Linker-FosW-Linker-IgMCm2-4) |
| 219 | RFYKTLRAEQASQCGGGGSGGGGSGSGDTRPRFLWQLKFECHFFNGTERVRLLERCIYNQEESVRFDS |
| | DVGEYRAVTELGRPDAEYWNSQKDLLEQRRAAVDTYCRHNYGVGESFTVQRRVEPKVTVYPSKTQPLQ |
| | HHNLLVCSVSGFYPGSIEVRWFRNGQEEKAGVVSTGLIQNGDWTFQTLVMLETVPRSGEVYTCQVEHP |
| | SVTSPLTVEWRARSESAQSKGGGGSGGGGSGGGGSIAELPPKVSVFVPPRDGFFGNPRKSKLICQATG |
| | FSPRQIQVSWLREGKQVGSGVTTDQVQAEAKESGPTTYKVTSTLTIKESDWLGQSMFTCRVDHRGLTF |
| | QQNASSMCVPDQDTAIRVFAIPPSFASIFLTKSTKLTCLVTDLTTYDSVTISWTRQNGEAVKTHTNIS |
| | ESHPNATFSAVGEASICEDDWNSGERFTCTVTHTDLPSPLKQTISRPKGVALHRPDVYLLPPAREQLN |
| | LRESATITCLVTGFSPADVFVQWMQRGQPLSPEKYVTSAPMPEPQAPGRYFAHSILTVSEEEWNTGET |
| | YTCVVAHEALPNRVTERTVDKSTGKPTLYNVSLVMSDTAGTCY |
| | (Cys-trap HIV RQ13(C14)-Linker-DRB1*01:01-Linker-IgMCm2-4) |
| 220 | ENPVVHFFKNIVTCGGGGSGGGGSGSGDTRPRFLWQPKRECHFFNGTERVRFLDRYFYNQEESVRFDS |
| | DVGEFRAVTELGRPDAEYWNSQKDILEQARAAVDTYCRHNYGVVESFTVQRRVQPKVTVYPSKTQPLQ |
| | HHNLLVCSVSGFYPGSIEVRWFLNGQEEKAGMVSTGLIQNGDWTFQTLVMLETVPRSGEVYTCQVEHP |
| | SVTSPLTVEWRARSESAQSKGGGGSGGGGSLDELQAEIEQLEERNYALRKEIEDLQKQLEKLGAPGGG |
| | GSGIAELPPKVSVFVPPRDGFFGNPRKSKLICQATGFSPRQIQVSWLREGKQVGSGVTTDQVQAEAKE |
| | SGPTTYKVTSTLTIKESDWLGQSMFTCRVDHRGLTFQQNASSMCVPDQDTAIRVFAIPPSFASIFLTK |
| | STKLTCLVTDLTTYDSVTISWTRQNGEAVKTHTNISESHPNATFSAVGEASICEDDWNSGERFTCTVT |
| | HTDLPSPLKQTISRPKGVALHRPDVYLLPPAREQLNLRESATITCLVTGFSPADVFVQWMQRGQPLSP |
| | EKYVTSAPMPEPQAPGRYFAHSILTVSEEEWNTGETYTCVVAHEALPNRVTERTVDKSTGKPTLYNVS |
| | LVMSDTAGTCY |
| | (Cys-trap MBP(C14)-Linker-DRB1*15:01-Linker-FosW-Linker-IgMCm2-4) |
| 221 | ENPVVHFFKNIVTPGGGGSGGGGSGSGDTRPRFLWQPKRECHFFNGTERVRFLDRYFYNQEESVRFDS |
| | DVGEFRAVTELGRPDAEYWNSQKDILEQARAAVDTYCRHNYGVVESFTVQRRVQPKVTVYPSKTQPLQ |
| | HHNLLVCSVSGFYPGSIEVRWFLNGQEEKAGMVSTGLIQNGDWTFQTLVMLETVPRSGEVYTCQVEHP |
| | SVTSPLTVEWRARSESAQSKGGGGSGGGGSLDELQAEIEQLEERNYALRKEIEDLQKQLEKLGAPGSG |
| | LNDIFEAQKIEWHEGSLPETGGHHHHHHHH |
| | (MBP-Linker-DRB1*15:01-Linker-FosW-Linker-Avitag-Linker-Sortag-His) |
| 222 | MSQIMYNYPAMMAHAGGGGSGGGGSGSGDTRPRFLWQPKRECHFFNGTERVRFLDRYFYNQEESVRFD |
| | SDVGEFRAVTELGRPDAEYWNSQKDILEQARAAVDTYCRHNYGVVESFTVQRRVQPKVTVYPSKTQPL |
| | QHHNLLVCSVSGFYPGSIEVRWFLNGQEEKAGMVSTGLIQNGDWTFQTLVMLETVPRSGEVYTCQVEH |
| | PSVTSPLTVEWRARSESAQSKGGGGSGGGGSLDELQAEIEQLEERNYALRKEIEDLQKQLEKLGAPGS |
| | GLNDIFEAQKIEWHEGSLPETGGHHHHHHHH |
| | (MTB-Linker-DRB1*15:01-Linker-FosW-Linker-Avitag-Linker-Sortag-His) |
| 223 | RFYKTLRAEQASQGGGGSGGGGSGSGDTRPRFLWQLKFECHFFNGTERVRLLERCIYNQEESVRFDSD |
| | VGEYRAVTELGRPDAEYWNSQKDLLEQRRAAVDTYCRHNYGVGESFTVQRRVEPKVTVYPSKTQPLQH |
| | HNLLVCSVSGFYPGSIEVRWFRNGQEEKAGVVSTGLIQNGDWTFQTLVMLETVPRSGEVYTCQVEHPS |
| | VTSPLTVEWRARSESAQSKGGGGSGGGGSLDELQAEIEQLEERNYALRKEIEDLQKQLEKLGAPGSGL |
| | NDIFEAQKIEWHEGSLPETGGHHHHHHHH |
| | (HIV RQ13-Linker-DRB1*15:01-Linker-FosW-Linker-Avitag-Linker- |
| | Sortag-His) |
| 224 | VKQIKVRVDMVRHRIGGGGSGGGGSGSGDTRPRFLEYSTSECHFFNGTERVRYLDRYFHNQEENVRFD |
| | SDVGEFRAVTELGRPDAEYWNSQKDLLEQKRGRVDNYCRHNYGVVESFTVQRRVHPKVTVYPSKTQPL |
| | QHHNLLVCSVSGFYPGSIEVRWFRNGQEEKTGVVSTGLIHNGDWTFQTLVMLETVPRSGEVYTCQVEH |
| | PSVTSPLTVEWRARSESAQSKGGGGSGGGGSLDELQAEIEQLEERNYALRKEIEDLQKQLEKLGAPGS |
| | GLNDIFEAQKIEWHEGSLPETGGHHHHHHHH |
| | (CMV-Linker-DRB1*03:01-Linker-FosW-Linker-Avitag-Linker-Sortag-His) |
| 225 | GPKYVKQNTLKLATGGGGSGGGGSGSGDTRPRFLEQVKHECHFFNGTERVRFLDRYFYHQEEYVRFDS |
| | DVGEYRAVTELGRPDAEYWNSQKDLLEQKRAAVDTYCRHNYGVGESFTVQRRVYPEVTVYPAKTQPLQ |
| | HHNLLVCSVNGFYPGSIEVRWFRNGQEEKTGVVSTGLIQNGDWTFQTLVMLETVPRSGEVYTCQVEHP |
| | SLTSPLTVEWRARSESAQSKGGGGSGGGGSLDELQAEIEQLEERNYALRKEIEDLQKQLEKLGAPGSG |
| | LNDIFEAQKIEWHEGSLPETGGHHHHHHHH |
| | (HA-Linker-DRB1*04:01-Linker-FosW-Linker-Avitag-Linker-Sortag- |
| | Histag) |
| 226 | GPKYVKQNTLKLATGGGGSGGGGSGSGDTRPRFLEQVKHECHFFNGTERVRFLDRYFYHQEEYVRFDS |
| | DVGEYRAVTELGRPDAEYWNSQKDLLEQKRAAVDTYCRHNYGVGESFTVQRRVYPEVTVYPAKTQPLQ |
| | HHNLLVCSVNGFYPGSIEVRWFRNGQEEKTGVVSTGLIQNGDWTFQTLVMLETVPRSGEVYTCQVEHP |
| | SLTSPLTVEWRARSESAQSKGSGLNDIFEAQKIEWHEGSLPETGGHHHHHHHH |
| | (HA-Linker-DRB1*04:01-Linker-Avitag-Linker-Sortag-Histag) |
| 227 | ENPVVHFFKNIVTCGGGGSGGGGSGSGDTRPRFLWQPKRECHFFNGTERVRFLDRYFYNQEESVRFDS |
| | DVGEFRAVTELGRPDAEYWNSQKDILEQARAAVDTYCRHNYGVVESFTVQRRVQPKVTVYPSKTQPLQ |
| | HHNLLVCSVSGFYPGSIEVRWFLNGQEEKAGMVSTGLIQNGDWTFQTLVMLETVPRSGEVYTCQVEHP |
| | SVTSPLTVEWRARSESAQSKGGGGSGGGGSLDELQAEIEQLEERNYALRKEIEDLQKQLEKLGAPGSG |
| | LNDIFEAQKIEWHEGSLPETGGHHHHHHHH |
| | (MBP(C14)-Linker-DRB1*15:01-Linker-FosW-Linker-Avitag-Linker- |
| | Sortag-Histag) |
| 228 | MSQIMYNYPAMMAHCGGGGSGGGGSGSGDTRPRFLWQPKRECHFFNGTERVRFLDRYFYNQEESVRFD |
| | SDVGEFRAVTELGRPDAEYWNSQKDILEQARAAVDTYCRHNYGVVESFTVQRRVQPKVTVYPSKTQPL |
| | QHHNLLVCSVSGFYPGSIEVRWFLNGQEEKAGMVSTGLIQNGDWTFQTLVMLETVPRSGEVYTCQVEH |
| | PSVTSPLTVEWRARSESAQSKGGGGSGGGGSLDELQAEIEQLEERNYALRKEIEDLQKQLEKLGAPGS |
| | GLNDIFEAQKIEWHEGSLPETGGHHHHHHHH |
| | (MTB(C15)-Linker-DRB1*15:01-Linker-FosW-Linker-Avitag-Linker- |
| | Sortag-Histag) |
| 229 | VKQIKVRVDMVRHRCGGGGSGGGGSGSGDTRPRFLEYSTSECHFFNGTERVRYLDRYFHNQEENVRFD |
| | SDVGEFRAVTELGRPDAEYWNSQKDLLEQKRGRVDNYCRHNYGVVESFTVQRRVHPKVTVYPSKTQPL |
| | QHHNLLVCSVSGFYPGSIEVRWFRNGQEEKTGVVSTGLIHNGDWTFQTLVMLETVPRSGEVYTCQVEH |
| | PSVTSPLTVEWRARSESAQSKGSGLNDIFEAQKIEWHEGSLPETGGHHHHHHHH |
| | (CMV(C14)-DRB1*03:01-Linker-Avitag-Linker-Sortag-Histag) |
| 230 | GPKYVKQNTLKLCTGGGGSGGGGSGSGDTRPRFLEQVKHECHFFNGTERVRFLDRYFYHQEEYVRFDS |
| | DVGEYRAVTELGRPDAEYWNSQKDLLEQKRAAVDTYCRHNYGVGESFTVQRRVYPEVTVYPAKTQPLQ |
| | HHNLLVCSVNGFYPGSIEVRWFRNGQEEKTGVVSTGLIQNGDWTFQTLVMLETVPRSGEVYTCQVEHP |
| | SLTSPLTVEWRARSESAQSKGGGGSGGGGSLDELQAEIEQLEERNYALRKEIEDLQKQLEKLGAPGSG |
| | LNDIFEAQKIEWHEGSLPETGGHHHHHHHH |
| | (HA(C13)-Linker-DRB1*04:01-Linker-FosW-Linker-Avitag-Linker-Sortag- |
| | Histag) |
| 231 | GPKYVKQNTLKLACGGGGSGGGGSGSGDTRPRFLEQVKHECHFFNGTERVRFLDRYFYHQEEYVRFDS |
| | DVGEYRAVTELGRPDAEYWNSQKDLLEQKRAAVDTYCRHNYGVGESFTVQRRVYPEVTVYPAKTQPLQ |
| | HHNLLVCSVNGFYPGSIEVRWFRNGQEEKTGVVSTGLIQNGDWTFQTLVMLETVPRSGEVYTCQVEHP |
| | SLTSPLTVEWRARSESAQSKGSGLNDIFEAQKIEWHEGSLPETGGHHHHHHHH |
| | (HA(C14)-Linker-DRB1*04:01-Linker-Avitag-Linker-Sortag-Histag) |
| 232 | HHHHHHHH |
| | (His8 tag) |
| 233 | NLVPMIATV |
| 234 | MTINYQFGDVDAHGA |
| 235 | ALWEIQQVVGGGGSGGGGSGGGGSIQRTPKIQVYSRHPAENGKSNFLNCYVSGFHPSDIEVDLLKNGE |
| | RIEKVEHSDLSFSKDWSFYLLYYTEFTPTEKDEYACRVNHVTLSQPKIVKWDRDMGGGGSGGGGSGGG |
| | GSGGGGSGSHSMRYFFTSVSRPGRGEPRFIAVGYVDDTQFVRFDSDAASQRMEPRAPWIEQEGPEYWD |
| | GETRKVKAHSQTHRVDLGTLRGAYNQSEAGSHTVQRMYGCDVGSDWRFLRGYHQYAYDGKDYIALKED |
| | LRSWTAADMAAQTTKHKWEAAHVAEQLRAYLEGTCVEWLRRYLENGKETLQRTDAPKTHMTHHAVSDH |
| | EATLRCWALSFYPAEITLTWQRDGEDQTQDTELVETRPAGDGTFQKWAAVVVPSGQEQRYTCHVQHEG |
| | LPKPLTLRWEPSSGGGGSGGGGSGGGGSIAELPPKVSVFVPPRDGFFGNPRKSKLICQATGFSPRQIQ |
| | VSWLREGKQVGSGVTTDQVQAEAKESGPTTYKVTSTLTIKESDWLGQSMFTCRVDHRGLTFQQNASSM |
| | CVPDQDTAIRVFAIPPSFASIFLTKSTKLTCLVTDLTTYDSVTISWTRQNGEAVKTHTNISESHPNAT |
| | FSAVGEASICEDDWNSGERFTCTVTHTDLPSPLKQTISRPKGVALHRPDVYLLPPAREQLNLRESATI |
| | TCLVTGFSPADVFVQWMQRGQPLSPEKYVTSAPMPEPQAPGRYFAHSILTVSEEEWNTGETYTCVVAH |
| | EALPNRVTERTVDKSTGKPTLYNVSLVMSDTAGTCY |
| | (ALW_B2M_HLAA2_IgM2-4) |
| 236 | KLWAQCVQLGGGGSGGGGSGGGGSIQRTPKIQVYSRHPAENGKSNFLNCYVSGFHPSDIEVDLLKNGE |
| | RIEKVEHSDLSFSKDWSFYLLYYTEFTPTEKDEYACRVNHVTLSQPKIVKWDRDMGGGGSGGGGSGGG |
| | GSGGGGSGSHSMRYFFTSVSRPGRGEPRFIAVGYVDDTQFVRFDSDAASQRMEPRAPWIEQEGPEYWD |
| | GETRKVKAHSQTHRVDLGTLRGAYNQSEAGSHTVQRMYGCDVGSDWRFLRGYHQYAYDGKDYIALKED |
| | LRSWTAADMAAQTTKHKWEAAHVAEQLRAYLEGTCVEWLRRYLENGKETLQRTDAPKTHMTHHAVSDH |
| | EATLRCWALSFYPAEITLTWQRDGEDQTQDTELVETRPAGDGTFQKWAAVVVPSGQEQRYTCHVQHEG |
| | LPKPLTLRWEPSSGGGGSGGGGSGGGGSIAELPPKVSVFVPPRDGFFGNPRKSKLICQATGFSPRQIQ |
| | VSWLREGKQVGSGVTTDQVQAEAKESGPTTYKVTSTLTIKESDWLGQSMFTCRVDHRGLTFQQNASSM |
| | CVPDQDTAIRVFAIPPSFASIFLTKSTKLTCLVTDLTTYDSVTISWTRQNGEAVKTHTNISESHPNAT |
| | FSAVGEASICEDDWNSGERFTCTVTHTDLPSPLKQTISRPKGVALHRPDVYLLPPAREQLNLRESATI |
| | TCLVTGFSPADVFVQWMQRGQPLSPEKYVTSAPMPEPQAPGRYFAHSILTVSEEEWNTGETYTCVVAH |
| | EALPNRVTERTVDKSTGKPTLYNVSLVMSDTAGTCY |
| | (KLW_B2M_HLAA2_IgM2-4) |
| 237 | YMLDLQPETTGGGGSGGGGSGGGGSIQRTPKIQVYSRHPAENGKSNFLNCYVSGFHPSDIEVDLLKNG |
| | ERIEKVEHSDLSFSKDWSFYLLYYTEFTPTEKDEYACRVNHVTLSQPKIVKWDRDMGGGGSGGGGSGG |
| | GGSGGGGSGSHSMRYFFTSVSRPGRGEPRFIAVGYVDDTQFVRFDSDAASQRMEPRAPWIEQEGPEYW |
| | DGETRKVKAHSQTHRVDLGTLRGAYNQSEAGSHTVQRMYGCDVGSDWRFLRGYHQYAYDGKDYIALKE |
| | DLRSWTAADMAAQTTKHKWEAAHVAEQLRAYLEGTCVEWLRRYLENGKETLQRTDAPKTHMTHHAVSD |
| | HEATLRCWALSFYPAEITLTWQRDGEDQTQDTELVETRPAGDGTFQKWAAVVVPSGQEQRYTCHVQHE |
| | GLPKPLTLRWEPSSGGGGSGGGGSGGGGSIAELPPKVSVFVPPRDGFFGNPRKSKLICQATGFSPRQI |
| | QVSWLREGKQVGSGVTTDQVQAEAKESGPTTYKVTSTLTIKESDWLGQDMFTCRVDHRGLTFQQNASS |
| | MCVPDQDTAIRVFAIPPSFASIFLTKSTKLTCLVTDLTTYDSVTISWTRQNGEAVKTHTNISESHPNA |
| | TFSAVGEASICEDDWNSGERFTCTVTHTDLPSPLKQTISRPKGVALHRPDVYLLPPAREQLNLRESAT |
| | ITCLVTGFSPADVFVQWMQRGQPLSPEKYVTSAPMPEPQAPGRYFAHSILTVSEEEWNTGETYTCVVA |
| | HEALPNRVTERTVDKSTGKPTLYNVSLVMSDTAGTCY |
| | (YML_B2M_HLAA2_IgM2-4) |
| 238 | NLVPMVATVGCGGSGGGGSGGGGSIQRTPKIQVYSRHPAENGKSNFLNCYVSGFHPSDIEVDLLKNGE |
| | RIEKVEHSDLSFSKDWSFYLLYYTEFTPTEKDEYACRVNHVTLSQPKIVKWDRDMGGGGSGGGGSGGG |
| | GSGGGGSGSHSMRYFFTSVSRPGRGEPRFIAVGYVDDTQFVRFDSDAASQRMEPRAPWIEQEGPEYWD |
| | GETRKVKAHSQTHRVDLGTLRGCYNQSEAGSHTVQRMYGCDVGSDWRFLRGYHQYAYDGKDYIALKED |
| | LRSWTAADMAAQTTKHKWEAAHVAEQLRAYLEGTCVEWLRRYLENGKETLQRTDAPKTHMTHHAVSDH |
| | EATLRCWALSFYPAEITLTWQRDGEDQTQDTELVETRPAGDGTFQKWAAVVVPSGQEQRYTCHVQHEG |
| | LPKPLTLRWEPSSGGGGSGGGGSGGGGSIAELPPKVSVFVPPRDGFFGNPRKSKLICQATGFSPRQIQ |
| | VSWLREGKQVGSGVTTDQVQAEAKESGPTTYKVTSTLTIKESDWLGQSMFTCRVDHRGLTFQQNASSM |
| | CVPDQDTAIRVFAIPPSFASIFLTKSTKLTCLVTDLTTYDSVTISWTRQNGEAVKTHTNISESHPNAT |
| | FSAVGEASICEDDWNSGERFTCTVTHTDLPSPLKQTISRPKGVALHRPDVYLLPPAREQLNLRESATI |
| | TCLVTGFSPADVFVQWMQRGQPLSPEKYVTSAPMPEPQAPGRYFAHSILTVSEEEWNTGETYTCVVAH |
| | EALPNRVTERTVDKSTGKPTLYNVSLVMSDTAGTCY |
| | (cys-trap NLV_B2M_HLAA2 IgM2-4) |
| 239 | ELAGIGILTVGCGGSGGGGSGGGGSIQRTPKIQVYSRHPAENGKSNFLNCYVSGFHPSDIEVDLLKNG |
| | ERIEKVEHSDLSFSKDWSFYLLYYTEFTPTEKDEYACRVNHVTLSQPKIVKWDRDMGGGGSGGGGSGG |
| | GGSGGGGSGSHSMRYFFTSVSRPGRGEPRFIAVGYVDDTQFVRFDSDAASQRMEPRAPWIEQEGPEYW |
| | DGETRKVKAHSQTHRVDLGTLRGCYNQSEAGSHTVQRMYGCDVGSDWRFLRGYHQYAYDGKDYIALKE |
| | DLRSWTAADMAAQTTKHKWEAAHVAEQLRAYLEGTCVEWLRRYLENGKETLQRTDAPKTHMTHHAVSD |
| | HEATLRCWALSFYPAEITLTWQRDGEDQTQDTELVETRPAGDGTFQKWAAVVVPSGQEQRYTCHVQHE |
| | GLPKPLTLRWEPSSGGGGSGGGGSGGGGSIAELPPKVSVFVPPRDGFFGNPRKSKLICQATGFSPRQI |
| | QVSWLREGKQVGSGVTTDQVQAEAKESGPTTYKVTSTLTIKESDWLGQSMFTCRVDHRGLTFQQNASS |
| | MCVPDQDTAIRVFAIPPSFASIFLTKSTKLTCLVTDLTTYDSVTISWTRQNGEAVKTHTNISESHPNA |
| | TFSAVGEASICEDDWNSGERFTCTVTHTDLPSPLKQTISRPKGVALHRPDVYLLPPAREQLNLRESAT |
| | ITCLVTGFSPADVFVQWMQRGQPLSPEKYVTSAPMPEPQAPGRYFAHSILTVSEEEWNTGETYTCVVA |
| | HEALPNRVTERTVDKSTGKPTLYNVSLVMSDTAGTCY |
| | (cys-trap ELA_B2M_HLAA2_IgM2-4) |
| 240 | ALWEIQQVVGCGGSGGGGSGGGGSIQRTPKIQVYSRHPAENGKSNFLNCYVSGFHPSDIEVDLLKNGE |
| | RIEKVEHSDLSFSKDWSFYLLYYTEFTPTEKDEYACRVNHVTLSQPKIVKWDRDMGGGGSGGGGSGGG |
| | GSGGGGSGSHSMRYFFTSVSRPGRGEPRFIAVGYVDDTQFVRFDSDAASQRMEPRAPWIEQEGPEYWD |
| | GETRKVKAHSQTHRVDLGTLRGCYNQSEAGSHTVQRMYGCDVGSDWRFLRGYHQYAYDGKDYIALKED |
| | LRSWTAADMAAQTTKHKWEAAHVAEQLRAYLEGTCVEWLRRYLENGKETLQRTDAPKTHMTHHAVSDH |
| | EATLRCWALSFYPAEITLTWQRDGEDQTQDTELVETRPAGDGTFQKWAAVVVPSGQEQRYTCHVQHEG |
| | LPKPLTLRWEPSSGGGGSGGGGSGGGGSIAELPPKVSVFVPPRDGFFGNPRKSKLICQATGESPRQIQ |
| | VSWLREGKQVGSGVTTDQVQAEAKESGPTTYKVTSTLTIKESDWLGQSMFTCRVDHRGLTFQQNASSM |
| | CVPDQDTAIRVFAIPPSFASIFLTKSTKLTCLVTDLTTYDSVTISWTRQNGEAVKTHTNISESHPNAT |
| | FSAVGEASICEDDWNSGERFTCTVTHTDLPSPLKQTISRPKGVALHRPDVYLLPPAREQLNLRESATI |
| | TCLVTGFSPADVFVQWMQRGQPLSPEKYVTSAPMPEPQAPGRYFAHSILTVSEEEWNTGETYTCVVAH |
| | EALPNRVTERTVDKSTGKPTLYNVSLVMSDTAGTCY |
| | (cys-trap ALW_B2M_HLAA2_IgM2-4) |
| 241 | KLWAQCVQLGCGGSGGGGSGGGGSIQRTPKIQVYSRHPAENGKSNFLNCYVSGFHPSDIEVDLLKNGE |
| | RIEKVEHSDLSFSKDWSFYLLYYTEFTPTEKDEYACRVNHVTLSQPKIVKWDRDMGGGGSGGGGSGGG |
| | GSGGGGSGSHSMRYFFTSVSRPGRGEPRFIAVGYVDDTQFVRFDSDAASQRMEPRAPWIEQEGPEYWD |
| | GETRKVKAHSQTHRVDLGTLRGCYNQSEAGSHTVQRMYGCDVGSDWRFLRGYHQYAYDGKDYIALKED |
| | LRSWTAADMAAQTTKHKWEAAHVAEQLRAYLEGTCVEWLRRYLENGKETLQRTDAPKTHMTHHAVSDH |
| | EATLRCWALSFYPAEITLTWQRDGEDQTQDTELVETRPAGDGTFQKWAAVVVPSGQEQRYTCHVQHEG |
| | LPKPLTLRWEPSSGGGGSGGGGSGGGGSIAELPPKVSVFVPPRDGFFGNPRKSKLICQATGFSPRQIQ |
| | VSWLREGKQVGSGVTTDQVQAEAKESGPTTYKVTSTLTIKESDWLGQSMFTCRVDHRGLTFQQNASSM |
| | CVPDQDTAIRVFAIPPSFASIFLTKSTKLTCLVTDLTTYDSVTISWTRQNGEAVKTHTNISESHPNAT |
| | FSAVGEASICEDDWNSGERFTCTVTHTDLPSPLKQTISRPKGVALHRPDVYLLPPAREQLNLRESATI |
| | TCLVTGFSPADVFVQWMQRGQPLSPEKYVTSAPMPEPQAPGRYFAHSILTVSEEEWNTGETYTCVVAH |
| | EALPNRVTERTVDKSTGKPTLYNVSLVMSDTAGTCY |
| | (cys-trap KLW_B2M_HLAA2_IgM2-4) |
| 242 | YMLDLQPETTGCGGSGGGGSGGGGSIQRTPKIQVYSRHPAENGKSNFLNCYVSGFHPSDIEVDLLKNG |
| | ERIEKVEHSDLSFSKDWSFYLLYYTEFTPTEKDEYACRVNHVTLSQPKIVKWDRDMGGGGSGGGGSGG |
| | GGSGGGGSGSHSMRYFFTSVSRPGRGEPRFIAVGYVDDTQFVRFDSDAASQRMEPRAPWIEQEGPEYW |
| | DGETRKVKAHSQTHRVDLGTLRGCYNQSEAGSHTVQRMYGCDVGSDWRFLRGYHQYAYDGKDYIALKE |
| | DLRSWTAADMAAQTTKHKWEAAHVAEQLRAYLEGTCVEWLRRYLENGKETLQRTDAPKTHMTHHAVSD |
| | HEATLRCWALSFYPAEITLTWQRDGEDQTQDTELVETRPAGDGTFQKWAAVVVPSGQEQRYTCHVQHE |
| | GLPKPLTLRWEPSSGGGGSGGGGSGGGGSIAELPPKVSVFVPPRDGFFGNPRKSKLICQATGFSPRQI |
| | QVSWLREGKQVGSGVTTDQVQAEAKESGPTTYKVTSTLTIKESDWLGQSMFTCRVDHRGLTFQQNASS |
| | MCVPDQDTAIRVFAIPPSFASIFLTKSTKLTCLVTDLTTYDSVTISWTRQNGEAVKTHTNISESHPNA |
| | TFSAVGEASICEDDWNSGERFTCTVTHTDLPSPLKQTISRPKGVALHRPDVYLLPPAREQLNLRESAT |
| | ITCLVTGFSPADVFVQWMQRGQPLSPEKYVTSAPMPEPQAPGRYFAHSILTVSEEEWNTGETYTCVVA |
| | HEALPNRVTERTVDKSTGKPTLYNVSLVMSDTAGTCY |
| | (cys-trap YML_B2M_HLAA2_IgM2-4) |
| 243 | ELAGIGILTVGGGGSGGGGSGGGGSIQRTPKIQVYSRHPAENGKSNFLNCYVSGFHPSDIEVDLLKNG |
| | ERIEKVEHSDLSFSKDWSFYLLYYTEFTPTEKDEYACRVNHVTLSQPKIVKWDRDMGGGGSGGGGSGG |
| | GGSGGGGSGSHSMRYFFTSVSRPGRGEPRFIAVGYVDDTQFVRFDSDAASQRMEPRAPWIEQEGPEYW |
| | DGETRKVKAHSQTHRVDLGTLRGAYNQSEAGSHTVQRMYGCDVGSDWRFLRGYHQYAYDGKDYIALKE |
| | DLRSWTAADMAAQTTKHKWEAAHVAEQLRAYLEGTCVEWLRRYLENGKETLQRTDAPKTHMTHHAVSD |
| | HEATLRCWALSFYPAEITLTWQRDGEDQTQDTELVETRPAGDGTFQKWAAVVVPSGQEQRYTCHVQHE |
| | GLPKPLTLRWEPSSGSGSAGGSGSGGGSLPETGGHHHHHH |
| | (single-chain ELA-A2 LPETGG monomer) |
| 244 | GGGGSGGGGSGGGGSIAELPPKVSVFVPPRDGFFGNPRKSKLICQATGFSPRQIQVSWLREGKQVGSG |
| | VTTDQVQAEAKESGPTTYKVTSTLTIKESDWLGQSMFTCRVDHRGLTFQQNASSMCVPDQDTAIRVFA |
| | IPPSFASIFLTKSTKLTCLVTDLTTYDSVTISWTRQNGEAVKTHTNISESHPNATFSAVGEASICEDD |
| | WNSGERFTCTVTHTDLPSPLKQTISRPKGVALHRPDVYLLPPAREQLNLRESATITCLVTGFSPADVF |
| | VQWMQRGQPLSPEKYVTSAPMPEPQAPGRYFAHSILTVSEEEWNTGETYTCVVAHEALPNRVTERTVD |
| | KSTGKPTLYNVSLVMSDTAGTCY |
| | (GGG-IgM Fc for sortase mediated conjugation) |
| 245 | FTVTVPKDLYVVEYGSNMTIECKFPVEKQLDLAALIVYWEMEDKNIIQFVHGEEDLKVQHSSYRQRAR |
| | LLKDQLSLGNAALQITDVKLQDAGVYRCMISYGGADYKRITVKVNAPYNKINQRILVVDPVTSEHELT |
| | CQAEGYPKAEVIWTSSDHQVLSGKTTTTNSKREEKLFNVTSTLRINTTTNEIFYCTFRRLDPEENHTA |
| | ELVIPELPLAHPPNERGGGGSGGGGSGGGGSGGGGSGGGGSQEDERIVLVDNKCKCARITSRIIRSSE |
| | DPNEDIVERNIRIIVPLNNRENISDPTSPLRTRFVYHLSDLCKKCDPTEVELDNQIVTATQSNICDED |
| | SATETCYTYDRNKCYTAVVPLVYGGETKMVETALTPDACYPDGGGGSGGGGSDYKDDDDKHHHHHHHH |
| | (PD-L1-ECD-J-chain-Flag-His) |
| 246 | FTVTVPKDLYVVEYGSNMTIECKFPVEKQLDLAALIVYWEMEDKNIIQFVHGEEDLKVQHSSYRQRAR |
| | LLKDQLSLGNAALQITDVKLQDAGVYRCMISYGGADYKRITVKVNAGGGGSGGGGSGGGGSGGGGSGG |
| | GGSQEDERIVLVDNKCKCARITSRIIRSSEDPNEDIVERNIRIIVPLNNRENISDPTSPLRTRFVYHL |
| | SDLCKKCDPTEVELDNQIVTATQSNICDEDSATETCYTYDRNKCYTAVVPLVYGGETKMVETALTPDA |
| | CYPDGGGGSGGGGSDYKDDDDKHHHHHHHH |
| | (PD-L1-D1-Linker-Jchain-Flag-His) |
| 247 | QVQLQQSGAELARPGASVKMSCKASGYTFTRYTMHWVKQRPGQGLEWIGYINPSRGYTNYNQKFKDKA |
| | TLTTDKSSSTAYMQLSSLTSEDSAVYYCARYYDDHYSLDYWGQGTTLTVSSGGGGSGGGGSGGGGSQI |
| | VLTQSPAIMSASPGEKVTMTCSASSSVSYMNWYQQKSGTSPKRWIYDTSKLASGVPAHFRGSGSGTSY |
| | SLTISGMEAEDAATYYCQQWSSNPFTFGSGTKLEIKGGGGSGGGGSGGGGSQEDERIVLVDNKCKCAR |
| | ITSRIIRSSEDPNEDIVERNIRIIVPLNNRENISDPTSPLRTRFVYHLSDLCKKCDPTEVELDNQIVT |
| | ATQSNICDEDSATETCYTYDRNKCYTAVVPLVYGGETKMVETALTPDACYPDGGGGSGGGGSDYKDDD |
| | DKHHHHHHHH |
| | (Anti-CD3scFv-Linker-Jchain-Flag-His) |
| 248 | NLVPMVATVGGGGSGGGGSGGGGSIQRTPKIQVYSRHPAENGKSNFLNCYVSGFHPSDIEVDLLKNGE |
| | RIEKVEHSDLSFSKDWSFYLLYYTEFTPTEKDEYACRVNHVTLSQPKIVKWDRDMGGGGSGGGGSGGG |
| | GSGGGGSGSHSMRYFFTSVSRPGRGEPRFIAVGYVDDTQFVRFDSDAASQRMEPRAPWIEQEGPEYWD |
| | GETRKVKAHSQTHRVDLGTLRGAYNQSEAGSHTVQRMYGCDVGSDWRFLRGYHQYAYDGKDYIALKED |
| | LRSWTAADMAAQTTKHKWEAAHVAEQLRAYLEGTCVEWLRRYLENGKETLQRTDAPKTHMTHHAVSDH |
| | EATLRCWALSFYPAEITLTWQRDGEDQTQDTELVETRPAGDGTFQKWAAVVVPSGQEQRYTCHVQHEG |
| | LPKPLTLRWEPSSGGGGSGGGGSGGGGSCVPDQDTAIRVFAIPPSFASIFLTKSTKLTCLVTDLTTYD |
| | SVTISWTRQNGEAVKTHTNISESHPNATFSAVGEASICEDDWNSGERFTCTVTHTDLPSPLKQTISRP |
| | KGVALHRPDVYLLPPAREQLNLRESATITCLVTGFSPADVFVQWMQRGQPLSPEKYVTSAPMPEPQAP |
| | GRYFAHSILTVSEEEWNTGETYTCVVAHEALPNRVTERTVDKSTGKPTLYNVSLVMSDTAGTCY |
| | (NLV_B2M_HLAA2_IgM3-4_var2) |
| 249 | ELAGIGILTVGGGGSGGGGSGGGGSIQRTPKIQVYSRHPAENGKSNFLNCYVSGFHPSDIEVDLLKNG |
| | ERIEKVEHSDLSFSKDWSFYLLYYTEFTPTEKDEYACRVNHVTLSQPKIVKWDRDMGGGGSGGGGSGG |
| | GGSGGGGSGSHSMRYFFTSVSRPGRGEPRFIAVGYVDDTQFVRFDSDAASQRMEPRAPWIEQEGPEYW |
| | DGETRKVKAHSQTHRVDLGTLRGAYNQSEAGSHTVQRMYGCDVGSDWRFLRGYHQYAYDGKDYIALKE |
| | DLRSWTAADMAAQTTKHKWEAAHVAEQLRAYLEGTCVEWLRRYLENGKETLQRTDAPKTHMTHHAVSD |
| | HEATLRCWALSFYPAEITLTWQRDGEDQTQDTELVETRPAGDGTFQKWAAVVVPSGQEQRYTCHVQHE |
| | GLPKPLTLRWEPSSGGGGSGGGGSGGGGSCVPDQDTAIRVFAIPPSFASIFLTKSTKLTCLVTDLTTY |
| | DSVTISWTRQNGEAVKTHTNISESHPNATFSAVGEASICEDDWNSGERFTCTVTHTDLPSPLKQTISR |
| | PKGVALHRPDVYLLPPAREQLNLRESATITCLVTGFSPADVFVQWMQRGQPLSPEKYVTSAPMPEPQA |
| | PGRYFAHSILTVSEEEWNTGETYTCVVAHEALPNRVTERTVDKSTGKPTLYNVSLVMSDTAGTCY |
| | (ELA_B2M_HLAA2_IgM3-4_var2) |
| 250 | QVQLQQSGAELARPGASVKMSCKASGYTFTRYTMHWVKQRPGQGLEWIGYINPSRGYTNYNQKFKDKA |
| | TLTTDKSSSTAYMQLSSLTSEDSAVYYCARYYDDHYSLDYWGQGTTLTVSSGGGGSGGGGSGGGGSQI |
| | VLTQSPAIMSASPGEKVTMTCSASSSVSYMNWYQQKSGTSPKRWIYDTSKLASGVPAHFRGSGSGTSY |
| | SLTISGMEAEDAATYYCQQWSSNPFTFGSGTKLEIK |
| | (Anti-CD3 scFv) |
| 251 | QEDERIVLVDNKCKCARITSRIIRSSEDPNEDIVERNIRIIVPLNNRENISDPTSPLRTRFVYHLSDL |
| | CKKCDPTEVELDNQIVTATQSNICDEDSATETCYTYDRNKCYTAVVPLVYGGETKMVETALTPDACYP |
| | DGGGGSGGGGSDYKDDDDKHHHHHHHH |
| | (Jchain-(G4S)2-Flag-His) |
| 252 | QEDERIVLVDNKCKCARITSRIIRSSEDPNEDIVERNIRIIVPLNNRENISDPTSPLRTRFVYHLSDL |
| | CKKCDPTEVELDNQIVTATQSNICDEDSATETCYTYDRNKCYTAVVPLVYGGETKMVETALTPDACYP |
| | DGGGGSGGGGSGGGGSQVQLQQSGAELARPGASVKMSCKASGYTFTRYTMHWVKQRPGQGLEWIGYIN |
| | PSRGYTNYNQKFKDKATLTTDKSSSTAYMQLSSLTSEDSAVYYCARYYDDHYSLDYWGQGTTLTVSSG |
| | GGGSGGGGSGGGGSQIVLTQSPAIMSASPGEKVTMTCSASSSVSYMNWYQQKSGTSPKRWIYDTSKLA |
| | SGVPAHFRGSGSGTSYSLTISGMEAEDAATYYCQQWSSNPFTFGSGTKLEIK |
| | (Human Ig J chain with anti-CD3 at C-terminus) |
| 253 | QVQLQQSGAELARPGASVKMSCKASGYTFTRYTMHWVKQRPGQGLEWIGYINPSRGYTNYNQKFKDKA |
| | TLTTDKSSSTAYMQLSSLTSEDSAVYYCARYYDDHYSLDYWGQGTTLTVSSGGGGSGGGGSGGGGSQI |
| | VLTQSPAIMSASPGEKVTMTCSASSSVSYMNWYQQKSGTSPKRWIYDTSKLASGVPAHFRGSGSGTSY |
| | SLTISGMEAEDAATYYCQQWSSNPFTFGSGTKLEIKGGGGSGGGGSGGGGSQEDERIVLVDNKCKCAR |
| | ITSRIIRSSEDPNEDIVERNIRIIVPLNNRENISDPTSPLRTRFVYHLSDLCKKCDPTEVELDNQIVT |
| | ATQSNICDEDSATETCYTYDRNKCYTAVVPLVYGGETKMVETALTPDACYPD |
| | (Human Ig J chain with anti-CD3 at N-terminus) |
| 254 | CVPDQDTAIRVFAIPPSFASIFLTKSTKLTCLVTDLTTYDSVTISWTRQNGEAVKTHTNISESHPNAT |
| | FSAVGEASICEDDWNSGERFTCTVTHTDLPSPLKQTISRPKGVALHRPDVYLLPPAREQLNLRESATI |
| | TCLVTGFSPADVFVQWMQRGQPLSPEKYVTSAPMPEPQAPGRYFAHSILTVSEEEWNTGETYTCVVAH |
| | EALPNRVTERTVDKSTGKPTLYNVSLVMSDTAGTCY |
| | (IgM Cm3-Cm4_var2) |
| 255 | LDELQAEIEQLEERNYALRKEIEDLQKQLEKLGAP |
| | (optimized FosW) |
| 256 | IARLEEKVKTLKAQNYELASTANMLREQVAQLGAP |
| | (optimized cJun) |
| 257 | ALWEIQQVV |
| | (HLA-A*02:01 restricted SARS-COV-2 Orflab epitope) |
| 258 | KLWAQCVQL |
| | (HLA-A*02:01 restricted SARS-COV-2 Orflab epitope) |
| 259 | GGGGSGGGGSGGGGSIQRTPKIQVYSRHPAENGKSNFLNCYVSGFHPSDIEVDLLKNGERIEKVEHSD |
| | LSFSKDWSFYLLYYTEFTPTEKDEYACRVNHVTLSQPKIVKWDRDMGGGGSGGGGSGGGGSGGGGSGS |
| | HSMRYFFTSVSRPGRGEPRFIAVGYVDDTQFVRFDSDAASQRMEPRAPWIEQEGPEYWDGETRKVKAH |
| | SQTHRVDLGTLRGAYNQSEAGSHTVQRMYGCDVGSDWRFLRGYHQYAYDGKDYIALKEDLRSWTAADM |
| | AAQTTKHKWEAAHVAEQLRAYLEGTCVEWLRRYLENGKETLQRTDAPKTHMTHHAVSDHEATLRCWAL |
| | SFYPAEITLTWQRDGEDQTQDTELVETRPAGDGTFQKWAAVVVPSGQEQRYTCHVQHEGLPKPLTLRW |
| | EPSSGGGGSGGGGSGGGGSIAELPPKVSVFVPPRDGFFGNPRKSKLICQATGFSPRQIQVSWLREGKQ |
| | VGSGVTTDQVQAEAKESGPTTYKVTSTLTIKESDWLGQSMFTCRVDHRGLTFQQNASSMCVPDQDTAI |
| | RVFAIPPSFASIFLTKSTKLTCLVTDLTTYDSVTISWTRQNGEAVKTHTNISESHPNATFSAVGEASI |
| | CEDDWNSGERFTCTVTHTDLPSPLKQTISRPKGVALHRPDVYLLPPAREQLNLRESATITCLVTGFSP |
| | ADVFVQWMQRGQPLSPEKYVTSAPMPEPQAPGRYFAHSILTVSEEEWNTGETYTCVVAHEALPNRVTE |
| | RTVDKSTGKPTLYNVSLVMSDTAGTCY |
| | (GS Linker-b2m-GS linker-HLA A2-linker-IgM2-4) |
| 260 | GGGGSGGGGSGGGGSIQRTPKIQVYSRHPAENGKSNFLNCYVSGFHPSDIEVDLLKNGERIEKVEHSD |
| | LSFSKDWSFYLLYYTEFTPTEKDEYACRVNHVTLSQPKIVKWDRDMGGGGSGGGGSGGGGSGGGGSGS |
| | HSMRYFFTSVSRPGRGEPRFIAVGYVDDTQFVRFDSDAASQRMEPRAPWIEQEGPEYWDGETRKVKAH |
| | SQTHRVDLGTLRGAYNQSEAGSHTVQRMYGCDVGSDWRFLRGYHQYAYDGKDYIALKEDLRSWTAADM |
| | AAQTTKHKWEAAHVAEQLRAYLEGTCVEWLRRYLENGKETLQRTDAPKTHMTHHAVSDHEATLRCWAL |
| | SFYPAEITLTWQRDGEDQTQDTELVETRPAGDGTFQKWAAVVVPSGQEQRYTCHVQHEGLPKPLTLRW |
| | EPSSIAELPPKVSVFVPPRDGFFGNPRKSKLICQATGFSPRQIQVSWLREGKQVGSGVTTDQVQAEAK |
| | ESGPTTYKVTSTLTIKESDWLGQSMFTCRVDHRGLTFQQNASSMCVPDQDTAIRVFAIPPSFASIFLT |
| | KSTKLTCLVTDLTTYDSVTISWTRQNGEAVKTHTNISESHPNATFSAVGEASICEDDWNSGERFTCTV |
| | THTDLPSPLKQTISRPKGVALHRPDVYLLPPAREQLNLRESATITCLVTGFSPADVFVQWMQRGQPLS |
| | PEKYVTSAPMPEPQAPGRYFAHSILTVSEEEWNTGETYTCVVAHEALPNRVTERTVDKSTGKPTLYNV |
| | SLVMSDTAGTCY |
| | (GS Linker-b2m-GS linker-HLA A2-IgM2-4) |
| 261 | GSHSMRYFFTSVSRPGRGEPRFIAVGYVDDTQFVRFDSDAASRRMEPRAPWIEQEGPEYWDGETRKVK |
| | AHSQTHRVDLGTLRGYYNQSEAGSHTLQRMYGCDVGSDWRFLRGYHQYAYDGKDYIALKEDLRSWTAA |
| | DMAAQTTKHKWEAAHVAEQWRAYLEGTCVEWLRRYLENGKETLQRTDAPKTHMTHHAVSDHEATLRCW |
| | ALSFYPAEITLTWQRDGEDQTQDTELVETRPAGDGTFQKWAAVVVPSGQEQRYTCHVQHEGLPKPLTL |
| | RWEPSS |
| | HLA-A*02:02 |
| 262 | GSHSMRYFYTSVSRPGRGEPRFIAVGYVDDTQFVRFDSDAASQRMEPRAPWIEQEGPEYWDGETRKVK |
| | AHSQTHRVDLGTLRGYYNQSEAGSHTVQRMYGCDVGSDWRFLRGYHQYAYDGKDYIALKEDLRSWTAA |
| | DMAAQTTKHKWEAAHVAEQLRAYLEGTCVEWLRRYLENGKETLQRTDAPKTHMTHHAVSDHEATLRCW |
| | ALSFYPAEITLTWQRDGEDQTQDTELVETRPAGDGTFQKWAAVVVPSGQEQRYTCHVQHEGLPKPLTL |
| | RWEPSS |
| | HLA-A*02:06 |
| 263 | GSHSMRYFYTSVSRPGRGEPRFIAVGYVDDTQFVRFDSDAASQRMEPRAPWIEQEGPEYWDRNTRNVK |
| | AHSQTDRVDLGTLRGYYNQSEAGSHTIQMMYGCDVGSDGRFLRGYRQDAYDGKDYIALKEDLRSWTAA |
| | DMAAQTTKHKWEAAHVAEQWRAYLEGTCVEWLRRYLENGKETLQRTDAPKTHMTHHAVSDHEATLRCW |
| | ALSFYPAEITLTWQRDGEDQTQDTELVETRPAGDGTFQKWVAVVVPSGQEQRYTCHVQHEGLPKPLTL |
| | RWEPSS |
| | HLA-A*68:03 |
| 264 | GSHSMRYFYTAVSRPGRGEPRFIAVGYVDDTQFVQFDSDAASPRGEPRAPWVEQEGPEYWDRETQKYK |
| | RQAQTDRVSLRNLRGYYNQSEAGSHTLQRMYGCDLGPDGRLLRGYNQFAYDGKDYIALNEDLRSWTAA |
| | DTAAQITQRKWEAARTAEQLRAYLEGTCVEWLRRYLENGKKTLQRAEHPKTHVTHHPVSDHEATLRCW |
| | ALGFYPAEITLTWQRDGEDQTQDTELVETRPAGDGTFQKWAAVVVPSGEEQRYTCHVQHEGLPEPLTL |
| | RWGPSS |
| | HLA-C*08:01 |
| 265 | GDTRPRFLEQVKHECHFFNGTERVRFLDRYFYHQEEYVRFDSDVGEYRAVTELGRPDAEYWNSQKDLL |
| | EQRRAEVDTYCRHNYGVVESFTVQRRVYPEVTVYPAKTQPLQHHNLLVCSVNGFYPGSIEVRWFRNGQ |
| | EEKTGVVSTGLIQNGDWTFQTLVMLETVPRSGEVYTCQVEHPSLTSPLTVEWRARSESAQSK |
| | HLA-DRB1*04:03 |
| 266 | GDTRPRFLEQVKHECHFFNGTERVRFLDRYFYHQEEYVRFDSDVGEYRAVTELGRPDAEYWNSQKDLL |
| | EQRRAEVDTYCRHNYGVGESFTVQRRVYPEVTVYPAKTQPLQHHNLLVCSVNGFYPGSIEVRWFRNGQ |
| | EEKTGVVSTGLIQNGDWTFQTLVMLETVPRSGEVYTCQVEHPSLTSPLTVEWRARSESAQSK |
| | HLA-DRB1*04:07 |
| 267 | GDTRPRFLEYSTGECYFFNGTERVRFLDRYFYNQEEYVRFDSDVGEYRAVTELGRPDAEYWNSQKDFL |
| | EDRRALVDTYCRHNYGVGESFTVQRRVHPKVTVYPSKTQPLQHHNLLVCSVSGFYPGSIEVRWFRNGQ |
| | EEKTGVVSTGLIHNGDWTFQTLVMLETVPRSGEVYTCQVEHPSVTSPLTVEWSARSESAQSK |
| | HLA-DRB1*08:02 |
| 268 | GDTQPRFLKQDKFECHFFNGTERVRYLHRGIYNQEENVRFDSDVGEYRAVTELGRPVAESWNSQKDFL |
| | ERRRAEVDTVCRHNYGVGESFTVQRRVHPEVTVYPAKTQPLQHHNLLVCSVSGFYPGSIEVRWFRNGQ |
| | EEKAGVVSTGLIQNGDWTFQTLVMLETVPRSGEVYTCQVEHPSVMSPLTVEWRARSESAQSK |
| | HLA-DRB1*09:01 |
| 269 | GDTRPRFLEYSTGECYFFNGTERVRLLERHFHNQEELLRFDSDVGEFRAVTELGRPVAESWNSQKDIL |
| | EDRRAAVDTYCRHNYGAVESFTVQRRVHPKVTVYPSKTQPLQHHNLLVCSVSGFYPGSIEVRWFRNGQ |
| | EEKTGVVSTGLIHNGDWTFQTLVMLETVPRSGEVYTCQVEHPSVTSPLTVEWRARSESAQSK |
| | HLA-DRB1*12:01 |
| 270 | GDTRPRFLEYSTSECHFFNGTERVRFLDRYFYNQEEYVRFDSDVGEYRAVTELGRPSAEYWNSQKDIL |
| | EDKRAAVDTYCRHNYGVGESFTVQRRVHPKVTVYPSKTQPLQHHNLLVCSVSGFYPGSIEVRWFRNGQ |
| | EEKTGVVSTGLIHNGDWTFQTLVMLETVPRSGEVYTCQVEHPSVTSPLTVEWRARSESAQSK |
| | HLA-DRB1*13:03 |
| 271 | GDTRPRFLWQPKRECHFFNGTERVRFLDRYFYNQEESVRFDSDVGEFRAVTELGRPDAEYWNSQKDIL |
| | EQARAAVDTYCRHNYGVGESFTVQRRVQPKVTVYPSKTQPLQHHNLLVCSVSGFYPGSIEVRWFLNGQ |
| | EEKAGMVSTGLIQNGDWTFQTLVMLETVPRSGEVYTCQVEHPSVTSPLTVEWRARSESAQSK |
| | HLA-DRB1*15:02 |
| 272 | GDTRPRFLQQDKYECHFFNGTERVRFLHRDIYNQEEDLRFDSDVGEYRAVTELGRPDAEYWNSQKDFL |
| | EDRRAAVDTYCRHNYGVGESFTVQRRVEPKVTVYPARTQTLQHHNLLVCSVNGFYPGSIEVRWFRNSQ |
| | EEKAGVVSTGLIQNGDWTFQTLVMLETVPRSGEVYTCQVEHPSVTSPLTVEWRAQSESAQSK |
| | HLA-DRB5*01:01 |
| 273 | IKADHVSTYAAFVQTHRPTGEFMFEFDEDEMFYVDLDKKETVWHLEEFGQAFSFEAQGGLANIAILNN |
| | NLNTLIQRSNHTQATNDPPEVTVFPKEPVELGQPNTLICHIDKFFPPVLNVTWLCNGELVTEGVAESL |
| | FLPRTDYSFHKFHYLTFVPSAEDFYDCRVEHWGLDQPLLKHWEAQEPIQMPETTE |
| | HLA-DPA1*01:03 |
| 274 | GRATPENYLFQGRQECYAFNGTQRFLERYIYNREEFARFDSDVGEFRAVTELGRPAAEYWNSQKDILE |
| | EKRAVPDRMCRHNYELGGPMTLQRRVQPRVNVSPSKKGPLQHHNLLVCHVTDFYPGSIQVRWFLNGQE |
| | ETAGVVSTNLIRNGDWTFQILVMLEMTPQQGDVYTCQVEHTSLDSPVTVEEWKAQSDSARSK |
| | HLA-DPB1*04:01 |
| 275 | QEDERIVLVDNKCKCARITSRIIRSSEDPNEDIVERNIRIIVPLNNRENISDPTSPLRTRFVYHLSDL |
| | CKKCDPTEVELDNQIVTATQSNICDEDSATETCYTYDRNKCYTAVVPLVYGGETKMVETALTPDACYP |
| | DGGGGSGGGGSGGGGSQVQLQQSGAELARPGASVKMSCKASGYTFTRYTMHWVKQRPGQGLEWIGYIN |
| | PSRGYTNYNQKFKDKATLTTDKSSSTAYMQLSSLTSEDSAVYYCARYYDDHYSLDYWGQGTTLTVSSG |
| | GGGSGGGGSGGGGSQIVLTQSPAIMSASPGEKVTMTCSASSSVSYMNWYQQKSGTSPKRWIYDTSKLA |
| | SGVPAHFRGSGSGTSYSLTISGMEAEDAATYYCQQWSSNPFTFGSGTKLEIKGGGGSDYKDDDDKHHH |
| | HHH |
| | (J-chain-Linker-anti-CD3scFv-Flag-His) |

1. A multimer comprising a Major Histocompatibility Complex (MHC) moiety operatively linked to a multimerization moiety, wherein:
(a) the MHC moiety comprises an MHC molecule optionally bound with a cognate peptide; and
(b) the multimerization moiety comprises (i) an IgM Cμ4 constant region and a tailpiece region; or (ii) an IgA Cα3 constant region and a tailpiece region, wherein the tailpiece region in (b)(i) or (b)(ii) is an IgM tailpiece region or an IgA tailpiece region.

2. The multimer of claim 1, wherein the MHC moiety comprises (i) an MHC class I molecule, optionally bound with an MHC Class I binding peptide; or (ii) an MHC class II molecule, optionally bound with an MHC Class II binding peptide.

3. The multimer of claim 1 or 2, wherein the multimerization moiety comprises an IgM Cμ4 constant region and an IgM tailpiece region and further comprises an IgM Cμ3 constant region.

4. The multimer of claim 1 or claim 2, wherein the multimerization moiety comprises an IgM Cμ4 constant region and an IgM tailpiece region and further comprises an IgM Cμ2 constant region.

5. The multimer of claim 1 or claim 2, wherein the multimerization moiety comprises an IgM Cμ4 constant region and an IgM tailpiece region and further comprises an IgM Cμ1 constant region.

6. The multimer of claim 1 or claim 2, wherein the multimerization moiety comprises an IgM Cμ4 constant region and an IgM tailpiece region and further comprises IgM Cμ2 and Cμ3 constant regions.

7. The multimer of claim 1 or claim 2, wherein the multimerization moiety comprises an IgM Cμ4 constant region and an IgM tailpiece region and further comprises IgM Cμ1 and Cμ3 constant regions.

8. The multimer of claim 1 or claim 2, wherein the multimerization moiety comprises an IgM Cμ4 constant region and an IgM tailpiece region and further comprises IgM Cμ1 and Cμ2 constant regions.

9. The multimer of claim 1 or claim 2, wherein the multimerization moiety comprises an IgM Cμ4 constant region and an IgM tailpiece region and further comprises IgM Cμ1, Cμ2 and Cμ3 constant regions.

10. The multimer of claim 1 or claim 2, wherein the multimerization moiety comprises an IgA Cα3 constant region and an IgA tailpiece region and further comprises an IgA Cα2 constant region.

11. The multimer of claim 1 or claim 2, wherein the multimerization moiety comprises an IgA Cα3 constant region and an IgA tailpiece region and further comprises an IgA Cal constant region.

12. The multimer of claim 1 or claim 2, wherein the multimerization moiety comprises an IgA Cα3 constant region and an IgA tailpiece region and further comprises IgA Cal and Cα2 constant regions.

13. A multimer comprising a Major Histocompatibility Complex (MHC) moiety operatively linked to a multimerization moiety, wherein:
(a) the MHC moiety comprises (i) an MHC Class I molecule, optionally bound with an MHC Class I-binding peptide; or (ii) an MHC Class II molecule, optionally bound with an MHC Class II-binding peptide; and
(b) the multimerization moiety comprises an IgM tailpiece region or an IgA tailpiece region.

14. The multimer of claim 12, wherein the multimerization moiety comprises an IgM tailpiece region.

15. The multimer of claim 12, wherein the multimerization moiety comprises an IgA tailpiece region.

16. The multimer of any one of claims 1-15, wherein the MHC moiety comprises an MHC Class I molecule bound with an MHC Class I-binding peptide.

17. The multimer of any one of claims 1-16, wherein the MHC moiety is operatively linked to the multimerization moiety through an MHC Class I α chain.

18. The multimer of claim 16 or 17, wherein the MHC moiety is in a single polypeptide comprising, from the N-terminus to the C-terminus, the MHC Class I-binding peptide, a first linker, a β2M chain, a second linker, and an MHC Class I α chain, wherein the MHC Class I α chain optionally comprises a Y84A substitution.

19. The multimer of claim 18, wherein the MHC Class I α chain comprises a Y84C substitution, and the first linker comprises a cysteine residue, such that a disulfide bond forms between the cysteine residue corresponding to position 84 of the MHC Class I α chain and the cysteine residue of the first linker.

20. The multimer of any one of claims 1-15, wherein the MHC moiety comprises an MHC Class II molecule bound with an MHC Class II-binding peptide.

21. The multimer of any one of claims 1-15 or 20, wherein the MHC moiety is operatively linked to the multimerization moiety through an MHC Class II β chain or an MHC Class II α chain.

22. The multimer of any one of claims 1-15 or 20-21, further comprising a first heterodimerization motif linked to an α chain of the MHC Class II molecule and a second heterodimerization motif linked to a β chain of the MHC Class II molecule, wherein the first and second heterodimerization motifs form a heterodimer.

23. The multimer of claim 22, wherein the first and/or second heterodimerization motifs are linked to the multimerization moiety.

24. The multimer of 22 or 23, wherein the first and/or second heterodimerization motifs comprise a leucine zipper.

25. The multimer of claim 23, wherein the leucine zipper comprises a variant of Fos and Jun.

26. The multimer of any one of claims 1-25, which further comprises an immunoglobulin J chain.

27. The multimer of any one of claims 1-26, wherein the multimer is soluble.

28. The multimer of any one of claims 1-27, wherein the MHC moiety is a single chain molecule.

29. The multimer of any one of claims 1-28, which is a dimer.

30. The multimer of any one of claims 1-28, which is a trimer.

31. The multimer of any one of claims 1-28, which is a tetramer.

32. The multimer of any one of claims 1-28, which is a pentamer.

33. The multimer of any one of claims 1-28, which is a hexamer.

34. The multimer of any one of claims 1-33, wherein the MHC moiety and the multimerization moiety are separated by a linker.

35. The multimer of claim 34, wherein the linker is a flexible linker, a rigid linker or a semi-rigid linker.

36. The multimer of any one of claims 26-35, which further comprises an effector moiety operatively linked to the immunoglobulin J chain.

37. The multimer of any one of claims 1-36, which further comprises an effector moiety, optionally wherein the effector moiety is operatively linked to the multimerization moiety.

38. The multimer of claim 36 or 37, wherein the effector moiety comprises a checkpoint protein agonist.

39. The multimer of claim 38, wherein the checkpoint protein agonist comprises a PD-L1 extracellular domain or domain 1, an anti-PD1 agonist antibody, an anti-CTLA4 agonist antibody, an anti-Lag3 agonist antibody, an anti-TIM3 agonist antibody, an anti-TIGIT agonist antibody, an anti-LILRB1 agonist antibody, an anti-LILRB2 agonist antibody, or any portion or antigen-binding fragment thereof.

40. The multimer of claim 36 or 37, wherein the effector moiety comprises a TNFR family protein agonist.

41. The multimer of claim 40, wherein the TNFR family protein agonist comprises 4-1BBL (CD137L), an anti-CD137 agonist antibody or any portion or antigen-binding fragment thereof, or an anti-TNFR2 agonist antibody or any portion or antigen-binding fragment thereof.

42. The multimer of claim 36 or 37, wherein the effector moiety comprises an anti-inflammatory agent, which, optionally, may comprise an anti-inflammatory cytokine.

43. The multimer of claim 42, wherein the anti-inflammatory agent comprises an extracellular region of HLA-G, an anti-LILRB1 agonist antibody, an anti-LILRB2 agonist antibody, or any fragments or variants thereof or an anti-inflammatory cytokine selected from IL10, TGFβ, or any fragments or variants thereof.

44. The multimer of claim 36 or 37, wherein the effector moiety comprises an immune cell engaging agent.

45. The multimer of claim 44, wherein the immune cell engaging agent comprises an anti-CD3 antibody, an anti-NKp46 antibody, an anti-CD16a antibody, an anti-CD56 antibody, an anti-NKG2D antibody, an anti-NKp30 antibody, an anti-CD64 antibody, or any portion or antigen-binding fragments thereof.

46. The multimer of claim 36 or 37, wherein the effector moiety comprises a cytokine.

47. The multimer of claim 46, wherein the cytokine comprises IL-12. IL-15, or any variants or fragments thereof.

48. The multimer of claim 46, wherein the cytokine comprises IL2, TGFβ, or any variants or fragments thereof.

49. The multimer of claim 48, wherein the IL2 comprises an IL2 mutein that has reduced or no binding affinity for CD25/IL2Rα.

50. The multimer of claim 36 or 37, wherein the effector moiety comprises a cell-death-inducing agent.

51. The multimer of claim 50, wherein the cell-death-inducing agent comprises a tubulin inhibitor, a DNA damaging agent, FasL, anti-Fas agonist antibody or any portion or antigen-binding fragment thereof.

52. The multimer of claim 38 or 39, wherein the effector moiety comprises a PD-1 binding moiety.

53. The multimer of claim 52, wherein the PD-1 binding moiety comprises PD-L1.

54. The multimer of any one of claims 36-53, wherein the effector moiety is attached to the multimer through a linker.

55. The multimer of claim 54, wherein the linker is a flexible linker, a rigid linker or a semi-rigid linker.

56. The multimer of any one of claims 1-55, which further comprises one or more heterologous functional domains.

57. A method of treating an autoimmune disease comprising administering to a subject in need thereof the multimer of any of claims 38-45 and 48-53.

58. A method of treating a cancer comprising administering to a subject in need thereof the multimer of any one of claims 40-41 and 46-47.

59. A composition comprising a mixture, which mixture comprises (i) at least two multimers of any one of claims 1-56; and (ii) wherein the at least two multimers are selected from the group consisting of dimers, trimers, tetramers, pentamers, and hexamers.

60. An isolated nucleic acid or plurality of nucleic acid molecules encoding the multimer or multimers of any one of claims 1-56.

61. An expression vector comprising the one or more isolated nucleic acid molecules of claim 60.

62. An isolated nucleic acid molecule, encoding a Major Histocompatibility Complex (MHC) moiety operatively linked to a multimerization moiety, wherein:
(a) the MHC moiety comprises an MHC molecule and optionally a cognate peptide; and
(b) the multimerization moiety comprises (i) an IgM Cμ4 constant region and a tailpiece region; or (ii) an IgA Cα3 constant region and a tailpiece region, wherein the tailpiece region in (b)(i) or (b)(ii) is an IgM tailpiece region or an IgA tailpiece region.

63. The nucleic acid of claim 62, wherein the MHC moiety comprises (i) an MHC class I molecule and optionally an MHC Class I binding peptide; or (ii) an MHC class II molecule, and an MHC Class II binding peptide.

64. An isolated nucleic acid molecule, encoding a Major Histocompatibility Complex (MHC) moiety and, optionally, an MHC binding peptide, and which MHC moiety is operatively linked to a multimerization moiety, wherein:
(a) the MHC moiety comprises (i) an MHC Class I molecule, and, optionally an MHC Class II binding peptide; or (ii) an MHC Class II molecule and, optionally, an MHC Class II binding peptide; and
(b) the multimerization moiety comprises (i) an IgM Cμ4 constant region and a tailpiece region; or (ii) an IgA Cα3 constant region and a tailpiece region, wherein the tailpiece region in (b)(i) or (b)(ii) is an IgM tailpiece region or an IgA tailpiece region.

65. The isolated nucleic acid molecule of any one of claims 61-64, wherein the multimerization moiety comprises an IgM Cμ4 constant region and an IgM tailpiece region and further comprises an IgM Cμ3 constant region.

66. The isolated nucleic acid molecule of any one of claims 61-64, wherein the multimerization moiety comprises an IgM Cμ4 constant region and an IgM tailpiece region and further comprises an IgM Cμ2 constant region.

67. The isolated nucleic acid molecule of any one of claims 61-64, wherein the multimerization moiety comprises an IgM Cμ4 constant region and an IgM tailpiece region and further comprises an IgM Cμ1 constant region.

68. The isolated nucleic acid molecule of any one of claims 61-64, wherein the multimerization moiety comprises an IgM Cμ4 constant region and an IgM tailpiece region and further comprises IgM Cμ2 and Cμ3 constant regions.

69. The isolated nucleic acid molecule of any one of claims 61-64, wherein the multimerization moiety comprises an IgM Cμ4 constant region and an IgM tailpiece region and further comprises IgM Cμ1 and Cμ3 constant regions.

70. The isolated nucleic acid molecule of any one of claims 61-64, wherein the multimerization moiety comprises an IgM Cμ4 constant region and an IgM tailpiece region and further comprises IgM Cμ1 and Cμ2 constant regions.

71. The isolated nucleic acid molecule of any one of claims 61-64, wherein the multimerization moiety comprises an IgM Cμ4 constant region and an IgM tailpiece region and further comprises IgM Cμ1, Cμ2 and Cμ3 constant regions.

72. The isolated nucleic acid molecule of any one of claims 61-64, wherein the multimerization moiety comprises an IgA Cα3 constant region and an IgA tailpiece region and further comprises an IgA Cα2 constant region.

73. The isolated nucleic acid molecule of any one of claims 61-64, wherein the multimerization moiety comprises an IgA Cα3 constant region and an IgA tailpiece region and further comprises an IgA Cal constant region.

74. The isolated nucleic acid molecule of any one of claims 61-64, wherein the multimerization moiety comprises an IgA Cα3 constant region and an IgA tailpiece region and further comprises IgA Cal and Cα2 constant regions.

75. An isolated nucleic acid molecule encoding a Major Histocompatibility Complex (MHC) moiety and, optionally, an MHC binding peptide, wherein:
(a) the MHC moiety comprises (i) an MHC Class I molecule and, optionally, an MHC Class I binding peptide; or (ii) an MHC Class II molecule and, optionally, an MHC Class II binding peptide; and
(b) the multimerization moiety comprises an IgM tailpiece region or an IgA tailpiece region.

76. The isolated nucleic acid molecule of claim 75, wherein the multimerization moiety comprises an IgM tailpiece region.

77. The isolated nucleic acid molecule of claim 75, wherein the multimerization moiety comprises an IgA tailpiece region.

78. The isolated nucleic acid molecule of any one of claims 62-77, wherein the MHC moiety is operatively linked to the multimerization moiety through an MHC Class I α chain.

79. The isolated nucleic acid molecule of any one of claims 62-77, wherein the MHC moiety is operatively linked to the multimerization moiety through an MHC Class II β chain or an MHC Class II α chain.

80. The isolated nucleic acid molecule of any one of claims 62-79, wherein the MHC moiety and the multimerization moiety are separated by a linker.

81. The isolated nucleic acid molecule of claim 80, wherein the linker is a flexible linker, a rigid linker or a semi-rigid linker.

82. An expression vector comprising the one or more nucleic acid molecules of any one of claims 62-81.

83. A host cell comprising the one or more nucleic acid molecules of any one of claim 60 or 62-81 or the expression vector of claim 61 or 82.

84. The host cell of claim 83, which further comprises a nucleic acid molecule or an expression vector encoding a cognate MHC chain that pairs with the MHC moiety.

85. The host cell of claim 83 or 84, which comprises an expression vector encoding an immunoglobulin J chain and a separate expression vector encoding the multimer.

86. The host cell of any of claims 83-85, wherein, in the expression vector, the immunoglobulin J chain is operatively linked to an effector moiety.

87. The host cell of claim 86, wherein the effector moiety comprises a PD-1 binding moiety.

88. The host cell of claim 87, wherein the PD-1 binding moiety comprises PD-L1.

89. A method of preparing a Major Histocompatibility Complex (MHC) multimer comprising culturing the host cell of any one of claims 83-88.

90. The method of claim 89, further comprising isolating the MHC multimer from the host cells or the host cell culture supernatant.

91. A pharmaceutical composition comprising the multimer of any one of claims 1-38 and a pharmaceutically acceptable carrier.

92. A kit comprising the multimer of any one of claims 1-56 packaged in a container with instructions for use in detecting peptide-MHC-TCR interactions and/or modulating an immune response.

93. A method of detecting binding of a T cell receptor (TCR) to a peptide-MHC complex, the method comprising contacting a TCR with the multimer of any one of claims 1-56 and detecting binding of the TCR to the multimer to thereby detect binding of the TCR to a peptide-MHC complex.

94. The method of claim 93, wherein the multimer comprises a detectable moiety.

95. The method of claim 93, wherein the TCR is on the surface of a T cell and the multimer is contacted with the T cell.

96. A method of modulating an immune response in a subject, the method comprising administering to the subject an effective amount of the multimer of any one of claims 1-38, or the pharmaceutical composition of claim 91, such that an immune response is modulated in the subject.

97. The method of claim 96, wherein the subject has an autoimmune disorder and the immune response to the autoimmune disorder is modulated.

98. The method of claim 96, wherein the subject has an inflammatory disorder and the immune response to the inflammatory disorder is modulated.

99. A method of treating an autoimmune disorder in a subject, the method comprising administering to the subject the multimer of any one of claims 1-56, or the pharmaceutical composition of claim 91, wherein the MHC moiety of the multimer comprises an autoantigen bound to an MHC Class I or Class II molecule.

100. The method of claim 99, wherein the autoimmune disorder is Type I diabetes.

101. The method of claim 99, wherein the autoantigen is an insulin autoantigen.

102. The method of any one of claims 96-101, wherein the subject is further treated with one or more additional therapies.

103. A method of treating an infectious disease in a subject, the method comprising administering to the subject the multimer of any one of claims 1-56, or the pharmaceutical composition of claim 91.

104. The method of claim 103, wherein the infectious disease is a viral infection.

105. Use of the MHC multimer of any one of claims 1-56 in the manufacture of a medicament for treating an autoimmune disorder, inflammatory disorder, infectious disease, or cancer.

106. A method of treating cancer using an immunotherapy comprising administering to a subject diagnosed with cancer the multimer of any one of claims 1-56 or the pharmaceutical composition of claim 91.

107. The method of claim 106, wherein the subject is further treated with one or more additional therapies.
